SEQUENCE LISTING (38 sequences)

SEQ ID NO: 1 - DNA, 5749 bp, Homo sapiens
cgcgggagccaacttcaggctgctcagaggaagcccgtgcagtcagtcacctgggtgcaagagcgttgctgcctcgggctctcccgctgcagggagagcggcactcgctggcctggatgtggttggatttaggggggctccgcagcaggggtttcgtggcggtggcaagcgctgcaacaggtagacggcgagagacggaccccggccgaggcagggatggagaccaaaggctaccacagtctccctgaaggtctagatatggaaagacggtggggtcaagtttctcaggctgtggagcgttcttccctgggacctacagagaggaccgatgagaataactacatggagattgtcaacgtaagctgtgtttccggtgctattccaaacaacagtactcaaggaagcagcaaagaaaaacaagaactactcccttgccttcagcaagacaataatcggcctgggattttaacatctgatattaaaactgagctggaatctaaggaactttcagcaactgtagctgagtccatgggtttatatatggattctgtaagagatgctgactattcctatgagcagcagaaccaacaaggaagcatgagtccagctaagatttatcagaatgttgaacagctggtgaaattttacaaaggaaatggccatcgtccttccactctaagttgtgtgaacacgcccttgagatcatttatgtctgactctgggagctccgtgaatggtggcgtcatgcgcgccattgttaaaagccctatcatgtgtcatgagaaaagcccgtctgtttgcagccctctgaacatgacatcttcggtttgcagccctgctggaatcaactctgtgtcctccaccacagccagctttggcagttttccagtgcacagcccaatcacccagggaactcctctgacatgctcccctaatgctgaaaatcgaggctccaggtcgcacagccctgcacatgctagcaatgtgggctctcctctctcaagtccgttaagtagcatgaaatcctcaatttccagccctccaagtcactgcagtgtaaaatctccagtctccagtcccaataatgtcactctgagatcctctgtgtctagccctgcaaatattaacaactcaaggtgctctgtttccagcccttcgaacactaataacagatccacgctttccagtccggcagccagtactgtgggatctatctgtagccctgtaaacaatgccttcagctacactgcttctggcacctctgctggatccagtacattgcgggatgtggttcccagtccagacacgcaggagaaaggtgctcaagaggtcccttttcctaagactgaggaagtagagagtgccatctcaaatggtgtgactggccagcttaatattgtccagtacataaaaccagaaccagatggagcttttagcagctcatgtctaggaggaaatagcaaaataaattcggattcttcattctcagtaccaataaagcaagaatcaaccaagcattcatgttcaggcacctcttttaaagggaatccaacagtaaacccgtttccatttatggatggctcgtatttttcctttatggatgataaagactattattccctatcaggaattttaggaccacctgtgcccggctttgatggtaactgtgaaggcagcggattcccagtgggtattaaacaagaaccagatgacgggagctattacccagaggccagcatcccttcctctgctattgttggggtgaattcaggtggacagtccttccactacaggattggtgctcaaggtacaatatctttatcacgatcggctagagaccaatctttccaacacctgagttcctttcctcctgtcaatactttagtggagtcatggaaatcacacggcgacctgtcgtctagaagaagtgatgggtatccggtcttagaatacattccagaaaatgtatcaagctctactttacgaagtgtttctactggatcttcaagaccttcaaaaatatgtttggtgtgtggggatgaggcttcaggatgccattatggggtagtcacctgtggcagctgcaaagttttcttcaaaagagcagtggaagggcaacacaactatttatgtgctggaagaaatgattgcatcattgataagattcgacgaaagaattgtcctgcttgcagacttcagaaatgtcttcaagctggaatgaatttaggagcacgaaagtcaaagaagttgggaaagttaaaagggattcacgaggagcagccacagcagcagcagcccccacccccacccccacccccgcaaagcccagaggaagggacaacgtacatcgctcctgcaaaagaaccctcggtcaacacagcactggttcctcagctctccacaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgtgtctatcatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaagtgactgcccgctgcccagaagaactttgccttaagtttccctgtgttgttccacacccagaaggacccaagaaaacctgtttttaacatgtgatggttgattcacacttgttcaacagtttctcaagtttaaagtcatgtcagaggtttggagccgggaaagctgtttttccgtggatttggcgagaccagagcagtctgaaggattccccacctccaatcccccagcgcttagaaacatgttcctgttcctcgggatgaaaagccatatctagtcaataactctgattttgatattttcacagatggaagaagttttaactatgccgtgtagtttctggtatcgttcgcttgttttaaaagggttcaaggactaacgaacgttttaaagcttacccttggtttgcacataaaacgtatagtcaatatggggcattaatattcttttgttattaaaaaaacacaaaaaaataataaaaaaatatatacagattcctgttgtgtaataacagaactcgtggcgtggggcagcagctgcctctgagccctcgctcgtccacggtcttctgcatcactggtatacacactcgttagcgtccatttcttatttaattagaatggataagatgatgttaaatgccttggtttgatttctagtatctattgtgttggctttacaaataattttttgcagtcttttgctgtgctgtacattactgtatgtataaattatgaaggacctgaaataaggtataaggatcttttgtaaatgagacacatacaaaaaaaatctttaatggttaataggatgaatgggaaagtatttttgaaagaattctattttgctggagactatttaagtactatctttgtctaaacaaggtaatttttttttgtaaagtgcaatgtcctgcatgcataatgaaccgtttacagtgtatttaagaaagggaaagctgtgccttttttagcttcatatctaatttaccattattttacagtctctgttgtaaataaccacactgaaacctcttcggttgtcttgaaacctttctactttttctgtactttttgttttgttcttggtctcccgcttggggcatttgtgggactccagcacgttttctggcttctgcttcatcctgctccatcggggaatgacacactgcggtgtctgcagctcctggaaggtgtcatttgacaacacatgtgggagaggaggtccttggagtgctgcagctttgggaaagcctgcctcgtttcccttttcctctagaagcagaaccagctctacgagagtgagactgggaacttgatggctcagagagcatcttttcctcccattttagaaaatcagattttctcctgtgggaaaaaaaaattccatgcactctctctctgttaaagatcagctattcccttctgatcttggaaagaggttctgcactcctggaaccggtcacaggaacgcacagatcatggcaggatgcgctgggacggcccatcttggcaaggttcagtctgaatggcatggagaccgggagatagaggggttttagatttttaaaaggtaggttttaaaaataagttttatacataaacagttttggagaaaaattacagatcatataagcaagacagtggcactaaaatgtttaattcattaatctgtttgtttggcactgatgcaatgtatggcttttctcttgccccaaatcacaaacatatgtatctttggggaaactaacaatatgattgcactaaataaactactttgaatagaggccaaattaatcttttaaaaatgatgataatcatcaggtttactcagtgaaatcatattaattattttccaaaatctaaaagctgtagctggagaagcccatggccacgaggaagcagcaattaattagatcaacacttttctccagggttcaccatgcaggcaacattaccttgtctttcaaaagacacctgccttagtgcaaggggaaacctgtgaaagctgcactcagagggaggagtctttcttacataatttgcaatttcaggaatttaatttataggcagatctttaaatacagtcaacttacggtgcacagtaatatgaaagccacactttgaaggtaataaatacacagcatgcagactgggagttgctagcaaacaaatggcttacttacaaaagcagcttttagttcagacttagtttttataaaatgagaattctgacttacttaaccaggtttgggatggagatggtctgcatcagctttttgtattaacaaagttactggctctttgtgtgtctccaggtaactttgcttgattaaacagcaaagccatattctaaattcactgttgaatgcctgtcccagtccaaattgtctgtctgctcttatttttgtaccatattgctcttaaaaatcttggtttggtacagttcataattcaccaaaaagttcatataatttaaagaaacactaaattagtttaaaatgaagcaatttatatctttatgcaaaaacatatgtctgtctttgcaaaggactgtaagcagattacaataaatcctttacttt

SEQ ID NO: 2 - protein, 984 aa, Homo sapiens
METKGYHSLPEGLDMERRWGQVSQAVERSSLGPTERTDENNYMEIVNVSCVSGAIPNNSTQGSSKEKQELLPCLQQDNNRPGILTSDIKTELESKELSATVAESMGLYMDSVRDADYSYEQQNQQGSMSPAKIYQNVEQLVKFYKGNGHRPSTLSCVNTPLRSFMSDSGSSVNGGVMRAIVKSPIMCHEKSPSVCSPLNMTSSVCSPAGINSVSSTTASFGSFPVHSPITQGTPLTCSPNAENRGSRSHSPAHASNVGSPLSSPLSSMKSSISSPPSHCSVKSPVSSPNNVTLRSSVSSPANINNSRCSVSSPSNTNNRSTLSSPAASTVGSICSPVNNAFSYTASGTSAGSSTLRDVVPSPDTQEKGAQEVPFPKTEEVESAISNGVTGQLNIVQYIKPEPDGAFSSSCLGGNSKINSDSSFSVPIKQESTKHSCSGTSFKGNPTVNPFPFMDGSYFSFMDDKDYYSLSGILGPPVPGFDGNCEGSGFPVGIKQEPDDGSYYPEASIPSSAIVGVNSGGQSFHYRIGAQGTISLSRSARDQSFQHLSSFPPVNTLVESWKSHGDLSSRRSDGYPVLEYIPENVSSSTLRSVSTGSSRPSKICLVCGDEASGCHYGVVTCGSCKVFFKRAVEGQHNYLCAGRNDCIIDKIRRKNCPACRLQKCLQAGMNLGARKSKKLGKLKGIHEEQPQQQQPPPPPPPPQSPEEGTTYIAPAKEPSVNTALVPQLSTISRALTPSPVMVLENIEPEIVYAGYDSSKPDTAENLLSTLNRLAGKQMIQVVKWAKVLPGFKNLPLEDQITLIQYSWMCLSSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 3 - DNA, 5749 bp, Homo sapiens
cgcgggagccaacttcaggctgctcagaggaagcccgtgcagtcagtcacctgggtgcaagagcgttgctgcctcgggctctcccgctgcagggagagcggcactcgctggcctggatgtggttggatttaggggggctccgcagcaggggtttcgtggcggtggcaagcgctgcaacaggtagacggcgagagacggaccccggccgaggcagggatggagaccaaaggctaccacagtctccctgaaggtctagatatggaaagacggtggggtcaagtttctcaggctgtggagcgttcttccctgggacctacagagaggaccgatgagaataactacatggagattgtcaacgtaagctgtgtttccggtgctattccaaacaacagtactcaaggaagcagcaaagaaaaacaagaactactcccttgccttcagcaagacaataatcggcctgggattttaacatctgatattaaaactgagctggaatctaaggaactttcagcaactgtagctgagtccatgggtttatatatggattctgtaagagatgctgactattcctatgagcagcagaaccaacaaggaagcatgagtccagctaagatttatcagaatgttgaacagctggtgaaattttacaaaggaaatggccatcgtccttccactctaagttgtgtgaacacgcccttgagatcatttatgtctgactctgggagctccgtgaatggtggcgtcatgcgcgccgttgttaaaagccctatcatgtgtcatgagaaaagcccgtctgtttgcagccctctgaacatgacatcttcggtttgcagccctgctggaatcaactctgtgtcctccaccacagccagctttggcagttttccagtgcacagcccaatcacccagggaactcctctgacatgctcccctaatgctgaaaatcgaggctccaggtcgcacagccctgcacatgctagcaatgtgggctctcctctctcaagtccgttaagtagcatgaaatcctcaatttccagccctccaagtcactgcagtgtaaaatctccagtctccagtcccaataatgtcactctgagatcctctgtgtctagccctgcaaatattaacaactcaaggtgctctgtttccagcccttcgaacactaataacagatccacgctttccagtccggcagccagtactgtgggatctatctgtagccctgtaaacaatgccttcagctacactgcttctggcacctctgctggatccagtacattgcgggatgtggttcccagtccagacacgcaggagaaaggtgctcaagaggtcccttttcctaagactgaggaagtagagagtgccatctcaaatggtgtgactggccagcttaatattgtccagtacataaaaccagaaccagatggagcttttagcagctcatgtctaggaggaaatagcaaaataaattcggattcttcattctcagtaccaataaagcaagaatcaaccaagcattcatgttcaggcacctcttttaaagggaatccaacagtaaacccgtttccatttatggatggctcgtatttttcctttatggatgataaagactattattccctatcaggaattttaggaccacctgtgcccggctttgatggtaactgtgaaggcagcggattcccagtgggtattaaacaagaaccagatgacgggagctattacccagaggccagcatcccttcctctgctattgttggggtgaattcaggtggacagtccttccactacaggattggtgctcaaggtacaatatctttatcacgatcggctagagaccaatctttccaacacctgagttcctttcctcctgtcaatactttagtggagtcatggaaatcacacggcgacctgtcgtctagaagaagtgatgggtatccggtcttagaatacattccagaaaatgtatcaagctctactttacgaagtgtttctactggatcttcaagaccttcaaaaatatgtttggtgtgtggggatgaggcttcaggatgccattatggggtagtcacctgtggcagctgcaaagttttcttcaaaagagcagtggaagggcaacacaactatttatgtgctggaagaaatgattgcatcattgataagattcgacgaaagaattgtcctgcttgcagacttcagaaatgtcttcaagctggaatgaatttaggagcacgaaagtcaaagaagttgggaaagttaaaagggattcacgaggagcagccacagcagcagcagcccccacccccacccccacccccgcaaagcccagaggaagggacaacgtacatcgctcctgcaaaagaaccctcggtcaacacagcactggttcctcagctctccacaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgtgtctatcatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaagtgactgcccgctgcccagaagaactttgccttaagtttccctgtgttgttccacacccagaaggacccaagaaaacctgtttttaacatgtgatggttgattcacacttgttcaacagtttctcaagtttaaagtcatgtcagaggtttggagccgggaaagctgtttttccgtggatttggcgagaccagagcagtctgaaggattccccacctccaatcccccagcgcttagaaacatgttcctgttcctcgggatgaaaagccatatctagtcaataactctgattttgatattttcacagatggaagaagttttaactatgccgtgtagtttctggtatcgttcgcttgttttaaaagggttcaaggactaacgaacgttttaaagcttacccttggtttgcacataaaacgtatagtcaatatggggcattaatattcttttgttattaaaaaaacacaaaaaaataataaaaaaatatatacagattcctgttgtgtaataacagaactcgtggcgtggggcagcagctgcctctgagccctcgctcgtccacggtcttctgcatcactggtatacacactcgttagcgtccatttcttatttaattagaatggataagatgatgttaaatgccttggtttgatttctagtatctattgtgttggctttacaaataattttttgcagtcttttgctgtgctgtacattactgtatgtataaattatgaaggacctgaaataaggtataaggatcttttgtaaatgagacacatacaaaaaaaatctttaatggttaataggatgaatgggaaagtatttttgaaagaattctattttgctggagactatttaagtactatctttgtctaaacaaggtaatttttttttgtaaagtgcaatgtcctgcatgcataatgaaccgtttacagtgtatttaagaaagggaaagctgtgccttttttagcttcatatctaatttaccattattttacagtctctgttgtaaataaccacactgaaacctcttcggttgtcttgaaacctttctactttttctgtactttttgttttgttcttggtctcccgcttggggcatttgtgggactccagcacgttttctggcttctgcttcatcctgctccatcggggaatgacacactgcggtgtctgcagctcctggaaggtgtcatttgacaacacatgtgggagaggaggtccttggagtgctgcagctttgggaaagcctgcctcgtttcccttttcctctagaagcagaaccagctctacgagagtgagactgggaacttgatggctcagagagcatcttttcctcccattttagaaaatcagattttctcctgtgggaaaaaaaaattccatgcactctctctctgttaaagatcagctattcccttctgatcttggaaagaggttctgcactcctggaaccggtcacaggaacgcacagatcatggcaggatgcgctgggacggcccatcttggcaaggttcagtctgaatggcatggagaccgggagatagaggggttttagatttttaaaaggtaggttttaaaaataagttttatacataaacagttttggagaaaaattacagatcatataagcaagacagtggcactaaaatgtttaattcattaatctgtttgtttggcactgatgcaatgtatggcttttctcttgccccaaatcacaaacatatgtatctttggggaaactaacaatatgattgcactaaataaactactttgaatagaggccaaattaatcttttaaaaatgatgataatcatcaggtttactcagtgaaatcatattaattattttccaaaatctaaaagctgtagctggagaagcccatggccacgaggaagcagcaattaattagatcaacacttttctccagggttcaccatgcaggcaacattaccttgtctttcaaaagacacctgccttagtgcaaggggaaacctgtgaaagctgcactcagagggaggagtctttcttacataatttgcaatttcaggaatttaatttataggcagatctttaaatacagtcaacttacggtgcacagtaatatgaaagccacactttgaaggtaataaatacacagcatgcagactgggagttgctagcaaacaaatggcttacttacaaaagcagcttttagttcagacttagtttttataaaatgagaattctgacttacttaaccaggtttgggatggagatggtctgcatcagctttttgtattaacaaagttactggctctttgtgtgtctccaggtaactttgcttgattaaacagcaaagccatattctaaattcactgttgaatgcctgtcccagtccaaattgtctgtctgctcttatttttgtaccatattgctcttaaaaatcttggtttggtacagttcataattcaccaaaaagttcatataatttaaagaaacactaaattagtttaaaatgaagcaatttatatctttatgcaaaaacatatgtctgtctttgcaaaggactgtaagcagattacaataaatcctttacttt

SEQ ID NO: 4 - protein, 984 aa, Homo sapiens
METKGYHSLPEGLDMERRWGQVSQAVERSSLGPTERTDENNYMEIVNVSCVSGAIPNNSTQGSSKEKQELLPCLQQDNNRPGILTSDIKTELESKELSATVAESMGLYMDSVRDADYSYEQQNQQGSMSPAKIYQNVEQLVKFYKGNGHRPSTLSCVNTPLRSFMSDSGSSVNGGVMRAVVKSPIMCHEKSPSVCSPLNMTSSVCSPAGINSVSSTTASFGSFPVHSPITQGTPLTCSPNAENRGSRSHSPAHASNVGSPLSSPLSSMKSSISSPPSHCSVKSPVSSPNNVTLRSSVSSPANINNSRCSVSSPSNTNNRSTLSSPAASTVGSICSPVNNAFSYTASGTSAGSSTLRDVVPSPDTQEKGAQEVPFPKTEEVESAISNGVTGQLNIVQYIKPEPDGAFSSSCLGGNSKINSDSSFSVPIKQESTKHSCSGTSFKGNPTVNPFPFMDGSYFSFMDDKDYYSLSGILGPPVPGFDGNCEGSGFPVGIKQEPDDGSYYPEASIPSSAIVGVNSGGQSFHYRIGAQGTISLSRSARDQSFQHLSSFPPVNTLVESWKSHGDLSSRRSDGYPVLEYIPENVSSSTLRSVSTGSSRPSKICLVCGDEASGCHYGVVTCGSCKVFFKRAVEGQHNYLCAGRNDCIIDKIRRKNCPACRLQKCLQAGMNLGARKSKKLGKLKGIHEEQPQQQQPPPPPPPPQSPEEGTTYIAPAKEPSVNTALVPQLSTISRALTPSPVMVLENIEPEIVYAGYDSSKPDTAENLLSTLNRLAGKQMIQVVKWAKVLPGFKNLPLEDQITLIQYSWMCLSSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 5 - DNA, 22 bp, Homo sapiens
cccctgccctcttctttctagt

SEQ ID NO: 6 - DNA, 26 bp, Homo sapiens
cgtattttctgaacacgcctaaataa

SEQ ID NO: 7 - DNA, 25 bp, Homo sapiens
tctatcctttgggtcctacggccgt

SEQ ID NO: 8 - DNA, 23 bp, Homo sapiens
tctatcctttgggccctacggcc

SEQ ID NO: 9 - DNA, 24 bp, Bacteriophage M13
cgccagggttttcccagtcacgac

SEQ ID NO: 10 - DNA, 24 bp, Bacteriophage M13
agcggataacaatttcacacagga

SEQ ID NO: 11 - DNA, 43 bp, Homo sapiens
gaatggtggcgtcatgcgcgccattgttaaaagccctatcatg

SEQ ID NO: 12 - DNA, 43 bp, Homo sapiens
catgatagggcttttaacaatggcgcgcatgacgccaccattc

SEQ ID NO: 13 - DNA, 1863 bp, Homo sapiens
actgatgtcttgactcatgggtgtattcacaaattctgttacttcaagtctttttcttttaacggattgatcttttgctagatagagacaaaatatcagtgtgaattacagcaaacccctattccatgctgttatgggtgaaactctgggagattctcctattgacccagaaagcgattccttcactgatacactgtctgcaaacatatcacaagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactagcagagagtcctgagccactgccaacatttcccttcttccagttgcactattctgagggaaaatctgacacctaagaaatttactgtgaaaaagcattttaaaaagaaaaggttttagaatatgatctattttatgcatattgtttataaagacacatttacaatttacttttaatattaaaaattaccatattatgaaaaaaaaaaaaaaaa

SEQ ID NO: 14 - protein, 505 aa, Homo sapiens
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 15 - DNA, 1863 bp, Homo sapiens
actgatgtcttgactcatgggtgtattcacaaattctgttacttcaagtctttttcttttaacggattgatcttttgctagatagagacaaaatatcagtgtgaattacagcaaacccctattccatgctgttatgggtgaaactctgggagattctcctattgacgcagaaagcgattccttcactgatacactgtctgcaaacatatcacaagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactagcagagagtcctgagccactgccaacatttcccttcttccagttgcactattctgagggaaaatctgacacctaagaaatttactgtgaaaaagcattttaaaaagaaaaggttttagaatatgatctattttatgcatattgtttataaagacacatttacaatttacttttaatattaaaaattaccatattatgaaaaaaaaaaaaaaaa

SEQ ID NO: 16 - protein, 505 aa, Homo sapiens
MGETLGDSPIDAESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 17 - DNA, 28 bp, Homo sapiens
agacaaaatatcagtgtgaattacagca

SEQ ID NO: 18 - DNA, 28 bp, Homo sapiens
gtgatatgtttgcagacagtgtatcagt

SEQ ID NO: 19 - DNA, 31 bp, Homo sapiens
agattctcctattgacccagaaagcgattcc

SEQ ID NO: 20 - DNA, 30 bp, Homo sapiens
attctcctattgacgcagaaagcgattcct

SEQ ID NO: 21 - DNA, 24 bp, Bacteriophage M13
cgccagggttttcccagtcacgac

SEQ ID NO: 22 - DNA, 24 bp, Bacteriophage M13
agcggataacaatttcacacagga

SEQ ID NO: 23 - DNA, 39 bp, Homo sapiens
gggagattctcctattgacgcagaaagcgattccttcac

SEQ ID NO: 24 - DNA, 39 bp, Homo sapiens
gtgaaggaatcgctttctgcgtcaataggagaatctccc

SEQ ID NO: 25 - DNA, 85389 bp, Homo sapiens
aagtgagaccctttgaggacatgaggacaggtggaaggactgcatctgtgtgttgccaaagagtaacagagactggtataggacttgagttcaattcagtcatatccttaagtattgtgccatcgtgtctggattactttgtagtataagtaaatttccaagcatggggaggaaaggagaagctcctttattgagaattcaactgaatatagagaaaactaattttacacaactgtaatcactgtagtcatttggacaaattagcaaacccaagttttgctttaacttggattgccttaataaagatgttttggggcttaatggcacagttgctcaactcccccactttattccgtgatgttcagacccagccagcatttccccatcaggctcttgcaccatgattgacagggacacttttactagtccccttgaagaatgaatagttactcaatggagattaaccagatatatatttattttactcagaatatcacgataagtataattcagagaattattgccttctaatatactgccctgtgtgggggcgtctttgaaagtccgcaaagtcactgcaattctaataggccactcatgtgacaagacctgctcccacatcggtaatttggcacagctagtatttctccttgccaaaaagggcaaaggccttgagcaagaagccagctttttcctgattacaaaactgaccacaattcctcgccaacctaacagcgtaagtctatttttttctggtggtgtgttattcttctcatagagaactccattttttcattatgacatagcacttatcgtttaaacatcaattgatgttcaaacatcagctggtgtaacattgctgcagttgctattgatggataagctgaagtttttaagaaagcaaacccgatgtataaaattgaaaccatatcaaacccttcttcattctctcagctatttaattttacagaatttagatagcagtcagtatcattttgggcttcacaaatcagtagagtaagtaccttaggaatataacatttcagtagcatgctgataccaacgtttaaactatggatacatatttgaattccaaatttttcttcaaataatgtgattagagattcaaccaggaatagacaccgaaagaaaactttgcccaaataagctttctggtatttcataagcaagagatttaagttttccatttaagaagcaattgtgaattttacaacaataaaaaatgcaagtggatattgaacagtctctgctctgataattctaaatacagtacagttcacgcccctcacaagacactgaacatgtgggtcaccggcgagacagtgtggcaatattttccctgtaatgtaccaagtcttgccaaagcagtgaacattatgacacaactttttgtcacagctggctcctaataggacagtgccagccaattcaagcccagtcctttctgtgtttattcccatctctcccaaatatttggaaactgatgtcttgactcatgggtgtattcacaaattctgttacttcaagtctttttcttttaacggattgatcttttgctagatagagacaaaatatcagtgtgaattacagcaaacccctattccatgctgttatgggtgaaactctgggagattctcctattgacccagaaagcgattccttcactgatacactgtctgcaaacatatcacaaggtaaagttccttccagatacggctattggggacgtgggggcatttatgtaagggtaaaattgctcttgtagtttgtcttccaggttgtgtttgttttaatactatcatgtgtacactccagtattttaatgcttagctcgttgctatcgcgttcatttaaaaacatgttcagaaccttaaaaaaggaaacctaacctaatctattttatctctgtgcatggctcccatttcctgaattttaagcattaaaggtatagttatatccaaaaacaatcctgttcatttttatttcctgagtttgcatagatttcccaagaatacataagggctttttagacttgaagggtcactttttcctcttttcatctcatatgttagagatctctcataactgtttttatccctcttgcagcacttttattcctcttgaagtactctcagctcttttctgttctattttgaaatctaggtattgtgtgtgcacttcagctctcccaaagaattgtaaattcccagagtgtaggaccaagtggtgctctttattagatttcttaggatacttcctagcatagtgcctaatgcattgcagaatagattgtcggaaccttgaaaaatatctgttccaagcatcacattttatgcataaagaaaatgaggtccctaaaacagtaaatgcctattgcaagtgactacctattaacttctcttcagatgtgtcagaatcagatacctccttttcaatatttctttcttggccattgcaaaatataagttctccatttctatagctccttctactggagtttcttagcacagccttttctgttttgtattgtctcgtattgatgtctatatttctattcattttatttttctcttctcagattctgtgtaaaaatagattgttagctagtgtgattcatgttttataatataagtgctaaagcattaaaattaagtcacttaattgctgaaatggatggctttacattttaaccttttaattttcctggaagcctgaagtttgaacatattagtcaccgtttttaaaatataaagctttttaaaaatgagtaagaaaatgtttcctgaagtactgttttattttattacagtaatttgattcaacatgtatttctatcatctaagcctcttaactaggagttttgttttattccataaattgttgttattctgtttttctgcaagccataaaaattgttgctatctttcttcagttattaatttagcctgcctcagggcagaatgtcctggctatactgtgaaatagttcttgagtcaaaatttgatagttttgtctattttgaaagaacatgtctagaatgcagtgcaacaaatatcaacaaataggttaactgagaggcaagtactgtttgtggtaagggatggttatgtgattcaagttagttttcattctgtgctaatctctaattttatgctttgaatttactctgagtttaatttttttgagtctcaatttcctctaaaagaatgatccagtacttttcttcctaattaacatagtgcagaatgaaattattagcaattaaaacattatattttgtaccaataatttaacccttgtcttatagggtctctggctactacttcttcataagcactgtttctctgttaaaaatgtgagctcctaaaggcccaagacctatgattgttactcctattgcattttgtaaatgacctttaaataattactccacctcttccaaaacagacagacttaaaaataaattagctatatgatcatcattagccgaggccagcagagttgaacactgtggagttgaggtatttggcatgggtacagtagtcctcttaggtgctccttcttcctcatcataggcaaagtgaaattgggggtgttgcaaatggttgggtactgctatttaagaaaaagtggcacatttagtgtgaatgactcagatatccaggtagattggtcatattcatgagattaaaaatctcgactacacttagccactatgcccaaagaggacttccatactttttccaaggctccatcattgctgtgacccctttatattattcccctttctctttttatcaagagctcctcaagcaaccccaagtaacctcatgctggctctgaaaccatttccactagtaaaaattggcaagattgaagggaagcttccacaagctcaagctgaatatgcctcctgcttttagtgccagacttagtcctgtgtgaattctacaattttaagtcgttgcaccagagccttgaaagatgatgacacacttgtcctttggagctgattgagcaatggaaatatgccttagagacatgataaatgctgcaatagacaatccttcaaaacgagtctctcctgagataggagtgagtgattgattgcatgccatttttattgagggatacttaatctgtggttacatatctccaggcaactgtatattcagaaacatttagataactgaccaaagttttcacctttccagaagtttaaagggacccatgtagtaccagggaatataaggaccagtcctttgttggccacatatttgctgtgtgaatttaagcacgccatttttcttctctggccttctgtttccacatatattaacagaatacacaggattaatgggaggattaaatgacacaatacttgtgcaagtagatctaaagccctatatggaatttattgctttagccttgtagcgttaatcacttgtggctagaatagaaggctccgtttaaaaaaagaaaaggctgggtacagtggctcacatctgtaatcccacaactttgggaggccgaggcgggcagatcacgaggtcaggaggtcaagaggtcaagaccatcctggctaacacgatgaaaccctgtctctactaaaaatacaaaaaattagccaggcgtggtggtgggcgcctgtagtcccagctactcaggaggctgaggtaggagaatggcgtgaacccaggaggcagaggttgcagtgaggctgagattgcgccactgcactccagcctgggcgacagagcgagactccatctcaaaaaaaaaaaaaaaaaagaaaagaaacctacattttaaacactttatggatatggaaaaacaaaaaccgcagaactctttattgcacaagagtaagtggcagttatatgaatgaagcggggcatccgcatgatctcctgtgagtgttttgagaagatgatcaaacttagagaaagcatctttgtgtctaatttttttaaatgatgaaattatttcccactttcattaatattttcctgtgaagaaattgaagttattctttgggcctaaataaaagtgacaattacatgttttcttctcagaggcattgagaaacttggattataatgtttttcgtacactgaagtagaagtccagaaaagatttgagttattgacacagatcttcttttttttttgagacagagcctcactctgtcacccaggctagagtgcagaggcctgatatcatatttcaccacagcctcaacctccctgggcttcggtgatcctcccacctcagcctcactagtagctgggactacaggcgtaccttaccacacccagcaaatttttgtaatttttatagagacaggatttcaccatgttgtccaagctggtcttgaactcctgggctcaaggaattctcccgcctcgacctcccaaagtgctgggattacaggcgtgagccatcgcagcctgcccctgaattccataatttcacgtgtaattagaattcaagacctttttcttgaatattactcttgtccatattaatttttttctgagtatggctctagctttatcatttttttctgacttaactctgattaacctcttttttcatcaccagtacttggcagattttgtagatttctaaactctgaaaatcactgtttttctgaaaactgtcatttgtctcatactttcctccaaaattatgaattataggaaacaccccaaagaagtatcatttacattagctgtttacaagaaaatacagggtttcatttttaattttattttcccctactatctctaaggaatacaacttgtcttgaaagctccctgacagcagctatccatttaatctgccaattgcagtataaaaattggttattaaatgaaaaacaattgtatacctctcacagaggttgtcatatattactatttttgtcagcattctcacttataactctgttaggataatcctgggaaaatttctaaaatcttcatagctctttcgttaggtacatattccttgagacagaggaaatctgagcaaagtaaatttcttcattatcttagatcaacaaaagtgatacagagcttctgtcctgttagctccctttggacgcttaaaataggcactagaggttgtccagtaaatgcttcatatccatctactagtgctgaatatttgtgaaaatataggtgataatgaattagactcgtttgaatggaattaagtttctgtaaagttttaccagattgtcttttttttgttttttgttttttgttttggtaaaagcagttttgcacctgtaactaatttaaagacgaagaaattgcacatgtaactgatagcaactgccatccaactcttataacctcagaatcaagatcacgtggctatttgagtacacagtgttttaaaccgtgtaatttttatttgagcaagggattgccaacacaagatcgaggttcccacaagtggtgaatggcacttgtggttaacactgtaaagcagatttaaaacagtcaactgtcaattgcctacaagggaattattcctcatcacctcatgccaggtttttatatggccttttagctgaccttctaaatgagaaactttgccatccagtgcaaactgccaatagtcctaaaatgcctatctttattatcagtctaataatatttattgagtgccgattttcaaggcaccatagtgaatgctgtgaaatctagacactttttccctggctttcaaagctctttataatccaggcatattttatatattttaccttatttctcaccacattccaggcattggcctgtcttgctctgttcacactaatctctttgtcccaagagtaaggccgacccacccttgctccaagcctagtatgtcttgaaatgcctttcctaattcttccatcttttcaaatcgtccctgtccttcaaggtcagcttcaagtaacctctactccgtgaagcattctctgaattcattgctgttcattgctccctctctcctctaaactgaaaatgtgttcagaatatctctcttctgccgcttccactgccttcagcccagtccaggacaccgtctgtgcctggctgctagtctcttgatggggttttcagcttccatcctaacctcacaacagcctagtctcaaccggtcacccacagagatgatgctcaaatgtgagtcagatccgtcactccacccatagccctgcagtggttccccatttcacttgtaacaaaagtccaagtctggcccctacctcctgccctaccccagctgacccctctggtcttctctctctcttccacccactctgctccaagcacgccggcctccttgctgttccttccacattccagatgctctcctgccttcgtgcacctgccccagctcttgttcctgtccaggaccctcttcctctggcattctctgggccacctctgtcaattccctataatcttggctcaaatcttaccttagagtgagggtgaccttggccttccctatcctaggacaggggaattacatatcctccccattgcccttcctcctctactctacaatttcctatttaaaaagcatttatcgtcttctaacatgccatgtaatttattggtggattctatgtattgcttactgtctgcttcacctccctggaatgttaaccgcatgaaggcagagatctttgtctagtttgttcacccactgatgtattccaaatacctgaatagtattgctcattaatacttgctaaatgaaagaatagaatcctctaacacttacggcttctatcacacaatttagccctgagttcaaactgtctggtattatttcctgttttatgtattccctaagccaaatcatatagtcactgaagaaaagatccatgttttattctatattccacaaatgccaaacacataataagtataatcaggtcttgttgacttattaataccaagctgatctcctaaggtcagagactggaatatatttagcaaataaatacaaaattttagtgtatgttaaatattattctaaaaccaatgctaatataaacaaaccaagtactggatatgctacaaaccaggcacaaatatgttttgaattatctgttagttaatttgcaccgtgtccctccattagtttgagtagcattcattgaatatcatttgaatattaatttaaacatcatttgtttgtgagttacccaaatccctggcttcttctgcaaatctacccaccttttgatacttccattatttatttgaacatccaaaacacttatttgctagcaatgtttaaaaaagagtagcttatttaattagctaatatctaatatatgaagcacttaaagtattaattgcctgatatgtaataggtgccaaacacgtcagttctgtcttcttctcatctttaaaatggaagttaatacagacataatgtctccccttgggtcttttaagtcagatctttaggaaaaagtaaaatattctcattattcttattattttgcttgcattagcaataaagtatgcctgatttaagttcactggacattatcttactattatctatactttacctaaagccctgcctttagttttgtaaataaaattatagacctattagatagattgagcaagtttgcttagtggattaaaatatgatgctaataagagccaaagtgagtttgataatgggttggccagatttacacagaagtctttgccatttacaaagagttctgtttgccattgatacagtcacatttctagccacaattacccaagatatgtaaacagttaactcaggagaaaccaactcaattttcaggtatatgccatatacaaaatctcatcacctttgctattatatacacttagtattatttcttaagcaatatcatgtaatataaggggcacgttgatgacagggacatgtacctttttcttgggatttcaatatgaatgaccatccaaatcttgatcatcccaatggctccacataatttggacaccatgtctaccagaaagctatccctgattatttcaggacatgttagtatctttcttttccacatttctaaagtacttactatctctatcacgagtctggtctctgtacttccttctgctactaaacatctctcaccttggtaagcttctcaagaaccacaatactatgtgtttcttgtaaggcctgaaaaacaccttgcacgtagtattaggtgattagctatcttgatttaaggttttagattactgattgaacatttactgagctgaagatatagagaaagaaaaatgccaaagaaaataaaatgaggaagtttggaaaagagaaacacactagtaaaatatgatggtaacttctaagaagcaaggtttataaaatagattgttgatacaaagaaaacattataaaaaaatgggtagcttgaatggatttaacccccaactttatttaatttaaatggaatgcctcaaaagactactcagctgatccaaacatagtaacttcccaaacacaagactcttgacgtaggcctcagaatttggggaaaaaaattcctgcattgtttaaaaatacagtgtccttgtaaagcagaataggccttatatacaagccaaatatatcagttctaaagcaaaaacaaaacttttcctacttactcttttatttcttttttttttttttttttgagacagagtctcactctgtcacccaggctgaagtgcagtggcgccgtctcagctcactgcaacctctgccccctgggttcacacaatcctcctgcctcagcctcccgagtaactgggattagaggcacccaccaccacgcctggctaattttgtatttttagtagagacggggtttcaccatgttggcagggtggtctcaaactcctgatctcaagtgatccgactgcctgcacttttcaaagtgctggattgcagatgtgagccactatgcccagcctgcttcactcttttagataaaatgtacaccctttgaatttttacttccgccatttacactgatttgaagtccttggttgaatatgtatttagccttcactgtgcaaggaagctggttacaaaaacagatagtttattaaaatacttgtgttggtttttaaaaaagaataattttaatacagtttttctcttctttctttttgctctttttgttattttttaatagagacacggtctggctatgttgttcagactggtttcgaactctggcctcaagtggtcctcccatcttggcttcccaaagtgctgggattacagatgtgagccaccatggctggccttaatactgttttatgagttctgaattgaataatttttaagtgaatgtcctaaaattctatgacaatccttttaacacaactggaagagtttttgttggcttacttggtttgtctgcatgtttccttctgtcatcccccactcctaggacccatatggaacattctctcttaattaactcttttattgaagtataatatacttagaggaaaaatgtccaaatcattttcacaagtgaacatacttgcgaaattagcacccagacaaaaaaaaaaaaaaacagaacaataccagcactccagaagccctctgactattctaatttctaactcattgttaagttttgcccagttttgaattttatataaataaaattacacattgtttactcttttgtgtctggattcttttgcttagcatttattcgtgagattaatcacactattgaatgtaattgtacttcattctcattactgtatagtgtttcattgtataaatatatcacaacatatttatcctactgttgatgggcattcagatggttttcagtattgcaaatagtactgctatgaacattgtcctgtaggtctcttagtgaaggagcgtatgcatttctggtagctatatgcttcagccaataatagagtatgttgagcttcaggagacgttgccaacagttctgaacaggactctagaaatctatacttctactagaagtgcatgagtgttctagctcttctacaccatcactggtacttggcattctctgtctttttcacgttagccattctgatgggtgtgttggtatcatattctaattttaatctccccttttctgatgtctaaaaagttaaacactttaaaaagcgtttgttgtccatttggctatcctttcttgtaaagtacctgttcaagtctttcacccatttttatattgagtactttaggtaaacgttctaaagcataccttgtcagatacatgtattgtaaatatctcctcttactctgtgtgctttttcgtttacttaataaacagatatttctaatcaatatagtctgatatactagatttttcttttacagtaagtgctttttatatcctttggaagaaagctttgcctactccaaggttgataaagtgtgtttttattttaaaagttttctggttttatatttcacatttagatccataatccacacagaattgacttttgtgtatgttgtaaggcaagggtttatctctttcatggggatatccagttgacctgcttcgtttattgaaaagatcatcctttgcccactccccagcagtgacatttctgtcataaaccaggtgaccatataggtctgggatttattctggacttactcttctattgcattgtatttttgcaccaattaagattgccttaagtactgtaactttataaatagtttgatatctggtagtataaatcctccagcgttgctcttccccttcaagatttccttgccacttggcctcttgaatttccttataaattttagactcagcatatcaattaccacacaaaagtgccgagattttgattggaattgttctgaatctatagataaattgggagataattgaaagctttacaatattgagtccttcaatccatgaacatgaactatcctttcacttatttattctttaatttttttaaatagtatatttgagttttcattatggagatcttacacatattttgttagattttttcccctgggtatttgattgttttaatgctgttgcaaaaataatttttaaatttcattttctacttgttgctggcttatagaaatataattggtttttgaatattgaccttgtacccagcaactttgctaaattattttattaactataatgagtttttttgtggatttttaaaaacacagtctgcctaacatggtggctcatgcctgtaatcccagaactttgggaggctgaggtgggtggatcactttgagctcaggagttcgagaccagcctggccagcatagtgaaaccctgtctctactaaaaatacgaaaaattagccaggcatggtggcatgcccctgtaatcccagctacttgggaggctgaggcaggagaattgccctaatgccagaggtggacgttggagtgagctaagataatgccactgtactccagcctgggcaacagagtgagacttcgtctcaaaaataaataaataaataaataaataaataaataaataaataaataaataaaacgaacaacaacaacaaaatacagtcatgtcatctgtaagtaatgttagttttagttttatttcttcttttccaattattctttgtctttttcctttgttgcattggatggatttccagtacagtggtgagtagaagtaatgatagtatcattaccttgttgctgatctcaagaggaaggctttaaatatttcaccatcaggtatgatggctactctaggatttttgtttgtttatttgtttgttttttatagattttctttagcagactaatgaagttccctttccttcctaagaggttttttatttaaaaaaaaaattatgagtgaatgttgaattatatcaaatgctttcatagtttgttgtggtggtggttgtttttgagacagggtctcactttgtctccaagctggatcatggtgcattgcagcctcaacctcctgggctcaagtgatcctcccacctcagcctcttccagtagctgggaccacagcacatcaccacacccagctaatttttaaaaaattgttatagagacagggtctcactgtgttgtctaggctggtcttgaactcttaggctcaagtaatcctcccaccttggcctcccaaagtgctgggattacaggtgtgagccactgtatctagcctagaatattttttatagtgctcaggagaaagattaaaagtatgaaaagtgtctactatgtatactttaatatcatattgtaatacatttgaagatactatatggtataactaatgaaaatcttcaaatcagctcataaccactagagatctggctgtatctgttttgacacttttgttctcagtatctgtttagcttaagacccttgtcttctcattaagagtgcttgacacagtaagttgaaaacataatttcaaaagagcatagagtgtcacatggtttgccagactaaacaactctgagtaagaaagtggctttccccctatggtaatgccaagctactagaggaccttcagtggtaaagtatgatttaaaaccctgaccccatctgtcttttcccatttgcagtaagtcctttatagtgtttgaaggtgggcacagcagggaaacagtactctggcagatctgcggtggctccctcctttctgagtcattacacactcttctggatcattcctttcatttttccttttcctctcctctcccctattttcattcccctatcactgcaaagccaaaaccaaaacgtggaaggggacctagccgaggaaaacattccagcttcttttttctgatctaaacaccgttcttcttcttttttattttttaattaataactttatttttagggcagttttagtttcagagcagaattgagtaggaagtacaaagagttcccatatacttcctgtcccccaccacgcacaatctcccccactatcaacatcctgcacccaagtggtacacttgttaacagtcaataaacccccactgacacatcattatcactcaaagtccatagtctacattaagattcactcttcgtgttatattctctgggtttgtacaaatgtataaggacacatatccaccattgtagtgtcatacagaatattttcactgacctaaaaatcctctgggatccacctgttcattccgttctcccccaaacttctgactaccactgatctttttcctgttttgatagttttgcgcttcccagaatatcatatagttggaaccatacagtatgtggccttttcagtttgacttctttcacttaccaatatgcacttaaggttcttccatgtctttccatggcttgatagctcatttctttgcagaactgagtaatatttcattgtttggatgagccatagtttatccactcagctactgaagggtgtcttggtgcttccaagttttggcaattatgaatagagatgctataaacacctgtgtgtgggttttttgtggacttaagttttcaattgatttgtgtacctaccaaggagtgtgattactggattgtatggtaagaatatgtttagttttataagaaatggccacactgtcttccaaagtggctgtaccattcatcagcaacgaatgagagtttctgttgctccacatcctcatcagcatttggtggtgttagtgttttagattttagccattctatttgtagtggtatcttcttgttttagtttgcagtgctctcgtgacatatgatgttgaagatcttttcacatgttcacatgccatctatatatcttcttggatgaagtgtctattcagatattttgctcatttttaaaacagggttattcactttcttattattgagttttaagcgttctttgtatattttggataacagtcctttatcacagtagtccctgacctttttggcaccagggaccagtttcatggaagacagtttttccatggactggggtgggcaggaatggtttcaggtgaaactggtccacctcagatcatcaggtattagattctcatacggagtgtgcaacctagatcccttgcgtgcacagttcacaatagggtttgtggtcctatgagaatctaatgcagccactgatctgacaggaggtggagctcaggctataatgcttgcccatcagcagttcacctgctgtgcagcctgattcctcccaggccatggactggtactggtctggacctggaggttgggaacccctgctttatcagatgtgttttgtacagatacattctccccatctgtggcttgtttactcattctcttgacactgttttgcacagagcagactttttttattttaatgaagtccagataatcagttatttcatggttcatgtctctggtggtgtatctaaaaagtcctatccgtgctcaaggtcatctaggttttctcctatattgtcttcagggagttttatagttttgtactttacatttagatctatgttccattttgagttaatttttatgaagagaataagctctgtgtctaaattattattattattattgttattattatatttttttgcatgtggatgtccagttgctccagtcccagttgttgaaaagactgtctttgctccagtgtattaccctggtttcttcatcaaagatcagctgactacatttatgtggtcctatttctgggctctgtattctgttcctttgatctatttgtctattcttttgccagtaccgtactatcttaattactttaactttctagtaaggcttgaagtcaggtaatgtcagtccttcgactttattcttcttctttaatattgagttggctattacgggtcttttgcttctccatataaactgcagaaccagtttgccaatatccacaaaataacttgctgggattgttattgtgattgcattgaatctatagatcaagttgggatgaatgacatcttgacaatactgagtcttcctatccatgagcatgcagtgtctctccatttatttcattctttgatatctttcatcagagttttgtagttttcctatataggtttcatatatattttattaaatttatacttaagtattttacttttcggggtgctaatgtaaaacaaaacagacattatgaggaaaatgtaatgtctgtttgggggtaatgtgtttttaatattaaatttcgcttgttcattgctggtatataggaaagcattatgacttttgtatattaaccttgtatcctgcaaccttgctataatcacttactaggtccaggagattttttttttttctgttcttttggatttttttatgtagaagtcatgtcatttgcaaacaaagacagttttatttttaccttcccaatctgtatcccttttatttccttttcttgtcttgttacgttagctaagacttctagtatgatgttgaaaagcagtagtgagagaagatatccttgccttattcctaatcttagtgggaaagcttgagtttctcaccattacttatgatgttaactatatgttttgtttgtagatgttcctaagtttaggaagttttcttattttgtcaaattctttttctgcatttattgatatgatcatgtgatttttcttctttaggctatgggtgtgatggattacattaatttgttttctaatgttgaaccaaccccacataccttagctaaatctcacttggccatggtgtataattcttttcatacattatcggattcaatttgctgatattttgttaaagatttctgcatgtctgtccatgagagattggtctatagggtttattttccttgtaatgtctgttttgttttagtattagggtaattctagcctcattgaatgagttaggaagtattccctctgcttttatattctgaaagagatttcagagaattggtataatttcttccttaaatgtttggtagaattcaccagtgaacccagtccggtgccttctgtttcagaaggttattagttattgattcaatttcattaataaatgtaggcctattccatttctctatttcatcttgtatgagttttggaaaattatgtctttcaaggaatgagttcatttcacctagattatcaaatttgtgagcatagagcctattatcctttaatgtcaatgagatctgtaatgatacccctctttcatttctgaataactttcattcattcttcacgtactagacaaagtagaatttcaactaaaccaaagcaactgttcaacagatttagccttagtcctaatcagattattagtaatatctccctttagaaatatatgaaggtacttttagcagaggaaccaagcttgagctagtttgaccataatgaatttttgctgaggaagcagaagaaatgtttatattggtgagggcataatcctgtaatcttttctgtgtcctgatacctcaggaccagcgtaagcaaactccctagacaagatgtggctcatgcaagaggaaatatgcttttgaaaatgaacatgggccaagcatagtgactcatgcctgtaatcccaacactttgggaggctgaggcgggaggattgcttgagtccgggagtttgagaatagcctgggcaacataatgataccttgcctctacaaaaaaaaaaataaaaataaaaaaataaaaataaaaaaattagatgaatgtggtggtgaatgcctgtggtcccagctacttggaaggctgaagtggaaggatcacttcagcacagggaggtcgaggcttcagtgaaccataattgtgccactgcactccagcctgcatgacagagtgagaccctgcctcaaaaaataatagtaaaattaaataaaatataataaaaaacatggtctttctaataccctttgttgatctgaatttgaaaatttcctgtgagaccttctggcttattataaagaaatttgcatctaaagatacatattggaatgtttcaatctcaaagctatcaatgggaaatcaaacttcagaaacattgaatttggttttgaatatagtctaagtagacaataaaatgaaaatacatttgtttatattgaagaagcaaatgacacaccaaatagttaatttctaatagttttcatttttttctttttcttttttgtttttttttcagacagtgtctctctctgttgcccaggctggagtgcactggcatgaactcggctcactgcaacctccatctcccgagttcaagtgattctcctgcctcagtctcccaagtagctgggactacaggcctgcgccaccacgcccggctaatttttgtatttttggtagagacagggtttcaccatgtaggccaggctggtctcgaactcctgacctcaggtgatcctcccaccttgtcttcccaaagtgctgggattacaaacgtgagccaccacgcccagcctctaatcattttcttaattcatggtgcccaaaataaaagattctatattcagagaaaccatgacagaatttaaactctagccccaatacctatttaacattgggcaagtcacttaacctttttatgtctaatatttttaatctgttaggtggtaataatacttgtgtataacaacataggactgtttacaacaagcacttaataattgttagctattgttactaaattgtatttctgctgatgggttccagagtcttttcctccttcactaaacaaaactcaggatactatgtgaaggaaaagaagtgagttggtagaatttattttttaaatgcttgagtcaaaaggagagccatttgtggctgaagcatcaaaaccattttctgattttctttcattctacttgaaactacattaaaattttctccaattttccagatgattgcatgataattccttttaagaggactttaacacatgtaagaaatagagttactccctctgttaggagaaatcatggaaagatattataattaaaagatagtgaattttagcttaatttttacctttatctaaataacactcgtacacagctacagctttcagtgaagaagagatactcattcccacagctaaatcccgctcctcataggcagtgattttacctctttcacatatttcttctggcatttaccttcctatctctagaataacatacttacattaatggccaggcatagtgactcacgcctgtaatcccaacactttggtaggtcaagatgggcacatcgcttgagcccaggagtttgagatcagcctgggcaacatggtgaaacctcatcagtacagaaaatacaaaaattagccagcagtggtggtgcatgcctgtggtcccagctacttgggaggctgaagttggaggatttctcgagcccgggagtttgaggctacagtgagctgtgattgcgttacagcacgacagcctgggccacagagagagaccttgtctcaaaaataattataataataataacttatactattatttttaaaattgtattcttatccattgacttcttactgtgggagataataatttagtccttttactaccatattcccttttccaaaacacatacttctccctcccctccatcttcacaatgtagttacttgtaatttggggttaaatcagttgtctgtgcttacattgtagtaactgaaagtgctgcttaaggttgagccaagcagtgtgctatgattctgtttcctctcctgcttagctatttacttttctggagttaatttttgtcacttagtcattgctcattttgttgtcatttcttgctatgtactcatcactaattcatcctcaaactctctgacagagtaataaaaactcctctgaaagtggtcagacatatcgggcattctggtttctcaggtgatcccacctggccttcttcctcctgcattttctctaggctgctctgtatctgtagcctgggactaccttccacctctctcctgtctttcagatcctttgtttcctggtcccatcttttctcttcttggttctggtggagaactctgggaaagagtacgtgggaggtaacttttctggatgccttatcattctgaaaatgtctttactctcatacttgatggacgatttggatgagtctaaaattctaggttggaaatttttttttctcttgaattttggcaacattgttctattgattttctagcttccagtgttactgttgagaagttcagtgtcattctgattcctgatcctttctatggtactttttttttttcctgtcaggaagtacttatgatcatctctatagaaggggtattaatcttgacagtgttcctttctgtgtggtttaagaaatcctttctctcttgggttttgttactgttttataaatccttgatcttagtcattctaaggttaaagaaaaactgaggaacaactcaggaatacgtttcaagtacatgtgataaaaggcaaatatgcagaggatgataactatatgtttaaaaaatgggaaaaaagagatagtcgcaaacttcaacataggttagatcatcatttctcaaaccatattccatcacatattaagctcctcaaatattgcacactgtgcttttttctctcttttgtgtattctcgtgaacattagcctgttagtttaagaagtctcacagtaaacctatttaaccttgcttaacctagcattgcctaaatttacttgatcaccagattttttttgttttgtttttaattatttttaaaggaacattattaacatatgtataacacagttggggaaatgccagataagacatagactttcctggctgctaaagagcaattaagcattggaatgagtaaccaagaggagctgtggactctccttttccagaggcctttaaaacaggacatctgttttaggcgtagtcctgtttaaacaatagggctttgctggcggttcttcctcttcctgtcccctagtggtagtgttggaggcttttagcccaccttttatatgactgtagtactctggtgactaatgtgtacacaaattactgttatactattaatattaattatatttattgagaacttattaagtatccagccctatggtaagtgctctacatacattatctaatttaatcctcacaacaactctaggagggagacaatattaatattctacagatggagattagatatgaagagatacgaacttccagcaatagggttggattggaacctcgggcatctgacatggagctgtgcccatccttcttcaccgttaatgttcaaaacagcaaacggcacaaagcaagcaaacaacatttgtatatctgtacttactatgtaaaaacaaaccaagataatgcttactttattagactattcaagactattccctacgccttttgattaattcagagttcatgacttttgttagaaacaaattctgttacagatttagttcttcgcattttattctgtgataatttgccatccaggaaatggttccttacatgagactttttccctcataaatttgatgacattcatatacattcactaaagacccccaaaaaataaatctggatctgtgaaaagtggcaaaggttaaaagtcccattttcagcatatttacatgtagtaacaacttgataacacagcgctgagaatgccaggctcagaggagccaagctgggctcccgtccagtcagcctctagctcaccaggaaaccctgagcaggtcactttgcctcattttctttatctgtcaaatgagaactcagatgatctcaaaacagccttttctgctgtagtgtttcaggatgattcttgtctctggatgtcgctgtcatgatgggagaggaattagtaaattcccaggtcagtttacactgcagagtgatcttgatctgcatgaatcagtaaggatgggctggataaacaagttggaaccacagttatgagtaccatgacattaactgaaacaaatgagaagggattgaaaaatcggcggttaagtgtcttttaaggtcatttccaacaaaactacaaaggacctatgaaaggcttttcaggcacgtactcttgttgcctagctgaacacatttctccaataaagctttcatttgagagttactcttcatagtacatcccctgcccccctgcctgcctggcacattataggtattcatgaagatttgttggaactgttaaataattgtttaatatagaactggaagatcaggaagaaataatgtttttctaaccagtatccagtgtaaagtcacaagtgtgggctatggagcttcagaccatctggattcaatgccagttctactgcttagttactgcgtacttttaggcaagttacttacttccaggtccatgcctcagttttcccatctttaaagtaaattataaatgcacctacttcctcgggctggttgtgaggattaaatgacacaatccgggcacaatatttggcgtagtactttgcacagatttagtgtcctgttagtattagctaatgtaaatatgaagaagatgtaacattttaccagctattctaatcatagcttttcttttcttttcgtaagggacaacataaaaccccaaaggtaaagcatatatttctggtgttcaattttacaatttagcatcttttccaacccacttccatactcccaaataataatgcctatcagattaaagaagttactttatttttgcatgaaattggttgtatttttgttgcactacttcatcctttgagtattgtgaaaagtctgcgcattgactataatagcatttaaagccaagtttgaaggtctaagataatggcacacatccatgtcctttgctcacaagttctgcctaaagactttagcctgcttgaaacactctaaaggttgtgaaattaatggatataccactgatgcaacacaactcagtgtcaccagctagggagttaagacatgttcttgttactgagaagggctgttgttcatgcaaatattttgttcatttgttctttcatgggtaattgcacggagtaacaaaaaacacctcctcaacttacagcttggcagaaacagtagcttatgttttctagagacaaaattagaagcaggctttctggacccatctctaagtcaaaataaaataaaatgtctagggaaccactgtgcctcccagtaactatggtccatggaataatattgtttaggtcaaggtgtcccagagtaaaagggaagggagaagggcaggggggaaggggaaaagtgtgtttgggcaatgttttcatagagatgaagaggcaagctgtttttctttcttactttgtcctttcaacttagaaaaataagaaaaccatctaggaccacaaccttcctcttgtaatcagcaaaagagaaaaaactgaatatacatcgttaaggggaatggttatggcttaagttgtattttattgataatgttgaagattaacattttttggagagaacttaagtcttaacaaaactaaactattgagaaagtattgttatttttccatctttagtagcaaaatgaaaatttactctactcttctgcataaaatatgatcatcaattggcaagaatttggttaaaaaaaaaaactgtgcatagaatatgcacatgggaagatagaataataagatgctgtccttttactccagactggaagtctaatggtggagatagttatccagataattatatctgagttaaaggaggaggtacaaagtgctgtgggaacacagagaatggagtgtttttaagttggaggattcatagatggttttacagagaaagtagcatttggactgggttttgaaagacaaacaggcttgtggcatgaagaggtgaggagtaggaagaagatacagcagcctcagtaacctgtatgactaaaagaggcagggcatgtttaacctgggcagatagtatgatatgcccagccaccagatatgccaagaatgggagcgaggtctgggataaccagtggaaataagactgaaaaagtaagttagggctgagacatagagggccttgaatgccaagctaaaaggccagtgtttctcctgcttttccaccagtatgcccttactagtagaggacagcaaaaccataccattcttgggtggaggaagggaggaatgaataggtggagtatggggtatgtttggggcagtgaaactattctgtgtgatcctctaatgaatgcatgatattatgtctttgtcaaaacccatagaacttggctgggcatggtggctcacacccgtaatcccagcactttgggaggcagaggcaggtagatccattgaggtcaggagttcaagaccagcctggccaacatagtgaaaccccttctctactaaaaatacaaaaattagcagggcatggtggcacacgcctgtaatcccagctactcaggaggctggggcaggagaatcatatgaacctgggaggtggaggttgcagtgagccaagatcgtgccactgcattccagcctgggtgacagagcgagactccaccaaaaacaaaaacaaaacaaaacaaaaatgtagaactatgcaacagaaagagcaaaccttgatgtaaatcatggactttaggtggtaataatgtgtcaatattggttcatcagttatagcaggtatactacactgtgcaaggtgttagtaataggggaaaaccatgctttgtagagaggggacaggaggctggggaagggctattttaagagctctgtactttctgctcaatttttatgtaaccctaaaactgctctcacaaaagtaaaggctactatgaaaagcaaaacaaaacgaaaacagaaaacagcatccttttagccatgagatgtactacaagtacagagttttctaggaatttcatataatatttttaaaaattacatgaacttttccttctttccttaacacatagatggctattttcacattaaaaattttcccaaaatttccacaatgtaatacacgacccctagggatatgacttttgtaataagaagcatgactttggctgggcacagtggctcacgcctatattcccggaagtttgggaggctgagccaggaggaccacttgaacgcaggagtttgagaccagcctgggcaacacagtgagatgctgtctctttaaaagtgaggttggggaggtgggagagagagagagagagagagagaggaaagaaagcagcatggctgtgattgataggaagtcactgaaattgtatgttaaaaatgaaacaaagcaaaattaaaccagtaaaatcatgagcaaccaatttctctcccaccccagatatttgtgcatccttattttcatttcccagtattcatacaaaagcatatccactacctttaactcactttgtcagcctttattctgtatgcagaaaactttgcaatagcatagtgagaaataggtgataaacccacctttgtggatattacagtgtagacaagggaaatatactttaatatgaaaataagttatactaagaaaagtagatacagaaatgaaaattaagcctgccaaggagacagacaaagtaaatggaatgacctgggttagacaggtttggacgaattttgaaacaaaggacacagttggatgggtgccctgtccttaggagcacagacttttttctggatgtcattgagacctagaatgacctttaactatcaccagtacacattcccctttttggtgtgcagtgttgacactgaaacacagataacttaaggttgtttgggtacttatttatgtagatagacatgtgaacccatacggtacctaattagtcctcttgaaaccacaaacgcgcattcacaggaccagtcttgtttatattttcaaggctttttttttttttcttttgagttggagtttcactcttgttgcccaggctggagtgcagtggcgtgatctcagctcaccgcaacctctctctcctgggttcaagtgattctcctgcctcagcttcccgagtagctgggatcacaggcatgcaccaccatgaccagctaattttgtatttttagcagagatggggtttctccatgttgatcaggctggtctcgaactcctgacctcaggtgatccaccctctttggcctcccaaagtgctggaattacaggtgtgagccaccgcacccggcctcagggcattttttcttatgaagatgacagccgaaatcttcaatcttcaactcatgcaaaagtttgcctttggagtgaatttcttggcaatctgcttggtaagaaaaagcaagtggtgttaaagtgagagcattaaaataaaattgctccaaaaaaatgataaataaataaaagaaaagaaaaaggaaaagcaagaggcccatgcacagatacacagaggagagtggcccctcctccataagcctgtttcatcaatgagagaatcaagagagtatgtatatacagaagagagtttcagggtcagtcttgagtttgcatcttctacttcctagctagaggaccttgaacaattcactttacttctttggtcctagcttgcttatctataaagtgggaataatatttcagattatgcagaagaaaaggtaggatgtaccgggcacatgggaagtgcttgatcaataccaatttgcttccctccttccctgccccttctgctgaaacttcacagctatctcattaagtcatcaatgtatttagtatttaataaataaatatttaacgagcacctactatgagacaagccctttgttagccagtggggatatgactgcaaacaagaaagatacgcctatgatttgatgaatcctggagtttatgagcagctcatctctgtctcccgtgtgagcagtcctaaatttacacagctctccactggtgtttttgaccacagagttaacatttaaaatacctatgactgcttttttggcaccactttgaataatgaactggtgtcaggtaaacagagtccaggtacctattgcagaaagttttccagtctgcaccctcggtggctcacaagttcacatgaagtgaaccaaaagagttttgttgcacttactgtgacaataaatcatgtttagaaaaatgcatgtggcatacatgatattgggcaaatgattcatgaaacttgctattctgtggccttagaatccatgtaagcatttttcctttgtcttttatgtgttttcttgtttctatctctgttttgttttgtttttgttttggggatggtttaaaattaatgctgggctagagtctgattttgacactctcgggtgtgatcaaatgtttccacatatctctctaacctttctattctcaatgtggggaaaactttctccgctttaagaaaaggtgtatccacagaaaagattattgagtaatttttcatagtacagaagtaataattgagtgatttttccctttaaaatttttttatttttgtattattttaagaacaaaattattttaggatgcaattaaagaagaagttcatttacctaaatttaatttgccaacttttctattacacaaaagcattataaatacaagtagaatagcaaaaatttcatcctaaagagaaaaatattttgggcttctaatttcaagagagtaaaattgtcctggaaccctgtgcacactttaaaaacaatcattaacagattgcatcaaactagaaacttcttcaccacaaaggaaataatcaataaagtgaaaagccagcctacagaataggggaatctgtacatctgatatgaagttatggattgcaaatcgtacatctgatatggaattaatattccaaaatatgtaagaaactcaacaaactcaattgcaagaaaacaaataacccagttaaaaaatgataaaggaatatccatttctagaaagaagacatacaagtggccaacagatatatgaaaaggtactcaccatcactaatcatcagggaaatgcaaatcaaaaccacggtgagatatcatctcacacccattagaatggctgttatcaaaaacacagaagataagaagtgttggcaaggatgtggataagagggaacccttgtacactgttggcaggaatgtaaattggtatagtcattatggaaaacagaatggactgcctcaaaaaattaaaaatagaactagcatatgatctagcaatcctacttctgggtatatatccaagggaaatggaatcagtatgttgaagagctatctgctctcccatgttcactgcagcattattcgcaatagccaagatatgaaatcaacctaagtgtcaatggatgaatggatttttgaaaacacagtatatatctgtatatatatacacacatacatatatacacatacagatgtatatgtacacacccaatagaatattattcagccttaaaaagagaaggaaatcctgtcatttgcgacaacagagatgaacctagaggacgttatgctaagagaattaaactgggtggtatttgacacagaaagacaaataccacatgatccacgtggaatctaaaaaagtcgaactcataaaagcagtgagtaaaatggtggttgccaggagctggagggtgagagcaatggggagatatcagccagagtttcagttatgcaggataagctctggtcaccttctaaggtgatcatctgctaagtataggtcaccttcagcatggtgactatagttagtgatactgtatggtatatttgaaatttgctaagacagtagatgttaagtgttctcactacacatattcatactcaaaaaggtaactgtgaagtgatgaatatgttcattaccttgattatgataatcatttcatagtaaatacgtatattaaaacatcacattgaataccttaagtgttttgtttgttttttggttttttgggtttttttgttttgttttggtttaggttttttttgaggtggagtcttgctctgtcacccaggctggagtgcagtggtgtgatctcaccttactgctacctctgcctcccaggttcaagtgattctcctgtctcagtctcccaagtagctgggattacaggcgtgctaccacacccggccaatttttgtatttttagtagagatgaggtttcaccatgttggccaggctggtctcgaactcctgacctcaggtgatcctcctgcctccgcctcccaaagtgctgggattacaggtgtgagccactgtgcctggcctgtataccttaagtttaaaaatttttgtctattatactcaataaagctggacaaaattttaaataaataacagcagtcattaacagactcaattgatgacctaatgtagaagttaatgagagcaggcctgttggcaaaaaggcatttatatggatacactgtatgtatctgcactgtttcaggatcctctattatgatacctgggtaaagggtgacttcctttctatcataaaacagcctagacagcactaagaaggtggttatgttcttttctgttgttgtgagcgcccagatgagattactttgccaaagactcttttcatttctctttctgaaactctgtgagattgctgtgttctctaggacttaacttcacagctagtctatttttcctttcagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtatgatgtttattttcacttttcagactactaggactagaattggactcatctctcagtaaccctgtaataaataatgctccagagactagaaatctaatagagaaggcattcaccattcatttattcacccatttatccatgaaatattaggtatctatgaaatgccaggaagggagctggcacgaggtcaatcttgccttccccgccccctccttccttcctctcttctttcctttcttgcttacttccttcttagggacattttctaccttgattacactgtcagttgtatctcaggctaagagtccatcatccaaactttgaattattttggccccttataagttaataagtaacaattccaagtcactttgtattatataacttatcttcactatattactgctactacagtacattttttctagtatgtcctttcaggagtttatactgaaacctacttttaagcacaaatttgttttaacattgtagcctattgataaattttcatgccattaactaaatcaacatcaaaacatcagactcaagaaaaactagaactgcactttttgagataaaatgtatttctgtgatgctatttccttgatttttatcatgtgtgggctgacatgctggttttccaaagccaccactatgtaattggcaatcattacttgacaagtaagaacttcctgtatttctagtggcttttaaagataaataatatgcttagttatatttatctatgcggttttcagttagagcctaatatggaaatttagtagccctgagcccaactagtaaaaggtagggcttacttccataagttccgccacgctctgtcaaagattatttcacaaaccacctcctttgactgaacactaatggtaacctgggctcatatttttcatgcacctcaagctaggcataccatgaccttttcacagtggtgttccttctgtggttcttttgcaagttgtttcaaagtacttataaaatggaatatttaaataaaatgtataatttaatattgtgatgaataccaaagacagaacatttcctagcaaaataagcttccttgtctcaaatcctctatgttgtctgttacagctgtagtattgcaggctactgggggtcatagaaccacagacttaattctcattcccattttatatttacaggtgaggcttagagatgttataacttgcctgtgattacaaacctttctttttcttgccttaactccagtgttttttcatgttccactgtgcttttacactgaaacaatacaaatacagcatgaaggtgtactatggtggcttgccctgttgcctttttaggactgttttcatgggataattatcctctcacatgtctccatacacaggtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaaggtaattaaaaaaaaagtcttcaaagaaattgttgaaactttattatttcatttcagcagaaccccttttttaggtgatacaatatatgaattttttttcttcatggaagagtatcttgctctgtcacccagaccagagtgcagtggtgctatcctagcacactgcagccttgagcttctgggctcaagtgatcctcccacctcagcctcccaaaggatgatataagatttgaaagcctaagaaagtggataaaagttgggctgctttgccccacatttgtttctccaaattgattctttgagacactgatctaatttaatccttgcatcttgcagatttttacaaaaaggtgacttctccaaggtgtcatggcaagacagtgatgacctggaaccaggatgcagatttcctgcattctctagttgtttatttatttatttatttatttattttgctataccgtgtaatattgtatagacagaagattttacagattatttatatatgcatatctttggatccctagagcaatcctataagattggacttctttatggccacattgcagatgtagaaattacaggttagtaaaacttgccaaggtcatcccttagtaagtggcagaccgcagtctagaccctgactccttaggtggtgttggctctcaaagcagtgcctcatgtttccttattccagcatcaatgtgagccacatacacggacaggtaaggaggtataacagtgtgggaaaggagatactatttatcctcatctctttcctttgtgaaaagggtattttggaaaattcagacgtttagtaatttattgcttaattattaagaaaatatgtgaatccactcatattttgatatgcagtcttttaagataattttaatatacacaatgcaaaaatagtactaatttattatatacgcattgcacagaataaactgttcataaatattcactttataaaataaatacctgcttattgggctttttattcaacgtaaagatacagaacaacagttaggaaagttttaggacatcttggttcctaaatggatggtgggccaaaaaaggaaactgatttttattgaattcttactacatgccaaatactgtatgaggtaacgttttcatacatcagtatttaaccacatcctacccaggttttctcatctataaaatgattgtggctgagtgtggtggctcacgcctctaatcccaacactttgggaggccaaggcaggaggatcacttgagcccaggggttcaagaccagtctgggcagcatagtgagaccccatccctacaaaatttttaaaaaattaaccaggcatggtggcacatgcctgtagtcctagcttcttgggaggtttaggtaagaggatcccttgatcccagaagttcaaggctgcagtgagctatgatcataccactgcactccagcctgggtcacaaaagcaagaccctaactccaaaaataaataaataaataagtaaataaataaaatgacactgaattacatgatctctaaagtcccttcaattgaaacagtttttactctaaaggcaacctaatacagtgaaaagggacaaaccagaggtcagctctgccactttccaaagtcattagccagtcacatacacattgtatccttcatcaggccccccatatgtaaaatgacttggttagattggctacacaaggtcttattcatctctaagttctgtaatttcaaaatgacactttgaaacactctggcaagagatgctttcatttaaaacactaaaacccgttcgttctgtaatttaaaccaagttaaaatatttatgaccttatatgaaatcattgtattaagtcagttatctaagatcttgatgagtagtaagaacatgtcattttattgacttagattttttattgaatgttttgtggttttattgagttttattttagatttaatgagttagagtttatttttactgagttggataccatttttgagtgagtaaactgttgctaaattctgagccatatttatctgaagaaaccattctgttttggtcatgttgataatttacaactataaataaataaaaacagcatggttggtttaaggttccacaataagttattgttttttatgggctagtaaagcacttagaaaactaaaacttttaagcaaatagttccaccataatcctattagtcacaaatttgtggaaagctaacattactgaaaacagctgaaaatggaaagacaagaacattgtcagtagttggttatagtgataaatctgagttctgagggcactaaaacttaaaagcaagcagggaagaagagtagcaaaaagagaagagagaggaggaaatccaagtgccttcaacttaggattagaaagggtagaaagcagggagggagaaaaaatacttaataaaacctgaagactctaggggagtccttaacctatcatagattaaaaagcaaacatagaagaaaactaagcgtattgtatactaacctgcagtaatttgcaaaccatccaaaatgtggattccagaattcctagactaaaatgtaaggaaggttttactcccttggaaagggaaaatagacatctacacgttttaaaggtaagaataagaaaaatattattcttggaaaatttaaacatttagtaatatactacttaatataaagaaaatatgtgaatccacttatattttgatatgcagtctttcaaaataattttaatatactcaatgaataaaatagcatttattgtatactgattgcacagaataaatttttcataaatatttaccttataaaataaaccctgcttcggctggggtttttattcggcgtaaagatatagaacagcagttaggaaagaattaggacatcttgattcctaaatggatgatgggtcgaaaaaggaaactttttttgaattcttattctgatttttattgaattcttagtatgtaccagatactatgtgaggtacatttgcatatatcattatttaaccacatcctactcaggttttctcatctagaaaatgattcagatgatattggtaggatgtggttacataatgaaacattatcatttggcttcttcaagtatacatcagcagttcccaaagtgtggtctggggcttggaccctttcaaggcatccatgaagttgaaactatattcataataatgatttttattacttttttcactttcattctctcaggagtgtatgtggagtgttctggaagctacatgatgtaggatgatgacatagctctggtagctaatggaatgtttgcttatgtgttcttgtatataataatttctcagttgtaatttctagtgtgataaatgtcagtaaagatagcccacataaacaaaatctctttggggtcttctataattttaaaaagagtaaagggatcttgcaaccataaagccttcagtctcattctgaagaaatgcagttactcattctttgctactttgaaactagacaaaattgctgccacattctgaatactgctgcattagaccagtggctgtcagtaaagacatctccttagcttataaccaatacgcaataaaaagccaggaaaatggaattagcaaatgtgtttaactcatctgcatgtattttctgagaggtgttttggaagcttcacatttgctgttgtagtaaaatgtgggttatattcataattaaaatacagttgaccacaaagctgcaggcttatctatggattgaaaagaataaacacagcataaaacaacttttggtagatcttcctaaacaattttacaatgtaaatggaacaggtttatattagtgattcttgccaaaaattcagtcagacagatgagaatgagtagattgggtttgaattaaggtgaaatagttacagattcttgggtatgttaatattgcaaacttatgtaaaataaaacctttagcaactgaaagatatatataatttagagcttggaaagctatcatttatttgttgtttacatgttggagatatatttttttctttggtaagttccatcctattttttatctttagtgaaagatcattttggaaccatagccgtacctaaagatataagtaaatacagtctaccatacaatacaatgtaatctaagtttgtatttctgtatacaaaagaatgccgtatgcttgttagccacttgtatgtctattcatccctacttcagaaagagttttaattcttctcacctgccagtcgtagcttattaatccaacttccaggggccagactgcttcatgggaaagccaactgcaagcccagcacagggctgttaatttcattgtaaaatcaacaacaccattctgaggtctcattgtccctgatgttttgttaaaattaaagcaattaaggagagatcaaagaaaatttttcaaatgatatgaaaaatttgtctgttactttctagcagacaaatgtgcagacattagaataaatatatacacaaaacaattttctcagatggtgactgatgcatctctaacacaccacatcacagacttcctgatcatcagaagagtaagttgttgaaaatacattgttctatggactctaaaataataaaaaagtgttattagagtgtttcttcatcagtaatatgaagaaaatttggagaagccagccttgttctgtaaatcgataaataattttacagaaaagcactcgataatatccttacatcttcctctctagattccttgagtcacataacaagaaaacacttggttatcatgaactcggtgtaaaaaaagtaaaagcaagttccaagatgcatgctaggatctgtgtattcagttcaagattttatttacatatacagtgtataatgttcagaatgattattcattgaaagaccttggatcatcacaaaaaatgtgatacacttttgaaagtcatcagctgagtcatgtcagaaatttccattataagagttaaacttaattctctttttttttttttgaacatgtgtcaaagatttatttaaaacccattaacgagggaaaagcaggatggtaaatctagttcaaagaagaacttgaaggaccaagaatatatagtcactgaaagaaagaatcctgagataagattcagaatggttaatgttctacagggcaataaaaccataaccttgaggttatcttttcttttttttattattcttatactttaagttctagggtacatgtgcacaacgtgcaggtttgttacatagttacacatgtgtcatgttgatttgctgaacccatcaacttgtcatttacattaggtatttctcctaatgctatccctcccccaaccctccaccccctgataggccccactgtgtgatgttccccgccctgtgtccatgcattctcattgttccactcccacctgtgagtgagaacgtgcagtgtttggttttctgtccttgtgatagtttgcttaggatgatggtttccagctccatccatgcccctgcaaaggatatggactcatccttttttatggctgcatagtattctatgatgtatatgtgccacattttcttaatccagtcaatcattgatggacatttgggtcagttccaagtctctgctgttgtgaatagtgccataataaacatatgtgtgcatgtgtctttatagtagcatgacttataatcctttggatatatacccagtaatgggattgctgggtcaaatggtatttctagttctagatccttgaggaattgccacactgtcttccacaatggttgaactaatttacacttccaccaacagtgtaaaagcattcctatttctccacatcctctccagcgtctgtttttttcctgactttttaatgatcatcattctaagtggcgtgagatggtatctcattatggttttgatttgcatttctctgatggccagtgatgatgagcattttttcatatgtctgttggctgcataaatgtcttcttttgagaagtgtctgttcatatcccttacccactttttgatggggttgtttttttcttgtaaatttgtttaagttctttgtagattctggatattagccctttgtcagatgggtagattgcaaaaattttctcccattctgtaggttgcctgttcactctgatgatagtttcttttgctgtgcagaaactctttagtttaattagatcccatttgtcaattttggcttttgttgctattgcttttggtgttttagacatgaagtctttgcccatgcctatatcctgaatggtattgcctaggttttcttctagggtttttgtggtgttaggtcttacatttaagtctttaatccatcttgagttaattttgtatatggtgtaaggaagggatcaataattctcttatcaaaaaaaccttcctctcctagccatgccaaataactgagcgctctgtgagccatccagctgtcagctagccaagtagcattttcacccactaatattagtctttaaaaagcaagaatcaaaagagaagtcaaggaatatcataaaatatctatgatcacaatggcaaacatcaggattttcaccaacatttatcaatgagaacagttctatacattagcattctgttcatctgtgaaaaaaaaactttttgatatctgaaggtcctcagaaccctttgagaattaaagagacttcaactgccagcaaattctacatcgggcatattctttaagacattgctgatctcacaagaggtagtgaaaactctccaaaacaacatcaatgaaacaaaatgaaacaaacataccctcagttccaagctgtgagctgcaggcacacttagcaggtcagggttttccccatgatagatgcccagagaggtacagtggtgagcattgccccaaaggcagatggctgtagcagttttctgagcaataagtgagaaggagctaagtgtagggcttctgaattaagccaggattctcaatagtgtctgaaatagtcataagtcagtaacacccctggctactgaattcctaattcagactttgtaggagtcccccagtttaaggtcaagaaactagttccaaataatatcagcaagcatgtgagaagctaaccatcttgaatgacattcatcatcaacaagaaaactgatacagactccctgctcttcccctacctggggaaagcagaaattgggattgcctgatacagaatataaaaaagctattatgaattatttaaaggaagagggcataggcttgcaacaataaactattaggaataaccaggatgatttgaaaaccaagtgaaaaaaaggtgaagcttctaaaagttaaaagtataattattgaaataaaaattcagtggaagggttagatagcttactggacacagcagaagaaaaacctgaaactgaaagagatatgcaaagaaattaccagaatgaaagcaacagtgtcttaaagaatatgccctatccaggatctgctggaatttgaaggcttttagttcttgaaggattcataggatctttgaatattaaagaggtatgtcttcttcactgatcaatggaagagtaaagtgcagaactgttctcactaatgaactttagtgagaaatgtttgtttttacttttgccagtgtaattatatattttcatgttattttgcttctgttctgatttttactttggcaatcaatattagtgagtaagtagtgagaagaagatggaaaataaaaaagaggcattcagaaatgtgaaggctagaataagaaagtctagtacttatctaactggagtccagtactgatgaacatgtgaagccgtaatgcataaacaacatatccaagaaggtaaatgaaaagaaattcatgcccaaacacactgcggttaaactgccgaatactaaagacaaatagagagtataaaagcaaccaaagagaaaaggtagatcaccttagaagtaaattgacaagagacttctcaacagcaaaatggaagcagaaggataatggaattcaaagtgttaagagaaaatgactgagtacttggtaatctctcaagaatgagagtgagccaggcgtagtggcacatgtgtgtaatctcaggcctttgggaggccaaggtgggtggatcacttgaggctgggagttcaagaccagcctggccaacttggtgaaaacccatctctaccaaaaatacaacaaaaaaaaagagccaggtatggtggtgcatgcctataatcccagctacccagaaggctgaggcccaagaatcgcttgaacctgagaggtgaaggttgcagtgagccgagatagcaccactgcactccagcctgagcaagagagcgagactctgtctcaaaacaaaaaacaaacaaaacaaaaagagtgaaatgaaatatctattggttgaataaacaaaaatactgaaaaagtacatttcaaggatagtaagccagagaagatatttgaacatctgtaacagacaacagattaatatctagcatattttttaaaaaagtctaaaattcagttttaaggaaaagacagttagaaactgggaagtgaaaagccagggacatacaaatcaagatttaaattcaattttatacatttcagattgtcaaaattaagaagtctgacagcaccaaatgtggaagagactacaattttcataaagcgcttgtggggagtgtacattggtagccactttggaaaacaatttggcattatcttgtaaaattttaatactcacacattgtacaacccagaaatgccacttaaacatatttacaggaatatttagagaaactcttctcataatagcaaaaaattgatatttcaaacgtccatctgcatacaaataggttaataaattatagtatagtcatacaatggaatactatataattatgaaaatgagtaaatcatagtctttcactaacaagaacttaaatgttagaaacctcataccaaattttaatagtgtcccagaaaactacatatagtataataccatttaataaagcctccaaacaagcaaaattaaatagtatattattttgttttacatagtatgtgataaagctgtattttcttcctttttttttttttttttttttgagacaatgtctcactctgtcacccagactggagtgcagtggtgcaatcataacttggcttactgcagttagacctcctgggctcatgtgatccttccatctcagccgccagagtagtagctgtgactacaggcgcatgccaccacacctgcctaaattttttgtatttttttatagagacaaggtttcaccttgttgcccaggctagtctcgaactcctggactcaagcgatcaatccgcctcagtctgtcaaagtgttgggattacaggtgtgagccaccacacccagcctatattttcttaaagatcaaggaaatgacaaacccaaaattgagaataatgattactttgtcaaacctatcgcctttgtaacatctcagtcacaattgggaatcacaaactaccagagatggaagtgctttctcattttatagttgaaaaaggtgaggcccagaaaggaagttacgtgacatctgtattaattccacaaattttaatttagcactatcataggcatttggattccatccaggggtataagagactattcctgccgctgaggaacttataattttaattgagcagtgagggaggggcagaaaataaacactaaaacaataataagtaaattctgtggtgtgttagaaagtcataagtgctattggaaaggagaaaaactggagcagagtaaagaagatcaagagttccagaagagaggtggggcagggacaggccacagtattaaataggttggcttagccccattgagaaggtgagatgcaaaacttgaaggcgatgaaggaaataggcaagtgtgtatttagagaaagctttcttcttgaggaaagaaagaagtcaagagtgatttcaaggattttggcttgagaaactggaaggatggcgttgcgtctgctgagatggagaaagccgcaagtgaagtgagtttgtaggggagggtcaggacttcaatttggacaggctggcttagagatggctattaattatttaaatgaagctgtcaagtaatcagaacatactcatccagagttctaaatggacgtttggagaaagtcaaaatgcatgtcaatgaccaaagcaatactaatgcagtgctttttgcaccaaacagggccagatacatttgaaagaattagcaattaagttttatgaagatgaacacaatagaaatgaataggaatatgatgaacccaaaaaatggagtcagaaaacacgtccagttcgatttttttcagctctttccaaagatgtaaatatagcagaatattcagttggcctcaaattgacatatttgccattttgagatagcagtcgattttgtgaggatttatctgattacttctcatgttattcccatagtctctttctgtctgtaagccacaacaatgatggggcacactttaaacaacataaataagtgaaacaaattctgcaggaaattatgcaacatagagaattccaaaaaaattatccacatgtggatttttattggccctgatcatgttttagaatgaaatgattataactttgccaggctgcttagcacaattacaaaggctggggcatgctgttggaatgatttgcagagagacttaactgattacacggtgttttatttgcattacattaaatccagagcacttcttagagcatgccgaaaaccaaatgtagagccaagatccaatgaaagccgcagctggatgtggtcagtgtctaagagctaaaaaagggacctggagatcctctggttctttattttatagcagagaacatttagttccagagaggttaagtgacttgtctaaagtcaccagtgacagaatcgtgtcaagaacctgccttttctgattcccaggccagtatacctttcgctgtaggttgctgcttccatgtgtcataaagacttaaaatttgcttcttttttatccctttgcagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatggtaagtaaacagtcatcaccatatactttattattctcattatagctgccagaccagtggacactaaagccattgccaaaaatgtgtacagtttttccaccaaatgccagaatttagaatattgcatggcgataaaacatttctcttttaggtcagtgtttttaaagttttattatagaacctttctctctgtggttgggcatctgccatgaggagaaaagagacttgaaaaatctgggggattatgggaaaaactttagtgttagaacaaatataatgatcatcatgattaatttttatattgcctcatgcattaaaaacagaattaaaatttacttttgtgtatcagtagtgtcgttctgctttaaatgtaagaaaatcctgaagatgtctgggtctgaaactttaaatggcatatgagaatttagattcatttaattttttttttttttttttttttttgctgtttattatgcaaagacgagtattcgtttccttcacttcaaggaacgtccatgagttgtggaaagggtaaaacaaattatttttatagttctgcaaaacttcaaagccatagaaacataagagaataaaactttctacttttgaaacctgtgtctaaagtcaagcttatgactttgggtgtcaacttaagggaaatgatgtagaagacctgagaaaagacccatgttttcatgatctgtggaactaactcagtaaggtcctggaagacaaacgtgtttgaaaaaataattcttggtcactatttggttctgattggttgcctggctgacaaaataaattttaggtgtaacttgatagtattatatgtgtctagtattaatcagttattttcaatgcactcaaggaataaaaaatgccaacatgatcaaattaaatttgaagtttcatttcatcagctatcctattttttctttcttttgttttcatctctctcaaaactgatctagtaagaaggcagtcttcatctatatgtactctaaagcatctgagaaaattaaatttttgctctatcatgcagatacttggttttcacagtggaaaccatcaagatatttgagagtcactcattgattaataacctaccctttaaaaatagttcacaaaattctatctgtatgattactaatgggtcagttcctctaaaggcaagaatgtattttgaatagagctatcagcagtactgaacaggatggaaatttggtccaactttcctcaacaattgactacatctgctgtatacaactgtagtcatggattgcaaaataatttgctggttcagttccatcagtttcctttttatttcagacctcataaaacataagggcaagcttccaagtcaagggttgtattttaggtgatgcaatcaagttgcaataaacttatttcatcttcatgaagaaatcattgactttgaggtcattatcactgagacagcctaggaaagcatgccagttccgcttttaacactaatttttgcctattaaaaaattatcacttcacacattcatgtaaaatataattcctgttacttacaggcagtgcatacattgatgtaaaatatatttcctgttgcttacaggcattgcatacgtttacttaaaatcaatctgcaattgcttattgtttataggcaaatattatttaaaccccaacctatgtagtatggatggtataattttctcctcccataaatgtcaaagaaagctatcataaacttgataggtcatattttgacttcatatgacaatttattttatttctagagagggtcaatcttgatagggagtgtttgttcagttctagcaaaagcaagaaaatatatgaattaagtgtcatgacatcagagacttaggcagttcaacacttaaaaaattgcaaacataactaagaaaacctcagaggtttttatgggagtttgtttgctgcacagaaaataaacatcagactaacaacaaggttttgattagttttgcctgttagctgtatgaagaaaacatttctaaatacaaagttttctaggaagttgagtcactatttaaaatccattgctttgaccaatgtgaagccaacagggttttcattcatatagaatgcctttttatcaaaagaggctctgagtgttataccaaaatcagcacccagctgttttcagtatgcgcaagaaaacatgaacgtcccccaaagcaaatgtactccattccgtgttaaccttatttctgctccctggtctctgattagtctggttgatgaatgtagcttcccctttggccatgcactcacagctttgaaggaggcttctgaaggaactggtagggaaccctaggggctcttgctaatgttgttggatagtcactgaaatcagtgatcaccatgtcctttcactttgaaaattctatgattctgtataatcaaacaacctaactcactggatttttcacttttcaaccagctattccgtgtcttacagtgctctggtcttcacagataggtaggcacctggcatgtcagttgcctgaatttgaaagttttcacctgtatgttttggtacgataaaaataaaaatgtaatttatatatctgaatcaggtctgtatgttatgatcaattgctcagcaatttcgggcagttggtttgatggttatgtagtaatgtagcctgagagcagaaatacagagcctctgggctagagaaagtataaatggcatcctaggctatgtagggttacagctcttcagaaggaactttcattttcattgtgacacatcgtctacatgttgtagaagaacatagtttcagaattcttccagttagaaacatagtttctcaaatattcactttcagcattgggtagaaaaagtacccaggtgatggactgattgatatgcttcagaaatatgttttttcctgcaagtaatgaaaataaacacacgttcttaattattttggtcatttaacaacccatcatttctggaccatgggttaattccaccgattgctcagaagccttcctgagtcatctcaagcacaatcaattcactactgtgtctagacactgcccttagaggaaagctcaaaatgaaaatgaagcagaaatacagacccacttcctaggccactcagcggtgtatgtaacttaccaagtgatcattgcatcaactactctttgttagtgaagctggtgtctgcagaatcttgataaaaagactcatggataatgttgcttctttagtcaaatgggctcttgcatgcatttttatagattttcaaagtagtcatttttgttttaaaatcctgctgccaagtcctttatagtaattgcattttaaaaatatgtcttctttattttttattattatttttttttgagatggagtctcgctctgtcgcccagtctggagtacaatggtacaatcttggctcactgcaacctccgcctcccaggttcaagcaattctcctgccttagcctcctgagtagctgggattacaggtgcccgaaaccatacccagctgatttttgtatttttagtaaagatggggcttcaccatgttggccaagatggtctcgaactcctgacctcaggtgatccacccacctcagcctcccaaagtgctaggattacaggcgtgagctaccacactggccttgtcctctttatttttaataaaattatttagtcattaagaattttggcttgtgggccaggcatggtggctcatgcctataatcccagcactttgggaggccgaggtcagaggaccacttgagctgaggagttcgagaccagcctgggaaacatgacaagactccatatctactaaaactacaaaaaattagtcgggcgtgatggcttgtgcctgtggtttcagctactcgggagtctggagtgggaagatcgctttagcccagggggtggaagttgcagtgagccatgattgcaccactgcactccagtctgggtaacagagcaagacccagtctcaaaaaaaaaaaaaaaaaaagaattgtgactagtgaataattcatattcagagctctttttaatgtatgaactaaataataaaatagtaagtttaggctaagtgccctttttatttttaaaattttctgtatttattattactatttttagagatggagtctccctgtgttgcttaggctgggctcacctcagcatctggagttgctgaaactataggcatgtgccactgcacctgacctatttaagaaaataatctagagaagaattgacatgttatttatgaatcattcagttaggttttctctataacattgagatttggttaaaaatatgtttttcataaatttaaaaactattgaaatcttagctttgctgactaggatgtcttattatataaatgagagagagtttgtaattttttaaaaaaaactgacaactttcaaaatagtttctcagtggcttcataaaaatccagaaaaaaagatttcacaaatttttgtgacactcaacatttttaaggtagttctctcagtgccttgcaaacagcagctctagtgtatattgagaaaaggaaaaattctgaatggcaaatactagatttcaactttaacctattcctttttattattattattattattattattattattattattattattattattgagacaaagtctcgctctgttgcccaggctggagtgcagtggcgcgatctaggctcactgcaagctccacctcccggattcacaccattctcctgcctcaccctcctgagtagctgggactacaggcgcctgccaccatgcccggctaattttttgtatttttagtagagacggggtttcaccgtgttagccaggatggtcgcgatctcctgacctcatgatctgcccacctcagcctctcaaagtgctgggattacaggcgtgagccaccgcgtctggctaacctattccttttttttctttagtcttcttggttgtgtaaagaaaaattacccaaatcttaactttcagaaaaggtaacaaactcctaatgcatgtggcagtacatgtttgctccagctgccctgtccctctaccctgcgtggactccagaaccgcccatgttggagcccacctcctctccccacccctgcccactttccactcacacctcaggcaaaccctgcattttgctgttcctggaaaatggcttatgcttggatacctctgggcctctgttgatgcgtctgtctgttccttgtggaaattcttcctgacattcaaggctgcattcaaatgccttccttcccatgagacttttctgattccctgctcttccttttctatgtgtttcataccttcctgtgatatcacattctgccttaaaaagcaagagagtgatcagatacatattaccaaaaaaaaaaaaataccgatagcagtaaaaatgatagagaaaagactggaattgggaaatccatctgaagaccaatgcaatattaggaaataagacattgagaagggtgagaagaaaggaaattgagagaaatatcagagcagtgaccaatgaataaaagcgcctgaagggagagaagttgtcaaagattatttaggatatctaatttgggtgatagaatgaattaaccaagagaggaacacaggaataagagccagtttggaggagaaaattttaaattcagttttatattgtgttagagatgcctatagagtatccagcaactacgtatagaggaatataggaggatgtgaaaaataaggctccaaatttcagtgaagagagagcaggaatcatcagcacccattgtaggttagagaggtcacactagggaccacagaatacaagctaagggagaggcaccatcagagagatctggagagcagctggcagaatgttagaggacccagaacagcaggaaccaggacaagaaattttggaaataagagggcggagatggtcaccagaattaaaggctgggagaatactgaagagctcaagtctattgagaacttagaagaggccattagttctggccgagagaaaagcattcaggatttttacaaagttttggtaaatcccagtgagcgcaaagctagactgcagtagatcgagaagtgaatagaaagtgacaaacacagacggagtgaaaacaactctttcagtaagttcagtggtggaggaaagatagcttaaagaggaggtaatagtagagtcagaaccttcaacctgggcagaaaggccagagccagtacagagaggcacactagagaaagactcatctgtgtaatatacccagtctcccctaccagacatgtcctttccatcctttcccccagccccatctgtgcagcacagtgttgcatcaacgaagtgttcagtgaatgaattaaaggttgtcaggaagacttcagggaatcagagattgagttagaaggtgagctggtggtgttttgctcatcttcatttttgaaagaaacctaagaaaatgtttaaatgtcctctacaaatagtgtgaaaaatggtacctctgcataaaaataatttaatattagtgctaaagttgctccttgttttcctttagaggaactacaaaaggatagttattagattgccaaatcctgcagaacctccagcaggcacaagttggtttttctccctcaagaaagagaagtgagccaagcgtggtggtgcatgcctgtggtcccagctactcaggaggctgaggtaggagcttcccttgaacccaggtgttcaaggctgcagtgagccaagatcacactactgtgctccagcccaggcaagagaatgagaccctgtctctttaaaaaaaaaagaaagaaagaaagaaaagaaagagaaaggatggaagggaggaaggaaggaaggaagggagggagggagggaaaaaaggaaaaaggaagggaggaaaagaaggaaggaaagaaagagaggtgagccaggtgtagtggctcatacctgcaattccagcactttgggagactgaggcaggaggatcacttgggccaggagttttggcgagatcccatgtctaccaaaaaaaaaaaaaaaaaaaattagctgggcatggtcatgtgtgcctgtgggcccagccacttgggaggctgaggcagaatgattgcttgagcccagaaggttgcagtgagctacgatcatgccactacactcccacctgcgtgatcgagaaagaccctgtctctaaaaaaaaaaaaaaaattttaaagagagatgggaaaacaacttattggcaatagcacatgttcttgtttgtatgcaacaaacatataaaaagtagaaaaattgaccataagacatagccttactatttttcaaaagcctgataatgattgcaaattcatcccttagatgtggaaagacaaaaataagacttgtcttatttcattctgttattttctattgatttattattttgaaaacctgctttaattaaccttagtaattaagtagcatttgtctttaatgcttctacaattaggtagatgttttaaaaacatgccgattaattgtttcttagcgttatacccagatccaaaaagcaggacatctaactattccatgtgcttggtgtttgtgtgttcagcaatatcagtactaaatacacaatacaaatctataacatgctagtagtagctacattcagctcaatagaagtgtgttgtataaaggaatgccaatatgctttcccagaagttaatgagtctctatcaaacccagtcttcctgaagggagcctaccagctctgatatggataattttttttaccaagataccagatttctctgtaggtgatgaattattccaaataggaaactgcgtgccagtgttctggaagttgctctgtgtgacaggtgccagatctgtagaacagacagaatgtaattttttttgagcctcaaaaactagacaaatactgcttttaaaaagagagcagagcattttcttgagtactatgtagacatttgtagaactttagagctaaatttagagctagaggaaggaaactttagaatcttcttttctaagttcctcattcagtaaataaatcagttgaagaccagagaagttcagtaattttcttaaagccacatagctagtttgtcactgagctgaaactagaaattaagcttcctgactgcattcagtgcagtattcattcccttgctttccagagaggccaaaagttcttgctcagtgactcacagtaagacatgaagcattttccttttaagattaatcagcagtggtagaagatttggaaaaatacacttgtatatgagtacactcagcctttatagttctgacccccaatgcagaatgttcatcttctatttctaaggtctgacttcagtctttcaggaaacacctcccaacctccttcgctttcctcctcttggttgtcagattctccacatgaagggtagaacctaaggctcaagaattacttaactaaaaaacaccatctcacaaacaatttaaagatggaaagatagaaatagagataggcagacagatggacacatagattaggttttcagattctgttctttctctgtgggagaggtcagcgtgccatgactaccagcctggtctagaacccacagcagccatagttaccaagaccacagcaataaaagctgtgtttgttaagcattgccatgcacctagcactgtgttaaacacttcctgtgcctcctctcatcacctcacgtgaggtagataggtacaattatgtgggactcaaaagcaacgaaataataaaactggcttttttcccccttaaaaaaggtatgcgtgcctcagctgaaagacagaaaaaaaaaaaaaaagtaggcaaataaaaatccttgttttctgtacaatttcagatttgtgtatgttttatgggaatttagtctcaccaaatgggatcgatttttcattccttttatgaacttctggtgtataaaaatgttaaaatcacctatataattggttgcactggtagttaaatggaacctaatttgtagcatgggcatttaacaatacagataatttattcattagagttagcctttggggggaacttcaacttacaaaaatacgttttagtgagattatagcagagggatctcactttaatatatcctaattctaaaatggtttttcggggtattttttggttatatttgttaggtgatagaaaagcaagactagtaaatgtagtaagtggctatctttggctgtttgtccatttaaattaacttttttgattacccattaggaaaaaattgcttttctaggtgtgtaaagttttgcatatgattatagcctcttacctgtaaaaactgaatcagagcttagaaccatgcctggcgcacaatagtcattcaacaattattccttgaataaagagacagtattgtgtggtctgtgaaataacaaaagttcaggagcaacagacacttggtgattctagtcacctctattttgttataggataactggttttctctgagttttacaccagaatgccttcttcttttaatacgccttcttacttagtgcttttctcatgatcattttcccaaacttcctaatggacatagtgtctcattttctgtctgtttttaaacctgatactttggagtttaatccatcaaacatttattgttaacctactatgtgtattttgttacacctaggaacaaagatgagaaaacatgttactcatataatttatattcgagtagaataactgatataaaccaacatcatagtatagtaacagtttaatacatgagctaatgtatgcatacaacaggagcccagcatttaggaagctttttataaactgtcccaaaccccaggttttttatatactcctaattgagtggatggatggcccatcaaaatgtcccgtggaagacatataatgtcagaagttaccgagtgactctgataggctctgcctcccctaccctccgcactcctcaattaggaactactggtgaacaagaaatgaatgtagtacagccatccctccttattccctgggattggctccaggacccactcagataccaaaattcctaaatgtgcaagtctctgatataaaatggcatagtatttgcatgtaacctacacataccctcctgtatactttagatcatctctagatgacttataatatctaatacagtataaatactgtataaatagttcctatggccaggtgtcatggctcatgcctgtaatcccatctctttgggaggtggaggcaggaggatcccttgagcccaggagttcgagaccagcctgggcaacatggtgaaaccctgtctctacaaaaaaattaaaaattagccaggcgtggtggcacacacctgtagtcctagctacgcaggaggctgaggtgggagaatcgcttgaacccaggaggtcgaggctgcagtgagccatgattgtgccactgcactccagcctgggtgacagaacgagaccctgtctcaaaaaaaaaaaaaaagttgttatactgtatttttttatttgtcttattttttatcattgttttatttatttattttagagacagagtcttgctgtcacccaggctaaagtgtagtggctctatcacgactcactgcagcctcaaactcctgggctcaagggatccttccacctcagcctcctgagtagctgggattagaggcgcacaccaccacgcccagctatttatttttatttttatttttgtacagacagggtctctctatgttgcccaggctgctctcaaattcctggcctcaggcaatcctttcactttggcctcccaaagttctgggattacaggtgtgagctaccacacccagcccatattattttttattggttcttttttccaaatattttcaatccacagttggttgaatctgcagatgtggaacctgcgggtatggagggccaactgcataaggaattctatctgagcaaagtggtcaggaggtcacagaggattttgaaggaatatgctatctggggttttctttccattgtatttcaaaatacagtagacaaggacttaatatgtgccagaaattgtactaagtgctgaggatacaataatgaacaagattcctgcctttgtggagattatatggcaagggaggaaagaaaacattatagattaattcctgtgatcagtgctgtgagataaggtcatatcctgagggacccagacaagcctagcaccaggggctggggtcaccagtagataaaaatatgaacaaccataccccagccccatggaacaacacagaaagaaaatggggaagagtgagtagggaacaggatttttgaccaaaacctcctagtttaaaggtttctatatgaagaggccatgtaccaaggatatattcactaaaagtatctaaatataggctaattcaatttaaaggaatcccaactgagaggtaagattttcttcccctctaacacccaagtctggtctctaaggaggcccctaccaaacaatagttgtattagtcagcgataaaatttatttagggtcaatggaaatgaatttatgaatttgagactcaagggtacatgtcagtttcttcttgggaaaatccaaggtaatattcacagagtactcagatgggaaagttgtcgtgtgctgtcatgtctcatggtagaaatgctacccttagcataataacatataaaataactatctctggctgggcacagtggctcatgcctgtaaccccaacactttgggaggctgaggtgggtgaatcacttgaggtcaggagtttgagacccacctggacaacgtggtaaaaccctgtctctactaaaaatacaaaactgagccaggcatggtggcccaccccttgtaatcccagctactcagaggctgaggcaggagaatcgcttgaacccaggaggtggaggttgcagtgagctgggattgtgccactgcactccagcctgggcgacagagcaagactccatctcaaaatataaaataaaataaaataaaaatctctttgttctgggattcagcagagacagagtcttgctagagacataaacaaatatgacagtctctgcgctcgaggagcatatagtctagtgaagccgatcaccataaaaacgcatggctgtgataccacatcgtgagtgtaagaacagagctatgtatgcatggggaacagtggacatgatggccaggtacccttattatgggaaacatagaacccaaagtgtgttcccaaaggaacatgattcaaaataatattttgctgaacatattggtaagaaggaaagaaaacatatctgagttgaaaagttacagtttattttttccccaaactagtccccactaacccctcttacattttccttgttgcttttctttctttcattatcttctttgacacttcctcaccctacaggcagccagtttgtctttctgacaaacatcattaagtggtaactctggagagtcgggagataactatggcttctctccgctacatgtaaaagtgctctctggactagtaaaatgtgataaagcaccatccatatttctttttaacagagacaaacaaatcttataaactccctgagaacaaagcctatgtctgtacaactttgaattctgcacagtttcgtattttaattcgtgaaacgtgttgatccttctagtgcctgaccttaggtcaagtgctggggatacaaagaaggtgacctttgaattgggtcttgagggatgagtaggagttggttctcaattatttcacgtttaagtcgacatacttccctccctttgctaaactcgaattctttcactttctcagcaggagtatgcattaacttttaaaaatgaaagttaacggtttaatttttactgatggtctgtgctacttttgtgaaataaaaacatgagcaaagtggtagacagaaaccaggactcaagagcagtggaggaggagggcttctactgtgtgggaacgagggctgggagagcacagtgtgtgttcagagcagtagtaatccaatgattcatcctgtcattcctcttcctctatagccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcagttagttctcttctgctgtcttcattgggggaggcgggaagttgttttgggtttttgtttctttgagtaaatggtttactgcgctacaaatgcacacacagaatattgttcaactgttggctgttaacatattgcaggctgagtctgaaacgcaggaagtgttttcagatagcacaagtcaacattattttgaaaagggagaaaagtccataaagtttttgagaatagtgtaacaatgtattggtcatcctaactttaatgaatgaacttaagccccctgagcctatcaccctcttttaggatccatctcctttctcggcatcaaaataactggccacagcaatattcattcaagcagccttgctttcctctgggaagaacccgacctaggtttagaaaaggtattgaatcccactatttcacctttcaggagaactaccagagttacctctttttttttttttttttttttttttgagatgtcgtctcactttgtcgcccaggctggagtgcagtggcacaatctcggctcaccgcaacctccgcctcccaggttaaagcgattatcatgcctcagcatcccgagtagctgggattacaggtgtgcacgaccatgcctggctaatctttgtatttttagtagagacggggtttcaccttgttgatcaggctggtctagagttacctcttaagaagttcaacatgaggcataaaaatagattctagatattatgttacctttctttccttgtagcattcttgattattccggggcaactagaagctctatccttttattacatatgtgaattttatcttctgcaaggctctgagctgcctggactacttcagtggactctccaacatctgttcactcattgagcagcactgtttagaaaacccagaatgggttgtaattgttactgagttaggttttccaagcagcagagctcttctctgactggaaactgagaaaacaacatggaattatagaataaaattgtacctgaattctagtcccaattctgccatcagcttggatgacctggatcatgtctctgtacctcagtttgttgttgttgttgttgttgttgttttgagacagagtcttgctctgttgccaggctggaatgcagtggcgtgatctcggctcactgcaacctccacctccctggttcaagcaattctcctgcgtcagcctcccgagtagctgggattgcaggcgcatgccaccatgcccagctaatttttttgtatttttagtagagatgggttttcaccatgttggccaggatggtcttgatctcctgacctcgtgatctgcccgcctctgcctcccaaagtgctgggattacaggcatgagccaccgcgcccggctttgtagctcagtttttaatgacagggtttggctttgattctgatgtctgcacaacgctcccagggctgtaagaactgcagaacacactgagaagacaccgtcccagccatggaaagcccttctatgcagtcatttattttcacatgaacacctcccagagagctatgcaatgacttgatagatatgtgtaacttttcccctattcagatatattgttaggaaaatctcattaacctttcatgtattttgatgactatctcaagatggtagttcttctgtttagctaaactagtaagagtagaatcagttaatagaaaaaggaaacttatggcataaagccatatggtaataaggacaatgagttttctgatttgaaaataggtctttgatacctcaacaataatatacctggattcaattctttacagtataaaaaaaaccttgcacatttgttaggttcgagccacatgcacctgactactgctattattatcctctttattttagagatgaggaaaataaagtccagagaggttaaacattttgccctaagaccccacagcaagtgggtagaagacctgggactataatgcaggttttctggcctctagttcagagcattcgtagcatgctagcccctcagtagcatagtactttaggaagctagtctttcccacttgctggtagtaagccagggagacataatacactaagaaagcttcggtctgagaaaactttccatgattgttttttctcaatttttagaacagtaggaattcttcacctcatctgctatcccaacatattcacaattggaaaatttttgtgctgttctgttcttttcaaattacaaaagtccttgactaggactgctggcttttacatgcacaggactgcacaacactggtgtttataaagctcattcgttcaatgataaaattggccatagcaaatcaaaaataaatgaagacctctgcgtagctgattggactacagaaaataagccattcatatccaggaagaaaaaattaaaaggattgctgaaaaggaaagagccctaaagcaaactatctacagttttggttagttttcttttcttttcttttctttttttttttttttttttgaggtggggtcttgctttgttgcccaggtgggagtgtcttcttatattggtcaggctggccttgaactcggggctcaagccgtcctcccacctcagcctcccaaagagctggtattacaggtgtgagccaccagccatggttagttttcatcaactgctttctcaccttcttttcatcctctaattcatacctgcaatccttattagaaatcctgtcaacttaatagtaccagaaagtcaggcaaaagactgataaaaaaaaagcctcggtggtcaagaacttaaaattacactactggtgtgcaacgtgctcttatctgtgttactggaggacaccctcaagatacctcactccagttgactccttggctctaaatctttgagttaaaaaataaggaggttctttatggaataaacaggtattaagctatttaaaggagagggctgcgaaatgcttgattatgggcttttcaagttcagaggccaagcgtttgtcattttcttccagtaactaggactgtgtcttgcccctaatagttactcagactatatttgcttaattgaattaaacacagttgcctatgccctttgaaattctggactttcaacagagggcctctagcccaatatttgcttaccaaactggacatcattgatgatctggattcaggcagggtctggaaaaagagagactgggccaaattaaaataatccattcactgatgacacaaaactaaactacaattgtttggcaccctctcttctccttatcttgcaaaatcaaattaagcactagtggaaagaaacagttcagagaggaatatgggaaaggaaaaaaaaaaaccaaaatgtgatttccaacgagactagagatttgttctttatctacatggtcatgttactcatttgatagcatctatctcaggggtattatgttatctcttggccaggactcatgaaagttaagatttgcattgataggaaaagttttgcagaaatatggactcttgagagggtgggaggtatataaaagtggctatggctgtgcagcagagcaatttgcatttcttatacaccctgcttgagactgatgtcattagtgttggttaggccaaggctgggggaggctactcagaatagtgggtgaccaattacccagaacctttggaaaaggaaatgacttgattgaagaggccattctttaaatgtatcttactgctcaatttcaccattgcctattgtagaatgcgccctgcccgctcagcatttgcctctaggcagaccaggactccttcccctccccaactaatcttccttctcctgtatcttattccctagggccacttgcacctttcaaaatggataaattaaattcccctgctccccttctaccaactttaacatcatatccttggtgtatgatgccctacttttcattgttttgcctaaataagagtatgcttgggaattttaggcttagggctgggaaaagatgggatatggaatcgtcagctatagattgccggacaagaaattttaggagcagtagacctttcaaccgccctcaataggatggaactgactgtccccacaacctatttccctttcagtggcttgctccagccaaggccccttatttaagagatctttgtgcgctctgaaaaccatgcatatgccagaactaggtgctctgtttcatgaaggcagtttgataactgaatggactttggaagtgcccagtgtttgactattaccctggtgcgatgatcacactgggcgtatcattgtcacatgatcccatgtttgttgatgaaaataaacagtggttttcaaaaaccattttcagtgccatggtttgtcataaaacaataaatagaaaagcgcttgatgtacctgccaacttttcacccctatctaagagatggcgaagctccggctgcactctgtctgcaaataaggaataaaactttttatttgtgctatattggactttgggttccaaagcatcacattgtttatggtaggtacatgtggagtcactgtatttataattataagagcaaactggtgccattggaaaaaatggtctttctaaatagtgtgtgaaatatgaaactagtgaagaataaaatcaatatggaaatgtaaacttctatgaaaacatttccttacccatttaagtttgtgcattctgaaatttacaatggccatcaaacccagtgactcaaagctattggaaagatatctgtccttttaaatcctacacttacagctaaacaacgtatgggttcttttccctttatctctggaaaagcccgactgagctggaatttgctccccaaaggaaagacatcaggcagaatcctcagagaaaggggcttagggaccccagtagaaataaaatcaggatagtagtgccaattcatttctggggctgctggagaagttaggcttctgggatcattcatccatgtatcaatattaataaaataaattcttctattgccaggactgtcaggaatagctaaagatgtaaaagatctaaaccagtgtgttctttcagagaataagttaaaaataatattttatagagttaaaaagaaaagcacttggattttttttttccaatggctggactcagttggccaatacccttgtttgcctgctgagtaactctttggctggcaattgatttttttaagagctgtggttatgtgctagttcattagcatgtgaataattcttcatattaagttttcatttgtttgtcgtaaatctctaaaccctgcaggaaagcagaatatctttgacagctttcaaacttcacttctatcacacacagcactccaccagcacctctaggaaacagtggttcaaaaggccctgccccaagaccccagcatgttaagagcgttagtttaaaatctccagttcatcctagaacaatcattttaaaaggaaaaaaagaagtggggaagtcctatagaaagcaatggaaagggaaaagttctcaacccagaatcggggcttaggttctagttcctggtttgctataaatggcactttgactttggaatgtctctggcttccgtttatccattagtcagagaatgatgggacagattacttctgaagaccccttatgtcctataaatctcatagttctcaacaagagggaaacgggaacaacaggggcagaaaccaacgagaggctgaggaaaccaaaccaatgagttttggattgtagctaaccgaggagaaccaggtgtgtcaaaggtgatgaaagtcagatgtgtaaaaagcaagaaggaaacaaaatgaagaacatgagccctaagaattcagtgtctttccctggtaaaactaagaacatcaggaaagccacactcgatgaggcacacatttttccaagccagttttcactctgacagggttacctcaagtgattgtttgagagagggtagaggggttgactacactcattcaatctgccaaattagaaaagtcacccagaataccctgtgctttttgagtgattttcatgtttatctcccgtagcttgctattttgttattcaaaaggagtgtaaacttagggcattcatgtataatacctttcataactcatgttaaaatgctgatgttattggatttctcaggaacactactgttctatttctccattcaaaaaggtaaaaaaaaaattaattactctattttcatcattaatcccatttcccccattgtctttgtattctacataggaagcatgccatagatctataattgaatttactcatctatataaaacagtacttttaatcccatcctgaaaatcaaaatatatttttgaaaatcaaaatatgtcaaaattgaaaatcaaaatgtcaaaatattgaaaattgaaatatatcaaaattaattatatttgatgtatccctcaaaaaattgatttctccttacagaaatcattttgctttcctggaaaaacactgggttcaagtgcagctttcttcattccgttctggtctgtccagtatatgaaaagcacaagcacacaatgaccagtgacagattcaccatgaagctaatgaagcttcatggggcccttcccttgtgcagaagtccttccagtgcttggggaagggccgaagcaattttattctcataatttgtaatattttccttagagagactccactactgtgtatattttatgccccacaaaacctgaattcaccccatcatgtcccatttgtaattaagctttctttcctgttaaggagggacgcttacccagccctttgatgtacggtctgaagataaatacactgttagcccccaactagaagtgtaaaatcattaagacaaaatttgagaatgatacggctcctataatggcaatctttggatgatactgtccaagctgatggaaagtgtaaggactccactttgctttgtaaaaaatacaaaaaaacagccatggaagtgcagtacattttgtatggaacattgtaccacattgtaccaaatgtcatgttaaattaatgattcaagataaaatgcagaccaaatttgaccagggttttaagaggttctctctccataaagcaggctggccacgatcttctcactaggtttgagggtaaggaggtcccctgtgaaatatgaaagtgaactctctgctatctaagaattggtgaattggttgcaaagaaaggcagaccaggcccacccagggatcactcttgcagtttgacagatctacctggaggctgaaagaatctatgcagccttcggaaatgcagtgaaaacagtataatcagatatgactagggaaaaaataaaacaactggtgttcatttgcatgtcaacacccagaattctctgctagttcacctcttcttcagaatgcagaaaggattgaaaaagctggaatggggccgggcatggtggctcatgcctgtaatcccatcactttgtgaggcagaggtgggtggatcacctgaggccaggagttcaagaccagcctggccaacatggtgaaaccccgtctatactaaaaaataaaaaaatttaaaaaaaattgccgggcgtggtggtgggtgcctgtaatcccagctactctggaggctgaggagaatcacttgaacccaggaggtggaggttgcagtgagcctagattgcgccattgcactccagcctgggcaacaggagcaaaactccatctcaaaaaaaaaaaaaaaaaaaaatgaaatggtttggcatgatggtggggtgctgattcctctctctgtccttgagtatttactgccaggaaatcctgaccaacatcaattgccaaggggttaaagtcacattgttgttaaattgggagagaaaattagcaggattttgtgccctatgttcaactattacagagtttcaaaaacaggaacctggagctttatatgaggggacaggggaagtaaaggggaagagtgacaagggtgatttatcagggcatagttccagataaatcacttacatggaggtgaaagaatttcatgcatatgttctgtgccaagaagccaaatggttgagcagaagctctgaaagttgaagtgggcagttctgggtacagtctttccacatgtgaaaaggtgatgattttatattcatgttatttgtactcctgcatgtgcagttggtcttttcaggtgtccagatcatcattttctcatgacatgaaagaaagccaataaaggctagttgtggtggctcacacctgtaatcctagcacgcacttagggaggccaaagcaggacgatcacttgagcccaggagttcaagaccagcccaggcaacatacatagcaagacctcatctcaaaaaatgaaataatttttttttagaaaaagaaagtaaatatttcattaatataaattataatacactgtaaaatgcctggaggacaagaaacacgttttctctaggttctgtgaaagcatcattatccattcacatttttctcataattagaacctgacgttctggaaaaatacagtaaattttcatcgatgttacttgctctgccccccttgtattcctcccattaataatattaatcctgaaaagaaaaaaagaaaaaggaaagaaaccacccaagcaaaatgtttaagaaatacggtaaggacagtttggtcccaatttccttaaaaattagagccaccaagtaaacctgtttgggggtcctcacatctgtcctcttttggagtttgctaagtagtacagttgaagtgaattttttggtaacaaaagtaaaaacttccagaataactccagtaaagggtgttgatgtgacgtacaagtttggaaatatatttaatatctgagactcctcaatgttggtgttatttcctccagtttagaacaaacttaatcttttgatgaaaatcatagagttccatagatatgaatagaatctccaaccagtgcatcttgaagccggctgccacggccactcatgcaccttctctcctttcaggagagaattgtcagcatctctgctcaaaacaatctttacttttagtccatcagagacttcatgtttgtcgtgcatgcacactcacacctctccacacatatgcccactccccacacacatcctgcttctgagaaatgcaactagaatttcctccggagaagagatggaaacccctgattgagaaatttaaggcctaggaaaggtaaatgacttttcttaagtcacatggctcaccacagaaccaaagctggaacacacataccctcagctcttcttccagtgctttcaggtatacaagtatttgaacaggattcaaggtttaactccttatgtgtcattcagtgtatccagtgtaagtaaaatgttaccccttagtcagaacagccttgagaatttctcaatgactacaaaccttgcttagttctatacaagggaagtaaaagagagagaatgtcatgaccaaatgacttaacttttgaaggcagcctattgcaaacttgaataattggggcatgtattttattgattgtttgacctataaggacagatattttaaaatataaattcaaaattgcttttttgcagcattcacctgtaggccttctatatctgctctttgtgaaatagatggcttccagagcattaaaaaaagaaatagacattttcttggttcaagagctgtaatctgctttccgagtgatttttcatagttaagcatattttatgtgcacatagtttcatttgagatctgagaggatttcatctaaaacggaaaagttattacctgtgaaggagaacaaaggaaaaggaaaaagaaattttaagaaaaaaaaatttttttaaacctgtagttactttatgttcaagtgggtattttcaacgctgagttgctgaaatggttttttattgaaagaaggttaaaagtgtatttcattgaaagccaagataaacagtagattttgtatcattttaaaaatcaacaatcggccgggtgtggtggctcatgcctgtaatcccagccctttgggaggccaaggcaggcagatcacttgaggccaggagttcaagaccagcctggccaacatggtgaaaccccatctctactaaaaatacaaaaatcagctgggtgtggcagtgcatgccagtaatcccagctacccaggaggctgaggcacaagaattgcttgaacctgggaggtggaggttgcagtgagccctgattgtgccactgtaattcagtctgggcgcagagtgagactctgtctcaaaaacaaacaaacaaataatataaaaatcaacaatgaagactccccaaacaaaagaaatcaggattcttagccagtcataggtaaagtgttacagtgtaaattgaagggaaagaagaccaaaattggtgaaatatgtttggtcccagaagataattaagatgaataaaagaacttgagagtattttctcattattaagcatcttcagctttaaagattttagttagcaaagcaagtttacataaacagttttctgaacctgggatggcattcactgtgagttagaaatctccaagtcatccacgttttccctgttttatttgcagccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggaggtaagatttgtcttttgatcttctatgaaagagggtgggatgatggtggggtggccaaaagaattttggatttccttagtgttagtctgcattgtcactttaagtgccagtggcatgatgccatgaatcatcactacacgtgcaaaacactgtaccaagaaagagtgtcataggtttgcggtgtgaaggttataagtaacccaggatcttctctttcatccctcaacaatatattttttccagtcaaatgccctcttctcctttttctttttcttttcttttctttgtctctttcttttccttccagaaaggaagattagaggccagtctaatctttggttcctgtaagtaatggcatgtataactttgcttaagacaatgctcacttgatgtagagaagggaccactaagtatttctggtcaaatcatgctcctgccccaccatcagtaagatactactttgaattgcttttgtgatatcaggaattaaggaatacctactgacctccacccactctcttgtgtggattcctctgatggcttcccttaaagtacctttgtgaggtagttggttgtgattaagaatgcaatgtgaaccgaacacacaacatagaaataaatttagttatttagcaatttgttgtatttttggccctgcaagtagtacatgatctgactaaccaattccaaaagccagattttcttctgagtgtgacttttttcttttttttttcttttttttttttcctttttttttttttttttttttttgtgagacagggcctcactctgtcacccagactgaagtgcagttgcacagtcatagctcactgcagcttctaactgcagttagtaagctatgatcatgctacttcataaaagtaatacgtggccattttacagttgggctcaagtgattctcccacctcagcctcccaagtagacaggacaacagctgcacaccaacatgccctgctaattttaaaaatttttttgtagagatgaggtctcattatgttgcccaggctgatctcagattcctgacctcaagcaatcttctcaccttggtctcccaaagcgctgggattataggcataagccaccacgcacaatctaaacattactttcattagcctctcatgagtagaagaaagttttactttcatctctctgtataaaaattgtttttaagtggcaagttttcctattgaagtggactttaggtgagctttttgtatgtactctacagtgtatcctctgtgaaaaacccgtcgggggatagtggtaaactgtatataactgcacaatgtattataaagagcacgatccctttgtcatcttttgttgtccagttgaaaggagaatgtttctgaaatcaggtgcatagtgcagtagtcataagaatagcaaacatttatttaatacttgctgtgtgccaggcagtgttctaagcacttcatgcatatttattcaatcctcacaacaattcagggaagtaaggaccattttacaaatgaggaaaccaaagcactgagaggttttctcatttgcgcaaggttacgtggctagcaaaagatggagacaggattgtgagccaggcatgctggctcagactctacactctctaccatcacatcatgcggaataccggcctttaaatgcctagggtgcaaatagatcacagtatggcatagtttacctttggagctgtgaagtccagggtcaagtcgtgattcactttaaaataactacttgatattgtgggtttttgttgttgttgttgtttgttttttgtttttttgtttttgaggcgaagtctcactcttgttgcccaggctggagtgcaatggcacaatctcggctcactgcaatctccacctcccgggttcaagggattctcctgtctcagcctcccgagtagctgggattacaggcacatgccaccacactcggctaatttttgtatttttagtagagacggggtttcatcatattggtcaggctggtctcgaactcctgacctcaggtgatccgcctgcctcggcctcccaaagtgctgggattataagcatgagccaccatgcccggccgacgttatgtttaatagaacaaaaatatactacctggcatggtttaatatcttgaccaaatttacttcctaaattaatccctggctgtctggtatctttgattaattcagcaaaaaaggatgagattccagtatataagaggtttgccagaagaaaaatattaaattttgaaaaaccagattagaaaaacagttgatattatttatctagtattttattttacttcattttattttattttattttttgagatggagtctcgttctgtcttgcagtggtgcgatctcggctcactacaaccgggttttccgcctcccgggttcaagtgactctcctgcctcagcctccctagtagctgggattacaggaatgtaccactacacccagctaatttttgcatttttactagagacagggtttcacaatgttggccagtctggtctcaaactcctgacctcatgatccacctgcctcagcctcacaaagtgctgggattacaggcatgagccactgcacccagactatttatctactattttaaggcacattttatttttaataaaatttgaaaatatattaatatttaatttatttcaaatatttttcctaagcatactatatatcccatgtctattcttttcacttagtcaatttcccatgtcattacatactctttgttaatagtttttgttaactttttaaataaaagtattacatggtcattttataattgagagtacagaaaggtataagtgaaaagtaaagggctttgccctcaggataaaatagaattaacaaagtcctatttttcctatcaagaagtttacatgcacactcatgtagatagatacatacatacacatatgtgtacgttcatagcagtttgtattcctacccacatatacgaagggaagcatttgaaatgagccattcaaaggtacttagtttctactaggcaaggtctagacaagaaacagtcatgagaattctgggctttatgaagtgagatctgaatctcaggcatctatcgaggatgagatccacctccccgattgtgcttccttagagtaagacaaaatttctcaattagtttttcctcctattccagattttccattcagctaagcaggaggctttcatccactaaacaaatttctgatcctacccagtcttaacaagagtgattgttttctcctctgcaatttattcgaaagtctatcagttactttggtggtttcagaaagaagtgcttttgttggggatccagttggcctcattctgagctggctgtggattcacagaagaacttcacttcagcaaaggctcttagggccccagactaaaagaaagatgagcaagatgtgcgtcatctaagagtgggcgggggtaaggaattggggattaatgggggtgtccctagaggacccaaagaggagatttttagatgaaagtaactgatttgacccaactcagtatttgctggggaaactggagttgctacctaagattgctgccattgaacttcagccttaaaaactgctgataccatttgattggtacttagatttgcctgggcttagcgtctctgctgttcactcagctctcaaaccaagctgctgagaaagatgacctagttcttctcgagtctttctcctctctctctttctcccctgagcttgggggcaagaattagactaaatgaaaggaacagcaacctattctagaaccaaagttcaggacttggtacctttttaagggcatttcatgttcattctctcattccatcctgacagcgctcctgggcttttaattctcatttcacagaaagaaaagtgagggtcgtaaacttaagtgatttgtctgagatctgacaaactagtgccaccgtggaactaacactgaaacctgggtgtcctaactgtcccctctggccctggccagtggactgtggtcagcacactcactcttgtgttggtggctgcaacctgttctctattcactatatccctaaggtaggcccatacctagaagccaggaacagcgcttccttggtcatcatggtaaacaataacttcccttgcactccctagtcttactgacaggctgggcggtgtcaagagtaataaaactgcctccagcaaagccatgtttgcaactggcgccgcagcatcatgccagccaccctagcaaactcccaaaacctacccagcagccaaagccaagcgtaaagctcagtgaagctgacaagccccaggacactctggtggatgttttgcccccttgagtgtttgctgttttgtgaagaaaaagtgtaggagagttccagctggaactctgcagcaggcaaaagctctttttgttaattcaaaacagtttggaatccatttcagttcttcctaaacctccaagatacgggggaggaaattcactggattttacaatatatttttcaaggcaaattgccatcgccgtcctaatgacagagaagctgccgatatcactacaacggctgcagatggcaagtcatccagcctcctcctgtgccctcctgagtctggcttggtgacgcaaaccacctcaagggcagctccttctttttccagcagtcccagcactgttccttctctgtgaagccactttggatggctattcttggcatgatcatagacttttctgctgttgagtgggcaaggagaatagggtagaagcaggggaattgttctcattgctatgcatttatagatcaaagaggagcagatattttatgttaattggataacacctagttctatcaggtcagtaacctggggtccctggggctcccccatgggtcataccagatagatagattttttaaaaatttgttggaattaaagtagaatcaaagcctagtgctagcccagaataagttattcaataaatgcttgttgagtgaataatcatccgaaatgattataatactgcctaccattcgttgaaagcttcagtatgtgggagaatctgtcctgggcactttatatacacccataatctgtttggatttcttggtaggaagactacatggctgcatacatggtctttcattacctccattttatagatggagaaaatggaaatttaaagaggttaagcagtttgccgaaggtcacagagctactaatcggtgaggtcaaggttcagacccaggtctctcagactttaaaactggtgcacttaaccattagactctactttttgcgtattgagatgtggaagcatctcaattaattgcttcagtcttggagttctttggcggttatcaaaaactgcctatttccatattgcctccagatattgtaattaagaatttccaaggccaggcacagtggcttatatctgtaatcccaacattttgggaaaccaaagttggaagatcccttgagcccagaagtttgagaccagcctgggcaacatagggaggcttcgtctctacaaaaaatacaaaaattagccaggtgtggtggcgcatgcatgtgtagtccaagctactctggaggctgaggcaggaggattgcttaagcccaggaatttgaggctccagtgagctgtgattgtgccactgcactccagcctgggcaacagagtgagatcctgtctcaaaaaaaaaaaagaaaagaaaaagaaaaaagaatctccagattatagttcagatattacgtattaatatccaaagccctccttaagacaccattttaatctgtctaacaaaattaacaccctaggttttgacaattgcttgaggagggagtagggcctggtgatagagctaaagactagtgttctggttctttaatttgttcagccagcaggtacctggaaataatgatactgtttgcagaactccccacaaaaatggacagtcaactttaaataactaagtaaacacatctctgcttaatcagataccatcttatactaggttctaatatagataagtggttgcagaaacatttatgagaggaatgttaaattgtcgaaataccatccagtcccttcatttcttcctgcccttcagaaacaaacagaataaccctgatgcagctgtcacggcagaacactcattccagctcacagaacaccagtgttcaatcagcactgagtttgaggagcaccgggtttgggagtattcatctctcttccctgtcccatagctgcccttcaaactcttgagactgttcccagctctaaaatggaaagggtcgtcttctctctggaacaaaagacaggatgtacttgcttgaataggccagtgctttcctggaggaaactccggcttatggagcctctgcttctgcacagcccagtgccttccaccccctcttcatctgctagcatctacattactagttaactggagaggagaccaaacccaaaatattacaaaacatagatattaaaaatactgtgaacaacaatcagcccgctttctcccacctctcctgtaccagctaaatatttacctccctcatccgtagagggtttttggacaagacaggctggtgcctacatggtgaaagttgtttttctctcttttcttgtaatattgcaaataaacaaggatatctgctttctcttgagcaggacaacagccaaagagtattattgcatagttcctgtcccctatctgggctacgattgtgtctgagagtaatagcacttatttattaactggaagaaattttacaggcacctatgttgtttctgcctagcttctgaggctcctctcttaggccactcctctcaccatgcccccacagctagttggtaaaatggctttgaataatgactcgctttgagttatctagaaatgtcccattagttcactattttcataaccccatagacttggtttgacaagggtgaggtttatttatgagttctcaactggcacccaatttttaaaaaccacagagctgggggcggggatagaatgatggggaggagatgaaacatcctagaatcatctgatatactttaaacatatacatatacaggcttttgttgtttgtaaaatatagaagcatctttatctgtgactggttgagaaataaaatcccttccccagcccaggcatggtaggttgggatcatcccagtgagcgagcaagcaattggatttgccctcagagactgactatggggtcgaatgctgagtcattcctgtccccacaccagcagtcctggaagaggctcagtcgatcagtcagtgggctgctttctggagcactttatcattttgggctctcacaatgtgtgtttaaactctgagtgaccccaggcagccttccagactggcacagttcaccaagacgttcagacagatcatctgcaagtgaggaagaatgaaaacccagcactggttcaaatcactcctagcagaggtagtgaaggcatcactcagtcctggagaaatcttcggagggctcaccagcgtcacaagtaggtagaccagaagaggcaggctggcccattgcaaagccctggaatcagagggacaggagttccaaatccccactctctctcttcctagatgtgtgcttgcggcaattatttatctctctgagccttaatttccttatcaggaccctgagcaaaccaaataacatgatgtgaagcctctggcacaatgcctgggacatgttcagcacacaataaatgctaattttcccttttgccctatatgatatgatttgaagcagcgccactcaatcctgccagtcacattgctggaaatgccaccaacagtcattcttaagatcgcttattacaatataaatacccctaggggatatcagctaatcgctttatcaactatcgctttaaggggattaggtgtggattgagaacaaaacccagaaggaaagagatcctggaggaaggggagggcttggaagtggggaggattcggacgtcctgtggcccccgaaggagacggggaatggcaggtctgctggattcccagccttcaggaattcccatgaacttcttggggacttcatgtgtttagaccatatttagagagcgtgcatgtgttttgagatcacaggtttttctgagagacattgggaaggaggacagcgggggagtggagtggagagaagtcatacctggcagcttttaacttcacttactgcccttcatgtgctgttgccatttccagaagttgagggagtaaggaagggtaggagttcttggtattcccttatctcttcctttccctccccttatcctgcccagagcatcgcagggtagagccaccagacggattcagggcatggagatggcaggttagtggtagacttggaagggatcgttcttcctatttcagcaccacccttcttcctctacctcccaccctgcatttccagccccagtcagattccttcttggacagagaaggagcagaaatgtacgtacatcactaggcttaaggggggactgcagatggctcccctggtcctgacaaaaaggtggggagcaaaccgcatggctggacgtaaattgagtgacctggaatgactagaacgacagggggcctctaaccccagggtgcacagaggtagggacccaagggctttccggggtcggacagaaatctcgggttccattttcactctgagagacccagaaatgacagagcccatgctcagtttccactggtggtgctgttgagtggaggggctttgaacatggcacacaggaaagggttgcccaccctgtcaccagatgggcttgcactgttttgtggtttgggagttgcatattggaggtgggagtggagtgttgagatgaggcaagttgagatgtaatcgctatttaatgggcagatcaaagaaaacattggctggtttgatttttattatggttatcccgctccaccatcctggcccagtcatatatattattgttatcaggcatagataggggaagcaattaaatgtgaaactgttatttaaaccggcaagtgtcagaatgttaacccacttcagagcgaaggctctaccagcaacatgtgtgttcactgcagaatgtgctggttacagatgatctttatctgtttgtcaaagcatttgcaagatctctattttatgtcatcccctttccctgaaagcagcagcagtaaacgacattgctcctcaaatcatccatggctcagacctctgattaatttaagctggccctgctagcgttcttataaatccccagcaagatgcaggcccaggagtggaaaggggaaaacatccggaaagcagcggggggcagggagggggaaggctgaagggaaaagaaaaaggctgaagggagcaataattgaggaagaaagaataggaaaaatgtagaaaggacccaagaaaatcagagaaatgagaggaaaagaaaggtaagaagaaaatgaggcaagtttacagagcagtaatgaaaatagtaatagctaatttactgtacaacagacactgtgctaagcactttccgtatgtccctcagtcctctgaccaacactatgagttaaattaccgctacagctccaattgttaaagtgaggaaactgaagtatagaaaggttaaggaacttgcccaaggttacctagcttgcaaagttgtaattccaactgcgtctcactcagctggcagtgtgcacccttggccacacattagaatcacccaagagctattaaaaatcctgaaggtcataccccaggcaagtgaatcagaatctctagggatgggtcccaggcattcgtttgcttttttagtctccccagataattttaatatgtagccaaaattgataatccctgcactagaccaagctccgattcaaatttaaatattaagaggacacatgtataactttgctgaataattttttcctatcggtaggatggtgggaaggaattgctataggccacaccatgatactttgctgaattggatcagaaaagtaccctcaaattcccagcatcatttcttcaaggaagtagtgaattggggcaccgtgggttttttgttttgttattgttcttaaaccaccttgcctatgtgcactcaactacaggaacgagaacttgcctgaaaccccaaacgtgtctgcatcaggcattatttttcttgagctcctgaatttctgctctaaccaagaacaactcaaaaaccttcaatttcataactgttgatatgccacccactcctacttacactcctgaccccaaccccatggaaactgcctttattcgaacaaacacaagaattataatatgttctaaagttcctctacctatttcattctcaccatacgcttatgaaagaaatgtcattgtcttgaaaactaagcctaaacccagaaagattagatgatttgtccacaggcacacagaaactaagtggaagagaaacaggactcaagtcttcagtttcccaatccactgcctcctagaacatgcctgtgaactgagggagcagtgcaataaaggaaacttgagaggaagcgcaaaaggaacttaaattgcaaagtactgggttccctaagtagccctgcctcccctccagttttcagcccacacctgaactaaggtctttcattgtctgtggatcaatttacagctgccctgcaatcacatttgttttgtttgtttgtttgttttggttttgaacgccctagggacagtcggtttcagattagtctctatcagagagccaatagggaaagatgcagaaagccaggagagcagctcctagaatagagacaaaaccacctgcattttttctgcctcttcttcaggagactccactgtggtcatctggttccctaattgagacacctcctgagaaaatctgtgctagggaaacacccagcccaggtgggcagatctcaagaggaccagaacgtttgaatctgcttcagataaaccctccacttaacagtaccagatctggacgagatggaagctttgtagatttgcccataatccacagtgctctgtaatcaattccaggaccgtgtggatggagaccggaaaggtttctttcccaattcttcacagcctggggagtagggaactagtaattgactccagcctcctcgagtgctaagtcctggagtctgagtcacacacacttcccaaaagggccttctaagcagcctaccccagcagagaggacccttggccctggccctactccagactcaactgtaacctcagcaaggtgcagggagaggatctcaggtgggcctcctggcaacagtgccttttcaggcactaggctgttgggaatggagagagaagagaaaggccctggcactgtctgggtatttggatggatttctgtggcacaactgtggcacctggcttagctcagtaaaggactgagtgtgcgtggagcagaaaggggaaagagactagtggggagctggtgaccagttggagggtggagggtagggaacaaccctggcagatccattttgcccttgactagagcttaaagaccaaaggaagcgttcttgtttttaagtaagtcaggtgttctaagtttaatgttaatatgctttttcaattagatgtttgagtatatttccccagttgaattatgtagagggaattacaaatgagaatacatcttacaatttttgaattagaatctttgagattatctatttagtccggtctgttttgtttagacaagaaaattgagcccattgatcttaatcagtttgcccaaggtcacagagctggtgagtgaaagctttagatgtagaatatgcaacttttctctgctgccctccttccctaaaccatcttgccttaacggaggctcagtcagggggacaatgggactgatctgtaaatgatctaacttgtcattgcagaaaatatacttactccgtggccacaaaatttcagcccttcagccaagcttcatccatcacatcataaagaataacttgccaaagaggggtttgaaaatccaaaccagatccctctccactcctagagaggccagttccatgacactgcaccggagccaagtgtttccagcaagccctgatcagttgcatgttagtccttgtttgtatgataaacgggacctagtagattaacatcatctcctcagcccaagggctgaccccagtggtgctgctcaagaggaaggaggaggctgggagcggtggctcccgcttgtcatcccagcactttgaggggctgaggagggcaaatctcttgagcccagcattcaagagcagcctggaaaatgtagagattctgtctctaccaaaaaaaaaaaaaaaaaaaaagaagcaggtggaggaaggaggagctgcagaaaggctccgggagccagtgtctagtaagcagaggcctagcccaggctgagtggtcccagcccaggagggcctctctcggcacctgtctttagatgcacgtctgcctctggcctttgagatgcacacattcatcaagcaggaggatctgtagtcattaaaccaaggtgccagtctggggcccagccttcaatgaagcatcagtggctttttcattcccatagagcaactgcgaggaagaccccatgctcccaggggcccaattctccaggccaggaccagatcatttgttccttctcagaatctgagatcttttgctgatggttccaaaattgaatgctgaaatctaaaaagaaaattatacttactctggtcaatgggatatgttggagacctgagagtgaaaaaatcaaactgaagaccagtttccttctttaatggaagaaaagtaagaaagttaacttagaagaatatttagagttctgaaaatcccactctgagaccactctgccatctcacattgttctcttgagccttatctcaatggataaaggtcactagagtttattagacacaggaaaacatcgtttacagacttcttcattgtgtagggattctgccttcaggcctgttcaccagccaggagaattgtgctttttgatccagggctcttggccagaacagccttctcgctcattaactcagtcaaccaaatgcttcctgtgtttccactggtggttcagaacgtagtctctgccctctgatacctcacggtctaacgggggagaaacaagaaagtcagaacttacaatacaagataataaggaataagagcagagcctttggaaccagacagacttagatccaaatgtcagctctgcatttactagcaagtggtctatcttggtaagatgcaactcttttctctaagtcttaatttctccctctgtaaagtatgacctcatggggttattgtgaagattaaatggcatatcgtatataaggcccttggcacgatctttggaaaataataaccattcagtgaatgtaatctactagtatagtagcgattgaggtgtttatagggtgctatgagggcccagagaaaggagatgagatgaacaggagagaggaagggatactgttgaggaaggatttcaggatggaatcccaccacaggctgtattttgaagaaatggttgaagtcaataagaagagtgaagggaaggtgagaatgtttcaggcaggaagaataaaatctgaataagtgcagagtcctgacagaacatagggtgtttgaggaacttggtatggaaatagattaatatggcttaacatggagcaaaatgaaacagaactgagactagaggatgaccaggaccaggtcatgaaggacattaaaggtgggtaaaactcataccatgaaggagagaagagttacagtttttaaaagaaaataaatagccttcaggagctcaaatttgaaatcaatacgagatagatgatcaagtgaatgttctgtcttcttgatgagcctgactgagaaaaccttataatctaacccaaggcaacaaactgagaaattagaaaataaaacttagagattagaaaacaaaaactgaaacccaaactttttacttggaaatttaaaaacatgcttctatataactcttaggttaaaaatgaaatcaaaattgtaagacaagctgtttagaaaagaactatgagagtaccgtttatcaaagcccatgggatgaaaccaaagtggcactaagaagaaaatgtggaggcttcaatgcatttattagaatacaaagaataccaaatcaaatgaattaagcattcaactcaagaaaactagaaaaagagaagaaagtaaacctaaagaaagttttaaaaaggaattcaataaattaaaaacaaaacacacaatttttaaaaacagacttgattaataaaaccaaaagctatgtcttttttaaaaaagcaaaaaatagataaacctttgacaaatcagatttttttaatgagagaatgcacaaataaagagctctaagagctgggcatggtgactcatgcctatattcccagcactttgggaggctgaggcaggaggattggttgaggccaggagtttaagaccagcctgggctacgtagcaagaccccacctctacaaaaaatttcgaaattagttgggtgtggcggctcatgcctatagtcccagctacttgggaggctaaagcaggaggatcacctgaacccaggagttggaggctgcagtgagccatgatcatgccactgcactgggcaacagattaagactctgtcacaaaaaaaaaagcgttaagttaaggagcgtgaataaaaataatacatttcatattctaagacaatatcatctacaactttattccaatataaattactaaaactttaccatataagtagtagaaagcctgaatagataaaagctatggaagaaaaattaaagatagtgatagatttatcaccaagctcagatggttctgcaaacaaattctaagaaaccgtcagggaacagatcattactgcgttgacacagaaggcaaacctctcaacttggtgtataaggctagttaacattgttactaaaacaggcaaagaatggcccccaaaataactctaaaccagtatcacctacaaatacaaaaactctatataaaatattagcaagtgagtacaagagtataaggaactctattaatgtaattcactgtgctaggttaagataaaaccataactcttcagatatcatagctctttagatagaaaaatgtaaaagtatttgataaaaattcaacaacattcctgactaaaacaaattaaaattgtaaaacccctggaaagccaggagtaaaacatgttctccataatctatagcaaacatcatagttgctgataaaccattagaagcatttccttcaaagttaggaataaaacaagaattgctactattgctactattatttaatcccatattggaggttttagccaacacactatggcaagaagaggaaatgagaaaggttaatgctgcaaaagaagaaataaataaagctaccattgctgatataattatctatccaaaaaaagtagaaatccatgattaaaagctatttgaactgataaaagttcagaaaattggaattgtttcaatatactaaaatcaattagttttctgcacatcagtggggatatattagatgtaatgaaaatgaagggtccattcacaatagaagctataaagtatttgagaataaatctaacaagatctacataaagaaaaaagtaaactgcttaaaagtaatttaagttcaccatttgcaaattgaactaaagaaaaatctaaagaagaactagtgaaaattgcttgcaatcttaccactgaaaagaaacctctgttaaaatgtatcagtattttaaaactgtgttttgtcctttggaaatatgccatattaagcaaacatagcataagcaaattaagcaaaaagacaaacctttttttaaaagcatatttgtgaaaaaagcatatttgaagctatgataagaaactgagtagaagctataaaacgtagattcatcaggaaggagcagaagttgaaaaggagatttctggctgggcacagtggctcatgcctgtaatcccagcactttgggaggccaaggcgggtggatcacttgaggtcaggagtttgagacaagcctggccaacatggtgaaaccctatctctactaaaaatacaaaaattagaaaggcgtggtggtgagcacctatcatcccagctactcaggaggctgaggcaggagaatagcttgatcccgggaggcataggttgtagtgagccgacgtcatgccactgcactccagcctggatgacagagcaagactccatctcaaaaaaaaaaaaaaagaaaagaaaagaaaaggagatttctgagtagaaacacgttagcagaggaaacttcatttggggaacaggattgttttgatgccatgctccctcagctgaccacacagattacatcctggccagaaaaaggccctgcaaagagctctgatcattttggttctgattccatccttagttcttacccattatctgcttacccttcctccccacctatttaagatacaaagcaaaacaaaccaaaaatacagatgagttgcttggtagagctgcctaggcctccaaggcggggcccagaggattttttgactgaaccccctgttgtgttttccatatgtgcttccccagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactagcagagagtcctgagccactgccaacatttcccttcttccagttgcactattctgagggaaaatctgacacctaagaaatttactgtgaaaaagcattttaaaaagaaaaggttttagaatatgatctattttatgcatattgtttataaagacacatttacaatttacttttaatattaaaaattaccatattatgaaattgctgatagtatttgaagactgagtcttgtgtgtttcccaccctagcccccaggctttcttttttaccccttttccttctcccctccctccctccatccctctcactcttcctccctcccttccctcctttccttcttcctttatttttcttttctttcttagacattttaaaatatgtgagtggaactgctgatacactttcattctcagtaaattaattttttactcaatttacaacttttcggaactctgcatctgctttaacaatcagaactaagaagggaaggggaacttcagaatcagaaactgctcagagcatgtgttttgtagcctggatgtgctgatatgcagcatatccctgggattacccagaattcagtcttcttccacaatcctaaccgagattttcacgcaccaccagcagcctactcgtatactacctactaatcaatgccactgcgcacccacagagccagtggccagcaactgagcactgctagaaactctggggaacacagaacttttttaagatttggtatctgcgccaggcatggtgtctcaagcctttaatccacgcacgttaggaggccaaggtggatggatcgattgagctggtctcttgttcgagaccctgtctccacaacagttttaaatattagccatgtgtggtggcacacacctgtagtcccagctactcgcaggctgaggcgggaggattgcttgagcctgggagttgaggctgcagtgagctgtgatgatgccactgtattccagcctgggccacagagtgagaccctgtctctaaaaaaagataaaaaagatttgacacctgccaaaaagaatttaccgattataaactgaggagaaaagattaatcttttttcatacagattcaaatacgcagatgaaatcacatgccaggtgaatggtaagagtttgtaggaagtaagatgagtgaagattggaatcccagagactgaatctgtggatttgctgaagagg

SEQ ID NO: 26 - DNA, 4592 bp, Homo sapiens
aggctgttgaggctgggccatctcctcctcacttccattctgactgcagtctgtggttctgattccataccagaggggctcaggatgctgttgctgggagctgttctactgctattagctctgcccggtcatgaccaggaaaccacgactcaagggcccggagtcctgcttcccctgcccaagggggcctgcacaggttggatggcgggcatcccagggcatccgggccataatggggccccaggccgtgatggcagagatggcacccctggtgagaagggtgagaaaggagatccaggtcttattggtcctaagggagacatcggtgaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactgatcaccactaactcagagcctcctccaggccaaacagccccaaagtcaattaaaggctttcagtacggttaggaagttgattattatttagttggaggcctttagatattattcattcatttactcattcatttattcattcattcatcaagtaactttaaaaaaatcatatgctatgttcccagtcctggggagcttcacaaacatgaccagataactgactagaaagaagtagttgacagtgctattttgtgcccactgtctctcctgatgctcatatcaatcctataaggcacagggaacaagcattctcctgtttttacagattgtatcctgaggctgagagagttaagtgaatgtctaaggtcacacagtattaagtgacagtgctagaaatcaaacccagagctgtggactttgttcactagactgtgcccttttatagaggtacatgttctctttggagtgttggtaggtgtctgtttcccacctcacctgagagccattgaatttgccttcctcatgaattaaaacctcccccaagcagagcttcctcagagaaagtggttctatgatgaagtcctgtcttggaaggactactactcaatggcccctgcactactctacttcctcttacctatgtcccttctcatgcctttccctccaacggggaaagccaactccatctctaagtgctgaactcatccctgttcctcaaggccacctggccaggagcttctctgatgtgatatccactttttttttttttgagatggagtctcactctgtcacccaggctggagtacagtgacacgacctcggctcactgcagcctccttctcctgggtccaagcaattattgtgcctcagcctcccgagtagctgagacttcaggtgcattccaccacacatggctaatttttgtatttttagtagaaatggggtttcgtcatgttggccaggctggtctcgaactcctggcctaggtgatccacccgcctcgacctcccaaagtgctgggattacaggcatgagccaccatgcccagtcgatatctcactttttattttgccatggatgagagtcctgggtgtgaggaacacctcccaccaggctagaggcaactgcccaggaaggactgtgcttccgtcacctctaaatcccttgcagatccttgataaatgcctcatgaagaccaatctcttgaatcccatatctacccagaattaactccattccagtctctgcatgtaatcagttttatccacagaaacattttcattttaggaaatccctggttttaagtatcaatccttgttcagctggacaatatgaatcttttccactgaagttagggatgactgtgattttcagaacacgtccagaatttttcatcaagaaggtagcttgagcctgaaatgcaaaacccatggaggaattctgaagccattgtctccttgagtaccaacagggtcagggaagactgggcctcctgaatttattattgttctttaagaattacaggttgaggtagttgatggtggtaaacattctctcaggagacaataactccagtgatgttcttcaaagattttagcaaaaacagagtaaatagcattctctatcaatatataaatttaaaaaactatctttttgcttacagttttaaattctgaacaattctctcttatatgtgtattgctaatcattaaggtattattttttccacatataaagctttgtctttttgttgttgttgttgtttttaagatggagtttccctctgttgccaggctagagtgcagtggcatgatctcggcttactgcaacctttgcctcccaggttcaagcgattcttctgcctcagcctcccgagtagctgggaccacaggtgcctaccaccatgccaggctaatttttgtatttttagtaaagacagggtttcaccatattggccaggctggtctcgaactcctgaccttgtgatctgcccgcctccatttttgttgttattttttgagaaagatagatatgaggtttagagagggatgaagaggtgagagtaagccttgtgttagtcagaactctgtgttgtgaatgtcattcacaacagaaaacccaaaatattatgcaaactactgtaagcaagaaaaataaaggaaaaatggaaacatttattcctttgcataatagaaattaccagagttgttctgtctttagataaggtttgaaccaaagctcaaaacaatcaagacccttttctgtatgtccttctgttctgccttccgcagtgtaggctttaccctcaggtgctacacagtatagttctagggtttccctcccgatatcaaaaagactgtggcctgcccagctctcgtatccccaagccacaccatctggctaaatggacatcatgttttctggtgatgcccaaagaggagagaggaagctctctttcccagatgccccagcaagtgtaaccttgcatctcattgctctggctgagttgtgtgcctgtttctgaccaatcactgagtcaggaggatgaaatattcatattgacttaattgcagcttaagttaggggtatgtagaggtattttccctaaagcaaaattgggacactgttatcagaaataggagagtggatgatagatgcaaaataatacctgtccacaacaaactcttaatgctgtgtttgagctttcatgagtttcccagagagacatagctggaaaattcctattgattttctctaaaatttcaacaagtagctaaagtctggctatgctcacagtctcacatctggttggggtgggctccttacagaacacgctttcacagttaccctaaactctctggggcagggttattcctttgtggaaccagaggcacagagagagtcaactgaggccaaaagaggcctgagagaaactgaggtcaagatttcaggattaatggtcctgtgatgctttgaagtacaattgtggatttgtccaattctctttagttctgtcagcttttgcttcatatattttagcgctctattattagatatatacatgtttagtattatgtcttattggtgcatttactctcttatcattatgtaatgtccttctttatctgtgataattttctgtgttctgaagtctactttgtctaaaaataacatacgcactcaacttccttttctttcttccttcctttctttcttccttcctttctttctctctctctctctttccttccttccttcctccttttctttctctctctctctctctctctttttttgacagactctcgttctgtggccctggctggagttcagtggtgtgatcttggctcactgctacctctaccatgagcaattctcctgcctcagcctcccaagtagctggaactacaggctcatgccactgcgcccagctaatttttgtatttttcgtagagacggggtttcaccacattcgtcaggttggtttcaaactcctgactttgtgatccacccgcctcggcctcccaaagtgctgggattacaggcatgagccatcacacctggtcaactttcttttgattagtgtttttgtggtatatctttttccatcatgttactttaaatatatctatattattgtatttaaaatgtgtttcttacagactgcatgtagttgggtataatttttatccagtctaaaaatatctgtcttttaattggtgtttagacaatttatatttaataaaattgttgaatttaaaaaaaaaaaaaaaa

SEQ ID NO: 27 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 28 - DNA, 4592 bp, Homo sapiens
aggctgttgaggctgggccatctcctcctcacttccattctgactgcagtctgtggttctgattccataccagaggggctcaggatgctgttgctgggagctgttctactgctattagctctgcccggtcatgaccaggaaaccacgactcaagggcccggagtcctgcttcccctgcccaagggggcctgcacaggttggatggcgggcatcccagggcatccgggccataatggggccccaggccgtgatggcagagatggcacccctggtgagaagggtgagaaaggagatccaggtcttattggtcctaagggagacatcggtgaaaccggagtacccggggctgaaggtccccgaggctttccgggaatccaaggcaggaaaggagaacctggagaaggtgcctatgtacaccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactgatcaccactaactcagagcctcctccaggccaaacagccccaaagtcaattaaaggctttcagtacggttaggaagttgattattatttagttggaggcctttagatattattcattcatttactcattcatttattcattcattcatcaagtaactttaaaaaaatcatatgctatgttcccagtcctggggagcttcacaaacatgaccagataactgactagaaagaagtagttgacagtgctattttgtgcccactgtctctcctgatgctcatatcaatcctataaggcacagggaacaagcattctcctgtttttacagattgtatcctgaggctgagagagttaagtgaatgtctaaggtcacacagtattaagtgacagtgctagaaatcaaacccagagctgtggactttgttcactagactgtgcccttttatagaggtacatgttctctttggagtgttggtaggtgtctgtttcccacctcacctgagagccattgaatttgccttcctcatgaattaaaacctcccccaagcagagcttcctcagagaaagtggttctatgatgaagtcctgtcttggaaggactactactcaatggcccctgcactactctacttcctcttacctatgtcccttctcatgcctttccctccaacggggaaagccaactccatctctaagtgctgaactcatccctgttcctcaaggccacctggccaggagcttctctgatgtgatatccactttttttttttttgagatggagtctcactctgtcacccaggctggagtacagtgacacgacctcggctcactgcagcctccttctcctgggtccaagcaattattgtgcctcagcctcccgagtagctgagacttcaggtgcattccaccacacatggctaatttttgtatttttagtagaaatggggtttcgtcatgttggccaggctggtctcgaactcctggcctaggtgatccacccgcctcgacctcccaaagtgctgggattacaggcatgagccaccatgcccagtcgatatctcactttttattttgccatggatgagagtcctgggtgtgaggaacacctcccaccaggctagaggcaactgcccaggaaggactgtgcttccgtcacctctaaatcccttgcagatccttgataaatgcctcatgaagaccaatctcttgaatcccatatctacccagaattaactccattccagtctctgcatgtaatcagttttatccacagaaacattttcattttaggaaatccctggttttaagtatcaatccttgttcagctggacaatatgaatcttttccactgaagttagggatgactgtgattttcagaacacgtccagaatttttcatcaagaaggtagcttgagcctgaaatgcaaaacccatggaggaattctgaagccattgtctccttgagtaccaacagggtcagggaagactgggcctcctgaatttattattgttctttaagaattacaggttgaggtagttgatggtggtaaacattctctcaggagacaataactccagtgatgttcttcaaagattttagcaaaaacagagtaaatagcattctctatcaatatataaatttaaaaaactatctttttgcttacagttttaaattctgaacaattctctcttatatgtgtattgctaatcattaaggtattattttttccacatataaagctttgtctttttgttgttgttgttgtttttaagatggagtttccctctgttgccaggctagagtgcagtggcatgatctcggcttactgcaacctttgcctcccaggttcaagcgattcttctgcctcagcctcccgagtagctgggaccacaggtgcctaccaccatgccaggctaatttttgtatttttagtaaagacagggtttcaccatattggccaggctggtctcgaactcctgaccttgtgatctgcccgcctccatttttgttgttattttttgagaaagatagatatgaggtttagagagggatgaagaggtgagagtaagccttgtgttagtcagaactctgtgttgtgaatgtcattcacaacagaaaacccaaaatattatgcaaactactgtaagcaagaaaaataaaggaaaaatggaaacatttattcctttgcataatagaaattaccagagttgttctgtctttagataaggtttgaaccaaagctcaaaacaatcaagacccttttctgtatgtccttctgttctgccttccgcagtgtaggctttaccctcaggtgctacacagtatagttctagggtttccctcccgatatcaaaaagactgtggcctgcccagctctcgtatccccaagccacaccatctggctaaatggacatcatgttttctggtgatgcccaaagaggagagaggaagctctctttcccagatgccccagcaagtgtaaccttgcatctcattgctctggctgagttgtgtgcctgtttctgaccaatcactgagtcaggaggatgaaatattcatattgacttaattgcagcttaagttaggggtatgtagaggtattttccctaaagcaaaattgggacactgttatcagaaataggagagtggatgatagatgcaaaataatacctgtccacaacaaactcttaatgctgtgtttgagctttcatgagtttcccagagagacatagctggaaaattcctattgattttctctaaaatttcaacaagtagctaaagtctggctatgctcacagtctcacatctggttggggtgggctccttacagaacacgctttcacagttaccctaaactctctggggcagggttattcctttgtggaaccagaggcacagagagagtcaactgaggccaaaagaggcctgagagaaactgaggtcaagatttcaggattaatggtcctgtgatgctttgaagtacaattgtggatttgtccaattctctttagttctgtcagcttttgcttcatatattttagcgctctattattagatatatacatgtttagtattatgtcttattggtgcatttactctcttatcattatgtaatgtccttctttatctgtgataattttctgtgttctgaagtctactttgtctaaaaataacatacgcactcaacttccttttctttcttccttcctttctttcttccttcctttctttctctctctctctctttccttccttccttcctccttttctttctctctctctctctctctctttttttgacagactctcgttctgtggccctggctggagttcagtggtgtgatcttggctcactgctacctctaccatgagcaattctcctgcctcagcctcccaagtagctggaactacaggctcatgccactgcgcccagctaatttttgtatttttcgtagagacggggtttcaccacattcgtcaggttggtttcaaactcctgactttgtgatccacccgcctcggcctcccaaagtgctgggattacaggcatgagccatcacacctggtcaactttcttttgattagtgtttttgtggtatatctttttccatcatgttactttaaatatatctatattattgtatttaaaatgtgtttcttacagactgcatgtagttgggtataatttttatccagtctaaaaatatctgtcttttaattggtgtttagacaatttatatttaataaaattgttgaatttaaaaaaaaaaaaaaaa

SEQ ID NO: 29 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVHRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 30 - DNA, 18 bp, Homo sapiens
aggtccccgaggctttcc

SEQ ID NO: 31 - DNA, 19 bp, Homo sapiens
aagcgaatgggcatgttgg

SEQ ID NO: 32 - DNA, 29 bp, Homo sapiens
aggtgcctatgtataccgctcagcattca

SEQ ID NO: 33 - DNA, 27 bp, Homo sapiens
aggtgcctatgtacaccgctcagcatt

SEQ ID NO: 34 - DNA, 24 bp, Bacteriophage M13
cgccagggttttcccagtcacgac

SEQ ID NO: 35 - DNA, 24 bp, Bacteriophage M13
agcggataacaatttcacacagga

SEQ ID NO: 36 - DNA, 37 bp, Homo sapiens
ggagaaggtgcctatgtacaccgctcagcattcagtg

SEQ ID NO: 37 - DNA, 37 bp, Homo sapiens
cactgaatgctgagcggtgtacataggcaccttctcc

SEQ ID NO: 38 - DNA, 17788 bp, Homo sapiens
tcagatcctgcccttcaaaaacaaaacatgagcgtgccaagaaagtccaaggtgttgaatgttgccacttcaagcctaaactttctaggaacacctaagtgggtggcagcttccagttctccaggctgcttctaggccagagctgggttccacaagagacagaataggcatatatatgcttaaggaactggaaaaacaggctctctctctctcacaaacacacacacacacataccaaggtagctgtcaaaatgttatccgaaattttggaaccaaaaaatcttgaaagatggtattccaatatcacattttatgtaagttttctattatattagattcaaattacgattcgaggccacaagctttaagaattcagggcctttttaacttgccaagccccacaccactccaggaacttccccacaccccagttctcagaattcatgtgcaaggtctttcctaaatccagggtccaggtcagagagtggaggatgtgctctatttcttacctgattgcagacccctctgacagtgctcccttctgaagcactcactgtctgaacgtacacagtctcagacttaatcatgcacagtgagcaagactgtggtgtgataattggcgtccctgacttattagggcaaatctatgggagggggagacctcctggaccactgagcaattaattcatttacattaggaagtttctccgtcagatgcaggaaaaaaatcttgttttcctgctgtggttttgacttttgccccatcttctgttgctgttgtaggaggcaaaataagggtcaaggcctggaaacacaagtgctttgactgaagctccacttggcttccgaagcccaagctgggttgtaccaggttccctagggtgcaggctgtgggcaactgccagggacatgtgcctgcccaccggcctctggccctcactgagttggccaatgggaaatgacaattgtgaggtggggactgcctgcccccgtgagtaccaggctgttgaggctgggccatctcctcctcacttccattctgactgcagtctgtggttctgattccataccagagggtaagagcaattctgtgaagttccaggctgggtgggggatgcatgcatagcctctggctgggatcacccaggctctcccgtccgtagtagtgtgggagtggatacaggtggatactctggtcagagcagcactggtggaggcagatatgcactgggcttcttcctccgttctcccacagccccaagagagaaagggttatttcagacattccttctaagatgcatggaaccattctgaattttgcccagttcgctctgtagcaggatacctattgagaaaaagttagggtcagtaaggtggaagggtctgtccacagatgaagtccaattcgattaagggggataagggaatacattgcctcttagcttgaccaggtagggcaaaggaagaagcatatatgaaggcagcttcagaaaagtcaagctgagcactgacttcagactggaattaggaatccagctctgccactttattctactcagcaaatatttactgagcaaattctatgggctagacagtggattgggttcacaagatacaatgagtgtgacatggttgttgtctatggatttggggatatatgtaggtatagggatatcttacaaggtaatcaagaggttctaatgaggccagccatggtggctcacacctgtaatcccagcaatttgggagaccgaggcgggtggatcacctgaggtcaggagttccagactagcctgaccaacatggtgaaaccccgcctctaccaaaaatacaaaaattagttgggcgtgatggcaggtgcctgtaatcccagcttctcgggaggctgaggcaggagaattgtctgaacctgggaggcagaggttgcagtgagccgagattgttgccactgcattccagcctgggtgacagagcgagactttgtgtcaaaaaaaaaaaaaaaagaaagaaaagaaaaagaggctctaatgagataaaatgagaaaagcctggcatatagtggcaacttatgaaaaattgtaattaaaaaaaaacattttctgacagaagaaactggatctacctggtttttctgaagcctaatcctgctcgccccagtgagtgctgtttctgaggcatcctggttgttttgagctgtggatgctgaaggttagagtgggagggattttagaggttaggtctgcccctcttgtgttagaggacatggatccctggtctggagaggttctggtttttggatcaagcctcacaaggggtggcaccaactcactcctaggaactccgctagaaggaaggccagctctgcctaattcggttggggagatgggggtccctttatgctagcagaatatgtccgaaggagcatgatggtgtcagctttgttcatgaaggccagtggtacacagggagcccggcagcttcctcagcagtccctgctgccactcttccttaagtcttgaggagtctttttttggcacaatctcagctcactgcaacctccgcctcccaggttcaagcgattctcctgcctcagtctcccaagtagctgagactacaggcatgcgccaccacgcccagctaatttttatatttttagtagagatggggttcaccatattggccaggatggtctcgatctcttgacctcatattccacctgcctcggcctcccaaagtgctggtattacaggtgtgagccactgcgcctggccgaggagtcttaagctgagatcacagcattgcactccagcctgggcaaaaaaagcaaaactccatctcaaaaaaaaaaaaaaaaaaatagacacaagactggctccttgtcttttttggggacagggtctcactctatcacccaggctggagtgcagtggtgcaatcacagctcactgcagcctcgatttcccaggctcaagtgaccctcccatcttagcctcctgagtagctgggactacaggtgtgtgcaaccatgcctggctaatttttaaaaattttttgtagagatgaggtctcactatattggctggggggcctcaaactcctgggctcagcagtcctcccacctcagcctcccaaaaggctgggattatatgcttgctctttttaaggtggctgtagggacaaactttccacctactccttgtcaagccagtggaccggtggtcccagacatacggctaaagtcaagaggtgatgtcttttggagagatactttcaatcaggaatttcaatcagaaattcaatcatgtggagagagacttatcctaaaaatgtggtggtgcgtgggatgctctgttttattagttccttgacagtatgtatgtgtgtgagtgtgtgtgtgcgcgcgcgcacactcatttggatgggtgtgtatgtgtgggggggggtggtgcgtacgtatgtggatgtgtggatgtggtgtgtgggtgtgcgcgtgcataggtggaggtgtgtgtatgggtgcgggtatgtgtgtgtgttgggcatggagatattgacagctctcccagggctgagtgaaggctttcgggcaaagctcctgggagctaggcaaagctgagttgattcctggttatgccatttattattgggttgcaccgtgtgaaactgccaatattctacactttgacttttatttatttttatttttattttttttgagacagagtttcacacttgtcacctaggctggagtgcagtggcgcgatctcagctcactgcaacctctgcctcatggattcaagtgattctcctgcctcagcctcccaagtagctggaattacaggtgcccgctaccacgcctgactaatttttgtatttttggtagagacgggatttcaccatgttgtccaggctggtctgaaactcctgacatcaggtaatccacccacctcagcctcccaaagtgctgggattacaggcatgagccactgcgcccggcccattttgacttttaaaaatgggagtttgatataattcaatccagtggttgaattagctagcatcgttccctctccaagtctcaggttctcctacacgttagagtcaaaagcagggctatgggaagattaagtaaaataaattttgaaaatgccttatgaaaattacactccaaagaactcgcgccagtgtcagtgttctcatgttcctcatctcacatgatcacatttcgcggattaggaagctgagtctgagaagctccgtgtagtgctttttcggaggcaccgtgatgtgatggaaggctcactcgttaggaagtcagaacagagtctctgagggatcatctccttaatctgtcagtttcctcatctctgaagttgggctcatttccttccttcatggagttattgtaaagatgaagataaataacgtgtaaaatctagcatgggaactggcttctataaggttctaataagtgcattcctactccttcccctcagccttcccatttgtaaaagcaaggcaggggtgaggtgatttctggggctccttttggctctgacatttgaggattttgtatacttttttttttttgagacagagtcttgctctgtcacccaggttggagtgcagctcaatgcaaattccgcctcccaggctcaagcaattcttatgtctcagcctcctgagtacctgggattacaggcaggcaccaccacccccagctaatttttttgtattttcagtagagacggggttttgccatattggccaggctggtcttgaactcctgacttcatgtgacccacccatctcagcctcccaaagtgctgagatgacaggtgtgagctaccgtgcctggccaattttgtgtgctttaatgcccttttctgctggaagagttggcaccaggtttggtgatctctttcccccacacggctctgcctcctgccagtcccagaggggaccctgtccttgcatttcacaggattctgctgttgcaactgaaattccagtaggtcaaagtgaaatttctcatacactttaacatgaagataaatgatcacagtatggccctttaggatcctgagaacatcacggtcatcccctggtataattttaaaagcagatgaatccatgcctgtgcgaggtttgccaggaaagccagtgctgggattacagtggaagtctttttatgctacttttttcttgtatccctcaccccatggggtggcatattgaaaggcaggatgtgtgaccacgatacttttctcctcctggactatgtctaagagtctgttattgggttctgaagatcagagtttaatttccgactcctctctgtgtagctctgggatcttggaaagccacttaacctttctgaagtcccctttcctcatctctaaaatgcatacactcatcactaacatttactgagcactgacatgtgccagacaccattctaagcattttacacagactacaccatttgatcttccaacaaacagaacactgaaacgcattacaggtcagaacaaatgatttgtgcctaagcaccaagaccgtagagcccgtgctccctattctaccctatcctgtctctcaaaatgattgtgagaatcgaatgagacactaggtgagaaaagggttttataaatagcattttaaaaattttttaaagtccacaaaatttttaattttaatacagataaaatagatccctttgttttataaaaagtaacaaaatttgttatacaacaactatgttatttattaattttgcctttttgtatgctgccaggaaagaaacattaagaaatcttaaattgattatggtgaatcagaaggtctgcctggactttttattgctctaactgtacagctgatcatactacctcatttttttttatgacacttcaagggtgcgcttagcttcatcactccttcgttgccaaaagctttgtgaccaaaaacaattaagcagattcctgagtcactaaatgacacataaccagagttgagacttaggaacttttagtgccatgctaagcccacagggacacaacaaatagcattttacaaaggcaaagaattgtgacacttgagatttagcttgttgatccttgtaaaagttttctttttaggcataattgagttttagatcatagtactcactattacttagtaataatttttttctgatagaaatacagtgtaacaggccgggcgcagtggctcatgcctgtaatcccagcactttgggaggccgaggcgggcggatcacttgaggtcaggagtttgagaccagcccggccaacatggtgaaatcccatctctactaaaaatacaaaaaattagccaggtgtggtcgtggattcctgtgatcccagctacttgggagggtgaggcaggagcatcagttgaacccaggaggcggaggttgcagtgagccaagatggtgccattgcactccagcctgggccacaaagcgagactccacttcagaaacaaaaaaaaaagagagagagaaaagaaggaaggaaggaaggaaggaaggaaagaaggaaggaaggaaggaaagaaggaaggaaggaaggaaggaaagaaggaaggaaggaaagaaggaaggaaggaaagaaggaaggaaggaaggaagggtaacaagcaaagtgtaacaatggcaatatctaaaaaaataggtatttttatatgtttgtcgttttatatatatgacccccactttagagatgaggaaactgagagattaaggaaacgatccctgagagactctgttctgacttccaaatcggtgagctttccatcgcatcacggtgcctccgaaagcatgacacggagcttctcagactcagcttcctaatccgctaaacgggattatgtgagatgaggaacatgagaacgctgacatgggtgagggttccttggagtatcattttcatgtggcattttcaaaacttattttacctaatcttcccaaagccctgcttttgactctaatgtgtctcctgagacttggagagcgcaagatgctagcgacagagcaagactccatctccagataaataaataagtaaaataaaaaagaacacaaataattttgaaaatttttttgaaaattaggcacgtttgcactgaccttcaattgttattaattgctggtttcccacccagaattaagttggaatgcaactttcttttacaatcagagtccgttcttggtcttggaaacttctgaggctcctgtgctaatcacactcttgtatttttggcacctctaccccgtgccactgtcatggaacccaggctgatcgcacctattagtggagaaatctgtccataatactgaagtttggggacaaacagtgttcccttagggtaggagaaagagatctttatttttgacaaagggggaggagccagaaaactccagagacccctgagtttgccctctctccaaggtttggggtaagccccccgtcaccctttatctctggggctttcacatattctggattctctcctcctgtttcccagcagaaaaggatggagcctcacagattcttcccatttctggagaaaaacatgcatggagctcaaagttcttctcaggagttttattgccaaagccataataagaaagggtggaggtgacaagcagtgaggaagtttaaagatgcatgaaatctgtaaagtctcagaacaagaattctcctaaaatgcaaaaggggctttgctggtctccccttggcttctcatgtagctcacctcttttttcttatcttgagactagtcaaacctaagctgtttctcattttatttccagaagctattgagaacactctcctgaattcttcaaattcagtagagggcgacaaatgtacatataaatgatggtagtgggtcttaaataaagactcatgacacctaaaggggcagcacctgagtctgattgcacctgtttctgttgctgtttctgtctctcttctctctgtctgccatttcattatcaatggttactttacttataagatcatattagaacctgatatttgataaatgatgcatcagatctatagtgagagaaaaaattaatgcaattaaaggtgttgtaacagctagtcttcaagtggggagaaatcatttgagtaccttaggtcacagcttacatcaaaacaaaaaatcagagctacattaaaaagtgaaattttaactatatcaaacaatagaaaaaaacagaagaaaattgaatacttactaaatcttagcatgaataagaactgtttaacacttagaggcaaggactgggcgtggtggctcatgcttttaatcccaggactttgggagcccaaggcgggcggatcacctgaggtcaggagtttgagactagcctggccaacatggtgaaaccccgtctctactaaaaaatgcaaaaattagctgcgtgtggtggtgcatgcctgtaatctcagctacttgggaggctaaggcatgagaatcgcttgaacctgggaggtggaggctgtagtgagccgagattgtgccactgcactacagcctgggtgacagtgtgaaatcctctctctcaaaaaaaaaaaaaaaaaaaagcaaactagagcagtgaggtaccattatttcctttgctcactaaactgacaacacacaaatgttttttataatacccaaagctgatgagggtagttaaggtatgcccttttatacacacactaatgatgtactactggttggcagtataacatatgctgccatgtggggatatgtatcaggagacttaaaaatgtgcataccttttggtccagtaatttacttctgggaatctgtcataacagaataataatcttggggaaagctacatgcctaaggatatttaaaatattatttaaaaatcaaagtataatttcttacagaatataaaataatattttaaaatgaaaatatgctaaaagtttgatgaaatataaatggtcaaatatatattgattatatccacttactagactagcactcactctgagacgttaaaaatagtcattataaaaactagaaaatgccaaagacaaaataaaggaataaagttttacataaagtatgattccactatgtttaaaaataaacagagacattcttggagttgagtattgttttcttttctgtcatgtccaaagaactatataactattatttttaatgaactatatatgtaatatatacatatagtttatatgtatatacaaaatttatctcatatatatgataaagatgaaagatgagttggatgtgccacgtgaagagggtagtatagaaccccaggtaatggggcataggagtgggattccagataccaggcccatgtttttggggtgagattgccaatcacggtctttcttccatccctcacagaggagtaggtttgtcttcaacaaaccttcagttgtcctgaagacaaacctaattctggagacttcatataatctagaagagacaagcaaactgatgaaaaatagtgaatttttaaggtaaaataaagtacatggactacactttgtttagaatcagattcttgggattaaccacattaacccacagagggtcttagtgatgcctctaatccaggatcctaggacctatttctctctgtgagatgctttctcccaactccttggtgagagtgggaagactaagacctcagcaatctgaggtggaggcctaagatccccctaagatcggaggcagaatctgagaggggataaaagtccctatacctgtattgggcccttttctgggagggggatatcaaagaatgattttgagacagggaggcttttgactacctgtgccacttgagctctttgctagggctccagaatacatatttcaaatacattccccctccctccttccttccctcttccactcttcctttttatcttcctttcttcttttccttcctccttcccttcctttctctggctctctcatgatttcttttcctcattataaaagtgcttatttagtccctactctgctattagtgtgttagtctttgtcccctggtacttgctgtttaatggagaaatgggtgagcaaaacagaaattacagcagagtgcaatgatagagctaagccaggtgtataaatccattctcacactgctgtaaaaaactacttgagactgggtaatttataaagaaaagaggtttaattgactcacagttccacaggctgtacaggaagcatggctggggaggcctcagaaaacttacaatcatggtggaagaaagagcgaaggggaagcaagcacatcacacagcagcaggagagagagagagaaagagagagagagagaatataggggaagtgctacacactttcaaccagatcttgtgagaattcacctactatcatgagaacagcaagggataagtctgcctccatgattcagtcacctcctaccaggccccttctccaacacatgtcgacgtgctatttgggtggggacacagacccaaaccatattaccagggcactggagaaacacagaggggaaagaaccagccaaggagtgagatggagaacaaggaggacttcttgaaacagatgacatccaaactgggtcctgaaagctgaatagagattagacaggggaggaggggcagctaaagatggctcaggcaaacaaagggccaggggatatgttcatgggatgatgtgtctctcgttgtctgcttaacacaaggtgagtctctccctccctctctctctctttttctctgtgtgtgtttgtgtgtgtgcatgtgtgcaaatgtaatatacccaatagtcaaacatgtgccccaggagaggggtagaggaagaaagagaatgagagagtaagaaggaggaatagacacagaaaatgagagagaaggggggaaagaaaaagaagaaaggagccagaggagagaagctggttagcattgaatggagcaatctgtgtcatcgtacttgggaaacccaaggatggattcttggcaagtcgactcttggagctttccctgtgcttggtcctgtgctcagacatgggaaaattagaggagtgtcatctgtgcaatcactgaattcataatcttggtgaggaaaggagactacacacagggaataatgctaagtattacagatttcagggcagaaagagatcaaggtgggctgcaatattcagaaaagtcttcctggaaaagttgaatacttagaaagcagctcctagaagtagactctgctgagatggacggagtcctttgtaggtcccaactgggtgtgtgtgtggggtctgtctctccatggctgacagtgcacatgtggattccagggctcaggatgctgttgctgggagctgttctactgctattagctctgcccggtcatgaccaggaaaccacgactcaagggcccggagtcctgcttcccctgcccaagggggcctgcacaggttggatggcgggcatcccagggcatccgggccataatggggccccaggccgtgatggcagagatggcacccctggtgagaagggtgagaaaggagatccaggtaagaatgtttctggcctctttcatcacagacctcctacactgatataaactatatgaaggcattcattattaactaaggcctagacacagggagaaagcaaagcttttttatgttaaccataagcaacctgaagtgatttggggttggtcttccaaggatgagtgtagatggtgcctctataaccaagactttggctttgctgcatctgcagctccttttccatcccctttcccatcttcaccctcatccctattcccagtacattcatattctgattcctctttctgtctgcttaacttccatttcacccagtggcattcaaccacatttactgcacaccccctgaaaggctcagtcctgcctttggggaactcttgatctaggtaagatgtctaatgtgcaaggctctgttggtggttactacaagaaagtctactctaaaaatgtcaaactgaatgtgaacaagtattcaaagtatggagcatagagaaaatatactcaccgtggacctgatgaagaatgaaggcttcaaggaggaggcagagcttcagctaggccttgaatgatgggtaggcagaatagaggaggagagacatcctagatggagggggtagaattgcaaaaccagggttgatggtgccagcacataaagggctggcagggtggagggtctatgatagagacctataggagataaagatagagttgaaattatgggagcctccatgtctgtgggagatatagaaggaggaggtaacacctctctccttttgggagctcttattggtttcttgatctataagtcaagaaggttgtgagtgggagccacagggatggtaatttaggctgtaaccaacctaggcaggagttctgttctttgtagtcactgaggtcttctcattccttaggtcttattggtcctaagggagacatcggtgaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactgatcaccactaactcagagcctcctccaggccaaacagccccaaagtcaattaaaggctttcagtacggttaggaagttgattattatttagttggaggcctttagatattattcattcatttactcattcatttattcattcattcatcgagtaactttaaaaaaatcatatgctatgttcccagtcctggggagcttcacaaacatgaccagataactgactagaaagaagtagttgacagtgctattttgtgcccactgtctctcctgatgctcatatcaatcctataaggcacagggaacaagcattctcctgtttttacagattgtatcctgaggctgagagagttaagtgaatgtctaaggtcacacagtattaagtgacagtgctagaaatcaaacccagagctgtggactttgttcactagactgtgcccttttatagaggtacatgttctctttggagtgttggtaggtgtctgtttcccacctcacctgagagccattgaatttgccttcctcatgaattaaaacctcccccaagcagagcttcctcagagaaagtggttctatgatgacgtcctgtcttggaaggactactactcaatggcccctgcactactctacttcctcttacctatgtcccttctcatgcctttccctccaacggggaaagccaactccatctctaagtgccgaactcatccctgttcctcaaggccacctggccaggagcttctctgatgtgatatccactttttttttttttgagatggagtctcactctgtcacccaggctggagtacagtgacacgacctcggctcactgcagcctccttctcctgggtccaagcaattattgtgcctcagcctcccgagtagctgagacttcaggtgcattccaccacacatggctaatttttgtatttttagtagaaatggggtttcgtcatgttggccaggctggtctcgaactcctggcctaggtgatccacccgcctcgacctcccaaagtgctgggattacaggcatgagccaccatgcccagtcgatatctcactttttattttgccatggatgagagtcctgggtgtgaggaacacctcccaccaggctagaggcaactgcccaggaaggactgtgcttccgtcacctctaaatcccttgcagatccttgataaatgcctcatgaagaccaatctcttgaatcccatatctacccagaattaactccattccagtctctgcatgtaatcagttttatccacagaaacattttcattttaggaaatccctggttttaagtatcaatccttgttcagctggacaatatgaatcttttccactgaagttagggatgactgtgattttcagaacacgtccagaatttttcatcaagaaggtagcttgagcctgaaatgcaaaacccatggaggaattctgaagccattgtctccttgagtaccaacagggtcagggaagactgggcctcctgaatttattattgttctttaagaattacaggttgaggtagttgatggtggtaaacattctctcaggagacaataactccagtgatgttcttcaaagattttagcaaaaacagagtaaatagcattctctatcaatatataaatttaaaaaactatctttttgcttacagttttaaattctgaacaattctctcttatatgtgtattgctaatcattaaggtattattttttccacatataaagctttgtctttttgttgttgttgttgtttttaagatggagtttccctctgttgccaggctagagtgcagtggcatgatctcggcttactgcaacctttgcctcccaggttcaagcgattcttctgcctcagcctcccgagtagctgggaccacaggtgcctaccaccatgccaggctaatttttgtatttttagtaaagacagggtttcaccatattggccaggctggtctcgaactcctgaccttgtgatctgcccgcctccatttttgttgttattttttgagaaagatagatatgaggtttagagagggatgaagaggtgagagtaagccttgtgttagtcagaactctgtgttgtgaatgtcattcacaacagaaaacccaaaatattatgcaaactactgtaagcaagaaaaataaaggaaaaatggaaacatttattcctttgcataatagaaattaccagagttgttctgtctttagataaggtttgaaccaaagctcaaaacaatcaagacccttttctgtatgtccttctgttctgccttccgcagtgtaggctttaccctcaggtgctacacagtatagttctagggtttccctcccgatatcaaaaagactgtggcctgcccagctctcgtatccccaagccacaccatctggctaaatggacatcatgttttctggtgatgcccaaagaggagagaggaagctctctttcccagatgccccagcaagtgtaaccttgcatctcattgctctggctgagttgtgtgcctgtttctgaccaatcactgagtcaggaggatgaaatattcatattgacttaattgcagcttaagttaggggtatgtagaggtattttccctaaagcaaaattgggacactgttatcagaaataggagagtggatgatagatgcaaaataatacctgtccacaacaaactcttaatgctgtgtttgagctttcatgagtttcccagagagacatagctggaaaattcctattgattttctctaaaatttcaacaagtagctaaagtctggctatgctcacagtctcacatctggttggggtgggctccttacagaacacgctttcacagttaccctaaactctctggggcagggttattcctttgtggaaccagaggcacagagagagtcaactgaggccaaaagaggcctgagagaaactgaggtcaagatttcaggattaatggtcctgtgatgctttgaagtacaattgtggatttgtccaattctctttagttctgtcagcttttgcttcatatattttagcgctctattattagatatatacatgtttagtattatgtcttattggtgcatttactctcttatcattatgtaatgtccttctttatctgtgataattttctgtgttctgaagtctactttgtctaaaaataacatacgcactcaacttccttttctttcttccttcctttctttcttccttcctttctttctctctctctctctttccttccttccttcctccttttctttctctctctctctctctctctttttttgacagactctcgttctgtggccctggctggagttcagtggtgtgatcttggctcactgctacctctaccatgagcaattctcctgcctcagcctcccaagtagctggaactacaggctcatgccactgcgcccagctaatttttgtatttttcgtagagacggggtttcaccacattcgtcaggttggtttcaaactcctgactttgtgatccacccgcctcggcctcccaaagtgctgggattacaggcatgagccatcacacctggtcaactttcttttgattagtgtttttgtggtatatctttttccatcatgttactttaaatatatctatattattgtatttaaaatgtgtttcttacagactgcatgtagttgggtataatttttatccagtctaaaaatatctgtcttttaattggtgtttagacaatttatatttaataaaattgttgaatttaagatggatgactgttttatttgtttgctgttcaccacttctgttttattctctttccagaattcttttggattgtttaaatatttcataatattttatcttaatttatttattgggtatttgcctatatctctttgtggtattttttagtggttgcttgagggattacaatgtacttaacttttcacagtgtgcataaagttaatattttgccacttgcagtaaaccgtagaaggcttataatcatattagtacctctatccactttcttttatgttgtagttgtcatatatattacatctatatacactgaaacattataggcaatgttatgatttttgcattcgtcagtcatatatatattttaaagaatttaagaggagaaaaatacatattcagatattcatcatttcctttgttctttattaatttctaatgttctgtgtttctctctggtgtaatttctcctcaacctgaaaaactttattgagcatttattttaaagcaagtctgttgatgatgaattctcttagtttttcttcatctgagaatgccttcatcttttctacattcctgaaatatatttttactggatatagaattctcagttgacagttctttcttctagcattttattttatttagtttatttttttcagtgcattaaagatgtttctgtcttctgggacccatggtgtgtgtgtatatatatatatatttttttttctctttatctcaaaggctctatggtttcagatgagaaacctgcagtcatagttttcccttatgtgtaatttataattttttcttccagctgctttcaagatgctttctttacctctgattttcaatagtttgattatgatgtgtctgggtgttttcaactttcacccattttagggttcattaaacttcttgaatctataaatataggtctttcagcaaatttaggacattttctgcagacattttttatttttatttttgagatttgagtgtt

(19) United States
(12) Patent Application Publication
(10) Pub. No.: US 2007/0190539 A1
(43) Pub. Date: Aug. 16, 2007

(54) Human MLR single nucleotide polymorphisms associated with dose-dependent congestive heart failure and methods of use thereof

(75) Inventor: Koustubh Ranade, Princeton, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/498,489

(22) Filed: Aug. 3, 2006

Related U.S. Application Data
(60) Provisional application No. 60/705,996, filed on Aug. 5, 2005.
(60) Provisional application No. 60/707,802, filed on Aug. 12, 2005.
(60) Provisional application No. 60/707,729, filed on Aug. 12, 2005.

(51) Int. Cl. C12Q 1/68; C07H 21/04; C07K 14/705

(52) U.S. Cl. 435/6; 536/23.5; 530/350

(57) ABSTRACT

The invention provides novel polynucleotides and polypeptides associated with the incidence of PPAR-agonist induced congestive heart failure. The invention also provides polynucleotide fragments corresponding to the genomic and/or coding regions of these polynucleotides which comprise at least one polymorphic locus per fragment. Allele-specific primers and probes which hybridize to these regions, and/or which comprise at least one polymorphic locus are also provided. The polynucleotides, primers, and probes of the present invention are useful in phenotype correlations, medicine, and genetic analysis. Also provided are vectors, host cells, antibodies, and recombinant and synthetic methods for producing said polynucleotides and/or polypeptides. The invention further relates to diagnostic and therapeutic methods for applying these novel polynucleotides and polypeptides to the diagnosis, treatment, and/or prevention of various diseases and/or disorders, particularly PPAR-agonist induced congestive heart failure or related indications.

This application claims benefit to provisional application U.S. Ser. No. 60/705,996 filed Aug. 5, 2005; to provisional application U.S. Ser. No. 60/707,802 filed Aug. 12, 2005; and to provisional application U.S. Ser. No. 60/707,729, filed Aug. 12, 2005; under 35 U.S.C. 119(e). The entire teachings of the referenced applications are incorporated herein by reference.

### FIELD OF THE INVENTION

The invention provides novel polynucleotides and polypeptides associated with the incidence of PPAR-agonist induced congestive heart failure. The invention also provides polynucleotide fragments corresponding to the genomic and/or coding regions of these polynucleotides which comprise at least one polymorphic locus per fragment. Allele-specific primers and probes which hybridize to these regions, and/or which comprise at least one polymorphic locus are also provided. The polynucleotides, primers, and probes of the present invention are useful in phenotype correlations, medicine, and genetic analysis. The invention further relates to diagnostic methods for using these novel polynucleotides in the diagnosis, treatment, and/or prevention of various PPAR-related diseases and/or disorders.

### BACKGROUND OF THE INVENTION

The genomes of all organisms undergo spontaneous mutation in the course of their continuing evolution, generating variant forms of progenitor nucleic acid sequences (Gusella, Ann. Rev. Biochem., 55:831-854 (1986). The variant form may confer an evolutionary advantage or disadvantage relative to a progenitor form, or may be neutral. In some instances, a variant form confers a lethal disadvantage and is not transmitted to subsequent generations of the organism. In other instances, a variant form confers an evolutionary advantage to the species and is eventually incorporated into the DNA of many or most members of the species and effectively becomes the progenitor form. In many instances, both progenitor and variant form(s) survive and co-exist in a species population. The coexistence of multiple forms of a sequence gives rise to polymorphisms.

Several different types of polymorphism have been reported. A restriction fragment length polymorphism (RFLP) is a variation in DNA sequence that alters the length of a restriction fragment. The restriction fragment length polymorphism may create or delete a restriction site, thus changing the length of the restriction fragment. RFLPs have been widely used in human and animal genetic analyses. When a heritable trait can be linked to a particular RFLP, the presence of the RFLP in an individual can be used to predict the likelihood that the animal will also exhibit the trait.

Other polymorphisms take the form of short tandem repeats (STRs) that include tandem di-, tri- and tetra-nucleotide repeated motifs. These tandem repeats are also referred to as variable number tandem repeat (VNTR) polymorphisms. VNTRs have been used in identity and paternity analysis, and in a large number of genetic mapping studies.

Other polymorphisms take the form of single nucleotide variations between individuals of the same species. Such polymorphisms are far more frequent than RFLPs, STRs and VNTRs. Some single nucleotide polymorphisms (SNPs) occur in protein-coding nucleic acid sequences (coding sequence SNP (cSNP)), in which case, one of the polymorphic forms may give rise to the expression of a defective or otherwise variant protein and, potentially, a genetic disease. Examples of genes in which polymorphisms within coding sequences give rise to genetic disease include: globin (sickle cell anemia), apoE4 (Alzheimer's Disease), Factor V Leiden (thrombosis), and CFTR (cystic fibrosis). cSNPs can alter the codon sequence of the gene and therefore specify an alternative amino acid. Such changes are called “missense” when another amino acid is substituted, and “nonsense” when the alternative codon specifies a stop signal in protein translation. When the cSNP does not alter the amino acid specified the cSNP is called “silent”.

Other single nucleotide polymorphisms occur in noncoding regions. Some of these polymorphisms may also result in defective protein expression (e.g., as a result of defective splicing). Other single nucleotide polymorphisms have no phenotypic effects. Single nucleotide polymorphisms can be used in the same manner as RFLPs and VNTRs, but offer several advantages.

Single nucleotide polymorphisms occur with greater frequency and are spaced more uniformly throughout the genome than other forms of polymorphism. The greater frequency and uniformity of single nucleotide polymorphisms means that there is a greater probability that such a polymorphism will be found in close proximity to a genetic locus of interest than would be the case for other polymorphisms. The different forms of characterized single nucleotide polymorphisms are often easier to distinguish than other types of polymorphism (e.g., by use of assays employing allele-specific hybridization probes or primers).

Only a small percentage of the total repository of polymorphisms in humans and other organisms has been identified. The limited number of polymorphisms identified to date is due to the large amount of work required for their detection by conventional methods. For example, a conventional approach to identifying polymorphisms might be to sequence the same stretch of DNA in a population of individuals by dideoxy sequencing. In this type of approach, the amount of work increases in proportion to both the length of sequence and the number of individuals in a population and becomes impractical for large stretches of DNA or large numbers of persons.

Type 2 Diabetes mellitus is a chronic disorder characterized by impaired insulin action in target tissues (e.g. skeletal muscle, adipose and liver), impaired insulin secretion and elevated hepatic glucose production (Laasko et al). In addition to these hallmark symptoms, Type 2 diabetics often display dyslipidemia, hyperinsulinemia and hypertension (Laasko et al; and Skrumsager et al).

Insulin sensitivity and glucose homeostasis is regulated, in part, by a number of genes whose expression are dependent upon transcription factors known as peroxisome proliferator-activated receptors (PPARs). Three PPAR genes exist in humans, encoding PPARα, PPARγ and PPARδ. All three PPAR isoforms are ligand-dependant transcription factors that heterodimerize with retinoic acid-X-receptor (RXR) and bind to consensus sequences (PPAR response elements, or PPRE) within the promoters of target genes where they enhance transcription (Berger et al). Whereas PPARα is primarily involved in regulating genes involved in fatty acid oxidation, PPARγ targets include genes involved in adipocyte differentiation and lipogenesis as well as genes that control cellular energy homeostasis (Berger et al).

The thiazolidinedione (TZD) class of synthetic PPAR ligands have recently been developed for the treatment of Type 2 diabetes. This family of compounds preferentially targets PPARγ. Owing to their antidiabetic properties, TZDs lead to the induction of genes involved in insulin action and glucose homeostasis (Berger et al; Giles et al; and Inzucchi et al). As activation of PPARγ also induces genes involved in adipocyte function and differentiation, treatment with TZDs also results in an increase in lipogenic target genes. In addition to increased subcutaneous adiposity TZDs also result in dose-dependent congestive heart failure (Giles et al; Inzucchi et al; and Hollenberg et al). Other non-TZD PPAR agonists currently in development target both PPARα and PPARγ and result in improved glucose levels and insulin sensitivity as well as improvement in lipid levels in insulin resistant animal models and in humans (Skrumsager et al; Chakrabarti et al; and Berger and Wagner). However, as with TZDs, treatment with non-TZD, dual PPARα/γ agonists is also associated with congestive heart failure (Skrumsager et al). Although the mechanism of action of PPAR agonist-induced congestive heart failure is unknown, it is speculated to result from direct action on the kidneys (Hollenberg et al).

Aldosterone, by binding and activating the mineralocorticoid receptor (MLR) is a key hormone that regulates sodium and water re-absorption by the kidney. It is known that changes in aldosterone levels accompany congestive heart failure (CHF). In fact, spironolactone, a drug used to treat CHF, exerts its therapeutic action by inhibiting the mineralocorticoid receptor (Weber et al). Thus, MLR may potentially be involved in PPAR-agonist induced CHF mechanism of action.

PPAR-gamma is the target of PPAR-gamma agonists, and a polymorphism in this target that results in a Proline/Alanine substitution at residue 12 has been associated with risk of developing diabetes and is known to affect the function of the encoded protein (Deeb et al). Thus, PPAR-gamma may potentially be involved in PPAR-agonist induced CHF mechanism of action.

Adiponectin is a circulating hormone secreted by adipose tissue and is thought to be involved in sensitizing cells to insulin (Scherer P E et al; and Yamauchi T et al). The expression of adiponectin is regulated by adiponectin (Maeda N et al). The polymorphism Tyr111His was associated with reduced plasma adiponectin levels (Vasseur F et al). Adiponectin-deficient mice are more sensitive to cardiac hypertrophy in response to pressure-overload (Shibata R et al). Thus, adiponectin may potentially be involved in PPAR-agonist induced CHF mechanism of action.

Thus, genetic polymorphisms in MLR, PPAR-gamma, and/or adiponectin may cause alterations in the level of either of these proteins or their related peptides or variants, or affect downstream signal transduction. Such polymorphisms may genetically predispose certain individuals to an increased risk of developing congestive heart failure, particularly in response to PPAR-agonist induced therapy, or may be protective and decrease an individuals risk of developing congestive heart failure. Such polymorphisms are expected to show a significant difference in allele frequency between healthy individuals and congestive heart failure subjects. Genotypes of such polymorphisms can predict each individual's susceptibility to congestive heart failure, and thus will be useful in identifying a group of high risk individuals that may be subject to modified PPAR-directed treatment regimens. Alternatively, the identification of such a group may preclude one or more individuals within said group from being administered an PPAR-directed agonist or antagonist.

### SUMMARY OF THE INVENTION

The invention relates to a nucleic acid molecule which comprises, or alternatively consists of, at least one single nucleotide polymorphism within the mineralocorticoid receptor genomic sequence at a specific polymorphic locus. In a particular embodiment the invention relates to the variant allele of the mineralocorticoid receptor gene or polynucleotide having at least one single nucleotide polymorphism, which variant allele differs from a reference allele by one nucleotide at the site(s) identified in FIGS. 1A-F, and/or FIGS. 2A-F, or elsewhere herein. The complementary sequence of each of these nucleic acid molecules are also provided. The nucleic acid molecules can be comprised of DNA or RNA, can be double- or single-stranded, and may comprise fragments. Fragments can be, for example, about 5 to about 10, about 5 to about 15, about 10 to about 20, about 15 to about 25, about 10 to about 30, about 10 to about 50, or about 10 to about 100 bases long, and preferably comprise at least one polymorphic allele.

In another embodiment, the invention relates to the mineralocorticoid receptor reference or wild type allele of a gene or polynucleotide having a polymorphic locus, in which said reference or wild type allele differs from a variant allele by one nucleotide at the polymorphic site(s) identified in FIGS. 1A-F, and/or FIGS. 2A-F, or elsewhere herein. The complementary sequence of each of these nucleic acid molecules are also provided. The nucleic acid molecules can be comprised of DNA or RNA, can be double- or single-stranded, and may comprise fragments. Fragments can be, for example, about 5 to about 10, about 5 to about 15, about 10 to about 20, about 15 to about 25, about 10 to about 30, about 10 to about 50, or about 10 to about 100 bases long, and preferably comprise at least one polymorphic locus.

The invention further provides mineralocorticoid receptor variant and reference allele-specific oligonucleotides that hybridize to a nucleic acid molecule comprising at least one polymorphic locus, in addition to the complement of said oligonucleotide. These oligonucleotides can be probes or primers.

The invention further provides oligonucleotides that may be used to amplify a portion of either the mineralocorticoid receptor variant or reference sequences comprising at least one polymorphic locus of the present invention, in addition to providing oligonucleotides that may be used to sequence said amplified sequence. The invention further provides a method of analyzing a nucleic acid from a DNA sample using said amplification and sequencing primers to assess whether said sample contains the reference or variant nucleotide (allele) at the polymorphic locus, comprising the steps of amplifying a sequence using appropriate oligonucleotide primers for amplifying across a polymorphic locus, and sequencing the resulting amplified product using appropriate sequencing primers to sequence said product to determine whether the variant or reference base is present at the polymorphic locus.

The invention further provides a method of analyzing a nucleic acid from patient sample(s) using said amplification and sequencing primers to assess whether said sample(s) contain the mineralocorticoid receptor reference or variant nucleotide (allele) at the polymorphic locus in an effort to identify populations at risk of developing dose-dependent congestive heart failure upon administration of a PPAR-agonist, comprising the steps of amplifying a sequence using appropriate oligonucleotide primers for amplifying across a polymorphic locus, and sequencing the resulting amplified product using appropriate sequencing primers to sequence said product to determine whether the variant or reference nucleotide is present at the polymorphic locus.

The invention further provides oligonucleotides that may be used to genotype patient sample(s) to assess whether said sample(s) contain the mineralocorticoid receptor reference or variant nucleotide (allele) at the polymorphic site(s). The invention provide a method of using oligonucleotides that may be used to genotype a patient sample to assess whether said sample contains the reference or variant nucleotide (allele) at the polymorphic locus. An embodiment of the method comprises the steps of amplifying a sequence using appropriate oligonucleotide primers for amplifying across a polymorphic locus, and subjecting the product of said amplification to a genetic bit analysis (GBA) reaction.

The invention provides a method of using oligonucleotides that may be used to genotype patient sample(s) to identify individual(s) at risk of developing dose-dependent congestive heart failure upon administration of a PPAR-agonist to assess whether said sample(s) contains the mineralocorticoid receptor reference or variant nucleotide (allele) at one or more polymorphic loci. An embodiment of the method comprises the steps of amplifying a sequence using appropriate oligonucleotide primers for amplifying across a polymorphic locus, and subjecting the product of said amplification to a genetic bit analysis (GBA) reaction, and optionally determining the statistical association between either the reference or variant allele at the polymorphic site(s) to the incidence of dose-dependent congestive heart failure.

The invention provides a method of using oligonucleotides that may be used to genotype patient sample(s) to identify ethnic population(s) that may be at risk of developing dose-dependent congestive heart failure upon administration of a PPAR-agonist to assess whether said sample(s) contains the mineralocorticoid receptor reference or variant nucleotide (allele) at one or more polymorphic loci comprising the steps of amplifying a sequence using appropriate oligonucleotide primers for amplifying across a polymorphic locus, and subjecting the product of said amplification to a genetic bit analysis (GBA) reaction, and optionally determining the statistical association between either the reference or variant allele at the polymorphic site(s) to the incidence of dose-dependent congestive heart failure.

The invention further provides a method of analyzing a nucleic acid from one or more individuals. The method allows the determination of whether the mineralocorticoid receptor reference or variant base is present at any one, or more, of the polymorphic sites identified in FIGS. 1A-F, and/or FIGS. 2A-F, or elsewhere herein. Optionally, a set of nucleotides occupying a set of the polymorphic loci shown in FIGS. 1A-F, and/or FIGS. 2A-F, or elsewhere herein, is determined. This type of analysis can be performed on a number of individuals, who are also tested (previously, concurrently or subsequently) for the presence of a dose-dependent congestive heart failure phenotype or related disorder. The presence or absence of a dose-dependent congestive heart failure disease phenotype is then correlated with said nucleotide or set of nucleotides present at the polymorphic locus or loci in the individuals tested.

The invention further relates to a method of predicting the presence, absence, likelihood of the presence or absence, or severity of a dose-dependent congestive heart failure or related disorder associated with a particular mineralocorticoid receptor genotype. The method comprises obtaining a nucleic acid sample from an individual and determining the identity of one or more nucleotides at specific polymorphic loci of nucleic acid molecules described herein, wherein the presence of a particular base at that site is correlated with the incidence of dose-dependent congestive heart failure or related disorder, thereby predicting the presence, absence, likelihood of the presence or absence, or severity, of the dose-dependent congestive heart failure phenotype or related disorder in the individual.

The invention further relates to mineralocorticoid receptor polynucleotides having one or more polymorphic loci comprising one or more variant alleles. The invention also relates to said polynucleotides lacking a start codon. The invention further relates to polynucleotides of the present invention containing one or more variant alleles wherein said polynucleotides encode a polypeptide of the present invention. The invention relates to polypeptides of the present invention containing one or more variant amino acids encoded by one or more variant alleles.

The present invention relates to antisense oligonucleotides capable of hybridizing to the mineralocorticoid receptor polynucleotides of the present invention. Preferably, such antisense oligonucleotides are capable of discriminating between the reference or variant allele of the polynucleotide, preferably at one or more polymorphic sites of said polynucleotide.

The present invention relates to siRNA or RNAi oligonucleotides capable of hybridizing to the mineralocorticoid receptor polynucleotides of the present invention. Preferably, such siRNA or RNAi oligonucleotides are capable of discriminating between the reference or variant allele of the polynucleotide, preferably at one or more polymorphic sites of said polynucleotide.

The present invention also relates to zinc finger proteins capable of binding to the mineralocorticoid receptor polynucleotides of the present invention. Preferably, such zinc finger proteins are capable of discriminating between the reference or variant allele of the polynucleotide, preferably at one or more polymorphic sites of said polynucleotide.

The present invention relates to antibodies directed against the mineralocorticoid receptor polypeptides of the present invention. Preferably, such antibodies are capable of discriminating between the reference or variant allele of the polypeptide, preferably at one or more polymorphic sites of said polynucleotide.

The present invention also relates to recombinant vectors, which include the isolated mineralocorticoid receptor nucleic acid molecules of the present invention, and to host cells containing the recombinant vectors, as well as to methods of making such vectors and host cells, in addition to their use in the production of polypeptides or peptides provided herein using recombinant techniques. Synthetic methods for producing the polypeptides and polynucleotides of the present invention are provided. Also provided are diagnostic methods for detecting diseases, disorders, and/or conditions related to the polypeptides and polynucleotides provided herein, and therapeutic methods for treating such diseases, disorders, and/or conditions. The invention further relates to screening methods for identifying binding partners of the polypeptides.

The invention relates to a method of analyzing at least one nucleic acid sample, comprising the step of determining the mineralocorticoid receptor nucleic acid sequence from one or more samples at one or more polymorphic loci in the human mineralocorticoid receptor gene selected from the group consisting of SNP1, or any combination thereof, wherein the presence of the variable allele at said one or more polymorphic loci is indicative of an increased risk of developing dose-dependent congestive heart failure in a patient receiving PPAR-agonist therapy.

The invention relates to a method of analyzing at least one nucleic acid sample, comprising the step of determining the mineralocorticoid receptor nucleic acid sequence from one or more samples at one or more polymorphic loci in the human mineralocorticoid receptor gene selected from the group consisting of SNP1, or any combination thereof, wherein the presence of the reference allele at said one or more polymorphic loci is indicative of a decreased risk of developing dose-dependent congestive heart failure in a patient receiving PPAR-agonist therapy.

The invention further relates to a method of constructing mineralocorticoid receptor haplotypes using the isolated nucleic acids referred to in FIGS. 1A-F, and/or FIGS. 2A-F, or elsewhere herein, comprising the step of grouping at least two said nucleic acids.

The invention further relates to a method of constructing haplotypes further comprising the step of using said haplotypes to identify an individual for the presence of dose-dependent congestive heart failure or related disease phenotype, and correlating the presence of the disease phenotype with said haplotype.

The invention further relates to a library of nucleic acids, each of which comprises one or more polymorphic positions within a gene encoding the human mineralocorticoid receptor protein, wherein said polymorphic positions are selected from a group consisting of the polymorphic positions provided in FIGS. 1A-F, FIGS. 2A-F, and Table I.

The invention further relates to a library of nucleic acids, wherein the sequence at said aforementioned polymorphic position is selected from the group consisting of the polymorphic position identified in FIGS. 1A-F, and/or FIGS. 2A-F, or elsewhere herein, the complimentary sequence of said sequences, and/or fragments of said sequences.

The invention further relates to a kit for identifying an individual at risk of developing dose-dependent congestive heart failure or related disorder upon administration of a pharmaceutically acceptable amount of a PPAR-agonist, wherein said kit comprises oligonucleotide primers capable of identifying the nucleotide residing at one or more polymorphic loci of the human mineralocorticoid receptor gene, wherein the presence of the reference allele at said one or more polymorphic loci is indicative of a decreased risk of developing dose-dependent congestive heart failure in a patient receiving PPAR-agonist therapy, while the presence of the variable allele at said one or more polymorphic loci is indicative of an increased risk of developing dose-dependent congestive heart failure in a patient receiving PPAR-agonist therapy.

The invention further relates to a kit for identifying an individual at risk of developing dose-dependent congestive heart failure or related disorder upon administration of a pharmaceutically acceptable amount of a PPAR-agonist, wherein said kit comprises oligonucleotide primers capable of identifying the nucleotide residing at one or more polymorphic loci of the human mineralocorticoid receptor gene, wherein the presence of the reference allele at said one or more polymorphic loci is indicative of a decreased risk of developing dose-dependent congestive heart failure in a patient receiving PPAR-agonist therapy, while the presence of the variable allele at said one or more polymorphic loci is indicative of an increased risk of developing dose-dependent congestive heart failure in a patient receiving PPAR-agonist therapy, and wherein said oligonucleotides hybridize immediately adjacent to said one or more polymorphic positions, or wherein said primer(s) hybridizes to said polymorphic positions such that the central position of the primer aligns with the polymorphic position of said gene.

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the nucleotide present within at least one or more nucleic acid sample(s) from an individual to be assessed at one or more polymorphic position(s) of the human mineralocorticoid receptor gene sequence selected from the group consisting of: SEQ ID NOS:1, and/or 3, wherein the presence of the variable nucleotide at the one or more polymorphic position(s) indicates that the individual has an increased likelihood of being diagnosed as at risk of developing dose-dependent congestive heart failure or related disorder upon administration of a pharmaceutically acceptable amount of an PPAR-agonist as compared to an individual having the reference allele at said polymorphic position(s).

The invention further relates to a method for predicting the likelihood that an individual will be diagnosed as being at risk of developing a dose-dependent congestive heart failure or related disorder upon administration of a pharmaceutically acceptable amount of a PPAR-agonist comprising the step of determining the nucleotide present within at least one or more nucleic acid sample(s) from an individual to be assessed at one or more polymorphic position(s) of the human mineralocorticoid receptor gene sequence selected from the group consisting of: SEQ ID NOS:1, and/or 3, wherein the presence of the reference nucleotide at the one or more polymorphic position(s) indicates that the individual has a decreased likelihood of being diagnosed as at risk of developing dose-dependent congestive heart failure or related disorder upon administration of a pharmaceutically acceptable amount of an PPAR-agonist as compared to an individual having the variable allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the polypeptide present within at least one or more sample(s) from an individual to be assessed at one or more polymorphic position(s) of the human mineralocorticoid receptor polypeptide sequence selected from the group consisting of: SEQ ID NOS:2, and/or 4, wherein the presence of the variable amino acid at the one or more polymorphic position(s) indicates that the individual has an increased likelihood of being diagnosed as at risk of developing dose-dependent congestive heart failure or related disorder upon administration of a pharmaceutically acceptable amount of an PPAR-agonist as compared to an individual having the reference allele at said polymorphic position(s).

The invention further relates to a method for predicting the likelihood that an individual will be diagnosed as being at risk of developing a dose-dependent congestive heart failure or related disorder upon administration of a pharmaceutically acceptable amount of a PPAR-agonist comprising the step of determining the nucleotide present within at least one or more sample(s) from an individual to be assessed at one or more polymorphic position(s) of the human mineralocorticoid receptor polypeptide sequence selected from the group consisting of: SEQ ID NOS:1, and/or 3, wherein the presence of the reference amino acid at the one or more polymorphic position(s) indicates that the individual has a decreased likelihood of being diagnosed as at risk of developing dose-dependent congestive heart failure or related disorder upon administration of a pharmaceutically acceptable amount of an PPAR-agonist as compared to an individual having the variable allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the nucleotide present within at least one or more nucleic acid sample(s) from an individual to be assessed at one or more polymorphic position(s) of the human mineralocorticoid receptor gene sequence selected from the group consisting of: nucleotide position 754 of SEQ ID NOS:1 or 3, wherein the presence of the variable nucleotide at the one or more polymorphic position(s) indicates that the individual has an increased likelihood of being diagnosed as at risk of developing dose-dependent congestive heart failure or related disorder upon administration of a pharmaceutically acceptable amount of a PPAR-agonist as compared to an individual having the reference allele at said polymorphic position(s).

The invention further relates to a method for predicting the likelihood that an individual will be diagnosed as being at risk of developing a dose-dependent congestive heart failure or related disorder upon administration of a pharmaceutically acceptable amount of a PPAR-agonist comprising the step of determining the nucleotide present within at least one or more nucleic acid sample(s) from an individual to be assessed at one or more polymorphic position(s) of the human mineralocorticoid receptor gene sequence selected from the group consisting of: nucleotide position 754 of SEQ ID NOS:1 or 3, wherein the presence of the reference nucleotide at the one or more polymorphic position(s) indicates that the individual has a decreased likelihood of being diagnosed as at risk of developing dose-dependent congestive heart failure or related disorder upon administration of a pharmaceutically acceptable amount of a PPAR-agonist as compared to an individual having the variable allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the amino acid present within at least one or more sample(s) from an individual to be assessed at one or more polymorphic position(s) of the human mineralocorticoid receptor polypeptide sequence selected from the group consisting of: amino acid position 180 of SEQ ID NOS:2 or 4, wherein the presence of the variable amino acid at the one or more polymorphic position(s) indicates that the individual has an increased likelihood of being diagnosed as at risk of developing dose-dependent congestive heart failure or related disorder upon administration of a pharmaceutically acceptable amount of a PPAR-agonist as compared to an individual having the reference allele at said polymorphic position(s).

The invention further relates to a method for predicting the likelihood that an individual will be diagnosed as being at risk of developing a dose-dependent congestive heart failure or related disorder upon administration of a pharmaceutically acceptable amount of a PPAR-agonist comprising the step of determining the amino acid present within at least one or more sample(s) from an individual to be assessed at one or more polymorphic position(s) of the human mineralocorticoid receptor polypeptide sequence selected from the group consisting of: amino acid position 180 of SEQ ID NOS:2 or 4, wherein the presence of the reference amino acid at the one or more polymorphic position(s) indicates that the individual has a decreased likelihood of being diagnosed as at risk of developing dose-dependent congestive heart failure or related disorder upon administration of a pharmaceutically acceptable amount of a PPAR-agonist as compared to an individual having the variable allele at said polymorphic position(s).

The present invention is also directed to methods of predicting whether a patient administered a PPAR-agonist will respond to PPAR-agonist therapy; for predicting whether a patient will respond to specific doses of a PPAR-agonist; whether the level of the administered PPAR-agonist needs to be increased or decreased to achieve the desired level of human mineralocorticoid receptor expression identified as representing a responsive level; whether a patient has an increased risk of developing dose-dependent congestive heart failure upon the administration of a pharmaceutically acceptable level of a PPAR-agonist; whether said patient requires a lower level of administered PPAR agonist to limit the risk of developing said dose-dependent congestive heart failure; or whether said patient may be administered a higher level of administered PPAR agonist without the risk of developing said dose-dependent congestive heart failure, in order to limit the risk of developing said dose-dependent congestive heart failure, comprising the step of assessing the level of mineralocorticoid receptor expression resulting from the administration of a PPAR-agonist relative to a control compound.

The invention relates to a nucleic acid molecule which comprises, or alternatively consists of, at least one single nucleotide polymorphism within the PPAR-gamma genomic sequence at a specific polymorphic locus. In a particular embodiment the invention relates to the variant allele of the PPAR-gamma gene or polynucleotide having at least one single nucleotide polymorphism, which variant allele differs from a reference allele by one nucleotide at the site(s) identified in FIGS. 4A-B, and/or FIGS. 5A-B, or elsewhere herein. The complementary sequence of each of these nucleic acid molecules are also provided. The nucleic acid molecules can be comprised of DNA or RNA, can be double- or single-stranded, and may comprise fragments. Fragments can be, for example, about 5 to about 10, about 5 to about 15, about 10 to about 20, about 15 to about 25, about 10 to about 30, about 10 to about 50, or about 10 to about 100 bases long, and preferably comprise at least one polymorphic allele.

In another embodiment, the invention relates to the PPAR-gamma reference or wild type allele of a gene or polynucleotide having a polymorphic locus, in which said reference or wild type allele differs from a variant allele by one nucleotide at the polymorphic site(s) identified in FIGS. 4A-B, and/or FIGS. 5A-B, or elsewhere herein. The complementary sequence of each of these nucleic acid molecules are also provided. The nucleic acid molecules can be comprised of DNA or RNA, can be double- or single-stranded, and may comprise fragments. Fragments can be, for example, about 5 to about 10, about 5 to about 15, about 10 to about 20, about 15 to about 25, about 10 to about 30, about 10 to about 50, or about 10 to about 100 bases long, and preferably comprise at least one polymorphic locus.

The invention further provides PPAR-gamma variant and reference allele-specific oligonucleotides that hybridize to a nucleic acid molecule comprising at least one polymorphic locus, in addition to the complement of said oligonucleotide. These oligonucleotides can be probes or primers.

The invention further provides oligonucleotides that may be used to amplify a portion of either the PPAR-gamma variant or reference sequences comprising at least one polymorphic locus of the present invention, in addition to providing oligonucleotides that may be used to sequence said amplified sequence. The invention further provides a method of analyzing a nucleic acid from a DNA sample using said amplification and sequencing primers to assess whether said sample contains the reference or variant nucleotide (allele) at the polymorphic locus, comprising the steps of amplifying a sequence using appropriate oligonucleotide primers for amplifying across a polymorphic locus, and sequencing the resulting amplified product using appropriate sequencing primers to sequence said product to determine whether the variant or reference base is present at the polymorphic locus.

The invention further provides a method of analyzing a nucleic acid from patient sample(s) using said amplification and sequencing primers to assess whether said sample(s) contain the PPAR-gamma reference or variant nucleotide (allele) at the polymorphic locus in an effort to identify populations at risk of developing dose-dependent congestive heart failure upon administration of a PPAR-agonist, comprising the steps of amplifying a sequence using appropriate oligonucleotide primers for amplifying across a polymorphic locus, and sequencing the resulting amplified product using appropriate sequencing primers to sequence said product to determine whether the variant or reference nucleotide is present at the polymorphic locus.

The invention further provides oligonucleotides that may be used to genotype patient sample(s) to assess whether said sample(s) contain the PPAR-gamma reference or variant nucleotide (allele) at the polymorphic site(s). The invention provide a method of using oligonucleotides that may be used to genotype a patient sample to assess whether said sample contains the reference or variant nucleotide (allele) at the polymorphic locus. An embodiment of the method comprises the steps of amplifying a sequence using appropriate oligonucleotide primers for amplifying across a polymorphic locus, and subjecting the product of said amplification to a genetic bit analysis (GBA) reaction.

The invention provides a method of using oligonucleotides that may be used to genotype patient sample(s) to identify individual(s) at risk of developing dose-dependent congestive heart failure upon administration of a PPAR-agonist to assess whether said sample(s) contains the PPAR-gamma reference or variant nucleotide (allele) at one or more polymorphic loci. An embodiment of the method comprises the steps of amplifying a sequence using appropriate oligonucleotide primers for amplifying across a polymorphic locus, and subjecting the product of said amplification to a genetic bit analysis (GBA) reaction, and optionally determining the statistical association between either the reference or variant allele at the polymorphic site(s) to the incidence of dose-dependent congestive heart failure.

The invention provides a method of using oligonucleotides that may be used to genotype patient sample(s) to identify ethnic population(s) that may be at risk of developing dose-dependent congestive heart failure upon administration of a PPAR-agonist to assess whether said sample(s) contains the PPAR-gamma reference or variant nucleotide (allele) at one or more polymorphic loci comprising the steps of amplifying a sequence using appropriate oligonucleotide primers for amplifying across a polymorphic locus, and subjecting the product of said amplification to a genetic bit analysis (GBA) reaction, and optionally determining the statistical association between either the reference or variant allele at the polymorphic site(s) to the incidence of dose-dependent congestive heart failure.

The invention further provides a method of analyzing a nucleic acid from one or more individuals. The method allows the determination of whether the PPAR-gamma reference or variant base is present at any one, or more, of the polymorphic sites identified in FIGS. 4A-B, and/or FIGS. 5A-B, or elsewhere herein. Optionally, a set of nucleotides occupying a set of the polymorphic loci shown in FIGS. 4A-B, and/or FIGS. 5A-B, or elsewhere herein, is determined. This type of analysis can be performed on a number of individuals, who are also tested (previously, concurrently or subsequently) for the presence of a dose-dependent congestive heart failure phenotype or related disorder. The presence or absence of a dose-dependent congestive heart failure disease phenotype is then correlated with said nucleotide or set of nucleotides present at the polymorphic locus or loci in the individuals tested.

The invention further relates to a method of predicting the presence, absence, likelihood of the presence or absence, or severity of a dose-dependent congestive heart failure or related disorder associated with a particular PPAR-gamma genotype. The method comprises obtaining a nucleic acid sample from an individual and determining the identity of one or more nucleotides at specific polymorphic loci of nucleic acid molecules described herein, wherein the presence of a particular base at that site is correlated with the incidence of dose-dependent congestive heart failure or related disorder, thereby predicting the presence, absence, likelihood of the presence or absence, or severity, of the dose-dependent congestive heart failure phenotype or related disorder in the individual.

The invention further relates to PPAR-gamma polynucleotides having one or more polymorphic loci comprising one or more variant alleles. The invention also relates to said polynucleotides lacking a start codon. The invention further relates to polynucleotides of the present invention containing one or more variant alleles wherein said polynucleotides encode a polypeptide of the present invention. The invention relates to polypeptides of the present invention containing one or more variant amino acids encoded by one or more variant alleles.

The present invention relates to antisense oligonucleotides capable of hybridizing to the PPAR-gamma polynucleotides of the present invention. Preferably, such antisense oligonucleotides are capable of discriminating between the reference or variant allele of the polynucleotide, preferably at one or more polymorphic sites of said polynucleotide.

The present invention relates to siRNA or RNAi oligonucleotides capable of hybridizing to the PPAR-gamma polynucleotides of the present invention. Preferably, such siRNA or RNAi oligonucleotides are capable of discriminating between the reference or variant allele of the polynucleotide, preferably at one or more polymorphic sites of said polynucleotide.

The present invention also relates to zinc finger proteins capable of binding to the PPAR-gamma polynucleotides of the present invention. Preferably, such zinc finger proteins are capable of discriminating between the reference or variant allele of the polynucleotide, preferably at one or more polymorphic sites of said polynucleotide.

The present invention relates to antibodies directed against the PPAR-gamma polypeptides of the present invention. Preferably, such antibodies are capable of discriminating between the reference or variant allele of the polypeptide, preferably at one or more polymorphic sites of said polynucleotide.

The present invention also relates to recombinant vectors, which include the isolated PPAR-gamma nucleic acid molecules of the present invention, and to host cells containing the recombinant vectors, as well as to methods of making such vectors and host cells, in addition to their use in the production of polypeptides or peptides provided herein using recombinant techniques. Synthetic methods for producing the polypeptides and polynucleotides of the present invention are provided. Also provided are diagnostic methods for detecting diseases, disorders, and/or conditions related to the polypeptides and polynucleotides provided herein, and therapeutic methods for treating such diseases, disorders, and/or conditions. The invention further relates to screening methods for identifying binding partners of the polypeptides.

The invention relates to a method of analyzing at least one nucleic acid sample, comprising the step of determining the nucleic acid sequence from one or more samples at one or more polymorphic loci in the human PPAR-gamma gene selected from the group consisting of SNP1, or any combination thereof, wherein the presence of the variable allele at said one or more polymorphic loci is indicative of an increased risk of developing dose-dependent congestive heart failure in a patient receiving PPAR-agonist therapy.

The invention relates to a method of analyzing at least one nucleic acid sample, comprising the step of determining the nucleic acid sequence from one or more samples at one or more polymorphic loci in the human PPAR-gamma gene selected from the group consisting of SNP1, or any combination thereof, wherein the presence of the reference allele at said one or more polymorphic loci is indicative of a decreased risk of developing dose-dependent congestive heart failure in a patient receiving PPAR-agonist therapy.

The invention further relates to a method of constructing haplotypes using the isolated PPAR-gamma nucleic acids referred to in FIGS. 4A-B, and/or FIGS. 5A-B, or elsewhere herein, comprising the step of grouping at least two said nucleic acids.

The invention further relates to a method of constructing PPAR-gamma haplotypes further comprising the step of using said haplotypes to identify an individual for the presence of dose-dependent congestive heart failure or related disease phenotype, and correlating the presence of the disease phenotype with said haplotype.

The invention further relates to a library of nucleic acids, each of which comprises one or more polymorphic positions within a gene encoding the human PPAR-gamma protein, wherein said polymorphic positions are selected from a group consisting of the polymorphic positions provided in FIGS. 4A-B, FIGS. 5A-B, and Table I.

The invention further relates to a library of nucleic acids, wherein the sequence at said aforementioned polymorphic position is selected from the group consisting of the polymorphic position identified in FIGS. 4A-B, and/or FIGS. 5A-B, or elsewhere herein, the complimentary sequence of said sequences, and/or fragments of said sequences.

The invention further relates to a kit for identifying an individual at risk of developing dose-dependent congestive heart failure or related disorder upon administration of a pharmaceutically acceptable amount of a PPAR-agonist, wherein said kit comprises oligonucleotide primers capable of identifying the nucleotide residing at one or more polymorphic loci of the human PPAR-gamma gene, wherein the presence of the reference allele at said one or more polymorphic loci is indicative of a decreased risk of developing dose-dependent congestive heart failure in a patient receiving PPAR-agonist therapy, while the presence of the variable allele at said one or more polymorphic loci is indicative of an increased risk of developing dose-dependent congestive heart failure in a patient receiving PPAR-agonist therapy.

The invention further relates to a kit for identifying an individual at risk of developing dose-dependent congestive heart failure or related disorder upon administration of a pharmaceutically acceptable amount of a PPAR-agonist, wherein said kit comprises oligonucleotide primers capable of identifying the nucleotide residing at one or more polymorphic loci of the human PPAR-gamma gene, wherein the presence of the reference allele at said one or more polymorphic loci is indicative of a decreased risk of developing dose-dependent congestive heart failure in a patient receiving PPAR-agonist therapy, while the presence of the variable allele at said one or more polymorphic loci is indicative of an increased risk of developing dose-dependent congestive heart failure in a patient receiving PPAR-agonist therapy, and wherein said oligonucleotides hybridize immediately adjacent to said one or more polymorphic positions, or wherein said primer(s) hybridizes to said polymorphic positions such that the central position of the primer aligns with the polymorphic position of said gene.

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the nucleotide present within at least one or more nucleic acid sample(s) from an individual to be assessed at one or more polymorphic position(s) of the human PPAR-gamma gene sequence selected from the group consisting of: SEQ ID NOS:13, and/or 15, wherein the presence of the variable nucleotide at the one or more polymorphic position(s) indicates that the individual has an increased likelihood of being diagnosed as at risk of developing dose-dependent congestive heart failure or related disorder upon administration of a pharmaceutically acceptable amount of an PPAR-agonist as compared to an individual having the reference allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the nucleotide present within at least one or more nucleic acid sample(s) from an individual to be assessed at one or more polymorphic position(s) of the human PPAR-gamma gene sequence selected from the group consisting of: SEQ ID NOS:13, and/or 15, wherein the presence of the reference nucleotide at the one or more polymorphic position(s) indicates that the individual has a decreased likelihood of being diagnosed as at risk of developing dose-dependent congestive heart failure or related disorder upon administration of a pharmaceutically acceptable amount of an PPAR-agonist as compared to an individual having the variable allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the polypeptide present within at least one or more sample(s) from an individual to be assessed at one or more polymorphic position(s) of the human PPAR-gamma polypeptide sequence selected from the group consisting of: SEQ ID NOS:14, and/or 16, wherein the presence of the variable amino acid at the one or more polymorphic position(s) indicates that the individual has an increased likelihood of being diagnosed as at risk of developing dose-dependent congestive heart failure or related disorder upon administration of a pharmaceutically acceptable amount of an PPAR-agonist as compared to an individual having the reference allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the nucleotide present within at least one or more sample(s) from an individual to be assessed at one or more polymorphic position(s) of the human PPAR-gamma polypeptide sequence selected from the group consisting of: SEQ ID NOS:13, and/or 15, wherein the presence of the reference amino acid at the one or more polymorphic position(s) indicates that the individual has a decreased likelihood of being diagnosed as at risk of developing dose-dependent congestive heart failure or related disorder upon administration of a pharmaceutically acceptable amount of an PPAR-agonist as compared to an individual having the variable allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the nucleotide present within at least one or more nucleic acid sample(s) from an individual to be assessed at one or more polymorphic position(s) of the human PPAR-gamma gene sequence selected from the group consisting of: nucleotide position 167 of SEQ ID NOS:13 or 15, wherein the presence of the variable nucleotide at the one or more polymorphic position(s) indicates that the individual has an increased likelihood of being diagnosed as at risk of developing dose-dependent congestive heart failure or related disorder upon administration of a pharmaceutically acceptable amount of a PPAR-agonist as compared to an individual having the reference allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the nucleotide present within at least one or more nucleic acid sample(s) from an individual to be assessed at one or more polymorphic position(s) of the human PPAR-gamma gene sequence selected from the group consisting of: nucleotide position 167 of SEQ ID NOS:13 or 15, wherein the presence of the reference nucleotide at the one or more polymorphic position(s) indicates that the individual has a decreased likelihood of being diagnosed as at risk of developing dose-dependent congestive heart failure or related disorder upon administration of a pharmaceutically acceptable amount of a PPAR-agonist as compared to an individual having the variable allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the amino acid present within at least one or more sample(s) from an individual to be assessed at one or more polymorphic position(s) of the human PPAR-gamma polypeptide sequence selected from the group consisting of: amino acid position 12 of SEQ ID NOS:14 or 16, wherein the presence of the variable amino acid at the one or more polymorphic position(s) indicates that the individual has an increased likelihood of being diagnosed as at risk of developing dose-dependent congestive heart failure or related disorder upon administration of a pharmaceutically acceptable amount of a PPAR-agonist as compared to an individual having the reference allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the amino acid present within at least one or more sample(s) from an individual to be assessed at one or more polymorphic position(s) of the human PPAR-gamma polypeptide sequence selected from the group consisting of: amino acid position 12 of SEQ ID NOS:14 or 16, wherein the presence of the reference amino acid at the one or more polymorphic position(s) indicates that the individual has a decreased likelihood of being diagnosed as at risk of developing dose-dependent congestive heart failure or related disorder upon administration of a pharmaceutically acceptable amount of a PPAR-agonist as compared to an individual having the variable allele at said polymorphic position(s).

The present invention is also directed to methods of predicting whether a patient administered a PPAR-agonist will respond to PPAR-agonist therapy; for predicting whether a patient will respond to specific doses of a PPAR-agonist; whether the level of the administered PPAR-agonist needs to be increased or decreased to achieve the desired level of human PPAR-gamma expression identified as representing a responsive level; whether a patient has an increased risk of developing dose-dependent congestive heart failure upon the administration of a pharmaceutically acceptable level of a PPAR-agonist; whether said patient requires a lower level of administered PPAR agonist to limit the risk of developing said dose-dependent congestive heart failure; or whether said patient may be administered a higher level of administered PPAR agonist without the risk of developing said dose-dependent congestive heart failure, inorder to limit the risk of developing said dose-dependent congestive heart failure, comprising the step of assessing the level of PPAR-gamma expression resulting from the administration of a PPAR-agonist relative to a control compound.

The invention relates to a nucleic acid molecule which comprises, or alternatively consists of, at least one single nucleotide polymorphism within the Adiponectin genomic sequence at a specific polymorphic locus. In a particular embodiment the invention relates to the variant allele of the Adiponectin gene or polynucleotide having at least one single nucleotide polymorphism, which variant allele differs from a reference allele by one nucleotide at the site(s) identified in FIGS. 7A-D, and/or FIGS. 8A-D, or elsewhere herein. The complementary sequence of each of these nucleic acid molecules are also provided. The nucleic acid molecules can be comprised of DNA or RNA, can be double- or single-stranded, and may comprise fragments. Fragments can be, for example, about 5 to about 10, about 5 to about 15, about 10 to about 20, about 15 to about 25, about 10 to about 30, about 10 to about 50, or about 10 to about 100 bases long, and preferably comprise at least one polymorphic allele.

In another embodiment, the invention relates to the Adiponectin reference or wild type allele of a gene or polynucleotide having a polymorphic locus, in which said reference or wild type allele differs from a variant allele by one nucleotide at the polymorphic site(s) identified in FIGS. 7A-D, and/or FIGS. 8A-D, or elsewhere herein. The complementary sequence of each of these nucleic acid molecules are also provided. The nucleic acid molecules can be comprised of DNA or RNA, can be double- or single-stranded, and may comprise fragments. Fragments can be, for example, about 5 to about 10, about 5 to about 15, about 10 to about 20, about 15 to about 25, about 10 to about 30, about 10 to about 50, or about 10 to about 100 bases long, and preferably comprise at least one polymorphic locus.

The invention further provides Adiponectin variant and reference allele-specific oligonucleotides that hybridize to a nucleic acid molecule comprising at least one polymorphic locus, in addition to the complement of said oligonucleotide. These oligonucleotides can be probes or primers.

The invention further provides oligonucleotides that may be used to amplify a portion of either the Adiponectin variant or reference sequences comprising at least one polymorphic locus of the present invention, in addition to providing oligonucleotides that may be used to sequence said amplified sequence. The invention further provides a method of analyzing a nucleic acid from a DNA sample using said amplification and sequencing primers to assess whether said sample contains the reference or variant nucleotide (allele) at the polymorphic locus, comprising the steps of amplifying a sequence using appropriate oligonucleotide primers for amplifying across a polymorphic locus, and sequencing the resulting amplified product using appropriate sequencing primers to sequence said product to determine whether the variant or reference base is present at the polymorphic locus.

The invention further provides a method of analyzing a nucleic acid from patient sample(s) using said amplification and sequencing primers to assess whether said sample(s) contain the Adiponectin reference or variant nucleotide (allele) at the polymorphic locus in an effort to identify populations at risk of developing dose-dependent congestive heart failure upon administration of a PPAR-agonist, comprising the steps of amplifying a sequence using appropriate oligonucleotide primers for amplifying across a polymorphic locus, and sequencing the resulting amplified product using appropriate sequencing primers to sequence said product to determine whether the variant or reference nucleotide is present at the polymorphic locus.

The invention further provides oligonucleotides that may be used to genotype patient sample(s) to assess whether said sample(s) contain the Adiponectin reference or variant nucleotide (allele) at the polymorphic site(s). The invention provide a method of using oligonucleotides that may be used to genotype a patient sample to assess whether said sample contains the reference or variant nucleotide (allele) at the polymorphic locus. An embodiment of the method comprises the steps of amplifying a sequence using appropriate oligonucleotide primers for amplifying across a polymorphic locus, and subjecting the product of said amplification to a genetic bit analysis (GBA) reaction.

The invention provides a method of using oligonucleotides that may be used to genotype patient sample(s) to identify individual(s) at risk of developing dose-dependent congestive heart failure upon administration of a PPAR-agonist to assess whether said sample(s) contains the Adiponectin reference or variant nucleotide (allele) at one or more polymorphic loci. An embodiment of the method comprises the steps of amplifying a sequence using appropriate oligonucleotide primers for amplifying across a polymorphic locus, and subjecting the product of said amplification to a genetic bit analysis (GBA) reaction, and optionally determining the statistical association between either the reference or variant allele at the polymorphic site(s) to the incidence of dose-dependent congestive heart failure.

The invention provides a method of using oligonucleotides that may be used to genotype patient sample(s) to identify ethnic population(s) that may be at risk of developing dose-dependent congestive heart failure upon administration of a PPAR-agonist to assess whether said sample(s) contains the Adiponectin reference or variant nucleotide (allele) at one or more polymorphic loci comprising the steps of amplifying a sequence using appropriate oligonucleotide primers for amplifying across a polymorphic locus, and subjecting the product of said amplification to a genetic bit analysis (GBA) reaction, and optionally determining the statistical association between either the reference or variant allele at the polymorphic site(s) to the incidence of dose-dependent congestive heart failure.

The invention further provides a method of analyzing a nucleic acid from one or more individuals. The method allows the determination of whether the Adiponectin reference or variant base is present at any one, or more, of the polymorphic sites identified in FIGS. 7A-D, and/or FIGS. 8A-D, or elsewhere herein. Optionally, a set of nucleotides occupying a set of the polymorphic loci shown in FIGS. 7A-D, and/or FIGS. 8A-D, or elsewhere herein, is determined. This type of analysis can be performed on a number of individuals, who are also tested (previously, concurrently or subsequently) for the presence of a dose-dependent congestive heart failure phenotype or related disorder. The presence or absence of a dose-dependent congestive heart failure disease phenotype is then correlated with said nucleotide or set of nucleotides present at the polymorphic locus or loci in the individuals tested.

The invention further relates to a method of predicting the presence, absence, likelihood of the presence or absence, or severity of a dose-dependent congestive heart failure or related disorder associated with a particular genotype. The method comprises obtaining a nucleic acid sample from an individual and determining the identity of one or more nucleotides at specific polymorphic loci of Adiponectin nucleic acid molecules described herein, wherein the presence of a particular base at that site is correlated with the incidence of dose-dependent congestive heart failure or related disorder, thereby predicting the presence, absence, likelihood of the presence or absence, or severity, of the dose-dependent congestive heart failure phenotype or related disorder in the individual.

The invention further relates to Adiponectin polynucleotides having one or more polymorphic loci comprising one or more variant alleles. The invention also relates to said polynucleotides lacking a start codon. The invention further relates to polynucleotides of the present invention containing one or more variant alleles wherein said polynucleotides encode a polypeptide of the present invention. The invention relates to polypeptides of the present invention containing one or more variant amino acids encoded by one or more variant alleles.

The present invention relates to antisense oligonucleotides capable of hybridizing to the Adiponectin polynucleotides of the present invention. Preferably, such antisense oligonucleotides are capable of discriminating between the reference or variant allele of the polynucleotide, preferably at one or more polymorphic sites of said polynucleotide.

The present invention relates to siRNA or RNAi oligonucleotides capable of hybridizing to the Adiponectin polynucleotides of the present invention. Preferably, such siRNA or RNAi oligonucleotides are capable of discriminating between the reference or variant allele of the polynucleotide, preferably at one or more polymorphic sites of said polynucleotide.

The present invention also relates to zinc finger proteins capable of binding to the Adiponectin polynucleotides of the present invention. Preferably, such zinc finger proteins are capable of discriminating between the reference or variant allele of the polynucleotide, preferably at one or more polymorphic sites of said polynucleotide.

The present invention relates to antibodies directed against the Adiponectin polypeptides of the present invention. Preferably, such antibodies are capable of discriminating between the reference or variant allele of the polypeptide, preferably at one or more polymorphic sites of said polynucleotide.

The present invention also relates to recombinant vectors, which include the Adiponectin isolated nucleic acid molecules of the present invention, and to host cells containing the recombinant vectors, as well as to methods of making such vectors and host cells, in addition to their use in the production of polypeptides or peptides provided herein using recombinant techniques. Synthetic methods for producing the polypeptides and polynucleotides of the present invention are provided. Also provided are diagnostic methods for detecting diseases, disorders, and/or conditions related to the polypeptides and polynucleotides provided herein, and therapeutic methods for treating such diseases, disorders, and/or conditions. The invention further relates to screening methods for identifying binding partners of the polypeptides.

The invention relates to a method of analyzing at least one nucleic acid sample, comprising the step of determining the nucleic acid sequence from one or more samples at one or more polymorphic loci in the human Adiponectin gene selected from the group consisting of SNP1, or any combination thereof, wherein the presence of the variable allele at said one or more polymorphic loci is indicative of an increased risk of developing dose-dependent congestive heart failure in a patient receiving PPAR-agonist therapy.

The invention relates to a method of analyzing at least one nucleic acid sample, comprising the step of determining the nucleic acid sequence from one or more samples at one or more polymorphic loci in the human Adiponectin gene selected from the group consisting of SNP1, or any combination thereof, wherein the presence of the reference allele at said one or more polymorphic loci is indicative of a decreased risk of developing dose-dependent congestive heart failure in a patient receiving PPAR-agonist therapy.

The invention further relates to a method of constructing haplotypes using the isolated nucleic acids referred to in FIGS. 7A-D, and/or FIGS. 8A-D, or elsewhere herein, comprising the step of grouping at least two said nucleic acids.

The invention further relates to a method of constructing haplotypes further comprising the step of using said haplotypes to identify an individual for the presence of dose-dependent congestive heart failure or related disease phenotype, and correlating the presence of the disease phenotype with said haplotype.

The invention further relates to a library of nucleic acids, each of which comprises one or more polymorphic positions within a gene encoding the human Adiponectin protein, wherein said polymorphic positions are selected from a group consisting of the polymorphic positions provided in FIGS. 7A-D, FIGS. 8A-D, and Table I.

The invention further relates to a library of nucleic acids, wherein the sequence at said aforementioned polymorphic position is selected from the group consisting of the polymorphic position identified in FIGS. 7A-D, and/or FIGS. 8A-D, or elsewhere herein, the complimentary sequence of said sequences, and/or fragments of said sequences.

The invention further relates to a kit for identifying an individual at risk of developing dose-dependent congestive heart failure or related disorder upon administration of a pharmaceutically acceptable amount of a PPAR-agonist, wherein said kit comprises oligonucleotide primers capable of identifying the nucleotide residing at one or more polymorphic loci of the human Adiponectin gene, wherein the presence of the reference allele at said one or more polymorphic loci is indicative of a decreased risk of developing dose-dependent congestive heart failure in a patient receiving PPAR-agonist therapy, while the presence of the variable allele at said one or more polymorphic loci is indicative of an increased risk of developing dose-dependent congestive heart failure in a patient receiving PPAR-agonist therapy.

The invention further relates to a kit for identifying an individual at risk of developing dose-dependent congestive heart failure or related disorder upon administration of a pharmaceutically acceptable amount of a PPAR-agonist, wherein said kit comprises oligonucleotide primers capable of identifying the nucleotide residing at one or more polymorphic loci of the human Adiponectin gene, wherein the presence of the reference allele at said one or more polymorphic loci is indicative of a decreased risk of developing dose-dependent congestive heart failure in a patient receiving PPAR-agonist therapy, while the presence of the variable allele at said one or more polymorphic loci is indicative of an increased risk of developing dose-dependent congestive heart failure in a patient receiving PPAR-agonist therapy, and wherein said oligonucleotides hybridize immediately adjacent to said one or more polymorphic positions, or wherein said primer(s) hybridizes to said polymorphic positions such that the central position of the primer aligns with the polymorphic position of said gene.

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the nucleotide present within at least one or more nucleic acid sample(s) from an individual to be assessed at one or more polymorphic position(s) of the human Adiponectin gene sequence selected from the group consisting of: SEQ ID NOS:26, and/or 28, wherein the presence of the variable nucleotide at the one or more polymorphic position(s) indicates that the individual has an increased likelihood of being diagnosed as at risk of developing dose-dependent congestive heart failure or related disorder upon administration of a pharmaceutically acceptable amount of an PPAR-agonist as compared to an individual having the reference allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the nucleotide present within at least one or more nucleic acid sample(s) from an individual to be assessed at one or more polymorphic position(s) of the human Adiponectin gene sequence selected from the group consisting of: SEQ ID NOS:26, and/or 28, wherein the presence of the reference nucleotide at the one or more polymorphic position(s) indicates that the individual has a decreased likelihood of being diagnosed as at risk of developing dose-dependent congestive heart failure or related disorder upon administration of a pharmaceutically acceptable amount of an PPAR-agonist as compared to an individual having the variable allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the polypeptide present within at least one or more sample(s) from an individual to be assessed at one or more polymorphic position(s) of the human Adiponectin polypeptide sequence selected from the group consisting of: SEQ ID NOS:27, and/or 29, wherein the presence of the variable amino acid at the one or more polymorphic position(s) indicates that the individual has an increased likelihood of being diagnosed as at risk of developing dose-dependent congestive heart failure or related disorder upon administration of a pharmaceutically acceptable amount of an PPAR-agonist as compared to an individual having the reference allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the nucleotide present within at least one or more sample(s) from an individual to be assessed at one or more polymorphic position(s) of the human Adiponectin polypeptide sequence selected from the group consisting of: SEQ ID NOS:26, and/or 28, wherein the presence of the reference amino acid at the one or more polymorphic position(s) indicates that the individual has a decreased likelihood of being diagnosed as at risk of developing dose-dependent congestive heart failure or related disorder upon administration of a pharmaceutically acceptable amount of an PPAR-agonist as compared to an individual having the variable allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the nucleotide present within at least one or more nucleic acid sample(s) from an individual to be assessed at one or more polymorphic position(s) of the human Adiponectin gene sequence selected from the group consisting of: nucleotide position 415 of SEQ ID NOS:26 or 28, wherein the presence of the variable nucleotide at the one or more polymorphic position(s) indicates that the individual has an increased likelihood of being diagnosed as at risk of developing dose-dependent congestive heart failure or related disorder upon administration of a pharmaceutically acceptable amount of a PPAR-agonist as compared to an individual having the reference allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the nucleotide present within at least one or more nucleic acid sample(s) from an individual to be assessed at one or more polymorphic position(s) of the human Adiponectin gene sequence selected from the group consisting of: nucleotide position 415 of SEQ ID NOS:26 or 28, wherein the presence of the reference nucleotide at the one or more polymorphic position(s) indicates that the individual has a decreased likelihood of being diagnosed as at risk of developing dose-dependent congestive heart failure or related disorder upon administration of a pharmaceutically acceptable amount of a PPAR-agonist as compared to an individual having the variable allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the amino acid present within at least one or more sample(s) from an individual to be assessed at one or more polymorphic position(s) of the human Adiponectin polypeptide sequence selected from the group consisting of: amino acid position 111 of SEQ ID NOS:27 or 29, wherein the presence of the variable amino acid at the one or more polymorphic position(s) indicates that the individual has an increased likelihood of being diagnosed as at risk of developing dose-dependent congestive heart failure or related disorder upon administration of a pharmaceutically acceptable amount of a PPAR-agonist as compared to an individual having the reference allele at said polymorphic position(s).

The invention further relates to a method for predicting the likelihood that an individual will be diagnosed as being at risk of developing dose-dependent congestive heart failure or related disorder upon administration of a pharmaceutically acceptable amount of a PPAR-agonist comprising the step of determining the amino acid present within at least one or more sample(s) from an individual to be assessed at one or more polymorphic position(s) of the human Adiponectin polypeptide sequence selected from the group consisting of: amino acid position 111 of SEQ ID NOS:27 or 29, wherein the presence of the reference amino acid at the one or more polymorphic position(s) indicates that the individual has a decreased likelihood of being diagnosed as at risk of developing dose-dependent congestive heart failure or related disorder upon administration of a pharmaceutically acceptable amount of a PPAR-agonist as compared to an individual having the variable allele at said polymorphic position(s).

The present invention is also directed to methods of predicting whether a patient administered a PPAR-agonist will respond to PPAR-agonist therapy; for predicting whether a patient will respond to specific doses of a PPAR-agonist; whether the level of the administered PPAR-agonist needs to be increased or decreased to achieve the desired level of human Adiponectin expression identified as representing a responsive level; whether a patient has an increased risk of developing dose-dependent congestive heart failure upon the administration of a pharmaceutically acceptable level of a PPAR-agonist; whether said patient requires a lower level of administered PPAR agonist to limit the risk of developing said dose-dependent congestive heart failure; or whether said patient may be administered a higher level of administered PPAR agonist without the risk of developing said dose-dependent congestive heart failure, inorder to limit the risk of developing said dose-dependent congestive heart failure, comprising the step of assessing the level of Adiponectin expression resulting from the administration of a PPAR-agonist relative to a control compound.

### BRIEF DESCRIPTION OF THE FIGURES/DRAWINGS

FIGS. 1A-F show the polynucleotide sequence (SEQ ID NO:1) of the SNP1 allele “a” of the human mineralocorticoid receptor (MLR) polynucleotide sequence comprising, or alternatively consisting of, a predicted polynucleotide polymorphic locus located at nucleotide 754 of SEQ ID NO:1, and the resulting encoded MLR polypeptide (SEQ ID NO:2), comprising, or alternatively consisting of, a predicted sense polymorphic locus located at amino acid 180 of SEQ ID NO:2. The polynucleotide sequence contains a sequence of 5749 nucleotides (SEQ ID NO:1), which encodes a polypeptide containing a sequence of 984 amino acids (SEQ ID NO:2). The nucleotide at the polymorphic locus for this allele is a “a” and is denoted in bold and double underlining, while the amino acid encoded by the “a” allele at the polymorphic locus is an “Ile” and is also denoted in bold and double underlining.

FIGS. 2A-F show the polynucleotide sequence (SEQ ID NO:3) of the SNP1 allele “g” of the human mineralocorticoid receptor (MLR) polynucleotide sequence comprising, or alternatively consisting of, a predicted polynucleotide polymorphic locus located at nucleotide 754 of SEQ ID NO:3, and the resulting encoded MLR polypeptide (SEQ ID NO:4), comprising, or alternatively consisting of, a predicted sense polymorphic locus located at amino acid 180 of SEQ ID NO:4. The polynucleotide sequence contains a sequence of 5749 nucleotides (SEQ ID NO:3), which encodes a polypeptide containing a sequence of 984 amino acids (SEQ ID NO:4). The nucleotide at the polymorphic locus for this allele is a “g” and is denoted in bold and double underlining, while the amino acid encoded by the “g” allele at the polymorphic locus is an “Val” and is also denoted in bold and double underlining.

FIG. 3 shows the statistical association between human mineralocorticoid receptor (MLR) SNP1 alleles “a” (“Ile”) and “g” (“Val”) with the incidence of congestive heart failure (CHF) in patients administered a PPAR alpha/gamma agonist. As shown, “g” allele homozygous patients (“V/V”) at the SNP1 locus have the highest incidence of CHF; while heterozygous patients (“I/V”) have a slighter lower incidence of CHF compared to homozygous “V/V” allele patients. Homozygous “a” allele patients (“I/I”) had the lowest incidence of CHF.

FIGS. 4A-B show the polynucleotide sequence (SEQ ID NO:13) of the SNP1 allele “c” of the human PPAR-gamma polynucleotide sequence comprising, or alternatively consisting of, a predicted polynucleotide polymorphic locus located at nucleotide 167 of SEQ ID NO:13, and the resulting encoded PPAR-gamma polypeptide (SEQ ID NO:14), comprising, or alternatively consisting of, a predicted sense polymorphic locus located at amino acid 12 of SEQ ID NO:14. The polynucleotide sequence contains a sequence of 1863 nucleotides (SEQ ID NO:13), which encodes a polypeptide containing a sequence of 505 amino acids (SEQ ID NO:14). The nucleotide at the polymorphic locus for this allele is a “c” and is denoted in bold and double underlining, while the amino acid encoded by the “c” allele at the polymorphic locus is a “P” and is also denoted in bold and double underlining.

FIGS. 5A-B show the polynucleotide sequence (SEQ ID NO:15) of the SNP1 allele “g” of the human PPAR-gamma polynucleotide sequence comprising, or alternatively consisting of, a predicted polynucleotide polymorphic locus located at nucleotide 167 of SEQ ID NO:15, and the resulting encoded PPAR-gamma polypeptide (SEQ ID NO:16), comprising, or alternatively consisting of, a predicted sense polymorphic locus located at amino acid 12 of SEQ ID NO:16. The polynucleotide sequence contains a sequence of 1863 nucleotides (SEQ ID NO:15), which encodes a polypeptide containing a sequence of 505 amino acids (SEQ ID NO:16). The nucleotide at the polymorphic locus for this allele is a “g” and is denoted in bold and double underlining, while the amino acid encoded by the “g” allele at the polymorphic locus is an “A” and is also denoted in bold and double underlining.

FIG. 6 shows the statistical association between human PPAR-gamma SNP1 alleles “c” (“Pro”) and “g” (“Ala”) with the incidence of congestive heart failure (CHF) in patients administered a PPAR alpha/gamma agonist. As shown, “g” allele homozygous patients (“Ala/Ala”) at the SNP1 locus have the highest incidence of CHF; while heterozygous patients (“Pro/Ala”) at the SNP1 locus have a slighter lower incidence of CHF compared to homozygous “Pro/Pro” allele patients.

FIGS. 7A-D show the polynucleotide sequence (SEQ ID NO:26) of the SNP1 allele “t” of the human Adiponectin polynucleotide sequence comprising, or alternatively consisting of, a predicted polynucleotide polymorphic locus located at nucleotide 415 of SEQ ID NO:26, and the resulting encoded Adiponectin polypeptide (SEQ ID NO:27), comprising, or alternatively consisting of, a predicted sense polymorphic locus located at amino acid 111 of SEQ ID NO:27. The polynucleotide sequence contains a sequence of 4592 nucleotides (SEQ ID NO:26), which encodes a polypeptide containing a sequence of 244 amino acids (SEQ ID NO:27). The nucleotide at the polymorphic locus for this allele is a “t” and is denoted in bold and double underlining, while the amino acid encoded by the “t” allele at the polymorphic locus is a “T” and is also denoted in bold and double underlining.

FIGS. 8A-D show the polynucleotide sequence (SEQ ID NO:28) of the SNP1 allele “c” of the human Adiponectin polynucleotide sequence comprising, or alternatively consisting of, a predicted polynucleotide polymorphic locus located at nucleotide 415 of SEQ ID NO:28, and the resulting encoded Adiponectin polypeptide (SEQ ID NO:29), comprising, or alternatively consisting of, a predicted sense polymorphic locus located at amino acid 111 of SEQ ID NO:29. The polynucleotide sequence contains a sequence of 4592 nucleotides (SEQ ID NO:28), which encodes a polypeptide containing a sequence of 244 amino acids (SEQ ID NO:29). The nucleotide at the polymorphic locus for this allele is a “c” and is denoted in bold and double underlining, while the amino acid encoded by the “c” allele at the polymorphic locus is a “H” and is also denoted in bold and double underlining.

FIG. 9 shows the statistical association between human Adiponectin SNP1 alleles “t” (“Tyr”) and “c” (“His”) with the incidence of congestive heart failure (CHF) in patients administered a PPAR alpha/gamma agonist. As shown, “c” allele heterozygous patients (“Tyr/His”) at the SNP1 locus have the highest incidence of CHF; while homozygous “t” allele patients (“Tyr/Tyr”) at the SNP1 locus have a significantly lower incidence of CHF. No homozygous “c” allele patients were observed in this study.

Table I provides a summary of the SNPs of the present invention.

### DETAILED DESCRIPTION OF THE INVENTION

The present invention relates to a nucleic acid molecule comprising a single nucleotide polymorphism (SNP) at a specific location, referred to herein as the polymorphic locus, and complements thereof. The nucleic acid molecule, e.g., a gene, which includes the SNP has at least two alleles, referred to herein as the reference allele and the variant allele. The reference allele (prototypical or wild type allele) typically corresponds to the nucleotide sequence of the native form of the nucleic acid molecule.

The present invention pertains to novel polynucleotides of the human mineralocorticoid receptor gene comprising at least one single nucleotide polymorphism (SNP) which has been shown to be associated with the incidence of dose-dependent congestive heart failure in patients administered PPAR-agonists. These mineralocorticoid receptor SNPs were identified by sequencing the mineralocorticoid receptor genomic sequence of a large number of individuals that were subjected to PPAR-agonist therapy, and comparing the mineralocorticoid receptor sequences of those individuals who developed dose-dependent congestive heart failure to those individuals who did not develop dose-dependent congestive heart failure. Each of the novel mineralocorticoid receptor SNPs were located in non-coding regions of the mineralocorticoid receptor gene and are thought to affect the expression levels of mineralocorticoid receptor in those patients containing one or more of these SNPs.

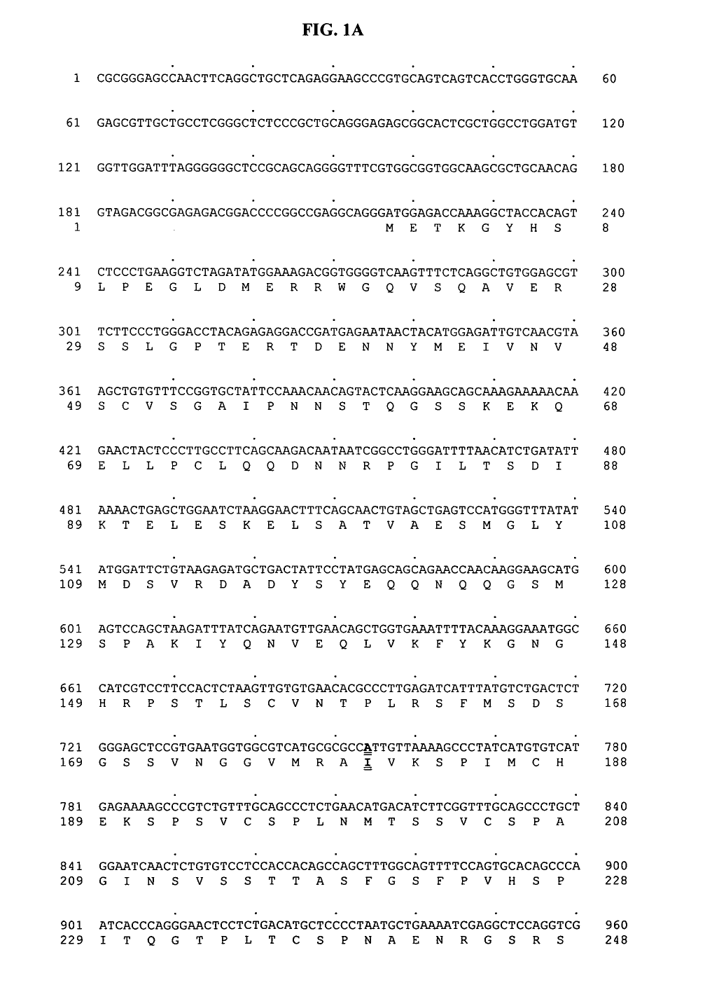

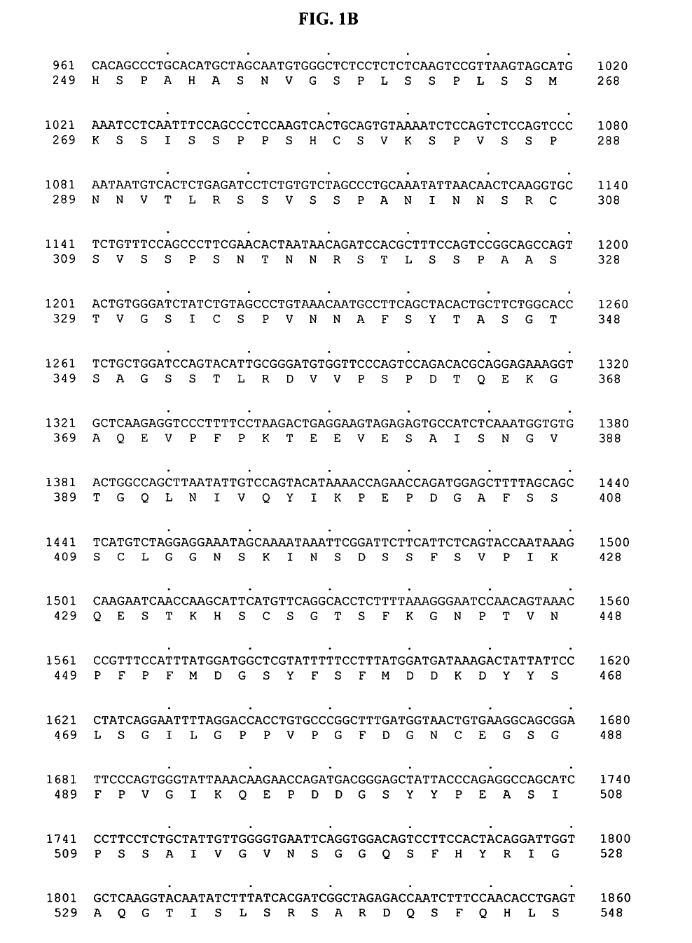

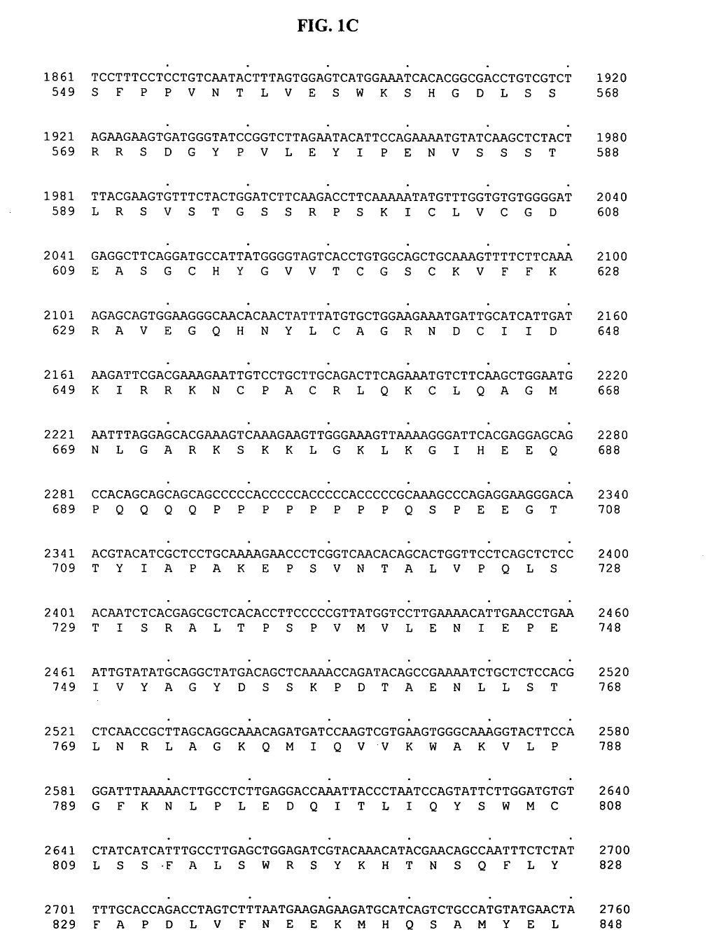

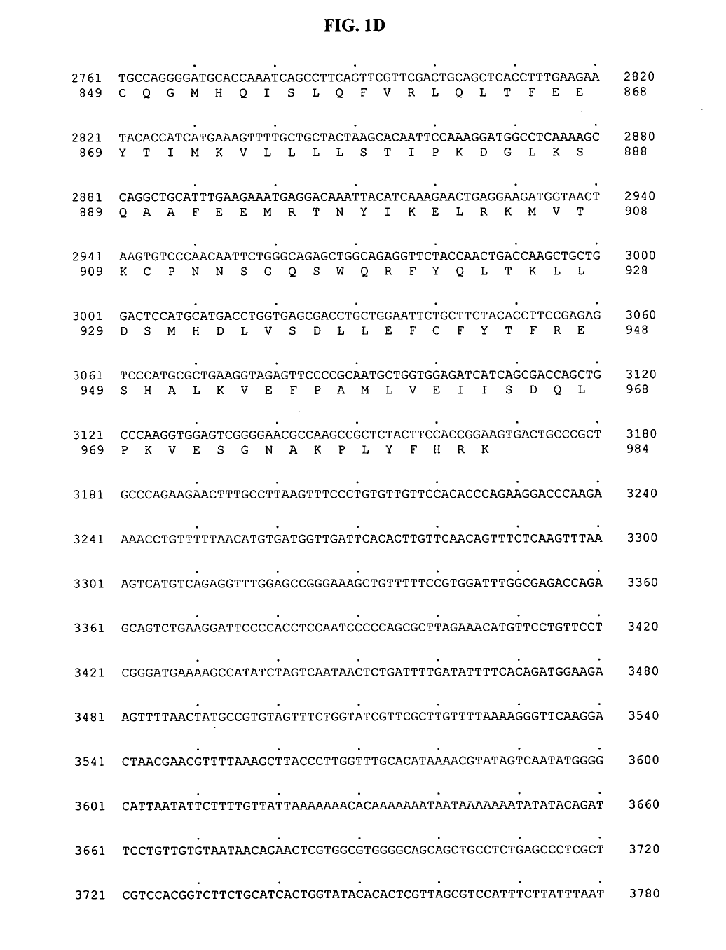

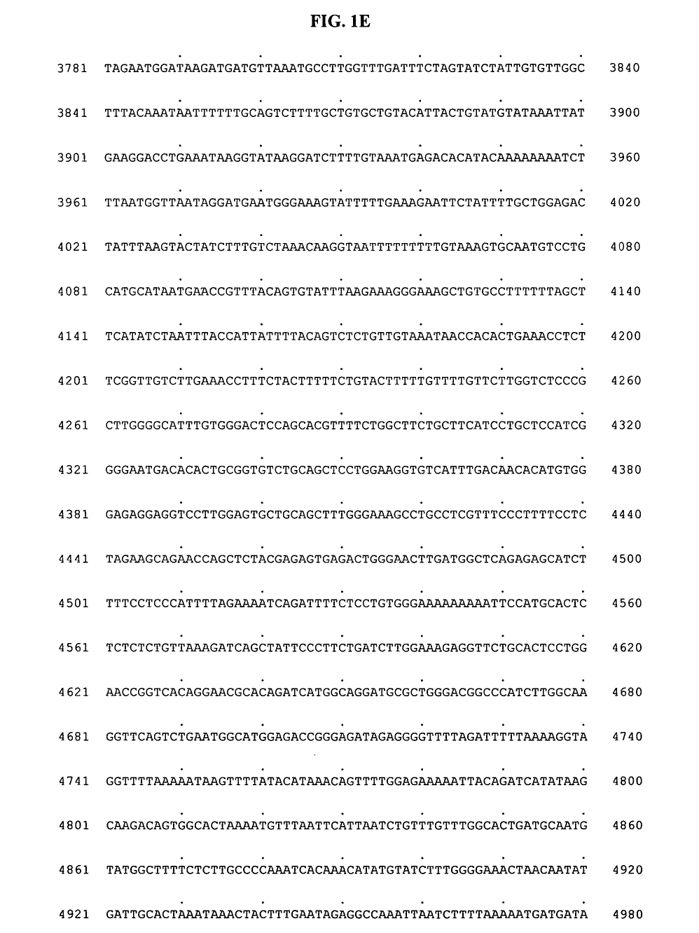

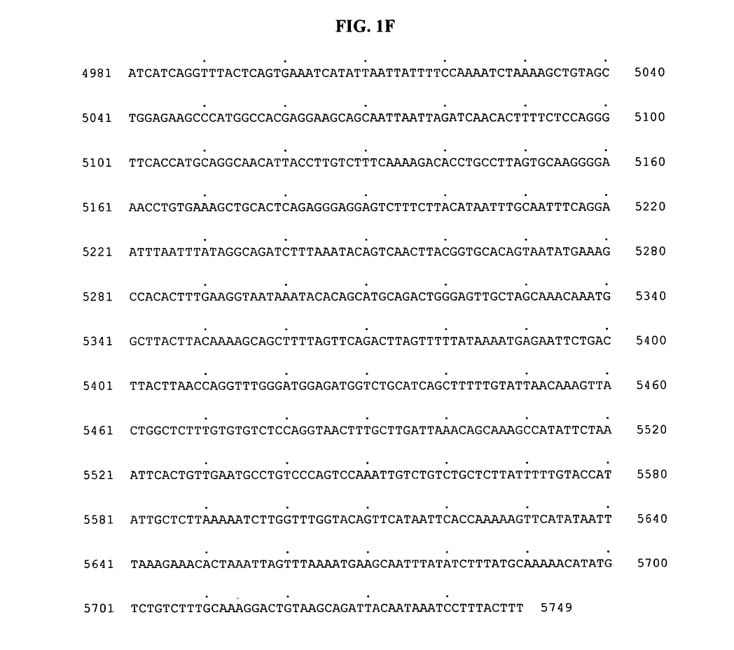

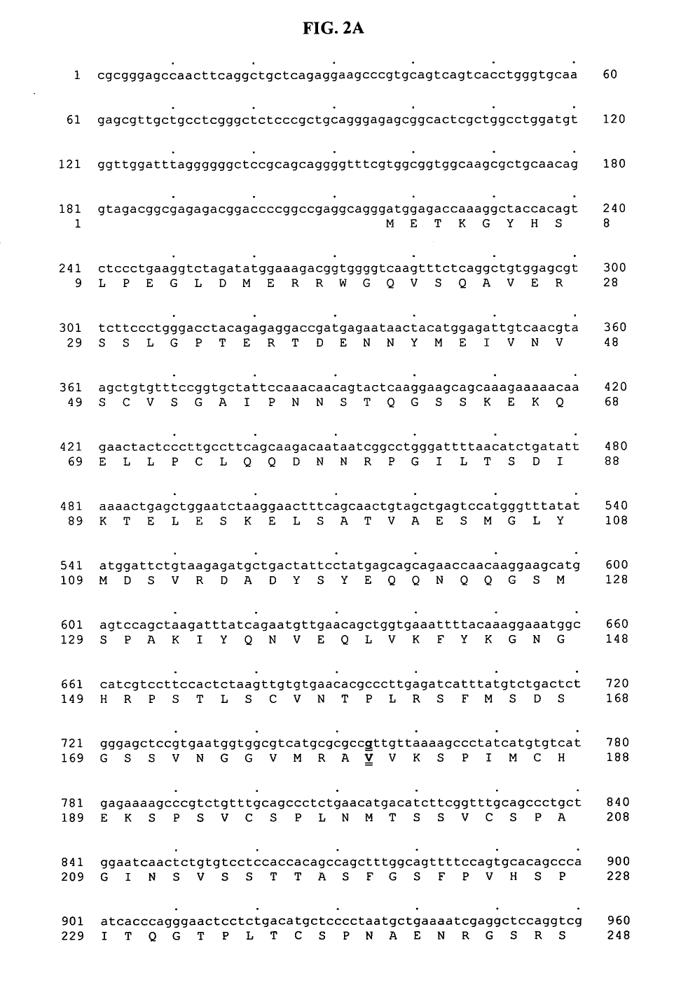

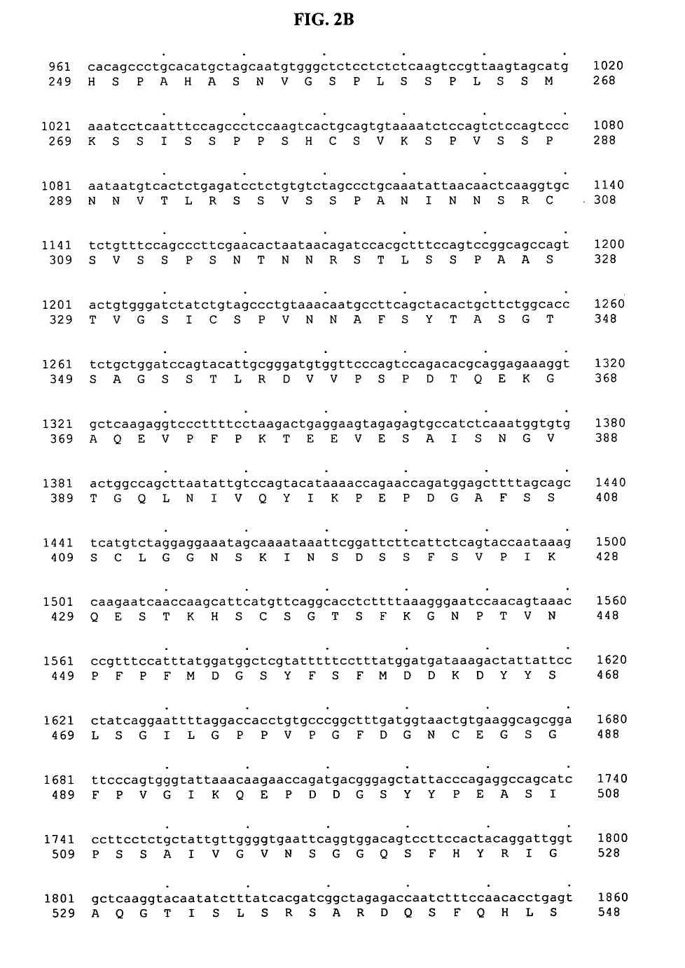

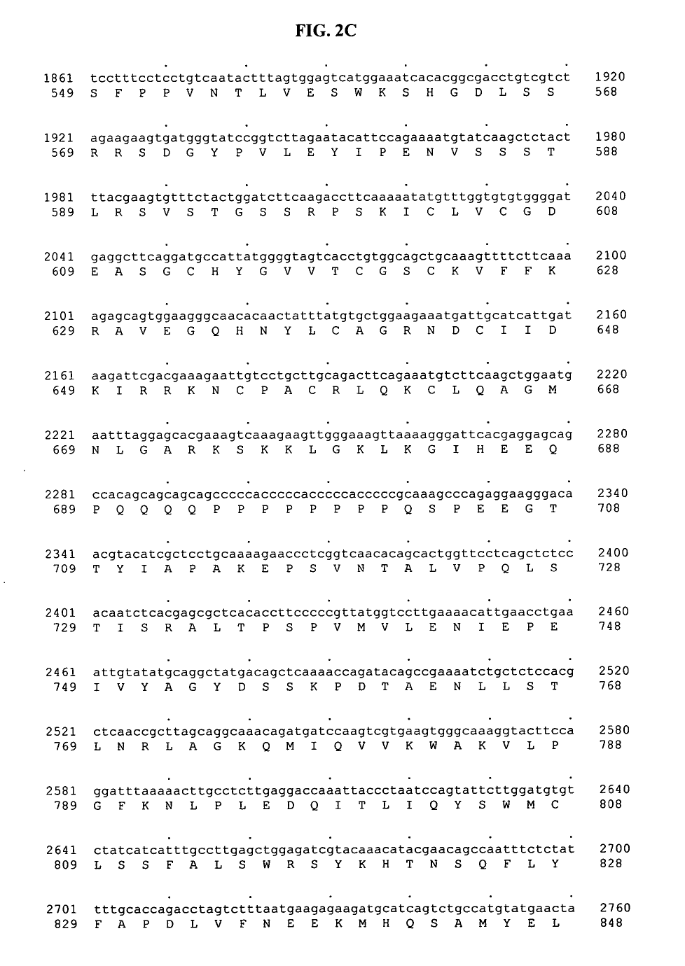

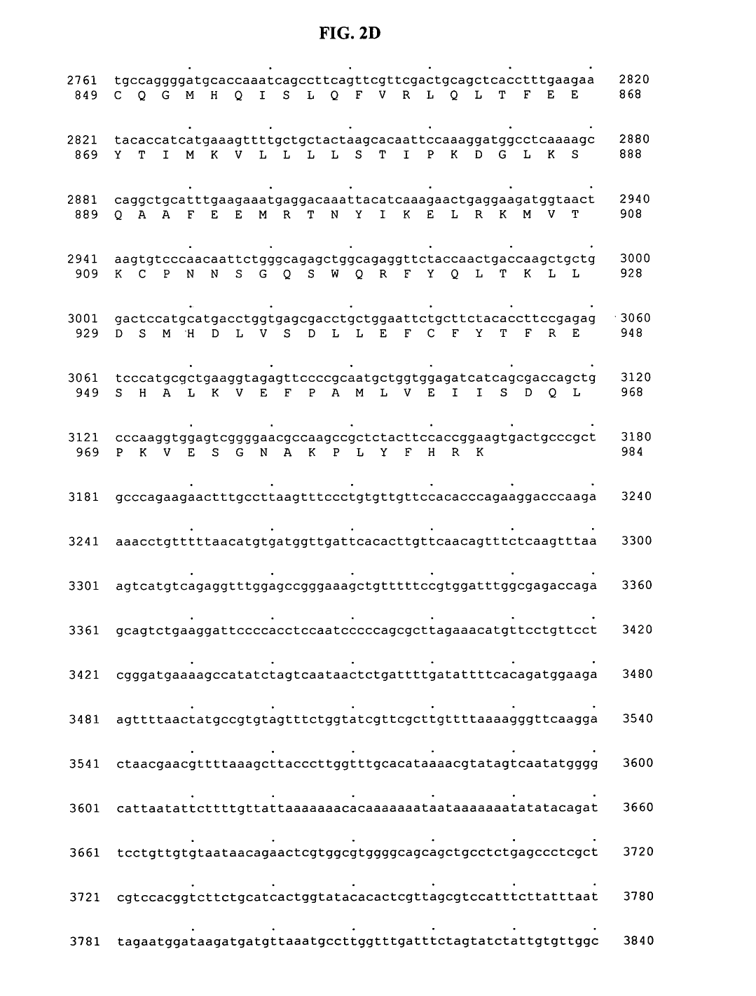

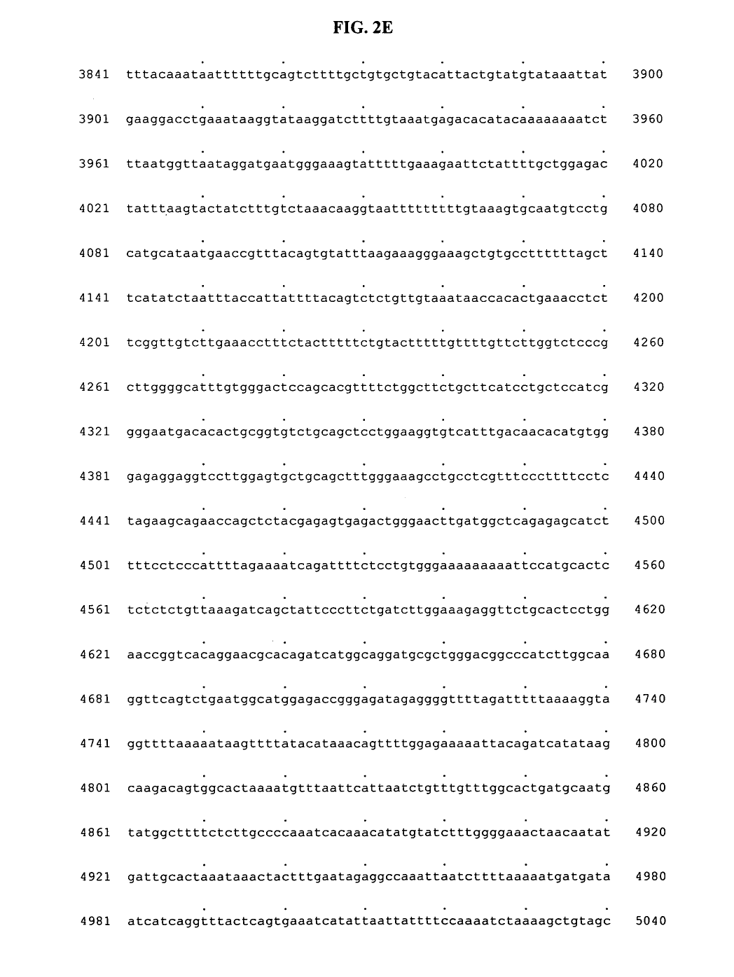

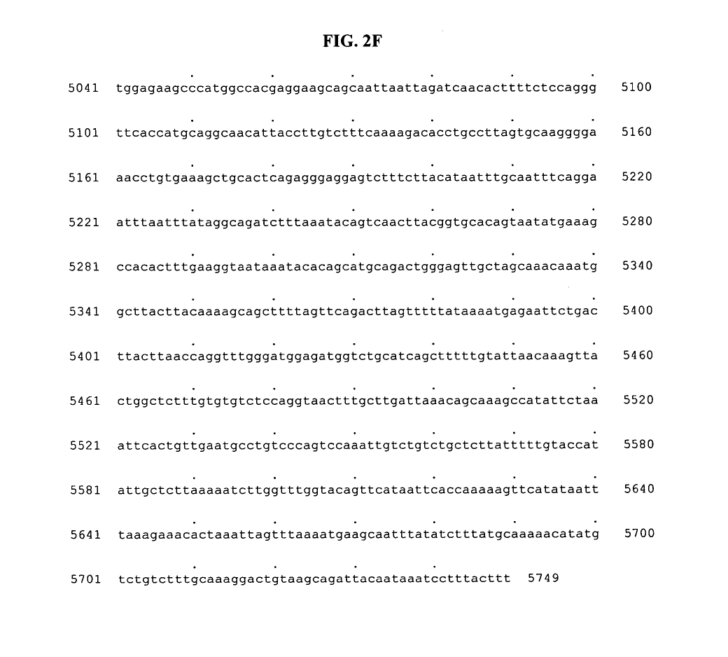

The present invention also relates to variant alleles of the described gene and to complements of the variant alleles. The variant allele differs from the reference allele by one nucleotide at the polymorphic locus identified in the FIGS. 1A-F, and/or FIGS. 2A-F.

The invention further relates to fragments of the variant alleles and portions of complements of the variant alleles which comprise the site of the SNP (e.g., polymorphic locus) and are at least 10 nucleotides in length. Fragments can be, for example, about 5-10, about 5-15, about 10-20, about 5-25, about 10-30, about 10-50 or about 10-100 bases long. For example, a portion of a variant allele which is about 10 nucleotides in length comprises at least one single nucleotide polymorphism (the nucleotide which differs from the reference allele at the polymorphic locus) and nine additional nucleotides which flank the site in the variant allele. These additional nucleotides can be on one or both sides of the polymorphism. Polymorphisms which are the subject of this invention are defmed in FIGS. 1A-F, and/or FIGS. 2A-F herein.

Specifically, the invention relates to the human mineralocorticoid receptor gene having a nucleotide sequence according to FIGS. 1A-F, or FIGS. 2A-F (SEQ ID NOs:1, or 3) comprising a single nucleotide polymorphism at a polymorphic locus selected from the group consisting of: nucleotide 754 of SEQ ID NOs:1 or 3. The reference nucleotide for the polymorphic locus at nucleotide 754 is “a”. The variant nucleotide for the polymorphic locus at nucleotide 754 is “g”. The nucleotide sequences of the present invention can be double- or single-stranded.

The invention also relates to the human mineralocorticoid receptor polypeptide having an amino acid sequence according to FIGS. 1A-F, or FIGS. 2A-F (SEQ ID NOs:2, or 4) comprising a polymorphism at a polymorphic locus selected from the group consisting of: amino acid 180 of SEQ ID NOs:2 or 4. The reference amino acid for the polymorphic locus at amino acid 180 is “Ile”. The variant amino acid for the polymorphic locus at amino acid 180 is “Val”.

The invention further relates to a portion of the human mineralocorticoid receptor gene comprising one or more polymorphic loci selected from the group consisting of: nucleotide 754 of SEQ ID NOs:1 or 3.

The single nucleotide polymorphisms described herein derive from the mineralocorticoid receptor gene that have been shown to be associated, for the first time, with the incidence of dose-dependent congestive heart failure or related disorders. Specifically, the variable single nucleotide polymorphism(s) of the human mineralocorticoid receptor gene described herein have been demonstrated to statistically increase an individuals susceptibility to developing dose-dependent congestive heart failure or a congestive heart failure-like event upon the administration of a PPAR-agonist.

The human mineralocorticoid receptor gene was chosen as a candidate gene to investigate the potential of it comprising one or more single nucleotide polymorphisms associated with dose-dependent congestive heart failure or related-congestive heart failure phenotype, and in particular, the potential of identifying a mineralocorticoid receptor SNP associated with the incidence of dose-dependent congestive heart failure or related-congestive heart failure phenotype upon the administration of either the prescribed dose, or an increased dose of a PPAR-agonist, based upon the role of mineralocorticoid receptor in regulating sodium and water re-absorption by the kidney.

The invention further provides allele-specific oligonucleotides that hybridize to the human mineralocorticoid receptor gene, or fragments or complements thereof, comprising one or more single nucleotide polymorphisms and/or polymorphic locus. Such oligonucleotides are expected to hybridize to one polymorphic allele of the nucleic acid molecules described herein but not to the other polymorphic allele(s) of the sequence. Thus, such oligonucleotides can be used to determine the presence or absence of particular alleles of the polymorphic sequences described herein and to distinguish between reference and variant allele for each form. These oligonucleotides can be probes or primers.

The invention further provides a method of analyzing a nucleic acid from an individual to identify the presence or absence of a particular nucleotide at a given polymorphic locus and to distinguish between the reference and variant allele at each locus. The method determines which base is present at any one of the polymorphic loci shown in FIGS. 1A-F, and/or FIGS. 2A-F (SEQ ID NOs:1, and/or 3), or elsewhere herein. Optionally, a set of bases occupying a set of the polymorphic loci shown in FIGS. 1A-F, and/or FIGS. 2A-F (SEQ ID NOs:1, and/or 3) is determined. This type of analysis can be performed on a number of individuals, who are also tested (previously, concurrently or subsequently) for the presence of dose-dependent congestive heart failure or congestive heart failure-like phenotype in the presence or absence of a PPAR-agonist. The presence or absence of dose-dependent congestive heart failure or congestive heart failure-like phenotype is then correlated with a base or set of bases present at the polymorphic locus or loci in the patient and/or sample tested.

The present invention pertains to novel polynucleotides of the human PPAR-gamma gene comprising at least one single nucleotide polymorphism (SNP) which has been shown to be associated with the incidence of dose-dependent congestive heart failure in patients administered PPAR-agonists. These PPAR-gamma SNPs were identified by sequencing the PPAR-gamma genomic sequence of a large number of individuals that were subjected to PPAR-agonist therapy, and comparing the PPAR-gamma sequences of those individuals who developed dose-dependent congestive heart failure to those individuals who did not develop dose-dependent congestive heart failure. Each of the novel PPAR-gamma SNPs were located in non-coding regions of the PPAR-gamma gene and are thought to affect the expression levels of PPAR-gamma in those patients containing one or more of these SNPs.

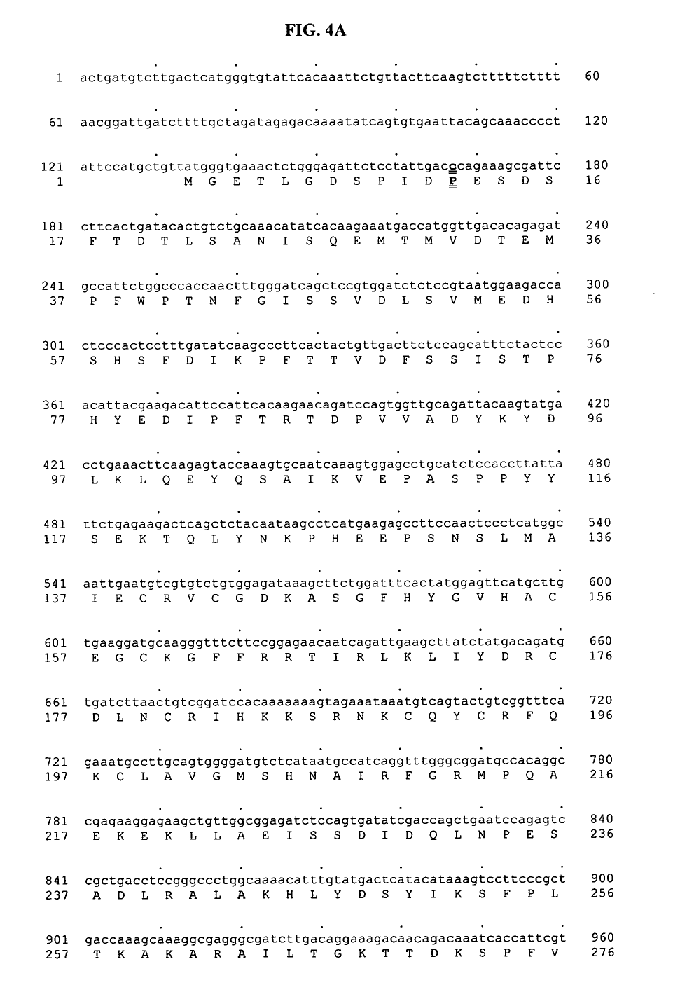

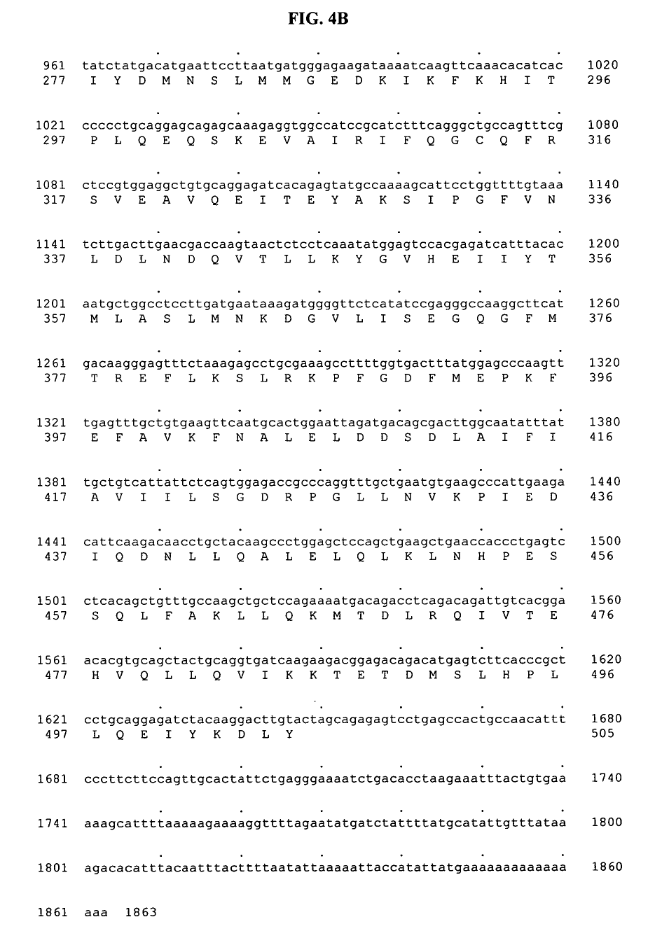

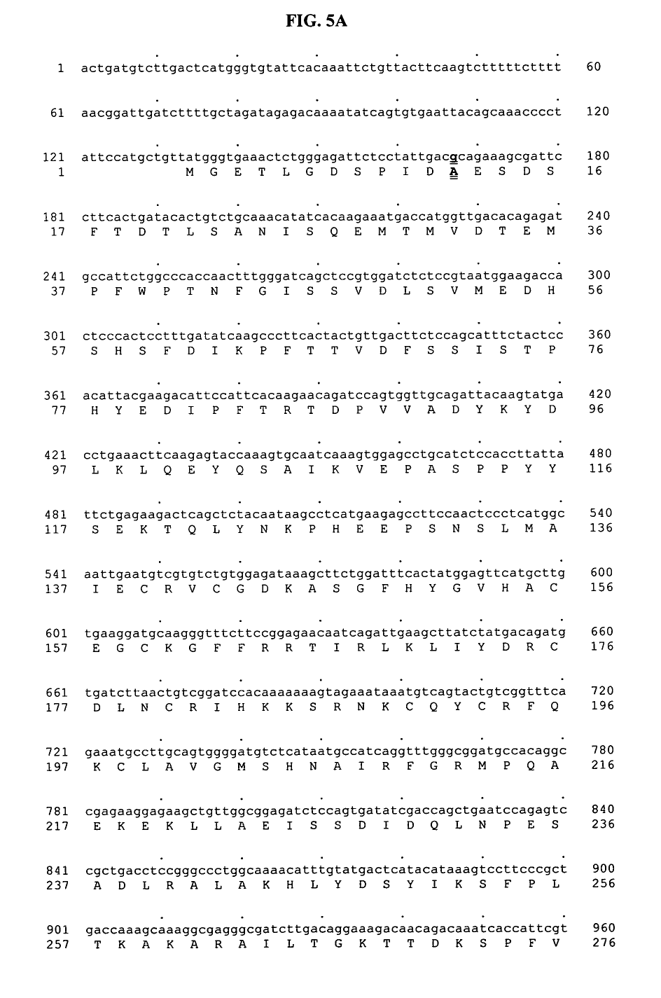

The present invention also relates to variant alleles of the described gene and to complements of the variant alleles. The variant allele differs from the reference allele by one nucleotide at the polymorphic locus identified in the FIGS. 4A-B, and/or FIGS. 5A-B.

The invention further relates to fragments of the variant alleles and portions of complements of the variant alleles which comprise the site of the SNP (e.g., polymorphic locus) and are at least 10 nucleotides in length. Fragments can be, for example, about 5-10, about 5-15, about 10-20, about 5-25, about 10-30, about 10-50 or about 10-100 bases long. For example, a portion of a variant allele which is about 10 nucleotides in length comprises at least one single nucleotide polymorphism (the nucleotide which differs from the reference allele at the polymorphic locus) and nine additional nucleotides which flank the site in the variant allele. These additional nucleotides can be on one or both sides of the polymorphism. Polymorphisms which are the subject of this invention are defined in FIGS. 4A-B, and/or FIGS. 5A-B herein.

Specifically, the invention relates to the human PPAR-gamma gene having a nucleotide sequence according to FIGS. 4A-B, or FIGS. 5A-B (SEQ ID NOs:13, or 15) comprising a single nucleotide polymorphism at a polymorphic locus selected from the group consisting of: nucleotide 167 of SEQ ID NOs:13 or 15. The reference nucleotide for the polymorphic locus at nucleotide 167 is “c”. The variant nucleotide for the polymorphic locus at nucleotide 167 is “g”. The nucleotide sequences of the present invention can be double- or single-stranded.

The invention also relates to the human PPAR-gamma polypeptide having an amino acid sequence according to FIGS. 4A-B, or FIGS. 5A-B (SEQ ID NOs:14, or 16) comprising a polymorphism at a polymorphic locus selected from the group consisting of: amino acid 12 of SEQ ID NOs:14 or 16. The reference amino acid for the polymorphic locus at amino acid 12 is “Pro”. The variant amino acid for the polymorphic locus at amino acid 12 is “Ala”.

The invention further relates to a portion of the human PPAR-gamma gene comprising one or more polymorphic loci selected from the group consisting of: nucleotide 167 of SEQ ID NOs:13 or 15.

The single nucleotide polymorphisms described herein derive from the PPAR-gamma gene that have been shown to be associated, for the first time, with the incidence of dose-dependent congestive heart failure or related disorders. Specifically, the variable single nucleotide polymorphism(s) of the human PPAR-gamma gene described herein have been demonstrated to statistically increase an individuals susceptibility to developing dose-dependent congestive heart failure or a congestive heart failure-like event upon the administration of a PPAR-agonist.

The human PPAR-gamma gene was chosen as a candidate gene to investigate the potential of it comprising one or more single nucleotide polymorphisms associated with dose-dependent congestive heart failure or related-congestive heart failure phenotype, and in particular, the potential of identifying a PPAR-gamma SNP associated with the incidence of dose-dependent congestive heart failure or related-congestive heart failure phenotype upon the administration of either the prescribed dose, or an increased dose of a PPAR-agonist, based upon the role of PPAR-gamma in regulating sodium and water re-absorption by the kidney.

The invention further provides allele-specific oligonucleotides that hybridize to the human PPAR-gamma gene, or fragments or complements thereof, comprising one or more single nucleotide polymorphisms and/or polymorphic locus. Such oligonucleotides are expected to hybridize to one polymorphic allele of the nucleic acid molecules described herein but not to the other polymorphic allele(s) of the sequence. Thus, such oligonucleotides can be used to determine the presence or absence of particular alleles of the polymorphic sequences described herein and to distinguish between reference and variant allele for each form. These oligonucleotides can be probes or primers.

The invention further provides a method of analyzing a nucleic acid from an individual to identify the presence or absence of a particular nucleotide at a given polymorphic locus and to distinguish between the reference and variant allele at each locus. The method determines which base is present at any one of the polymorphic loci shown in FIGS. 4A-B, and/or FIGS. 5A-B (SEQ ID NOs:13, and/or 15), or elsewhere herein. Optionally, a set of bases occupying a set of the polymorphic loci shown in FIGS. 4A-B, and/or FIGS. 5A-B (SEQ ID NOs:13, and/or 15) is determined. This type of analysis can be performed on a number of individuals, who are also tested (previously, concurrently or subsequently) for the presence of dose-dependent congestive heart failure or congestive heart failure-like phenotype in the presence or absence of a PPAR-agonist. The presence or absence of dose-dependent congestive heart failure or congestive heart failure-like phenotype is then correlated with a base or set of bases present at the polymorphic locus or loci in the patient and/or sample tested.

The present invention pertains to novel polynucleotides of the human Adiponectin gene comprising at least one single nucleotide polymorphism (SNP) which has been shown to be associated with the incidence of dose-dependent congestive heart failure in patients administered PPAR-agonists. These Adiponectin SNPs were identified by sequencing the Adiponectin genomic sequence of a large number of individuals that were subjected to PPAR-agonist therapy, and comparing the Adiponectin sequences of those individuals who developed dose-dependent congestive heart failure to those individuals who did not develop dose-dependent congestive heart failure. Each of the novel Adiponectin SNPs were located in non-coding regions of the Adiponectin gene and are thought to affect the expression levels of Adiponectin in those patients containing one or more of these SNPs.

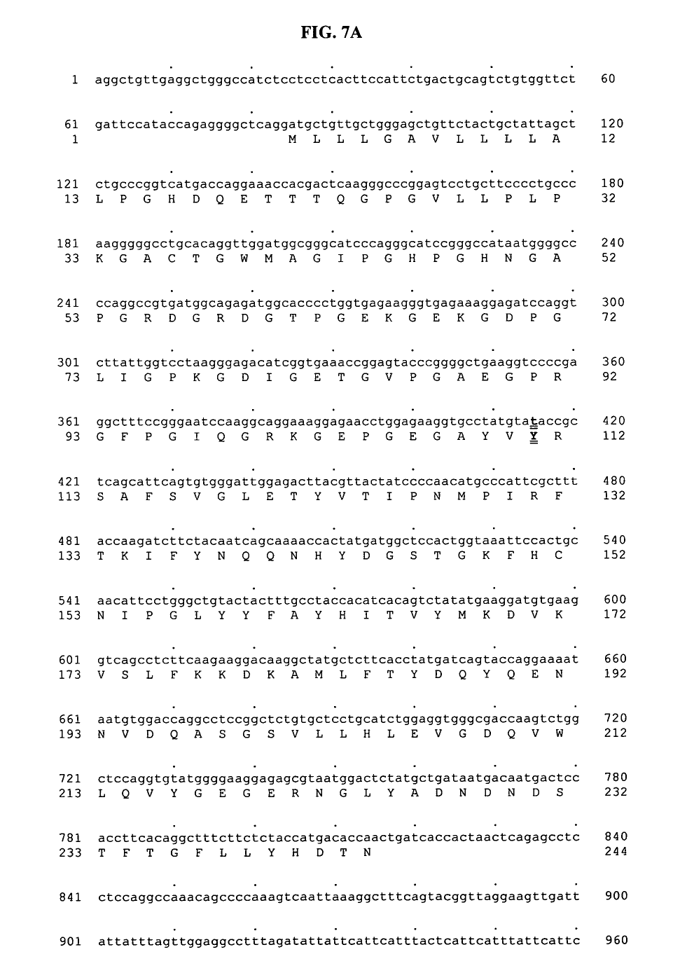

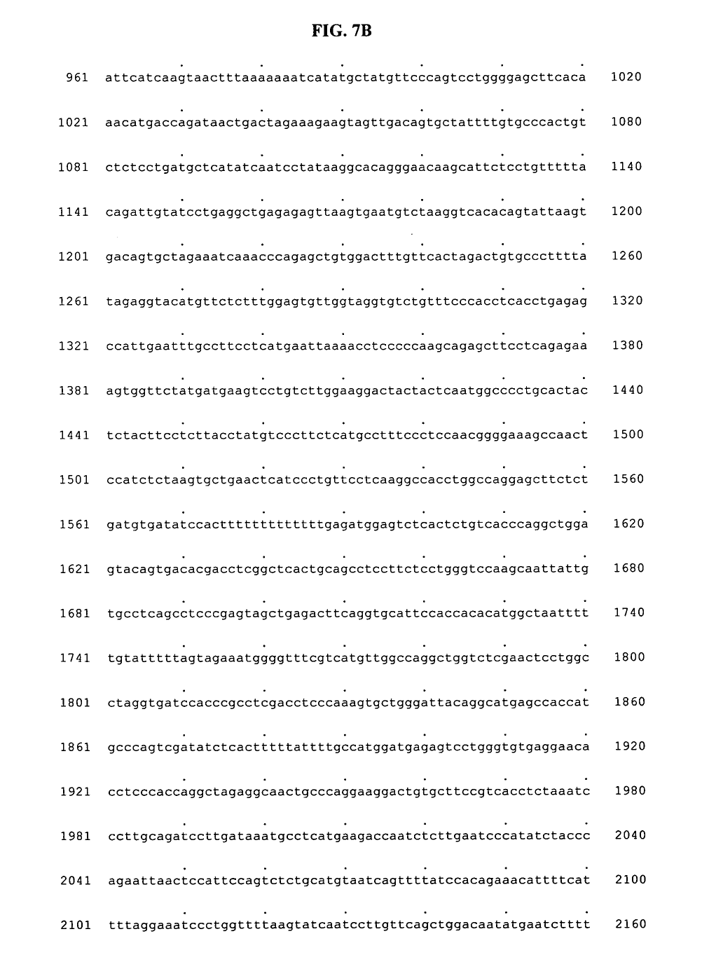

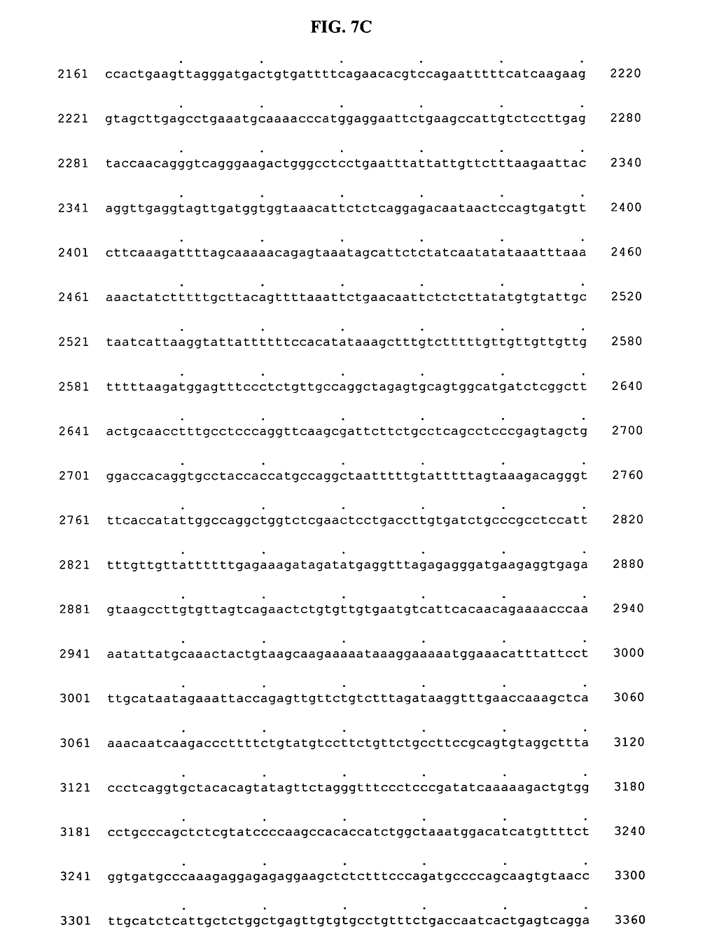

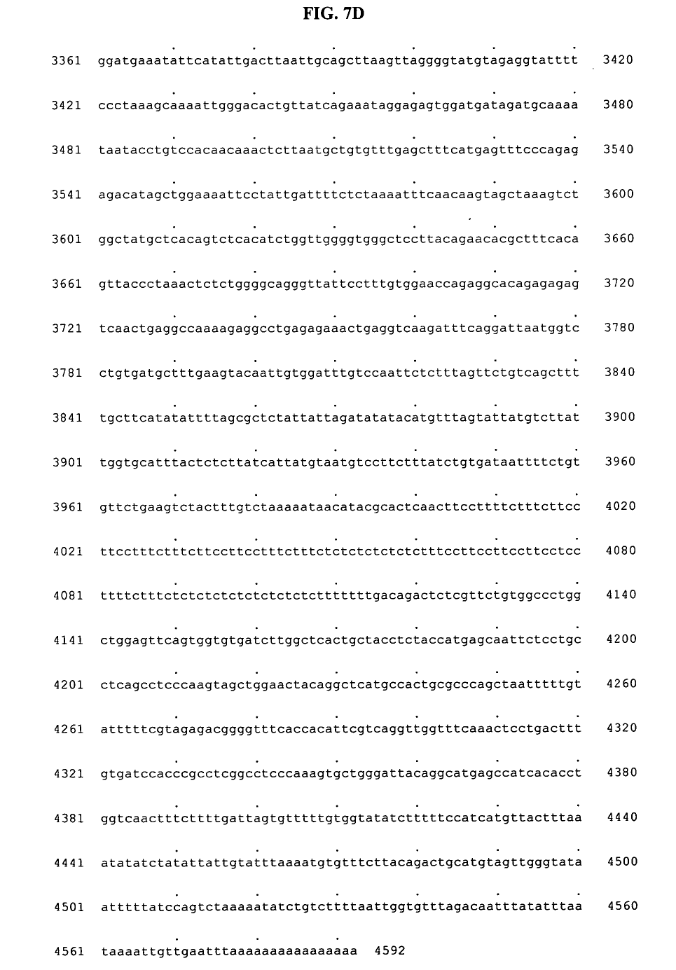

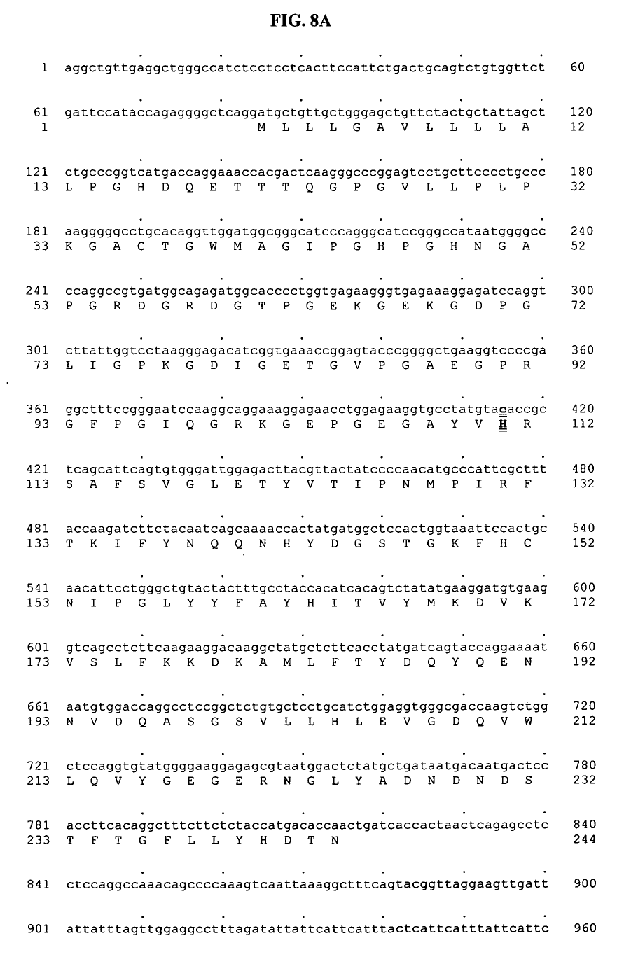

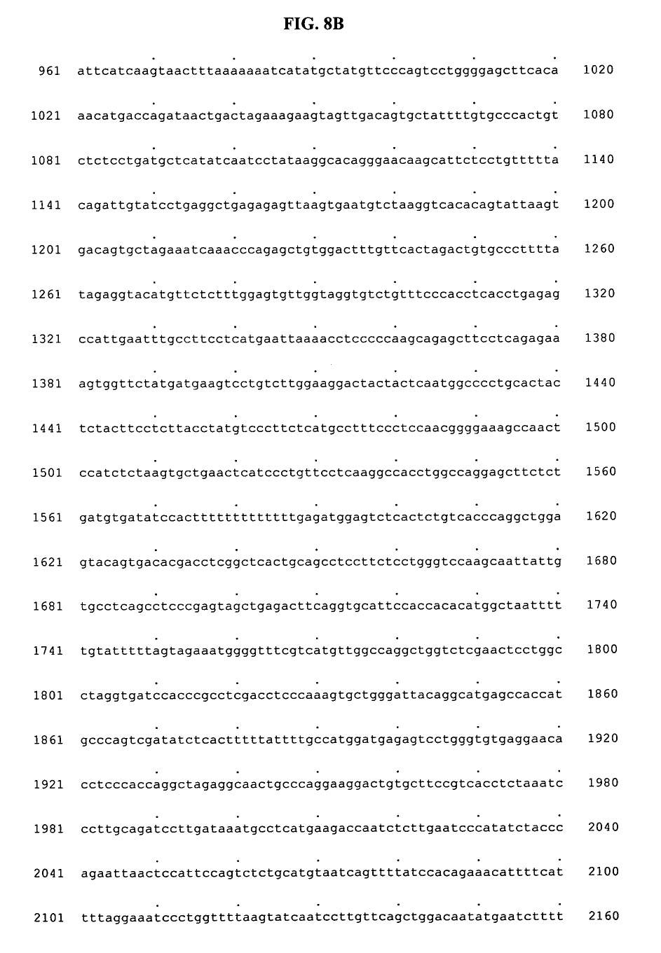

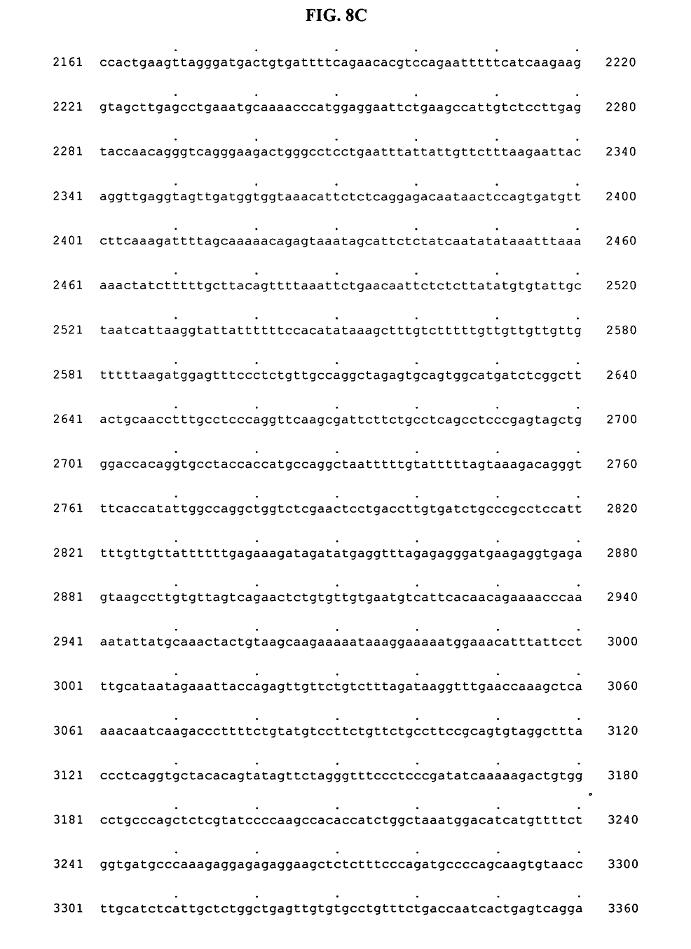

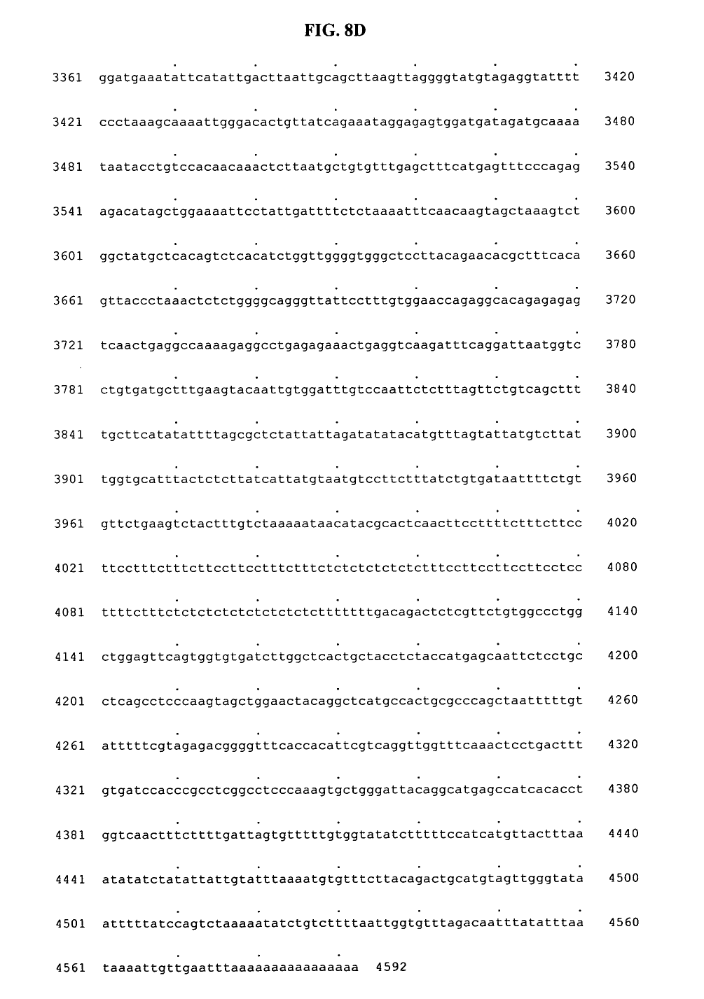

The present invention also relates to variant alleles of the described gene and to complements of the variant alleles. The variant allele differs from the reference allele by one nucleotide at the polymorphic locus identified in the FIGS. 7A-D, and/or FIGS. 8A-D.

The invention further relates to fragments of the variant alleles and portions of complements of the variant alleles which comprise the site of the SNP (e.g., polymorphic locus) and are at least 10 nucleotides in length. Fragments can be, for example, about 5-10, about 5-15, about 10-20, about 5-25, about 10-30, about 10-50 or about 10-100 bases long. For example, a portion of a variant allele which is about 10 nucleotides in length comprises at least one single nucleotide polymorphism (the nucleotide which differs from the reference allele at the polymorphic locus) and nine additional nucleotides which flank the site in the variant allele. These additional nucleotides can be on one or both sides of the polymorphism. Polymorphisms which are the subject of this invention are defmed in FIGS. 7A-D, and/or FIGS. 8A-D herein.

Specifically, the invention relates to the human Adiponectin gene having a nucleotide sequence according to FIGS. 7A-D, or FIGS. 8A-D (SEQ ID NOs:26, or 28) comprising a single nucleotide polymorphism at a polymorphic locus selected from the group consisting of nucleotide 415 of SEQ ID NOs:26 or 28. The reference nucleotide for the polymorphic locus at nucleotide 415 is “t”. The variant nucleotide for the polymorphic locus at nucleotide 415 is “c”. The nucleotide sequences of the present invention can be double- or single-stranded.

The invention also relates to the human Adiponectin polypeptide having an amino acid sequence according to FIGS. 7A-D, or FIGS. 8A-D (SEQ ID NOs:27, or 29) comprising a polymorphism at a polymorphic locus selected from the group consisting of: amino acid 111 of SEQ ID NOs:27 or 29. The reference amino acid for the polymorphic locus at amino acid 111 is “Tyr”. The variant amino acid for the polymorphic locus at amino acid 111 is “His”.

The invention further relates to a portion of the human Adiponectin gene comprising one or more polymorphic loci selected from the group consisting of:

nucleotide 415 of SEQ ID NOs:26 or 28.

The single nucleotide polymorphisms described herein derive from the Adiponectin gene that have been shown to be associated, for the first time, with the incidence of dose-dependent congestive heart failure or related disorders. Specifically, the variable single nucleotide polymorphism(s) of the human Adiponectin gene described herein have been demonstrated to statistically increase an individuals susceptibility to developing dose-dependent congestive heart failure or a congestive heart failure-like event upon the administration of a PPAR-agonist.

The human Adiponectin gene was chosen as a candidate gene to investigate the potential of it comprising one or more single nucleotide polymorphisms associated with dose-dependent congestive heart failure or related-congestive heart failure phenotype, and in particular, the potential of identifying a Adiponectin SNP associated with the incidence of dose-dependent congestive heart failure or related-congestive heart failure phenotype upon the administration of either the prescribed dose, or an increased dose of a PPAR-agonist, based upon the role of Adiponectin in regulating sodium and water re-absorption by the kidney.

The invention further provides allele-specific oligonucleotides that hybridize to the human Adiponectin gene, or fragments or complements thereof, comprising one or more single nucleotide polymorphisms and/or polymorphic locus. Such oligonucleotides are expected to hybridize to one polymorphic allele of the nucleic acid molecules described herein but not to the other polymorphic allele(s) of the sequence. Thus, such oligonucleotides can be used to determine the presence or absence of particular alleles of the polymorphic sequences described herein and to distinguish between reference and variant allele for each form. These oligonucleotides can be probes or primers.

The invention further provides a method of analyzing a nucleic acid from an individual to identify the presence or absence of a particular nucleotide at a given polymorphic locus and to distinguish between the reference and variant allele at each locus. The method determines which base is present at any one of the polymorphic loci shown in FIGS. 7A-D, and/or FIGS. 8A-D (SEQ ID NOs:26, and/or 28), or elsewhere herein. Optionally, a set of bases occupying a set of the polymorphic loci shown in FIGS. 7A-D, and/or FIGS. 8A-D (SEQ ID NOs:26, and/or 28) is determined. This type of analysis can be performed on a number of individuals, who are also tested (previously, concurrently or subsequently) for the presence of dose-dependent congestive heart failure or congestive heart failure-like phenotype in the presence or absence of a PPAR-agonist. The presence or absence of dose-dependent congestive heart failure or congestive heart failure-like phenotype is then correlated with a base or set of bases present at the polymorphic locus or loci in the patient and/or sample tested.

Thus, the invention further relates to a method of predicting the presence, absence, likelihood of the presence or absence, or severity of a particular dose-dependent congestive heart failure or congestive heart failure-like phenotype associated with a particular genotype in the presence or absence of either the prescribed dose, or an increased dose of a PPAR-agonist. The method comprises obtaining a nucleic acid sample from an individual and determining the identity of one or more bases (nucleotides) at on or more polymorphic loci of the nucleic acid molecules described herein, wherein the presence of a particular base is correlated with the incidence of dose-dependent congestive heart failure or congestive heart failure-like phenotype or an increased risk of developing dose-dependent congestive heart failure or congestive heart failure-like phenotype in the presence of a PPAR-agonist, thereby predicting the presence, absence, likelihood of the presence or absence, or severity of dose-dependent congestive heart failure or congestive heart failure-like in the individual or sample. The correlation between a particular polymorphic form of a gene and a phenotype can thus be used in methods of diagnosis of that phenotype, as well as in the development of treatments for the phenotype.

### Definitions

An “oligonucleotide” can be DNA or RNA, and single- or double-stranded. An oligonucleotide may be used as either a “primer” or a “probe”. Oligonucleotides can be naturally occurring or synthetic, but are typically prepared by synthetic means. An oligonucleotide primer, for example, may be designed to hybridize to the complementary sequence of either the sense or antisense strand of a specific target sequence, and may be used alone or as a pair, such as in DNA amplification reactions, and may or may not comprise one or more polymorphic loci of the present invention. An oligonucleotide probe may also be designed to hybridize to the complementary sequence of either the sense or antisense strand of a specific target sequence, and may be used alone or as a pair, such as in DNA amplification reactions, but necessarily will comprise one or more polymorphic loci of the present invention. Preferred oligonucleotides of the invention include fragments of DNA, or their complements thereof, of the human mineralocorticoid receptor, PPAR-gamma, or Adiponectin gene, and may comprise one or more of the polymorphic loci shown or described in FIGS. 1A-F, FIGS. 2A-F, FIGS. 4A-B, FIGS. 5A-B, FIGS. 7A-D, and/or FIGS. 8A-D or as described elsewhere herein. The fragments can be between 10 and 250 bases, and, in specific embodiments, are between about 5 to about 10, about 5 to about 15, about 10 to about 20, about 15 to about 25, about 10 to about 30, about 10 to about 50, or about 10 to about 100 bases in length. For example, the fragment can be 40 bases in length. The polymorphic locus can occur within any nucleotide position of the fragment, including at either terminus or directly in the middle, for example. The fragments can be from any of the allelic forms of DNA shown or described herein.

As used herein, the terms “nucleotide”, “base” and “nucleic acid” are intended to be equivalent. The terms “nucleotide sequence”, “nucleic acid sequence”, “nucleic acid molecule” and “segment” are intended to be equivalent.

Hybridization probes are oligonucleotides which bind in a base-specific manner to a complementary strand of nucleic acid and are designed to identify the allele at one or more polymorphic loci within the mineralocorticoid receptor, PPAR-gamma, or Adiponectin gene of the present invention. Such probes include peptide nucleic acids, as described in Nielsen et al., Science 254, 1497-1500 (1991). Probes can be any length suitable for specific hybridization to the target nucleic acid sequence. The most appropriate length of the probe may vary depending upon the hybridization method in which it is being used; for example, particular lengths may be more appropriate for use in microfabricated arrays, while other lengths may be more suitable for use in classical hybridization methods. Such optimizations are known to the skilled artisan. Suitable probes can range from about 12 nucleotides to about 25 nucleotides in length. For example, probes and primers can be about 4, 6, 8, 10, 12, 14, 16, 18, 20, 22, 24, 25, 26, or about 40 nucleotides in length. The probe preferably comprises at least one polymorphic locus occupied by any of the possible variant nucleotides. For comparison purposes, the present invention also encompasses probes that comprise the reference nucleotide at at least one polymorphic locus. The nucleotide sequence can correspond to the coding sequence of the allele or to the complement of the coding sequence of the allele, where applicable.

As used herein, the term “primer” refers to a single-stranded oligonucleotide which acts as a point of initiation of template-directed DNA synthesis under appropriate conditions. Such DNA synthesis reactions may be carried out in the traditional method of including all four different nucleoside triphosphates (e.g., in the form of phosphoramidates, for example) corresponding to adenine, guanine, cytosine and thymine or uracil nucleotides, and an agent for polymerization, such as DNA or RNA polymerase or reverse transcriptase in an appropriate buffer and at a suitable temperature. Alternatively, such a DNA synthesis reaction may utilize only a single nucleoside (e.g., for single base-pair extension assays). The appropriate length of a primer depends on the intended use of the primer, but typically ranges from about 10 to about 30 nucleotides. Short primer molecules generally require cooler temperatures to form sufficiently stable hybrid complexes with the template. A primer need not reflect the exact sequence of the template, but must be sufficiently complementary to hybridize with a template. The term “primer site” refers to the area of the target DNA to which a primer hybridizes. The term primer pair refers to a set of primers including a 5′ (upstream) primer that hybridizes with the 5′ end of the DNA sequence to be amplified and a 3′ (downstream) primer that hybridizes with the complement of the 3′ end of the sequence to be amplified.

As used herein, “linkage” describes the tendency of genes, alleles, loci or genetic markers to be inherited together as a result of their location on the same chromosome. It can be measured by percent recombination between the two genes, alleles, loci or genetic markers.

As used herein, “polymorphism” refers to the occurrence of two or more genetically determined alternative sequences or alleles in a population. A “polymorphic locus” is a marker or site at which divergence from a reference allele occurs. The phrase “polymorphic loci” is meant to refer to two or more markers or sites at which divergence from two or more reference alleles occurs. Preferred markers have at least two alleles, each occurring at frequency of greater than 1%, and more preferably greater than 10% or 20% of a selected population. A polymorphic locus may be as small as one base pair. Polymorphic loci include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the “reference form” or “reference allele” and other allelic forms are designated as alternative forms or “variant alleles”. The allelic form occurring most frequently in a selected population is sometimes referred to as the wild type form. Diploid organisms may be homozygous or heterozygous for allelic forms. A diallelic or biallelic polymorphism has two forms. A triallelic polymorphism has three forms.

As used herein, the term “genotype” is meant to encompass the particular allele present at a polymorphic locus of a DNA sample, a gene, and/or chromosome.

As used herein, the term “haplotype” is meant to encompass the combination of genotypes across two or more polymorphic loci of a DNA sample, a gene, and/or chromosome, wherein the genotypes are closely linked, may be inherited together as a unit, and may be in linkage disequilibrium relative to other haplotypes and/or genotypes of other DNA samples, genes, and/or chromosomes.

As used herein, the term “linkage disequilibrium” refers to a measure of the degree of association between two alleles in a population. For example, when alleles at two distinctive loci occur in a sample more frequently than expected given the known allele frequencies and recombination fraction between the two loci, the two alleles may be described as being in “linkage disequilibrium”.

As used herein, the terms “genotype assay” and “genotype determination”, and the phrase “to genotype” or the verb usage of the term “genotype” are intended to be equivalent and refer to assays designed to identify the allele or alleles at a particular polymorphic locus or loci in a DNA sample, a gene, and/or chromosome. Such assays may employ single base extension reactions, DNA amplification reactions that amplify across one or more polymorphic loci, or may be as simple as sequencing across one or more polymorphic loci. A number of methods are known in the art for genotyping, with many of these assays being described herein or refered to herein.

Work described herein pertains to the resequencing of the human mineralocorticoid receptor, PPAR-gamma, or Adiponectin gene in a large number of individuals to identify polymorphisms associated with the incidence of dose-dependent congestive heart failure or congestive heart failure-like disorders upon the administration of a PPAR-agonist, which may predispose individuals to developing such a disorder. For example, polymorphisms in the mineralocorticoid receptor, PPAR-gamma, or Adiponectin gene described herein are associated with the incidence of dose-dependent congestive heart failure or congestive heart failure-like disorders and are useful for predicting the likelihood that an individual will be susceptible to such a disorder, or that such an individual may have an increased susceptibility to such a disorder, upon the administration of a PPAR-agonist.

By altering amino acid sequence, SNPs may alter the function of the encoded proteins. The discovery of the SNP facilitates biochemical analysis of the variants and the development of assays to characterize the variants and to screen for pharmaceutical compounds that would interact directly with one or another form of the protein. SNPs (including silent SNPs) may also alter the regulation of the gene at the transcriptional or post-transcriptional level. SNPs (including silent SNPs) also enable the development of specific DNA, RNA, or protein-based diagnostics that detect the presence or absence of the polymorphism in particular conditions.

The phrase “PPAR-agonist” is meant to encompass compounds, including small molecules, antibodies, RNAi reagents, siRNA reagents, antisense compounds, or any compound in general capable of increasing the activity or expression of one or more peroxisome proliferator activator receptors (PPAR), including but not limited to, PPAR-alpha agonists, PPAR-beta agonists, PPAR-gamma agonists, and PPAR-delta agonists, including mono-PPAR-alpha agonists, mono-PPAR-beta agonists, mono-PPAR-gamma agonists, mono-PPAR-delta agonists, dual PPAR-alpha and gamma agonists, and any combination of the same. In addition, such PPAR-agonists are necessarily meant to encompass the following, non-limiting compounds: Muraglitazar, peliglitazar, Farglitazar, thiazolidinediones class of PPAR-agonists, Troglitazone, Pioglitazone, Rosiglitazone, MCC555, KRP297, JTT-501, BM 17.0744, L764486, GW501516, NN622, bezafibrate, gemfibrozil, fibrate class of PPAR-agonists, DRF 2725, WY 14,643, SB 213068, Tesaglitazar (AZ 242), Avandaryl, Naveglitazar, Ragaglitazar (NN622), PLX 204, PLX 134, PLX 203, CS 7017, DRF 10945, AVE 0847, AVE 8134, 641597 (GSK), 590735 (GSK), MK 767, AA 10090, LY 674, LY 929, T 131, DRF 4158, CLX 0921, NS 220, LY 293111, DRF 4832, GW 7282, 501516 (GSK), LG 100754, GW 544, AR H049020, AK-109, E-3030 (Eisai), CS-7017 (Sankyo), DRF-10945, KRP-101, ONO-5129, TY-51501, GSK-677954, LSN-862, LY-518674, GW-590735, KT6-207, K-111 (Roche), Bay-54-9801 (GSK), R-483 (Roche), EMD-336340 (Merck KGaA), LR-90 (Merck KGaA), CLX-0940, CLX-0921, LG-100754, GW-409890, SB-219994, NIP-223, T-174 (Tanabe Seiyaku), balaglitazone (DRF-2593), VDO-52, GW-1929, NC-2 100, netoglitazone, ciglitazone, LGD 1268, LG 101506, LGD 1324, GW 9578, Englitazone, and/or Darglitazone.

A single nucleotide polymorphism occurs at a polymorphic locus occupied by a single nucleotide, which is the site of variation between allelic sequences. The site is usually preceded by and followed by highly conserved sequences of the allele (e.g., sequences that vary in less than 1/100 or 1/1000 members of the populations).

A single nucleotide polymorphism usually arises due to substitution of one nucleotide for another at the polymorphic locus. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. Typically the polymorphic locus is occupied by a base other than the reference base. For example, where the reference allele contains the base “g” at the polymorphic site, the altered allele can contain a “c”, “t” or “a” at the polymorphic locus. In the instant case, the variable allele contains the base “t” at the polymorphic site.

For the purposes of the present invention the terms “polymorphic position”, “polymorphic site”, “polymorphic locus”, and “polymorphic allele” shall be construed to be equivalent and are defmed as the location of a sequence identified as having more than one nucleotide represented at that location in a population comprising at least one or more individuals, and/or chromosomes.

Probe hybridizations are usually performed under stringent conditions, for example, at a salt concentration of no more than 1 M and a temperature of at least 25° C. For example, conditions of 5×SSPE (750 mM NaCl, mM NaPhosphate, mM EDT A, pH 7.4) and a temperature of 25-30° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

Wherever the terms “compound A”, “Com. A” are used herein, it is understood (unless otherwise indicated) that the compound ‘N-[(4-methoxyphenoxy)carbonyl]-N-[[4-[2-(5-methyl-2-phenyl-4-oxazolyl)ethoxy]phenyl]methyl]glycine having the following structure (I): 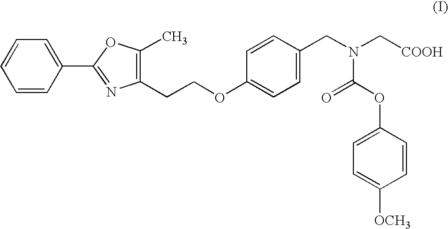
is intended (also referred hereinafter as “((4-methoxy-phenoxycarbonyl)-{4-[2-(5-methyl-2-phenyl-oxazol-4-yl)-ethoxy]-benzyl}-amino)-acetic acid)”, “muraglitazar”, Com. A or Pargluva®), as well as all pharmaceutically acceptable salts thereof Use of the term encompasses (unless otherwise indicated) solvates (including hydrates), crystal structures (including polymorphic forms of such structures) and salts of the compound (I). Pharmaceutical compositions of Com. A include all pharmaceutically acceptable Compositions comprising Com. A and one or more diluents, vehicles and/or excipients, such as those compositions described in U.S. Pat. No. 6,414,002 (described in Example 230) and U.S. Ser. No. 11/130048, filed May 16, 2005, incorporated herein by reference.

The term “isolated” is used herein to indicate that the material in question exists in a physical milieu distinct from that in which it occurs in nature, and thus is altered “by the hand of man” from its natural state. For the avoidance of doubt, the present invention is meant to encompass those compositions where the art demonstrates no distinguishing features of the polynucleotide/sequences of the present invention (e.g., the knowledge that a particular nucleotide position represents a polymorphic site, the knowledge of which allele represents the reference and/or variant nucleotide base, the association of a particular polymorphism with a disease or disorder, wherein such association was not appreciated heretofore, etc.).

On one hand, and in specific embodiments, the polynucleotides of the invention are at least 15, at least 30, at least 50, at least 100, at least 125, at least 500, or at least 1000 continuous nucleotides but are less than or equal to 300 kb, 200 kb, 100 kb, 50 kb, 15 kb, 10 kb, 7.5 kb, 5 kb, 2.5 kb, 2.0 kb, or 1 kb, in length. In a further embodiment, polynucleotides of the invention comprise a portion of the coding sequences, as disclosed herein, and may comprise all or a portion of an intron. In another embodiment, the polynucleotides preferentially do not contain the genomic sequence of the gene or genes flanking the human mineralocorticoid receptor, PPAR-gamma, or Adiponectin (i.e., 5′ or 3′ to the mineralocorticoid receptor, PPAR-gamma, or Adiponectin gene in the genome). In other embodiments, the polynucleotides of the invention do not contain the coding sequence of more than 1000, 500, 250, 100, 50, 25, 20, 15, 10, 5, 4, 3, 2, or 1 genomic flanking gene(s).

On the other hand, and in specific embodiments, the polynucleotides of the invention are at least 15, at least 30, at least 50, at least 100, at least 125, at least 500, or at least 1000 continuous nucleotides but are less than or equal to 300 kb, 200 kb, 100 kb, 50 kb, 15 kb, 10 kb, 7.5 kb, 5 kb, 2.5 kb, 2.0 kb, or 1 kb, in length. In a further embodiment, polynucleotides of the invention comprise a portion of the coding sequences, comprise a portion of non-coding sequences, comprise a portion of an intron sequence, etc., or any combination of the latter, as disclosed herein. Alternatively, the polynucleotides of the invention may comprise the entire coding sequence, the entire 5′ non-coding sequence, the entire 3′ non-coding sequence, an entire intron sequence, an entire exon sequence, or any combination of the latter, as disclosed herein. In another embodiment, the polynucleotides may correspond to a genomic sequence flanking a gene (i.e., 5′ or 3′ to the gene of interest in the genome). In other embodiments, the polynucleotides of the invention may contain the non-coding sequence of more than 1000, 500, 250, 100, 50, 25, 20, 15, 10, 5, 4, 3, 2, or 1 genomic flanking gene(s).

As used herein, a “polynucleotide” refers to a molecule comprising a nucleic acid of SEQ ID NO:1, 3, 13, 15, 26, and/or 28. For example, the polynucleotide can contain the nucleotide sequence of the full length cDNA sequence, including the 5′ and 3′ untranslated sequences, the coding region, with or without a signal sequence, the secreted protein coding region, and the genomic sequence with or without the accompanying promoter and transcriptional termination sequences, as well as fragments, epitopes, domains, and variants of the nucleic acid sequence. Moreover, as used herein, a “polypeptide” refers to a molecule having the translated amino acid sequence generated from the polynucleotide as defined.

Unless otherwise indicated, all nucleotide sequences determined by sequencing a DNA molecule herein were determined using an automated DNA sequencer (such as the Model 3730-XL from Applied Biosystems, Inc., and/or ther PE 9700 from Perkin Elmer), and all amino acid sequences of polypeptides encoded by DNA molecules determined herein were predicted by translation of a DNA sequence determined above. The nucleotide sequence can also be determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion. Since the present relates to the identification of single nucleotide polymorphisms whereby the novel sequence differes by as few as a single nucleotide from a reference sequence, identified SNPs were multiply verified to ensure each novel sequence represented a true SNP.

Using the information provided herein, a nucleic acid molecule of the present invention encoding a polypeptide of the present invention may be obtained using standard cloning and screening procedures, such as those for cloning cDNAs using mRNA as starting material.

A “polynucleotide” of the present invention also includes those polynucleotides capable of hybridizing, under stringent hybridization conditions, to sequences described herein, or the complement thereof. “Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65 degree C.

The polynucleotide of the present invention can be composed of any polyribonucleotide or polydeoxribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotide can be composed of triple-stranded regions comprising RNA or DNA or both RNA and DNA. A polynucleotide may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The polypeptide of the present invention can be composed of amino acids joined to each other by peptide bonds or modified peptide bonds, i.e., peptide isosteres, and may contain amino acids other than the gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

The term “organism” as referred to herein is meant to encompass any organism referenced herein, though preferably to eukaryotic organisms, more preferably to mammals, and most preferably to humans.

As used herein the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein.

### Polynucleotides and Polypeptides of the Invention

### Features of Gene No:1

The present invention relates to isolated nucleic acid molecules comprising, or alternatively consisting of, all or a portion of one or more alleles of SNP1 of the human mineralocorticoid receptor gene, as provided in FIGS. 1A-F (SEQ ID NO:1) comprising at least one polymorphic locus. The allele described for SNP1 in FIGS. 1A-F (SEQ ID NO:1) represents the reference allele for this SNP and is exemplified by an “a” at nucleotide position 754. Fragments of this polynucleotide are at least about 10, at least about 20, at least about 40, or at least about 100, contiguous nucleotides and comprise one or more reference alleles at the nucleotide position(s) provided in FIGS. 1A-F (SEQ ID NO:1).

The present invention further relates to isolated proteins or polypeptides comprising, or alternatively, consisting of all or a portion of the encoded variant amino acid sequence of human mineralocorticoid receptor (e.g., wherein reference to wildtype or reference mineralocorticoid receptor polypeptide is exemplified by SEQ ID NO:2). Preferred portions are at least 10, preferably at least 20, preferably at least 40, preferably at least 100, contiguous polypeptides and comprises an “Ile” at the amino acid position corresponding to amino acid 180 of the mineralocorticoid receptor polypeptide, or a portion of SEQ ID NO:2. The invention further relates to isolated nucleic acid molecules encoding such polypeptides or proteins, as well as to antibodies that bind to such proteins or polypeptides.

In one embodiment, the invention relates to a method for predicting the likelihood that an individual will have a disorder, particularly dose-dependent congestive heart failure or a congestive heart failure-like disorder, or be susceptible to developing dose-dependent congestive heart failure or a congestive heart failure-like disorder upon the administration of a pharmaceutically acceptable amount of a PPAR-agonist, comprising the step of identifying the nucleotide present at nucleotide position 754 of SEQ ID NO:1, from a DNA sample to be assessed, or the corresponding nucleotide at this position if only a fragment of the sequence provided as SEQ ID NO:1 is assessed. The presence of the reference allele at said position indicates that the individual from whom said DNA sample or fragment was obtained has a decreased likelihood of developing dose-dependent congestive heart failure or a congestive heart failure-like disorder upon the administration of a pharmaceutically acceptable amount of a PPAR-agonist, compared to an individual having the alternate (variant) allele(s) at said position(s); or at least a decreased likelihood of developing more severe congestive heart failure symptoms upon administration of the same.

Importantly, the presence of the reference allele at said position in a nucleic acid sample provided by an individual, indicates that said individual may be administered a correspondingly higher amount of a PPAR-agonist without increasing the likelihood of developing dose-dependent congestive heart failure or a congestive heart failure-like disorder relative to another individual having the variable allele(s) at said position. Therefore, such individuals may have the level of administered PPAR-agonist “titrated-up” or maintained in a safe manner.

Representative disorders which may be detected, diagnosed, identified, treated, prevented, and/or ameliorated by the SNPs and methods of the present invention include, the following, non-limiting diseases and disorders: dose-dependent congestive heart failure, congestive heart failure, susceptibility to developing dose-dependent congestive heart failure or a congestive heart failure-like disorder upon the administration of a pharmaceutically acceptable amount of a PPAR-agonist, susceptibility to developing dose-dependent congestive heart failure or a congestive heart failure-like disorder upon the administration of an increased level of a PPAR-agonist, adverse reactions associated with PPAR-agonist, disorders associated with aberrant mineralocorticoid receptor expression, disorders associated with aberrant mineralocorticoid receptor regulation, disorders associated with aberrant mineralocorticoid receptor activity, disorders associated with aberrant regulation of mineralocorticoid receptor by aldosterone, disorders associated with aberrant angiotensin II peptide levels, disorders associated with aberrant adrenal and renal vascular responses to angiotensin II, disorders associated with refractory responses to angiotensin II antagonists, hypertension, high blood pressure, hypotension, low-mineralocorticoid receptor essential hypertension, high-mineralocorticoid receptor essential hypertension, atherosclerosis, congestive heart failure, pulmonary congestive heart failure, beta blocker induced congestive heart failure, and beta-1 blocker induced congestive heart failure.

Additional disorders which may be detected, diagnosed, identified, treated, prevented, and/or ameliorated by the SNPs and methods of the present invention include, the following, non-limiting diseases and disorders: diabetes, especially Type 2 diabetes, and related diseases such as insulin resistance, hyperglycemia, hyperinsulinemia, elevated blood levels of fatty acids or glycerol, hyperlipidemia, obesity, hypertriglyceridemia, inflammation, Syndrome X, diabetic complications, dysmetabolic syndrome, and related diseases.

In one embodiment, the invention relates to a method for predicting the likelihood that an individual will have a disorder, particularly dose-dependent congestive heart failure or a congestive heart failure-like disorder, or be susceptible to developing dose-dependent congestive heart failure or a congestive heart failure-like disorder upon the administration of a pharmaceutically acceptable amount of a PPAR-agonist, comprising the step of identifying the amino acid present at amino acid position 180 of SEQ ID NO:2, from a sample to be assessed, or the corresponding amino acid at this position if only a fragment of the sequence provided as SEQ ID NO:2 is assessed. The presence of the reference allele (e.g., “Ile”) at said position indicates that the individual from whom said sample or fragment was obtained has a decreased likelihood of developing dose-dependent congestive heart failure or a congestive heart failure-like disorder upon the administration of a pharmaceutically acceptable amount of a PPAR-agonist, compared to an individual having the alternate (variant) allele(s) at said position(s); or at least a decreased likelihood of developing more severe congestive heart failure symptoms upon administration of the same.

Importantly, the presence of the reference allele at said amino acid position in a sample provided by an individual indicates that said individual may be administered a correspondingly higher amount of a PPAR-agonist without increasing the likelihood of developing dose-dependent congestive heart failure or a congestive heart failure-like disorder relative to another individual having the variable allele(s) at said position. Therefore, such individuals may have the level of administered PPAR-agonist “titrated-up” or maintained in a safe manner.

In preferred embodiments, the following N-terminal MLR deletion polypeptides are encompassed by the present invention: M1-K984, E2-K984, T3-K984, K4-K984, G5-K984, Y6-K984, H7-K984, S8-K984, L9-K984, P10-K984, E11-K984, G12-K984, L13-K984, D14-K984, M15-K984, E16-K984, R17-K984, R18-K984, W19-K984, G20-K984, Q21-K984, V22-K984, S23-K984, Q24-K984, A25-K984, V26-K984, E27-K984, R28-K984, S29-K984, S30-K984, L31 -K984, G32-K984, P33-K984, T34-K984, E35-K984, R36-K984, T37-K984, D38-K984, E39-K984, N40-K984, N41-K984, Y42-K984, M43-K984, E44-K984, I45-K984, V46-K984, N47-K984, V48-K984, S49-K984, C50-K984, V51 -K984, S52-K984, G53-K984, A54-K984, I55-K984, P56-K984, N57-K984, N58-K984, S59-K984, T60-K984, Q61-K984, G62-K984, S63-K984, S64-K984, K65-K984, E66-K984, K67-K984, Q68-K984, E69-K984, L70-K984, L71 -K984, P72-K984, C73-K984, L74-K984, Q75-K984, Q76-K984, D77-K984, N78-K984, N79-K984, R80-K984, P81-K984, G82-K984, I83-K984, L84-K984, T85-K984, S86-K984, D87-K984, I88-K984, K89-K984, T90-K984, E91-K984, L92-K984, E93-K984, S94-K984, K95-K984, E96-K984, L97-K984, S98-K984, A99-K984, T100-K984, V101-K984, A102-K984, E103-K984, S104-K984, M105-K984, G106-K984, L107-K984, Y108-K984, M109-K984, D110-K984, S111-K984, V112-K984, R113-K984, D114-K984, A115-K984, D116-K984, Y117-K984, S118-K984, Y119-K984, E120-K984, Q121-K984, Q122-K984, N123-K984, Q124-K984, Q125-K984, G126-K984, S127-K984, M128-K984, S129-K984, P130-K984, A131-K984, K132-K984, I133-K984, Y134-K984, Q135-K984, N136-K984, V137-K984, E138-K984, Q139-K984, L140-K984, V141-K984, K142-K984, F143-K984, Y144-K984, K145-K984, G146-K984, N147-K984, G148-K984, H149-K984, R150-K984, P151-K984, S152-K984, T153-K984, L154-K984, S155-K984, C156-K984, V157-K984, N158-K984, T159-K984, P160-K984, L161-K984, R162-K984, S163-K984, F164-K984, M165-K984, S166-K984, D167-K984, S168-K984, G169-K984, S170-K984, S171-K984, V172-K984, N173-K984, G174-K984, G175-K984, V176-K984, M177-K984, R178-K984, A179-K984, and/or I180-K984 of SEQ ID NO:2. Polynucleotide sequences encoding these polypeptides are also provided. The present invention also encompasses the use of these N-terminal MLR deletion polypeptides as immunogenic and/or antigenic epitopes as described elsewhere herein.

In preferred embodiments, the following C-terminal MLR deletion polypeptides are encompassed by the present invention: M1-K984, M1-R983, M1-H982, M1-F981, M1-Y980, M1-L979, M1-P978, M1-K977, M1-A976, M1-N975, M1-G974, M1-S973, M1-E972, M1-V971, M1-K970, M1-P969, M1-L968, M1-Q967, M1-D966, M1-S965, M1-I964, M1-I963, M1-E962, M1-V961, M1-L960, M1-M959, M1-A958, M1-P957, M1-F956, M1-E955, M1-V954, M1-K953, M1-L952, M1-A951, M1-H950, M1-S949, M1-E948, M1-R947, M1-F946, M1-T945, M1-Y944, M1-F943, M1-C942, M1-F941, M1-E940, M1-L939, M1-L938, M1-D937, M1-S936, M1-V935, M1-L934, M1-D933, M1-H932, M1-M931, M1-S930, M1-D929, M1-L928, M1-L927, M1-K926, M1-T925, M1-L924, M1-Q923, M1-Y922, M1-F921, M1-R920, M1-Q919, M1-W918, M1-S917, M1-Q916, M1-G915, M1-S914, M1-N913, M1-N912, M1-P911, M1-C910, M1-K909, M1-T908, M1-V907, M1-M906, M1-K905, M1-R904, M1-L903, M1-E902, M1-K901, M1-I900, M1-Y899, M1-N898, M1-T897, M1-R896, M1-M895, M1-E894, M1-E893, M1-F892, M1-A891, M1-A890, M1-Q889, M1-S888, M1-K887, M1-L886, M1-G885, M1-D884,M1-K883, M1-P882, M1-I881, M1-T880, M1-S879, M1-L878, M1-L877, M1-L876, M1-L875, M1-V874, M1-K873, M1-M872, M1-I871, M1-T870, M1-Y869, M1-E868, M1-E867, M1-F866, M1-T865, M1-L864, M1-Q863, M1-L862, M1-R861, M1-V860, M1-F859, M1-Q858, M1-L857, M1-S856, M1-I855, M1-Q854, M1-H853, M1-M852, M1-G851, M1-Q850, M1-C849, M1-L848, M1-E847, M1-Y846, M1-M845, M1-A844, M1-S843, M1-Q842, M1-H841, M1-M840, M1-K839, M1-E838, M1-E837, M1-N836, M1-F835, M1-V834, M1-L833, M1-D832, M1-P831, M1-A830, M1-F829, M1-Y828, M1-L827, M1-F826, M1-Q825, M1-S824, M1-N823, M1-T822, M1-H821, M1-K820, M1-Y819, M1-S818, M1-R817, M1-W816, M1-S815, M1-L814, M1-A813, M1-F812, M1-S811, M1-S810, M1-L809, M1-C808, M1-M807, M1-W806, M1-S805, M1-Y804, M1-Q803, M1-I802, M1-L801, M1-T800, M1-I799, M1-Q798, M1-D797, M1-E796, M1-L795, M1-P794, M1-L793, M1-N792, M1-K791, M1-F790, M1-G789, M1-P788, M1-L787, M1-V786, M1-K785, M1-A784, M1-W783, M1-K782, M1-V781, M1-V780, M1-Q779, M1-I778, M1-M777, M1-Q776, M1-K775, M1-G774, M1-A773, M1-L772, M1-R771, M1-N770, M1-L769, M1-T768, M1-S767, M1-L766, M1-L765, M1-N764, M1-E763, M1-A762, M1-T761, M1-D760, M1-P759, M1-K758, M1-S757, M1-S756, M1-D755, M1-Y754, M1-G753, M1-A752, M1-Y751, M1-V750, M1-I749, M1-E748, M1-P747, M1-E746, M1-I745, M1-N744, M1-E743, M1-L742, M1-V741, M1-M740, M1-V739, M1-P738, M1-S737, M1-P736, M1-T735, M1-L734, M1-A733, M1-R732, M1-S731, M1-I730, M1-T729, M1-S728, M1-L727, M1-Q726, M1-P725, M1-V724, M1-L723, M1-A722, M1-T721, M1-N720, M1-V719, M1-S718, M1-P717, M1-E716, M1-K715, M1-A714, M1-P713, M1-A712, M1-I711, M1-Y710, M1-T709, M1-T708, M1-G707, M1-E706, M1-E705, M1-P704, M1-S703, M1-Q702, M1-P701, M1-P700, M1-P699, M1-P698, M1-P697, M1-P696, M1-P695, M1-P694, M1-Q693, M1-Q692, M1-Q691, M1-Q690, M1-P689, M1-Q688, M1-E687, M1-E686, M1-H685, M1-I684, M1-G683, M1-K682, M1-L681, M1-K680, M1-G679, M1-L678, M1-K677, M1-K676, M1-S675, M1-K674, M1-R673, M1-A672, M1-G671, M1-L670, M1-N669, M1-M668, M1-G667, M1-A666, M1-Q665, M1-L664, M1-C663, M1-K662, M1-Q661, M1-L660, M1-R659, M1-C658, M1-A657, M1-P656, M1-C655, M1-N654, M1-K653, M1-R652, M1-R651, M1-I650, M1-K649, M1-D648, M1-I647, M1-I646, M1-C645, M1-D644, M1-N643, M1-R642, M1-G641, M1-A640, M1-C639, M1-L638, M1-Y637, M1-N636, M1-H635, M1-Q634, M1-G633, M1-E632, M1-V631, M1-A630, M1-R629, M1-K628, M1-F627, M1-F626, M1-V625, M1-K624, M1-C623, M1-S622, M1-G621, M1-C620, M1-T619, M1-V618, M1-V617, M1-G616, M1-Y615, M1-H614, M1-C613, M1-G612, M1-S611, M1-A610, M1-E609, M1-D608, M1-G607, M1-C606, M1-V605, M1-L604, M1 -C603, M1-I602, M1-K601, M1-S600, M1-P599, M1-R598, M1-S597, M1-S596, M1-G595, M1-T594, M1-S593, M1-V592, M1-S591, M1-R590, M1-L589, M1-T588, M1-S587, M1-S586, M1-S585, M1-V584, M1-N583, M1-E582, M1-P581, M1-I580, M1-Y579, M1-E578, M1-L577, M1-V576, M1-P575, M1-Y574, M1-G573, M1-D572, M1-S571, M1-R570, M1-R569, M1-S568, M1-S567, M1-L566, M1-D565, M1-G564, M1-H563, M1-S562, M1-K561, M1-W560, M1-S559, M1-E558, M1-V557, M1-L556, M1-T555, M1-N554, M1-V553, M1-P552, M1-P551, M1-F550, M1-S549, M1-S548, M1-L547, M1-H546, M1-Q545, M1-F544, M1-S543, M1-Q542, M1-D541, M1-R540, M1-A539, M1-S538, M1-R537, M1-S536, M1-L535, M1-S534, M1-I533, M1-T532, M1-G531, M1-Q530, M1-A529, M1-G528, M1-I527, M1-R526, M1-Y525, M1-H524, M1-F523, M1-S522, M1-Q521, M1-G520, M1-G519, M1-S518, M1-N517, M1-V516, M1-G515, M1-V514, M1-I513, M1-A512, M1-S511, M1-S510, M1-P509, M1-I508, M1-S507, M1-A506, M1-E505, M1-P504, M1-Y503, M1-Y502, M1-S501, M1-G500, M1-D499, M1-D498, M1-P497, M1-E496, M1-Q495, M1-K494, M1-I493, M1-G492, M1-V491, M1-P490, M1-F489, M1-G488, M1-S487, M1-G486, M1-E485, M1-C484, M1-N483, M1-G482, M1-D481, M1-F480, M1-G479, M1-P478, M1-V477, M1-P476, M1-P475, M1-G474, M1-L473, M1-I472, M1-G471, M1-S470, M1-L469, M1-S468, M1-Y467, M1-Y466, M1-D465, M1-K464, M1-D463, M1-D462, M1-M461, M1-F460, M1-S459, M1-F458, M1-Y457, M1-S456, M1-G455, M1-D454, M1-M453, M1-F452, M1-P451, M1-F450, M1-P449, M1-N448, M1-V447, M1-T446, M1-P445, M1-N444, M1-G443, M1-K442, M1-F441, M1-S440, M1-T439, M1-G438, M1-S437, M1-C436, M1-S435, M1-H434, M1-K433, M1-T432, M1-S431, M1-E430, M1-Q429, M1-K428, M1-I427, M1-P426, M1-V425, M1-S424, M1-F423, M1-S422, M1-S421, M1-D420, M1-S419, M1-N418, M1-I417, M1-K416, M1-S415, M1-N414, M1-G413, M1-G412, M1-L411, M1-C410, M1-S409, M1-S408, M1-S407, M1-F406, M1-A405, M1-G404, M1-D403, M1-P402, M1-E401, M1-P400, M1-K399, M1-I398, M1-Y397, M1-Q396, M1-V395, M1-I394, M1-N393, M1-L392, M1-Q391, M1-G390, M1-T389, M1-V388, M1-G387, M1-N386, M1-S385, M1-I384, M1-A383, M1-S382, M1-E381, M1-V380, M1-E379, M1-E378, M1-T377, M1-K376, M1-P375, M1-F374, M1-P373, M1-V372, M1-E371, M1-Q370, M1-A369, M1-G368, M1-K367, M1-E366, M1-Q365, M1-T364, M1- D363, M1-P362, M1-S361, M1-P360, M1-V359, M1-V358, M1-D357, M1-R356, M1-L355, M1-T354, M1-S353, M1-S352, M1-G351, M1-A350, M1-S349, M1-T348, M1-G347, M1-S346, M1-A345, M1-T344, M1-Y343, M1-S342, M1-F341, M1-A340, M1-N339, M1-N338, M1-V337, M1-P336, M1-S335, M1-C334, M1-I333, M1-S332, M1-G331, M1-V330, M1-T329, M1-S328, M1-A327, M1-A326, M1-P325, M1-S324, M1-S323, M1-L322, M1-T321, M1-S320, M1-R319, M1-N318, M1-N317, M1-T316, M1-N315, M1-S314, M1-P313, M1-S312, M1-S311, M1-V310, M1-S309, M1-C308, M1-R307, M1-S306, M1-N305, M1-N304, M1-I303, M1-N302, M1-A301, M1-P300, M1-S299, M1-S298, M1-V297, M1-S296, M1-S295, M1-R294, M1-L293, M1-T292, M1-V291, M1-N290, M1-N289, M1-P288, M1-S287, M1-S286, M1-V285, M1-P284, M1-S283, M1-K282, M1-V281, M1-S280, M1-C279, M1-H278, M1-S277, M1-P276, M1-P275, M1-S274, M1-S273, M1-I272, M1-S271, M1-S270, M1-K269, M1-M268, M1-S267, M1-S266, M1-L265, M1-P264, M1-S263, M1-S262, M1-L261, M1-P260, M1-S259, M1-G258, M1-V257, M1-N256, M1-S255, M1-A254, M1-H253, M1-A252, M1-P251, M1-S250, M1-H249, M1-S248, M1-R247, M1-S246, M1-G245, M1-R244, M1-N243, M1-E242, M1-A241, M1-N240, M1-P239, M1-S238, M1-C237, M1-T236, M1-L235, M1-P234, M1-T233, M1-G232, M1-Q231, M1-T230, M1-I229, M1-P228, M1-S227, M1-H226, M1-V225, M1-P224, M1-F223, M1-S222, M1-G221, M1-F220, M1-S219, M1-A218, M1-T217, M1-T216, M1-S215, M1-S214, M1-V213, M1-S212, M1-N211, M1-I210, M1-G209, M1-A208, M1-P207, M1-S206, M1-C205, M1-V204, M1-S203, M1-S202, M1-T201, M1-M200, M1-N199, M1-L198, M1-P197, M1-S196, M1-C195, M1-V194, M1-S193, M1-P192, M1-S191, M1-K190, M1-E189, M1-H188, M1-C187, M1-M186, M1-I185, M1-P184, M1-S183, M1-K182, M1-V181, and/or M1-I180 of SEQ ID NO:2. Polynucleotide sequences encoding these polypeptides are also provided. The present invention also encompasses the use of these C-terminal MLR deletion polypeptides as immunogenic and/or antigenic epitopes as described elsewhere herein.

Alternatively, preferred polypeptides of the present invention may comprise polypeptide sequences corresponding to, for example, internal regions of the mineralocorticoid receptor polypeptide (e.g., any combination of both N- and C-terminal mineralocorticoid receptor polypeptide deletions) of SEQ ID NO:2. For example, internal regions could be defined by the equation: amino acid NX to amino acid CX, wherein NX refers to any N-terminal deletion polypeptide amino acid of mineralocorticoid receptor (SEQ ID NO:2), and where CX refers to any C-terminal deletion polypeptide amino acid of mineralocorticoid receptor (SEQ ID NO:2). Polynucleotides encoding these polypeptides are also provided. The present invention also encompasses the use of these polypeptides as an immunogenic and/or antigenic epitope as described elsewhere herein and are useful for creating allele-specific antibodies to discriminate between the reference and variable allele in a given sample, among other uses described herein. In addition such fragments may also be useful in designing allele-specific hybridization or other means probes to identify the allele to discriminate between the reference and variable allele in a given sample, among other uses described herein.

### Features of Gene No:2

The present invention relates to isolated nucleic acid molecules comprising, or alternatively consisting of, all or a portion of one or more alleles of SNP1 of the human mineralocorticoid receptor gene, as provided in FIGS. 2A-F (SEQ ID NO:3) comprising at least one polymorphic locus. The allele described for SNP1 in FIGS. 2A-F (SEQ ID NO:3) represents the variable allele for this SNP and is exemplified by an “g” at nucleotide position 754. Fragments of this polynucleotide are at least about 10, at least about 20, at least about 40, at least about 100, contiguous nucleotides and comprise one or more variable alleles at the nucleotide position(s) provided in FIGS. 2A-F (SEQ ID NO:3).

The present invention further relates to isolated proteins or polypeptides comprising, or alternatively, consisting of all or a portion of the encoded variant amino acid sequence of human mineralocorticoid receptor (e.g., wherein reference to variant or variable human mineralocorticoid receptor polypeptide is exemplified by SEQ ID NO:4). Preferred portions are at least 10, preferably at least 20, preferably at least 40, preferably at least 100, contiguous polypeptides and comprises a “Val” at the amino acid position corresponding to amino acid 180 of the mineralocorticoid receptor polypeptide, or a portion of SEQ ID NO:4. The invention further relates to isolated nucleic acid molecules encoding such polypeptides or proteins, as well as to antibodies that bind to such proteins or polypeptides.

In one embodiment, the invention relates to a method for predicting the likelihood that an individual will have a disorder, particularly dose-dependent congestive heart failure or a congestive heart failure-like disorder, or be susceptible to developing dose-dependent congestive heart failure or a congestive heart failure-like disorder upon the administration of a pharmaceutically acceptable amount of a PPAR-agonist, comprising the step of identifying the nucleotide present at nucleotide position 754 of SEQ ID NO:3, from a DNA sample to be assessed, or the corresponding nucleotide at this position if only a fragment of the sequence provided as SEQ ID NO:3 is assessed. The presence of the variable allele at said position indicates that the individual from whom said DNA sample or fragment was obtained has an increased likelihood of developing dose-dependent congestive heart failure or a congestive heart failure-like disorder upon the administration of a pharmaceutically acceptable amount of a PPAR-agonist than an individual having the reference allele(s) at said position(s); or an increased likelihood of developing more severe congestive heart failure symptoms upon administration of the same.

Importantly, the presence of the variable allele at said polynucleotide and/or amino acid position in a sample provided by an individual indicates that said individual should be monitored more closely if an increased dosage of a PPAR-agonist is contemplated in order to avoid the potential of increasing the likelihood of developing dose-dependent congestive heart failure or a congestive heart failure-like disorder relative to another individual having the reference allele(s) at said position. In addition, a lower dose of a PPAR-agonist should be considered.

Representative disorders which may be detected, diagnosed, identified, treated, prevented, and/or ameliorated by the SNPs and methods of the present invention include, the following, non-limiting diseases and disorders: dose-dependent congestive heart failure, congestive heart failure, susceptibility to acquiring a congestive heart failure or a congestive heart failure-like disorder upon the administration of a pharmaceutically acceptable amount of a PPAR-agonist, adverse reactions associated with PPAR-agonist, disorders associated with aberrant mineralocorticoid receptor expression, disorders associated with aberrant mineralocorticoid receptor regulation, disorders associated with aberrant mineralocorticoid receptor activity, disorders associated with aberrant regulation of mineralocorticoid receptor by aldosterone, disorders associated with aberrant angiotensin II peptide levels, disorders associated with aberrant adrenal and renal vascular responses to angiotensin II, disorders associated with refractory responses to angiotensin II antagonists, hypertension, high blood pressure, hypotension, low-mineralocorticoid receptor essential hypertension, high-mineralocorticoid receptor essential hypertension, atherosclerosis, congestive heart failure, pulmonary congestive heart failure, beta blocker induced congestive heart failure, and beta-1 blocker induced congestive heart failure.

Additional disorders which may be detected, diagnosed, identified, treated, prevented, and/or ameliorated by the SNPs and methods of the present invention include, the following, non-limiting diseases and disorders: diabetes, especially Type 2 diabetes, and related diseases such as insulin resistance, hyperglycemia, hyperinsulinemia, elevated blood levels of fatty acids or glycerol, hyperlipidemia, obesity, hypertriglyceridemia, inflammation, Syndrome X, diabetic complications, dysmetabolic syndrome, and related diseases.

In one embodiment, the invention relates to a method for predicting the likelihood that an individual will have a disorder, particularly dose-dependent congestive heart failure or a congestive heart failure-like disorder, or be susceptible to developing dose-dependent congestive heart failure or a congestive heart failure-like disorder upon the administration of a pharmaceutically acceptable amount of a PPAR-agonist, comprising the step of identifying the amino acid present at amino acid position 180 of SEQ ID NO:4, from a sample to be assessed, or the corresponding amino acid at this position if only a fragment of the sequence provided as SEQ ID NO:4 is assessed. The presence of the reference allele (e.g., “V”) at said position indicates that the individual from whom said sample or fragment was obtained has an increased likelihood of developing dose-dependent congestive heart failure or a congestive heart failure-like disorder upon the administration of a pharmaceutically acceptable amount of a PPAR-agonist, compared to an individual having the reference allele(s) at said position(s); or at least an increased likelihood of developing more severe dose-dependent congestive heart failure symptoms upon administration of the same.

Importantly, the presence of the variable allele at said amino acid position in a sample provided by an individual indicates that said individual should be monitored more closely if an increased dosage of a PPAR-agonist is contemplated in order to avoid the potential of increasing the likelihood of developing dose-dependent congestive heart failure or a congestive heart failure -like disorder relative to another individual having the reference allele(s) at said position. In addition, a lower dose of a PPAR-agonist should be considered.

In preferred embodiments, the following N-terminal MLR deletion polypeptides are encompassed by the present invention: M1-K984, E2-K984, T3-K984, K4-K984, G5-K984, Y6-K984, H7-K984, S8-K984, L9-K984, P10-K984, E11-K984, G12-K984, L13-K984, D14-K984, M15-K984, E16-K984, R17-K984, R18-K984, W19-K984, G20-K984, Q21-K984, V22-K984, S23-K984, Q24-K984, A25-K984, V26-K984, E27-K984, R28-K984, S29-K984, S30-K984, L31-K984, G32-K984, P33-K984, T34-K984, E35-K984, R36-K984, T37-K984, D38-K984, E39-K984, N40-K984, N41-K984, Y42-K984, M43-K984, E44-K984, I45-K984, V46-K984, N47-K984, V48-K984, S49-K984, C50-K984, V51-K984, S52-K984, G53-K984, A54-K984, I55-K984, P56-K984, N57-K984, N58-K984, S59-K984, T60-K984, Q61-K984, G62-K984, S63-K984, S64-K984, K65-K984, E66-K984, K67-K984, Q68-K984, E69-K984, L70-K984, L71-K984, P72-K984, C73-K984, L74-K984, Q75-K984, Q76-K984, D77-K984, N78-K984, N79-K984, R80-K984, P81-K984, G82-K984, I83-K984, L84-K984, T85-K984, S86-K984, D87-K984, I88-K984, K89-K984, T90-K984, E91-K984, L92-K984, E93-K984, S94-K984, K95-K984, E96-K984, L97-K984, S98-K984, A99-K984, T100-K984, V101-K984, A102-K984, E103-K984, S104-K984, M105-K984, G106-K984, L107-K984, Y108-K984, M109-K984, D110-K984, S111-K984, V112-K984, R113-K984, D114-K984, A115-K984, D116-K984, Y117-K984, S118-K984, Y119-K984, E120-K984, Q121-K984, Q122-K984, N123-K984, Q124-K984, Q125-K984, G126-K984, S127-K984, M128-K984, S129-K984, P130-K984, A131-K984, K132-K984, I133-K984, Y134-K984, Q135-K984, N136-K984, V137-K984, E138-K984, Q139-K984, L140-K984, V141-K984, K142-K984, F143-K984, Y144-K984, K145-K984, G146-K984, N147-K984, G148-K984, H149-K984, R150-K984, P151-K984, S152-K984, T153-K984, L154-K984, S155-K984, C156-K984, V157-K984, N158-K984, T159-K984, P160-K984, L161-K984, R162-K984, S163-K984, F164-K984, M165-K984, S166-K984, D167-K984, S168-K984, G169-K984, S170-K984, S171-K984, V172-K984, N173-K984, G174-K984, G175-K984, V176-K984, M177-K984, R178-K984, A179-K984, and/or V180-K984 of SEQ ID NO:4. Polynucleotide sequences encoding these polypeptides are also provided. The present invention also encompasses the use of these N-terminal MLR deletion polypeptides as immunogenic and/or antigenic epitopes as described elsewhere herein.

In preferred embodiments, the following C-terminal MLR deletion polypeptides are encompassed by the present invention: M1-K984, M1-R983, M1-H982, M1-F981, M1-Y980, M1-L979, M1-P978, M1-K977, M1-A976, M1-N975, M1-G974, M1-S973, M1-E972, M1-V971, M1-K970, M1-P969, M1-L968, M1-Q967, M1-D966, M1-S965, M1-I964, M1-I963, M1-E962, M1-V961, M1-L960, M1-M959, M1-A958, M1-P957, M1-F956, M1-E955, M1-V954, M1-K953, M1-L952, M1-A951, M1-H950, M1-S949, M1-E948, M1-R947, M1-F946, M1-T945, M1-Y944, M1-F943, M1-C942, M1-F941, M1-E940, M1-L939, M1-L938, M1-D937, M1-S936, M1-V935, M1-L934, M1-D933, M1-H932, M1-M931, M1-S930, M1-D929, M1-L928, M1-L927, M1-K926, M1-T925, M1-L924, M1-Q923, M1-Y922, M1-F921, M1-R920, M1-Q919, M1-W918, M1-S917, M1-Q916, M1-G915, M1-S914, M1-N913, M1-N912, M1-P911, M1-C910, M1-K909, M1-T908, M1-V907, M1-M906, M1-K905, M1-R904, M1-L903, M1-E902, M1-K901, M1-I900, M1-Y899, M1-N898, M1-T897, M1-R896, M1-M895, M1-E894, M1-E893, M1-F892, M1-A891, M1-A890, M1-Q889, M1-S888, M1-K887, M1-L886, M1-G885, M1-D884, M1-K883, M1-P882, M1-I881, M1-T880, M1-S879, M1-L878, M1-L877, M1-L876, M1-L875, M1-V874, M1-K873, M1-M872, M1-I871, M1-T870, M1-Y869, M1-E868, M1-E867, M1-F866, M1-T865, M1-L864, M1-Q863, M1-L862, M1-R861, M1-V860, M1-F859, M1-Q858, M1-L857, M1-S856, M1-I855, M1-Q854, M1-H853, M1-M852, M1-G851, M1-Q850, M1-C849, M1-L848, M1-E847, M1-Y846, M1-M845, M1-A844, M1-S843, M1-Q842, M1-H841, M1-M840, M1-K839, M1-E838, M1-E837, M1-N836, M1-F835, M1-V834, M1-L833, M1-D832, M1-P831, M1-A830, M1-F829, M1-Y828, M1-L827, M1-F826, M1-Q825, M1-S824, M1-N823, M1-T822, M1-H821, M1-K820, M1-Y819, M1-S818, M1-R817, M1-W816, M1-S815, M1-L814, M1-A813, M1-F812, M1-S811, M1-S810, M1-L809, M1-C808, M1-M807, M1-W806, M1-S805, M1-Y804, M1-Q803, M1-I802, M1-L801, M1-T800, M1-I799, M1-Q798, M1-D797, M1-E796, M1-L795, M1-P794, M1-L793, M1-N792, M1-K791, M1-F790, M1-G789, M1-P788, M1-L787, M1-V786, M1-K785, M1-A784, M1-W783, M1-K782, M1-V781, M1-V780, M1-Q779, M1-I778, M1-M777, M1-Q776, M1-K775, M1-G774, M1-A773, M1-L772, M1-R771, M1-N770, M1-L769, M1-T768, M1-S767, M1-L766, M1-L765, M1-N764, M1-E763, M1-A762, M1-T761, M1-D760, M1-P759, M1-K758, M1-S757, M1-S756, M1-D755, M1-Y754, M1-G753, M1-A752, M1-Y751, M1-V750, M1-I749, M1-E748, M1-P747, M1-E746, M1-I745, M1-N744, M1-E743, M1-L742, M1-V741, M1-M740, M1-V739, M1-P738, M1-S737, M1-P736, M1-T735, M1-L734, M1-A733, M1-R732, M1-S731, M1-I730, M1-T729, M1-S728, M1-L727, M1-Q726, M1-P725, M1-V724, M1-L723, M1-A722, M1-T721, M1-N720, M1-V719, M1-S718, M1-P717, M1-E716, M1-K715, M1-A714, M1-P713, M1-A712, M1-I711, M1-Y710, M1-T709, M1-T708, M1-G707, M1-E706, M1-E705, M1-P704, M1-S703, M1-Q702, M1-P701, M1-P700, M1-P699, M1-P698, M1-P697, M1-P696, M1-P695, M1-P694, M1-Q693, M1-Q692, M1-Q691, M1-Q690, M1-P689, M1-Q688, M1-E687, M1-E686, M1-H685, M1-I684, M1-G683, M1-K682, M1-L681, M1-K680, M1-G679, M1-L678, M1-K677, M1-K676, M1-S675, M1-K674, M1-R673, M1-A672, M1-G671, M1-L670, M1-N669, M1-M668, M1-G667, M1-A666, M1-Q665, M1-L664, M1-C663, M1-K662, M1-Q661, M1-L660, M1-R659, M1-C658, M1-A657, M1-P656, M1-C655, M1-N654, M1-K653, M1-R652, M1-R651, M1-I650, M1-K649, M1-D648, M1-I647, M1-I646, M1-C645, M1-D644, M1-N643, M1-R642, M1-G641, M1-A640, M1-C639, M1-L638, M1-Y637, M1-N636, M1-H635, M1-Q634, M1-G633, M1-E632, M1-V631, M1-A630, M1-R629, M1-K628, M1-F627, M1-F626, M1-V625, M1-K624, M1-C623, M1-S622, M1-G621, M1-C620, M1-T619, M1-V618, M1-V617, M1-G616, M1-Y615, M1-H614, M1-C613, M1-G612, M1-S611, M1-A610, M1-E609, M1-D608, M1 -G607, M1-C606, M1-V605, M1-L604, M1-C603, M1-I602, M1-K601, M1-S600, M1-P599, M1-R598, M1-S597, M1-S596, M1-G595, M1-T594, M1-S593, M1-V592, M1-S591, M1-R590, M1-L589, M1-T588, M1-S587, M1-S586, M1-S585, M1-V584, M1-N583, M1-E582, M1-P581, M1-I580, M1-Y579, M1-E578, M1-L577, M1-V576, M1-P575, M1-Y574, M1-G573, M1-D572, M1-S571, M1-R570, M1-R569, M1-S568, M1-S567, M1-L566, M1-D565, M1-G564, M1-H563, M1-S562, M1-K561, M1-W560, M1-S559, M1-E558, M1-V557, M1-L556, M1-T555, M1-N554, M1-V553, M1-P552, M1-P551, M1-F550, M1-S549, M1-S548, M1-L547, M1-H546, M1-Q545, M1-F544, M1-S543, M1-Q542, M1-D541, M1-R540, M1-A539, M1-S538, M1-R537, M1-S536, M1-L535, M1-S534, M1-I533, M1-T532, M1-G531, M1-Q530, M1-A529, M1-G528, M1-I527, M1-R526, M1-Y525, M1-H524, M1-F523, M1-S522, M1-Q521, M1-G520, M1-G519, M1-S518, M1-N517, M1-V516, M1-G515, M1-V514, M1-I513, M1-A512, M1-S511, M1-S510, M1-P509, M1-I508, M1-S507, M1-A506, M1-E505, M1-P504, M1-Y503, M1-Y502, M1-S501, M1-G500, M1-D499, M1-D498, M1-P497, M1-E496, M1-Q495, M1-K494, M1-I493, M1-G492, M1-V491, M1-P490, M1-F489, M1-G488, M1-S487, M1-G486, M1-E485, M1-C484, M1-N483, M1-G482, M1-D481, M1-F480, M1-G479, M1-P478, M1-V477, M1-P476, M1-P475, M1-G474, M1-L473, M1-I472, M1-G471, M1-S470, M1-L469, M1-S468, M1-Y467, M1-Y466, M1-D465, M1-K464, M1-D463, M1-D462, M1-M461, M1-F460, M1-S459, M1-F458, M1-Y457, M1-S456, M1-G455, M1-D454, M1-M453, M1-F452, M1-P451, M1-F450, M1-P449, M1-N448, M1-V447, M1-T446, M1-P445, M1-N444, M1-G443, M1-K442, M1-F441, M1-S440, M1-T439, M1-G438, M1-S437, M1-C436, M1-S435, M1-H434, M1-K433, M1-T432, M1-S431, M1-E430, M1-Q429, M1-K428, M1-I427, M1-P426, M1-V425, M1-S424, M1-F423, M1-S422, M1-S421, M1-D420, M1-S419, M1-N418, M1-I417, M1-K416, M1-S415, M1-N414, M1-G413, M1-G412, M1-L 411, M1-C410, M1-S409, M1-S408, M1-S407, M1-F406, M1-A405, M1-G404, M1-D403, M1-P402, M1-E401, M1-P400, M1-K399, M1-I398, M1-Y397, M1-Q396, M1-V395, M1-I394, M1-N393, M1-L392, M1-Q391, M1-G390, M1-T389, M1-V388, M1-G387, M1-N386, M1-S385, M1-I384, M1-A383, M1-S382, M1-E381, M1-V380, M1-E379, M1-E378, M1-T377, M1-K376, M1-P375, M1-F374, M1-P373, M1-V372, M1-E371, M1-Q370, M1-A369, M1-G368, M1-K367, M1-E366, M1-Q365, M1-T364, M1-D363, M1-P362, M1-S361, M1-P360, M1-V359, M1-V358, M1-D357, M1-R356, M1-L355, M1-T354, M1-S353, M1-S352, M1-G351, M1-A350, M1-S349, M1-T348, M1-G347, M1-S346, M1-A345, M1-T344, M1-Y343, M1-S342, M1-F341, M1-A340, M1-N339, M1-N338, M1-V337, M1-P336, M1-S335, M1-C334, M1-I333, M1-S332, M1-G331, M1-V330, M1-T329, M1-S328, M1-A327, M1-A326, M1-P325, M1-S324, M1-S323, M1-L322, M1-T321, M1-S320, M1-R319, M1-N318, M1-N317, M1-T316, M1-N315, M1-S314, M1-P313, M1-S312, M1-S311, M1-V310, M1-S309, M1-C308, M1-R307, M1-S306, M1-N305, M1-N304, M1-I303, M1-N302, M1-A301, M1-P300, M1-S299, M1-S298, M1-V297, M1-S296, M1-S295, M1-R294, M1-L293, M1-T292, M1-V291, M1-N290, M1-N289, M1-P288, M1-S287, M1-S286, M1-V285, M1-P284, M1-S283, M1-K282, M1-V281, M1-S280, M1-C279, M1-H278, M1-S277, M1-P276, M1-P275, M1-S274, M1-S273, M1-I272, M1-S271, M1-S270, M1-K269, M1-M268, M1-S267, M1-S266, M1-L265, M1-P264, M1-S263, M1-S262, M1-L261, M1-P260, M1-S259, M1-G258, M1-V257, M1-N256, M1-S255, M1-A254, M1-H253, M1-A252, M1-P251, M1-S250, M1-H249, M1-S248, M1-R247, M1-S246, M1-G245, M1-R244, M1-N243, M1-E242, M1-A241, M1-N240, M1-P239, M1-S238, M1-C237, M1-T236, M1-L235, M1-P234, M1-T233, M1-G232, M1-Q231, M1-T230, M1-I229, M1-P228, M1-S227, M1-H226, M1-V225, M1-P224, M1-F223, M1-S222, M1-G221, M1-F220, M1-S219, M1-A218, M1-T217, M1-T216, M1-S215, M1-S214, M1-V213, M1-S212, M1-N211, M-I210, M1-G209, M1-A208, M1-P207, M1-S206, M1-C205, M1-V204, M1-S203, M1-S202, M1-T201, M1-M200, M1-N199, M1-L198, M1-P197, M1-S196, M1-C195, M1-V194, M1-S193, M1-P192, M1-S191, M1-K190, M1-E189, M1-H188, M1-C187, M1-M186, M1-I185, M1-P184, M1-S183, M1-K182, M1-V181, and/or M1-V180 of SEQ ID NO:4. Polynucleotide sequences encoding these polypeptides are also provided. The present invention also encompasses the use of these C-terminal MLR deletion polypeptides as immunogenic and/or antigenic epitopes as described elsewhere herein.

Alternatively, preferred polypeptides of the present invention may comprise polypeptide sequences corresponding to, for example, internal regions of the mineralocorticoid receptor polypeptide (e.g., any combination of both N- and C-terminal mineralocorticoid receptor polypeptide deletions) of SEQ ID NO:4. For example, internal regions could be defined by the equation: amino acid NX to amino acid CX, wherein NX refers to any N-terminal deletion polypeptide amino acid of mineralocorticoid receptor (SEQ ID NO:4), and where CX refers to any C-terminal deletion polypeptide amino acid of mineralocorticoid receptor (SEQ ID NO:4). Polynucleotides encoding these polypeptides are also provided. The present invention also encompasses the use of these polypeptides as an immunogenic and/or antigenic epitope as described elsewhere herein and are useful for creating allele-specific antibodies to discriminate between the reference and variable allele in a given sample, among other uses described herein. In addition such fragments may also be useful in designing allele-specific hybridization or other means probes to identify the allele to discriminate between the reference and variable allele in a given sample, among other uses described herein.

### Features of Gene No:3

The present invention relates to isolated nucleic acid molecules comprising, or alternatively consisting of, all or a portion of one or more alleles of SNP1 of the human PPAR-gamma gene, as provided in FIGS. 4A-B (SEQ ID NO:13) comprising at least one polymorphic locus. The allele described for SNP1 in FIGS. 4A-B (SEQ ID NO:13) represents the reference allele for this SNP and is exemplified by a “c” at nucleotide position 167. Fragments of this polynucleotide are at least about 10, at least about 20, at least about 40, or at least about 100, contiguous nucleotides and comprise one or more reference alleles at the nucleotide position(s) provided in FIGS. 4A-B (SEQ ID NO:13).

The present invention further relates to isolated proteins or polypeptides comprising, or alternatively, consisting of all or a portion of the encoded variant amino acid sequence of human PPAR-gamma (e.g., wherein reference to wildtype or reference PPAR-gamma polypeptide is exemplified by SEQ ID NO:14). Preferred portions are at least 10, preferably at least 20, preferably at least 40, preferably at least 100, contiguous polypeptides and comprises a “Pro” at the amino acid position corresponding to amino acid 12 of the PPAR-gamma polypeptide, or a portion of SEQ ID NO:14. The invention further relates to isolated nucleic acid molecules encoding such polypeptides or proteins, as well as to antibodies that bind to such proteins or polypeptides.

In one embodiment, the invention relates to a method for predicting the likelihood that an individual will have a disorder, particularly dose-dependent congestive heart failure or a congestive heart failure-like disorder, or be susceptible to developing dose-dependent congestive heart failure or a congestive heart failure-like disorder upon the administration of a pharmaceutically acceptable amount of a PPAR-agonist, comprising the step of identifying the nucleotide present at nucleotide position 167 of SEQ ID NO:13, from a DNA sample to be assessed, or the corresponding nucleotide at this position if only a fragment of the sequence provided as SEQ ID NO:13 is assessed. The presence of the reference allele at said position indicates that the individual from whom said DNA sample or fragment was obtained has a decreased likelihood of developing dose-dependent congestive heart failure or a congestive heart failure-like disorder upon the administration of a pharmaceutically acceptable amount of a PPAR-agonist, compared to an individual having the alternate (variant) allele(s) at said position(s); or at least a decreased likelihood of developing more severe congestive heart failure symptoms upon administration of the same.

Importantly, the presence of the reference allele at said position in a nucleic acid sample provided by an individual, indicates that said individual may be administered a correspondingly higher amount of a PPAR-agonist without increasing the likelihood of developing dose-dependent congestive heart failure or a congestive heart failure-like disorder relative to another individual having the variable allele(s) at said position. Therefore, such individuals may have the level of administered PPAR-agonist “titrated-up” or maintained in a safe manner.

Representative disorders which may be detected, diagnosed, identified, treated, prevented, and/or ameliorated by the SNPs and methods of the present invention include, the following, non-limiting diseases and disorders: dose-dependent congestive heart failure, congestive heart failure, susceptibility to developing dose-dependent congestive heart failure or a congestive heart failure-like disorder upon the administration of a pharmaceutically acceptable amount of a PPAR-agonist, susceptibility to developing dose-dependent congestive heart failure or a congestive heart failure-like disorder upon the administration of an increased level of a PPAR-agonist, adverse reactions associated with PPAR-agonist, disorders associated with aberrant PPAR-gamma expression, disorders associated with aberrant PPAR-gamma regulation, disorders associated with aberrant PPAR-gamma activity, disorders associated with aberrant regulation of PPAR-gamma by aldosterone, disorders associated with aberrant angiotensin II peptide levels, disorders associated with aberrant adrenal and renal vascular responses to angiotensin II, disorders associated with refractory responses to angiotensin II antagonists, hypertension, high blood pressure, hypotension, low-PPAR-gamma essential hypertension, high-PPAR-gamma essential hypertension, atherosclerosis, congestive heart failure, pulmonary congestive heart failure, beta blocker associated congestive heart failure, and beta-1 blocker associated congestive heart failure.

Additional disorders which may be detected, diagnosed, identified, treated, prevented, and/or ameliorated by the SNPs and methods of the present invention include, the following, non-limiting diseases and disorders: diabetes, especially Type 2 diabetes, and related diseases such as insulin resistance, hyperglycemia, hyperinsulinemia, elevated blood levels of fatty acids or glycerol, hyperlipidemia, obesity, hypertriglyceridemia, inflammation, Syndrome X, diabetic complications, dysmetabolic syndrome, and related diseases.

In one embodiment, the invention relates to a method for predicting the likelihood that an individual will have a disorder, particularly dose-dependent congestive heart failure or a congestive heart failure-like disorder, or be susceptible to developing dose-dependent congestive heart failure or a congestive heart failure-like disorder upon the administration of a pharmaceutically acceptable amount of a PPAR-agonist, comprising the step of identifying the amino acid present at amino acid position 12 of SEQ ID NO:14, from a sample to be assessed, or the corresponding amino acid at this position if only a fragment of the sequence provided as SEQ ID NO:14 is assessed. The presence of the reference allele (e.g., “Pro”) at said position indicates that the individual from whom said sample or fragment was obtained has a decreased likelihood of developing dose-dependent congestive heart failure or a congestive heart failure-like disorder upon the administration of a pharmaceutically acceptable amount of a PPAR-agonist, compared to an individual having the alternate (variant) allele(s) at said position(s); or at least a decreased likelihood of developing more severe congestive heart failure symptoms upon administration of the same.

Importantly, the presence of the reference allele at said amino acid position in a sample provided by an individual indicates that said individual may be administered a correspondingly higher amount of a PPAR-agonist without increasing the likelihood of developing dose-dependent congestive heart failure or a congestive heart failure-like disorder relative to another individual having the variable allele(s) at said position. Therefore, such individuals may have the level of administered PPAR-agonist “titrated-up” or maintained in a safe manner.

In preferred embodiments, the following N-terminal PPAR-gamma reference allele deletion polypeptides are encompassed by the present invention: M1-Y505, G2-Y505, E3-Y505, T4-Y505, L5-Y505, G6-Y505, D7-Y505, S8-Y505, P9-Y505, I10-Y505, D11-Y505, and/or P12-Y505 of SEQ ID NO:14. Polynucleotide sequences encoding these polypeptides are also provided. The present invention also encompasses the use of these N-terminal PPAR-gamma reference allele deletion polypeptides as immunogenic and/or antigenic epitopes as described elsewhere herein.

In preferred embodiments, the following C-terminal PPAR-gamma reference allele deletion polypeptides are encompassed by the present invention: M1-Y505, M1-L504, M1-D503, M1-K502, M1-Y501, M1-I500, M1-E499, M1-Q498, M1-L497, M1-L496, M1-P495, M1-H494, M1-L493, M1-S492, M1-M491, M1-D490, M1-T489, M1-E488, M1-T487, M1-K486, M1-K485, M1-I484, M1-V483, M1-Q482, M1-L481, M1-L480, M1-Q479, M1-V478, M1-H477, M1-E476, M1-T475, M1-V474, M1-I473, M1-Q472, M1-R471, M1-L470, M1-D469, M1-T468, M1-M467, M1-K466, M1-Q465, M1-L464, M1-L463, M1-K462, M1-A461, M1-F460, M1-L459, M1-Q458, M1-S457, M1-S456, M1-E455, M1-P454, M1-H453, M1-N452, M1-L451, M1-K450, M1-L449, M1-Q448, M1-L447, M1-E446, M1-L445, M1-A444, M1-Q443, M1-L442, M1-L441, M1-N440, M1-D439, M1-Q438, M1-I437, M1-D436, M1-E435, M1-I434, M1-P433, M1-K432, M1-V431, M1-N430, M1-L429, M1-L428, M1-G427, M1-P426, M1-R425, M1-D424, M1-G423, M1-S422, M1-L421, M1-I420, M1-I419, M1-V418, M1-A417, M1-I416, M1-F415, M1-I414, M1-A413, M1-L412, M1-D411, M1-S410, M1-D409, M1-D408, M1-L407, M1-E406, M1-L405, M1-A404, M1-N403, M1-F402, M1-K401, M1-V400, M1-A399, M1-F398, M1-E397, M1-F396, M1-K395, M1-P394, M1-E393, M1-M392, M1-F391, M1-D390, M1-G389, M1-F388, M1-P387, M1-K386, M1-R385, M1-L384, M1-S383, M1-K382, M1-L381, M1-F380, M1-E379, M1-R378, M1-T377, M1-M376, M1-F375, M1-G374, M1-Q373, M1-G372, M1-E371, M1-S370, M1-I369, M1-L368, M1-V367, M1-G366, M1-D365, M1-K364, M1-N363, M1-M362, M1-L361, M1-S360, M1-A359, M1-L358, M1-M357, M1-T356, M1-Y355, M1-I354, M1-I353, M1-E352, M1-H351, M1-V350, M1-G349, M1-Y348, M1-K347, M1-L346, M1-L345, M1-T344, M1-V343, M1-Q342, M1-D341, M1-N340, M1-L339, M1-D338, M1-L337, M1-N336, M1-V335, M1-F334, M1-G333, M1-P332, M1-I331, M1-S330, M1-K329, M1-A328, M1-Y327, M1-E326, M1-T325, M1-I324, M1-E323, M1-Q322, M1-V321, M1-A320, M1-E319, M1-V318, M1-S317, M1-R316, M1-F315, M1-Q314, M1-C313, M1-G312, M1-Q311, M1-F310, M1-I309, M1-R308, M1-I307, M1-A306, M1-V305, M1-E304, M1-K303, M1-S302, M1-Q301, M1-E300, M1-Q299, M1-L298, M1-P297, M1-T296, M1-I295, M1-H294, M1-K293, M1-F292, M1-K291, M1-I290, M1-K289, M1-D288, M1-E287, M1-G286, M1-M285, M1-M284, M1-L283, M1-S282, M1-N281, M1-M280, M1-D279, M1-Y278, M1-I277, M1-V276, M1-F275, M1-P274, M1-S273, M1-K272, M1-D271, M1-T270, M1-T269, M1-K268, M1-G267, M1-T266, M1-L265, M1-I264, M1-A263, M1-R262, M1-A261, M1-K260, M1-A259, M1-K258, M1-T257, M1-L256, M1-P255, M1-F254, M1-S253, M1-K252, M1-I251, M1-Y250, M1-S249, M1-D248, M1-Y247, M1-L246, M1-H245, M1-K244, M1-A243, M1-L242, M1-A241, M1-R240, M1-L239, M1-D238, M1-A237, M1-S236, M1-E235, M1-P234, M1-N233, M1-L232, M1-Q231, M1-D230, M1-I229, M1-D228, M1-S227, M1-S226, M1-I225, M1-E224, M1-A223, M1-L222, M1-L221, M1-K220, M1-E219, M1-K218, M1-E217, M1-A216, M1-Q215, M1-P214, M1-M213, M1-R212, M1-G211, M1-F210, M1-R209, M1-I208, M1-A207, M1-N206, M1-H205, M1-S204, M1-M203, M1-G202, M1-V201, M1-A200, M1-L199, M1-C198, M1-K197, M1-Q196, M1-F195, M1-R194, M1-C193, M1-Y192, M1-Q191, M1-C190, M1-K189, M1-N188, M1-R187, M1-S186, M1-K185, M1-K184, M1-H183, M1-I182, M1-R181, M1-C180, M1-N179, M1-L178, M1-D177, M1-C176, M1-R175, M1-D174, M1-Y173, M1-I172, M1-L171, M1-K170, M1-L169, M1-R168, M1-I167, M1-T166, M1-R165, M1-R164, M1-F163, M1-F162, M1-G161, M1-K160, M1-C159, M1-G158, M1-E157, M1-C156, M1-A155, M1-H154, M1-V153, M1-G152, M1-Y151, M1-H150, M1-F149, M1-G148, M1-S147, M1-A146, M1-K145, M1-D144, M1-G143, M1-C142, M1-V141, M1-R140, M1-C139, M1-E138, M1-I137, M1-A136, M1-M135, M1-L134, M1-S133, M1-N132, M1-S131, M1-P130, M1-E129, M1-E128, M1-H127, M1-P126, M1-K125, M1-N124, M1-Y123, M1-L122, M1-Q121, M1-T120, M1-K119, M1-E118, M1-S117, M1-Y116, M1-Y115, M1-P114, M1-P113, M1-S112, M1-A111, M1-P110, M1-E109, M1-V108, M1-K107, M1-I106, M1-A105, M1-S104, M1-Q103, M1-Y102, M1-E101, M1-Q100, M1-L99, M1-K98, M1-L97, M1-D96, M1-Y95, M1-K94, M1-Y93, M1-D92, M1-A91, M1-V90, M1-V89, M1-P88, M1-D87, M1-T86, M1-R85, M1-T84, M1-F83, M1-P82, M1-I81, M1-D80, M1-E79, M1-Y78, M1-H77, M1-P76, M1-T75, M1-S74, M1-I73, M1-S72, M1-S71, M1-F70, M1-D69, M1-V68, M1-T67, M1-T66, M1-F65, M1-P64, M1-K63, M1-I62, M1-D61, M1-F60, M1-S59, M1-H58, M1-S57, M1-H56, M1-D55, M1-E54, M1-M53, M1-V52, M1-S51, M1-L50, M1-D49, M1-V48, M1-S47, M1-S46, M1-I45, M1-G44, M1-F43, M1-N42, M1-T41, M1-P40, M1-W39, M1-F38, M1-P37, M1-M36, M1-E35, M1-T34, M1-D33, M1-V32, M1-M31, M1-T30, M1-M29, M1-E28, M1-Q27, M1-S26, M1-I25, M1-N24, M1-A23, M1-S22, M1-L21, M1-T20, M1-D19, M1-T18, M1-F17, M1-S16, M1-D15, M1-S14, M1-E13, and/or M1-P12 of SEQ ID NO:14. Polynucleotide sequences encoding these polypeptides are also provided. The present invention also encompasses the use of these C-terminal PPAR-gamma reference allele deletion polypeptides as immunogenic and/or antigenic epitopes as described elsewhere herein.

Alternatively, preferred polypeptides of the present invention may comprise polypeptide sequences corresponding to, for example, internal regions of the PPAR-gamma polypeptide (e.g., any combination of both N- and C- terminal PPAR-gamma polypeptide deletions) of SEQ ID NO:14. For example, internal regions could be defined by the equation: amino acid NX to amino acid CX, wherein NX refers to any N-terminal deletion polypeptide amino acid of PPAR-gamma (SEQ ID NO:14), and where CX refers to any C-terminal deletion polypeptide amino acid of PPAR-gamma (SEQ ID NO:14). Polynucleotides encoding these polypeptides are also provided. The present invention also encompasses the use of these polypeptides as an immunogenic and/or antigenic epitope as described elsewhere herein and are useful for creating allele-specific antibodies to discriminate between the reference and variable allele in a given sample, among other uses described herein. In addition such fragments may also be useful in designing allele-specific hybridization or other means probes to identify the allele to discriminate between the reference and variable allele in a given sample, among other uses described herein.

### Features of Gene No:4

The present invention relates to isolated nucleic acid molecules comprising, or alternatively consisting of, all or a portion of one or more alleles of SNP1 of the human PPAR-gamma gene, as provided in FIGS. 5A-B (SEQ ID NO:15) comprising at least one polymorphic locus. The allele described for SNP1 in FIGS. 5A-B (SEQ ID NO:15) represents the variable allele for this SNP and is exemplified by an “g” at nucleotide position 167. Fragments of this polynucleotide are at least about 10, at least about 20, at least about 40, at least about 100, contiguous nucleotides and comprise one or more variable alleles at the nucleotide position(s) provided in FIGS. 5A-B (SEQ ID NO:15).

The present invention further relates to isolated proteins or polypeptides comprising, or alternatively, consisting of all or a portion of the encoded variant amino acid sequence of human PPAR-gamma (e.g., wherein reference to variant or variable human PPAR-gamma polypeptide is exemplified by SEQ ID NO:16). Preferred portions are at least 10, preferably at least 20, preferably at least 40, preferably at least 100, contiguous polypeptides and comprises a “Ala” at the amino acid position corresponding to amino acid 12 of the PPAR-gamma polypeptide, or a portion of SEQ ID NO:16. The invention further relates to isolated nucleic acid molecules encoding such polypeptides or proteins, as well as to antibodies that bind to such proteins or polypeptides.

In one embodiment, the invention relates to a method for predicting the likelihood that an individual will have a disorder, particularly dose-dependent congestive heart failure or a congestive heart failure-like disorder, or be susceptible to developing dose-dependent congestive heart failure or a congestive heart failure-like disorder upon the administration of a pharmaceutically acceptable amount of a PPAR-agonist, comprising the step of identifying the nucleotide present at nucleotide position 167 of SEQ ID NO:15, from a DNA sample to be assessed, or the corresponding nucleotide at this position if only a fragment of the sequence provided as SEQ ID NO:15 is assessed. The presence of the variable allele at said position indicates that the individual from whom said DNA sample or fragment was obtained has an increased likelihood of developing dose-dependent congestive heart failure or a congestive heart failure-like disorder upon the administration of a pharmaceutically acceptable amount of a PPAR-agonist than an individual having the reference allele(s) at said position(s); or an increased likelihood of developing more severe congestive heart failure symptoms upon administration of the same.

Importantly, the presence of the variable allele at said polynucleotide and/or amino acid position in a sample provided by an individual indicates that said individual should be monitored more closely if an increased dosage of a PPAR-agonist is contemplated in order to avoid the potential of increasing the likelihood of developing dose-dependent congestive heart failure or a congestive heart failure-like disorder relative to another individual having the reference allele(s) at said position. In addition, a lower dose of a PPAR-agonist should be considered.

Representative disorders which may be detected, diagnosed, identified, treated, prevented, and/or ameliorated by the SNPs and methods of the present invention include, the following, non-limiting diseases and disorders: dose-dependent congestive heart failure, congestive heart failure, susceptibility to acquiring a congestive heart failure or a congestive heart failure-like disorder upon the administration of a pharmaceutically acceptable amount of a PPAR-agonist, adverse reactions associated with PPAR-agonist, disorders associated with aberrant PPAR-gamma expression, disorders associated with aberrant PPAR-gamma regulation, disorders associated with aberrant PPAR-gamma activity, disorders associated with aberrant regulation of PPAR-gamma by aldosterone, disorders associated with aberrant angiotensin II peptide levels, disorders associated with aberrant adrenal and renal vascular responses to angiotensin II, disorders associated with refractory responses to angiotensin II antagonists, hypertension, high blood pressure, hypotension, low-PPAR-gamma essential hypertension, high-PPAR-gamma essential hypertension, atherosclerosis, congestive heart failure, pulmonary congestive heart failure, beta blocker associated congestive heart failure, and beta-i blocker associated congestive heart failure.

Additional disorders which may be detected, diagnosed, identified, treated, prevented, and/or ameliorated by the SNPs and methods of the present invention include, the following, non-limiting diseases and disorders: diabetes, especially Type 2 diabetes, and related diseases such as insulin resistance, hyperglycemia, hyperinsulinemia, elevated blood levels of fatty acids or glycerol, hyperlipidemia, obesity, hypertriglyceridemia, inflammation, Syndrome X, diabetic complications, dysmetabolic syndrome, and related diseases.

In one embodiment, the invention relates to a method for predicting the likelihood that an individual will have a disorder, particularly dose-dependent congestive heart failure or a congestive heart failure-like disorder, or be susceptible to developing dose-dependent congestive heart failure or a congestive heart failure-like disorder upon the administration of a pharmaceutically acceptable amount of a PPAR-agonist, comprising the step of identifying the amino acid present at amino acid position 12 of SEQ ID NO:16, from a sample to be assessed, or the corresponding amino acid at this position if only a fragment of the sequence provided as SEQ ID NO:16 is assessed. The presence of the variable allele (e.g., “Ala”) at said position indicates that the individual from whom said sample or fragment was obtained has an increased likelihood of developing dose-dependent congestive heart failure or a congestive heart failure-like disorder upon the administration of a pharmaceutically acceptable amount of a PPAR-agonist, compared to an individual having the reference allele(s) at said position(s); or at least an increased likelihood of developing more severe peripheral dose-dependent congestive heart failure symptoms upon administration of the same.

Importantly, the presence of the variable allele at said amino acid position in a sample provided by an individual indicates that said individual should be monitored more closely if an increased dosage of a PPAR-agonist is contemplated in order to avoid the potential of increasing the likelihood of developing dose-dependent congestive heart failure or a congestive heart failure -like disorder relative to another individual having the reference allele(s) at said position. In addition, a lower dose of a PPAR-agonist should be considered.

In preferred embodiments, the following N-terminal PPAR-gamma variable allele deletion polypeptides are encompassed by the present invention: M1-Y505, G2-Y505, E3-Y505, T4-Y505, L5-Y505, G6-Y505, D7-Y505, S8-Y505, P9-Y505, I10-Y505, D11-Y505, and/or A12-Y505 of SEQ ID NO:16. Polynucleotide sequences encoding these polypeptides are also provided. The present invention also encompasses the use of these N-terminal PPAR-gamma variable allele deletion polypeptides as immunogenic and/or antigenic epitopes as described elsewhere herein.

In preferred embodiments, the following C-terminal PPAR-gamma variable allele deletion polypeptides are encompassed by the present invention: M1-Y505, M1-L504, M1-D503, M1-K502, M1-Y501, M1-I500, M1-E499, M1-Q498, M1-L497, M1-L496, M1-P495, M1-H494, M1-L493, M1-S492, M1-M491, M1-D490, M1-T489, M1-E488, M1-T487, M1-K486, M1-K485, M1-I484, M1-V483, M1-Q482, M1-L481, M1-L480, M1-Q479, M1-V478, M1-H477, M1-E476, M1-T475, M1-V474, M1-I473, M1-Q472, M1-R471, M1-L470, M1-D469, M1-T468, M1-M467, M1-K466, M1-Q465, M1-L464, M1-L463, M1-K462, M1-A461, M1-F460, M1-L459, M1-Q458, M1-S457, M1-S456, M1-E455, M1-P454, M1-H453, M1-N452, M1-L451, M1-K450, M1-L449, M1-Q448, M1-L447, M1-E446, M1-L445, M1-A444, M1-Q443, M1-L442, M1-L441, M1-N440, M1-D439, M1-Q438, M1-I437, M1-D436, M1-E435, M1-I434, M1-P433, M1-K432, M1-V431, M1-N430, M1-L429, M1-L428, M1-G427, M1-P426, M1-R425, M1-D424, M1-G423, M1-S422, M1-L421, M1-I420, M1-I419, M1-V418, M1-A417, M1-I416, M1-F415, M1-I414, M1-A413, M1-L412, M1-D411, M1-S410, M1-D409, M1-D408, M1-L407, M1-E406, M1-L405, M1-A404, M1-N403, M1-F402, M1-K401, M1-V400, M1-A399, M1-F398, M1-E397, M1-F396, M1-K395, M1-P394, M1-E393, M1-M392, M1-F391, M1-D390, M1-G389, M1-F388, M1-P387, M1-K386, M1-R385, M1-L384, M1-S383, M1-K382, M1-L381, M1-F380, M1-E379, M1-R378, M1-T377, M1-M376, M1-F375, M1-G374, M1-Q373, M1-G372, M1-E371, M1-S370, M1-I369, M1-L368, M1-V367, M1-G366, M1-D365, M1-K364, M1-N363, M1-M362, M1-L361, M1-S360, M1-A359, M1-L358, M1-M357, M1-T356, M1-Y355, M1-I354, M1-I353, M1-E352, M1-H351, M1-V350, M1-G349, M1-Y348, M1-K347, M1-L346, M1-L345, M1-T344, M1-V343, M1-Q342, M1-D341, M1-N340, M1-L339, M1-D338, M1-L337, M1-N336, M1-V335, M1-F334, M1-G333, M1-P332, M1-I331, M1-S330, M1-K329, M1-A328, M1-Y327, M1-E326, M1-T325, M1-I324, M1-E323, M1-Q322, M1-V321, M1-A320, M1-E319, M1-V318, M1-S317, M1-R316, M1-F315, M1-Q314, M1-C313, M1-G312, M1-Q311, M1-F310, M1-I309, M1-R308, M1-I307, M1-A306, M1-V305, M1-E304, M1-K303, M1-S302, M1-Q301, M1-E300, M1-Q299, M1-L298, M1-P297, M1-T296, M1-I295, M1-H294, M1-K293, M1-F292, M1-K291, M1-I290, M1-K289, M1-D288, M1-E287, M1-G286, M1-M285, M1-M284, M1-L283, M1-S282, M1-N281, M1-M280, M1-D279, M1-Y278, M1-I277, M1-V276, M1-F275, M1-P274, M1-S273, M1-K272, M1-D271, M1-T270, M1-T269, M1-K268, M1-G267, M1-T266, M1-L265, M1-I264, M1-A263, M1-R262, M1-A261, M1-K260, M1-A259, M1-K258, M1-T257, M1-L256, M1-P255, M1-F254, M1-S253, M1-K252, M1-I251, M1-Y250, M1-S249, M1-D248, M1-Y247, M1-L246, M1-H245, M1-K244, M1-A243, M1-L242, M1-A241, M1-R240, M1-L239, M1-D238, M1-A237, M1-S236, M1-E235, M1-P234, M1-N233, M1-L232, M1-Q231, M1-D230, M1-I229, M1-D228, M1-S227, M1-S226, M1-I225, M1-E224, M1-A223, M1-L222, M1-L221, M1-K220, M1-E219, M1-K218, M1-E217, M1-A216, M1-Q215, M1-P214, M1-M213, M1-R212, M1-G211, M1-F210, M1-R209, M1-I208, M1-A207, M1-N206, M1-H205, M1-S204, M1-M203, M1-G202, M1-V201, M1-A200, M1-L199, M1-C198, M1-K197, M1-Q196, M1-F195, M1-R194, M1-C193, M1-Y192, M1-Q191, M1-C190, M1-K189, M1-N188, M1-R187, M1-S186, M1-K185, M1-K184, M1-H183, M1-I182, M1-R181, M1-C180, M1-N179, M1-L178, M1-D177, M1-C176, M1-R175, M1-D174, M1-Y173, M1-I172, M1-L171, M1-K170, M1-L169, M1-R168, M1-I167, M1-T166, M1-R165, M1-R164, M1-F163, M1-F162, M1-G161, M1-K160, M1-C159, M1-G158, M1-E157, M1-C156, M1-A155, M1-H154, M1-V153, M1-G152, M1-Y151, M1-H150, M1-F149, M1-G148, M1-S147, M1-A146, M1-K145, M1-D144, M1-G143, M1-C142, M1-V141, M1-R140, M1-C139, M1-E138, M1-I137, M1-A136, M1-M135, M1-L134, M1-S133, M1-N132, M1-S131, M1-P130, M1-E129, M1-E128, M1-H127, M1-P126, M1-K125, M1-N124, M1-Y123, M1-L122, M1-Q121, M1-T120, M1-K119, M1-E118, M1-S117, M1-Y116, M1-Y115, M1-P114, M1-P113, M1-S112, M1-A111, M1-P110, M1-E109, M1-V108, M1-K107, M1-I106, M1-A105, M1-S104, M1-Q103, M1-Y102, M1-E101, M1-Q100, M1-L99, M1-K98, M1-L97, M1-D96, M1-Y95, M1-K94, M1-Y93, M1-D92, M1-A91, M1-V90, M1-V89, M1-P88, M1-D87, M1-T86, M1-R85, M1-T84, M1-F83, M1-P82, M1-I81, M1-D80, M1-E79, M1-Y78, M1-H77, M1-P76, M1-T75, M1-S74, M1-I73, M1-S72, M1-S71, M1-F70, M1-D69, M1-V68, M1-T67, M1-T66, M1-F65, M1-P64, M1-K63, M1-I62, M1-D61, M1-F60, M1-S59, M1-H58, M1-S57, M1-H56, M1-D55, M1-E54, M1-M53, M1-V52, M1-S51, M1-L50, M1-D49, M1-V48, M1-S47, M1-S46, M1-I45, M1-G44, M1-F43, M1-N42, M1-T41, M1-P40, M1-W39, M1-F38, M1-P37, M1-M36, M1-E35, M1-T34, M1-D33, M1-V32, M1-M31, M1-T30, M1-M29, M1-E28, M1-Q27, M1-S26, M1-I25, M1-N24, M1-A23, M1-S22, M1-L21, M1-T20, M1-D19, M1-T18, M1-F17, M1-S16, M1-D15, M1-S14, M1-E13, and/or M1-A12 of SEQ ID NO:16. Polynucleotide sequences encoding these polypeptides are also provided. The present invention also encompasses the use of these C-terminal PPAR-gamma variable allele deletion polypeptides as immunogenic and/or antigenic epitopes as described elsewhere herein.

Alternatively, preferred polypeptides of the present invention may comprise polypeptide sequences corresponding to, for example, internal regions of the PPAR-gamma polypeptide (e.g., any combination of both N- and C- terminal PPAR-gamma polypeptide deletions) of SEQ ID NO:16. For example, internal regions could be defmed by the equation: amino acid NX to amino acid CX, wherein NX refers to any N-terminal deletion polypeptide amino acid of PPAR-gamma (SEQ ID NO:16), and where CX refers to any C-terminal deletion polypeptide amino acid of PPAR-gamma (SEQ ID NO:16). Polynucleotides encoding these polypeptides are also provided. The present invention also encompasses the use of these polypeptides as an immunogenic and/or antigenic epitope as described elsewhere herein and are useful for creating allele-specific antibodies to discriminate between the reference and variable allele in a given sample, among other uses described herein. In addition such fragments may also be useful in designing allele-specific hybridization or other means probes to identify the allele to discriminate between the reference and variable allele in a given sample, among other uses described herein.

### Features of Gene No:5

The present invention relates to isolated nucleic acid molecules comprising, or alternatively consisting of, all or a portion of one or more alleles of SNP 1 of the human Adiponectin gene, as provided in FIGS. 7A-D (SEQ ID NO:26) comprising at least one polymorphic locus. The allele described for SNP1 in FIGS. 7A-D (SEQ ID NO:26) represents the reference allele for this SNP and is exemplified by an “t” at nucleotide position 415. Fragments of this polynucleotide are at least about 10, at least about 20, at least about 40, or at least about 100, contiguous nucleotides and comprise one or more reference alleles at the nucleotide position(s) provided in FIGS. 7A-D (SEQ ID NO:26).

The present invention further relates to isolated proteins or polypeptides comprising, or alternatively, consisting of all or a portion of the encoded variant amino acid sequence of human Adiponectin (e.g., wherein reference to wildtype or reference Adiponectin polypeptide is exemplified by SEQ ID NO:27). Preferred portions are at least 10, preferably at least 20, preferably at least 40, preferably at least 100, contiguous polypeptides and comprises a “Tyr” at the amino acid position corresponding to amino acid 111 of the Adiponectin polypeptide, or a portion of SEQ ID NO:27. The invention further relates to isolated nucleic acid molecules encoding such polypeptides or proteins, as well as to antibodies that bind to such proteins or polypeptides.

In one embodiment, the invention relates to a method for predicting the likelihood that an individual will have a disorder, particularly dose-dependent congestive heart failure or a congestive heart failure-like disorder, or be susceptible to developing dose-dependent congestive heart failure or a congestive heart failure-like disorder upon the administration of a pharmaceutically acceptable amount of a PPAR-agonist, comprising the step of identifying the nucleotide present at nucleotide position 415 of SEQ ID NO:26, from a DNA sample to be assessed, or the corresponding nucleotide at this position if only a fragment of the sequence provided as SEQ ID NO:26 is assessed. The presence of the reference allele at said position indicates that the individual from whom said DNA sample or fragment was obtained has a decreased likelihood of developing dose-dependent congestive heart failure or a congestive heart failure-like disorder upon the administration of a pharmaceutically acceptable amount of a PPAR-agonist, compared to an individual having the alternate (variant) allele(s) at said position(s); or at least a decreased likelihood of developing more severe congestive heart failure symptoms upon administration of the same.

Importantly, the presence of the reference allele at said position in a nucleic acid sample provided by an individual, indicates that said individual may be administered a correspondingly higher amount of a PPAR-agonist without increasing the likelihood of developing dose-dependent congestive heart failure or a congestive heart failure-like disorder relative to another individual having the variable allele(s) at said position. Therefore, such individuals may have the level of administered PPAR-agonist “titrated-up” or maintained in a safe manner.

Representative disorders which may be detected, diagnosed, identified, treated, prevented, and/or ameliorated by the SNPs and methods of the present invention include, the following, non-limiting diseases and disorders: dose-dependent congestive heart failure, congestive heart failure, susceptibility to developing dose-dependent congestive heart failure or a congestive heart failure-like disorder upon the administration of a pharmaceutically acceptable amount of a PPAR-agonist, susceptibility to developing dose-dependent congestive heart failure or a congestive heart failure-like disorder upon the administration of an increased level of a PPAR-agonist, adverse reactions associated with PPAR-agonist, disorders associated with aberrant Adiponectin expression, disorders associated with aberrant Adiponectin regulation, disorders associated with aberrant Adiponectin activity, disorders associated with aberrant regulation of Adiponectin by aldosterone, disorders associated with aberrant angiotensin II peptide levels, disorders associated with aberrant adrenal and renal vascular responses to angiotensin II, disorders associated with refractory responses to angiotensin II antagonists, hypertension, high blood pressure, hypotension, low-Adiponectin essential hypertension, high-Adiponectin essential hypertension, atherosclerosis, congestive heart failure, pulmonary congestive heart failure, beta blocker associated congestive heart failure, and beta-1 blocker associated congestive heart failure.

Additional disorders which may be detected, diagnosed, identified, treated, prevented, and/or ameliorated by the SNPs and methods of the present invention include, the following, non-limiting diseases and disorders: diabetes, especially Type 2 diabetes, and related diseases such as insulin resistance, hyperglycemia, hyperinsulinemia, elevated blood levels of fatty acids or glycerol, hyperlipidemia, obesity, hypertriglyceridemia, inflammation, Syndrome X, diabetic complications, dysmetabolic syndrome, and related diseases.

In one embodiment, the invention relates to a method for predicting the likelihood that an individual will have a disorder, particularly dose-dependent congestive heart failure or a congestive heart failure-like disorder, or be susceptible to developing dose-dependent congestive heart failure or a congestive heart failure-like disorder upon the administration of a pharmaceutically acceptable amount of a PPAR-agonist, comprising the step of identifying the amino acid present at amino acid position 111 of SEQ ID NO:27, from a sample to be assessed, or the corresponding amino acid at this position if only a fragment of the sequence provided as SEQ ID NO:27 is assessed. The presence of the reference allele (e.g., “Tyr”) at said position indicates that the individual from whom said sample or fragment was obtained has a decreased likelihood of developing dose-dependent congestive heart failure or a congestive heart failure-like disorder upon the administration of a pharmaceutically acceptable amount of a PPAR-agonist, compared to an individual having the alternate (variant) allele(s) at said position(s); or at least a decreased likelihood of developing more severe congestive heart failure symptoms upon administration of the same.

Importantly, the presence of the reference allele at said amino acid position in a sample provided by an individual indicates that said individual may be administered a correspondingly higher amount of a PPAR-agonist without increasing the likelihood of developing dose-dependent congestive heart failure or a congestive heart failure-like disorder relative to another individual having the variable allele(s) at said position. Therefore, such individuals may have the level of administered PPAR-agonist “titrated-up” or maintained in a safe manner.

In preferred embodiments, the following N-terminal Adiponectin reference allele deletion polypeptides are encompassed by the present invention: M1-N244, L2-N244, L3-N244, L4-N244, G5-N244, A6-N244, V7-N244, L8-N244, L9-N244, L10-N244, N244, L11-N244, A12-N244, L13-N244, P14-N244, G15-N244, H16-N244, D17-N244, Q18-N244, E19-N244, T20-N244, T21-N244, T22-N244, Q23-N244, G24-N244, P25-N244, G26-N244, V27-N244, L28-N244, L29-N244, P30-N244, L31-N244, P32-N244, K33-N244, G34-N244, A35-N244, C36-N244, T37-N244, G38-N244, W39-N244, M40-N244, A41-N244, G42-N244, I43-N244, P44-N244, G45-N244, H46-N244, P47-N244, G48-N244, H49-N244, N50-N244, G51-N244, A52-N244, P53-N244, G54-N244, R55-N244, D56-N244, G57-N244, R58-N244, D59-N244, G60-N244, T61-N244, P62-N244, G63-N244, E64-N244, K65-N244, G66-N244, E67-N244, K68-N244, G69-N244, D70-N244, P71-N244, G72-N244, L73-N244, I74-N244, G75-N244, P76-N244, K77-N244, G78-N244, D79-N244, I80-N244, G81-N244, E82-N244, T83-N244, G84-N244, V85-N244, P86-N244, G87-N244, A88-N244, E89-N244, G90-N244, P91-N244, R92-N244, G93-N244, F94-N244, P95-N244, G96-N244, 197-N244, Q98-N244, G99-N244, R100-N244, K101-N244, G102-N244, E103-N244, P104-N244, G105-N244, E106-N244, G107-N244, A108-N244, Y109-N244, V110-N244, and/or Y111-N244 of SEQ ID NO:27. Polynucleotide sequences encoding these polypeptides are also provided. The present invention also encompasses the use of these N-terminal Adiponectin reference allele deletion polypeptides as immunogenic and/or antigenic epitopes as described elsewhere herein.

In preferred embodiments, the following C-terminal Adiponectin reference allele deletion polypeptides are encompassed by the present invention: M1-N244, M1-T243, M1-D242, M1-H241, M1-Y240, M1-L239, M1-L238, M1-F237, M1-G236, M1-T235, M1-F234, M1-T233, M1-S232, M1-D231, M1-N230, M1-D229, M1-N228, M1-D227, M1-A226, M1-Y225, M1-L224, M1-G223, M1-N222, M1-R221, M1-E220, M1-G219, M1-E218, M1-G217, M1-Y216, M1-V215, M1-Q214, M1-L213, M1-W212, M1-V211, M1-Q210, M1-D209, M1-G208, M1-V207, M1-E206, M1-L205, M1-H204, M1-L203, M1-L202, M1-V201, M1-S200, M1-G199, M1-S198, M1-A197, M1-Q196, M1-D195, M1-V194, M1-N193, M1-N192, M1-E191, M1-Q190, M1-Y189, M1-Q188, M1-D187, M1-Y186, M1-T185, M1-F184, M1-L183, M1-M182, M1-A181, M1-K180, M1-D179, M1-K178, M1-K177, M1-F176, M1-L175, M1-S174, M1-V173, M1-K172, M1-V171, M1-D170, M1-K169, M1-M168, M1-Y167, M1-V166, M1-T165, M1-I164, M1-H163, M1-Y162, M1-A161, M1-F160, M1-Y159, M1-Y158, M1-L157, M1-G156, M1-P155, M1-I154, M1-N153, M1-C152, M1-H151, M1-F150, M1-K149, M1-G148, M1-T147, M1-S146, M1-G145, M1-D144, M1-Y143, M1-H142, M1-N141, M1-Q140, M1-Q139, M1-N138, M1-Y137, M1-F136, M1-I135, M1-K134, M1-T133, M1-F132, M1-R131, M1-I130, M1-P129, M1-M128, M1-N127, M1-P126, M1-I125, M1-T124, M1-V123, M1-Y122, M1-T121, M1-E120, M1-L119, M1-G118, M1-V117, M1-S116, M1-F115, M1-A114, M1-S113, M1-R112, and/or M1-Y111 of SEQ ID NO:27. Polynucleotide sequences encoding these polypeptides are also provided. The present invention also encompasses the use of these C-terminal Adiponectin reference allele deletion polypeptides as immunogenic and/or antigenic epitopes as described elsewhere herein.

Alternatively, preferred polypeptides of the present invention may comprise polypeptide sequences corresponding to, for example, internal regions of the Adiponectin polypeptide (e.g., any combination of both N- and C- terminal Adiponectin polypeptide deletions) of SEQ ID NO:27. For example, internal regions could be defined by the equation: amino acid NX to amino acid CX, wherein NX refers to any N-terminal deletion polypeptide amino acid of Adiponectin (SEQ ID NO:27), and where CX refers to any C-terminal deletion polypeptide amino acid of Adiponectin (SEQ ID NO:27). Polynucleotides encoding these polypeptides are also provided. The present invention also encompasses the use of these polypeptides as an immunogenic and/or antigenic epitope as described elsewhere herein and are useful for creating allele-specific antibodies to discriminate between the reference and variable allele in a given sample, among other uses described herein. In addition such fragments may also be useful in designing allele-specific hybridization or other means probes to identify the allele to discriminate between the reference and variable allele in a given sample, among other uses described herein.

### Features of Gene No:6

The present invention relates to isolated nucleic acid molecules comprising, or alternatively consisting of, all or a portion of one or more alleles of SNP1 of the human Adiponectin gene, as provided in FIGS. 8A-D (SEQ ID NO:28) comprising at least one polymorphic locus. The allele described for SNP1 in FIGS. 8A-D (SEQ ID NO:28) represents the variable allele for this SNP and is exemplified by a “c” at nucleotide position 415. Fragments of this polynucleotide are at least about 10, at least about 20, at least about 40, at least about 100, contiguous nucleotides and comprise one or more variable alleles at the nucleotide position(s) provided in FIGS. 8A-D (SEQ ID NO:28).

The present invention further relates to isolated proteins or polypeptides comprising, or alternatively, consisting of all or a portion of the encoded variant amino acid sequence of human Adiponectin (e.g., wherein reference to variant or variable human Adiponectin polypeptide is exemplified by SEQ ID NO:29). Preferred portions are at least 10, preferably at least 20, preferably at least 40, preferably at least 100, contiguous polypeptides and comprises a “His” at the amino acid position corresponding to amino acid 111 of the Adiponectin polypeptide, or a portion of SEQ ID NO:29. The invention further relates to isolated nucleic acid molecules encoding such polypeptides or proteins, as well as to antibodies that bind to such proteins or polypeptides.

In one embodiment, the invention relates to a method for predicting the likelihood that an individual will have a disorder, particularly dose-dependent congestive heart failure or a congestive heart failure-like disorder, or be susceptible to developing dose-dependent congestive heart failure or a congestive heart failure-like disorder upon the administration of a pharmaceutically acceptable amount of a PPAR-agonist, comprising the step of identifying the nucleotide present at nucleotide position 415 of SEQ ID NO:28, from a DNA sample to be assessed, or the corresponding nucleotide at this position if only a fragment of the sequence provided as SEQ ID NO:28 is assessed. The presence of the variable allele at said position indicates that the individual from whom said DNA sample or fragment was obtained has an increased likelihood of developing dose-dependent congestive heart failure or a congestive heart failure-like disorder upon the administration of a pharmaceutically acceptable amount of a PPAR-agonist than an individual having the reference allele(s) at said position(s); or an increased likelihood of developing more severe congestive heart failure symptoms upon administration of the same.

Importantly, the presence of the variable allele at said polynucleotide and/or amino acid position in a sample provided by an individual indicates that said individual should be monitored more closely if an increased dosage of a PPAR-agonist is contemplated in order to avoid the potential of increasing the likelihood of developing dose-dependent congestive heart failure or a congestive heart failure-like disorder relative to another individual having the reference allele(s) at said position. In addition, a lower dose of a PPAR-agonist should be considered.

Representative disorders which may be detected, diagnosed, identified, treated, prevented, and/or ameliorated by the SNPs and methods of the present invention include, the following, non-limiting diseases and disorders: dose-dependent congestive heart failure, congestive heart failure, susceptibility to acquiring a congestive heart failure or a congestive heart failure-like disorder upon the administration of a pharmaceutically acceptable amount of a PPAR-agonist, adverse reactions associated with PPAR-agonist, disorders associated with aberrant Adiponectin expression, disorders associated with aberrant Adiponectin regulation, disorders associated with aberrant Adiponectin activity, disorders associated with aberrant regulation of Adiponectin by aldosterone, disorders associated with aberrant angiotensin II peptide levels, disorders associated with aberrant adrenal and renal vascular responses to angiotensin II, disorders associated with refractory responses to angiotensin II antagonists, hypertension, high blood pressure, hypotension, low-Adiponectin essential hypertension, high-Adiponectin essential hypertension, atherosclerosis, congestive heart failure, pulmonary congestive heart failure, beta blocker associated congestive heart failure, and beta-1 blocker associated congestive heart failure.

Additional disorders which may be detected, diagnosed, identified, treated, prevented, and/or ameliorated by the SNPs and methods of the present invention include, the following, non-limiting diseases and disorders: diabetes, especially Type 2 diabetes, and related diseases such as insulin resistance, hyperglycemia, hyperinsulinemia, elevated blood levels of fatty acids or glycerol, hyperlipidemia, obesity, hypertriglyceridemia, inflammation, Syndrome X, diabetic complications, dysmetabolic syndrome, and related diseases.

In one embodiment, the invention relates to a method for predicting the likelihood that an individual will have a disorder, particularly dose-dependent congestive heart failure or a congestive heart failure-like disorder, or be susceptible to developing dose-dependent congestive heart failure or a congestive heart failure-like disorder upon the administration of a pharmaceutically acceptable amount of a PPAR-agonist, comprising the step of identifying the amino acid present at amino acid position 111 of SEQ ID NO:29, from a sample to be assessed, or the corresponding amino acid at this position if only a fragment of the sequence provided as SEQ ID NO:29 is assessed. The presence of the variable allele (e.g., “His”) at said position indicates that the individual from whom said sample or fragment was obtained has an increased likelihood of developing dose-dependent congestive heart failure or a congestive heart failure-like disorder upon the administration of a pharmaceutically acceptable amount of a PPAR-agonist, compared to an individual having the reference allele(s) at said position(s); or at least an increased likelihood of developing more severe peripheral dose-dependent congestive heart failure symptoms upon administration of the same.

Importantly, the presence of the variable allele at said amino acid position in a sample provided by an individual indicates that said individual should be monitored more closely if an increased dosage of a PPAR-agonist is contemplated in order to avoid the potential of increasing the likelihood of developing dose-dependent congestive heart failure or a congestive heart failure-like disorder relative to another individual having the reference allele(s) at said position. In addition, a lower dose of a PPAR-agonist should be considered.

In preferred embodiments, the following N-terminal Adiponectin variable allele deletion polypeptides are encompassed by the present invention: M1-N244, L2-N244, L3-N244, L4-N244, G5-N244, A6-N244, V7-N244, L8-N244, L9-N244, L10-N244, L11-N244, A12-N244, L13-N244, P14-N244, G15-N244, H16-N244, D17-N244, Q18-N244, E19-N244, T20-N244, T21-N244, T22-N244, Q23-N244, G24-N244, P25-N244, G26-N244, V27-N244, L28-N244, L29-N244, P30-N244, L31-N244, P32-N244, K33-N244, G34-N244, A35-N244, C36-N244, T37-N244, G38-N244, W39-N244, M40-N244, A41-N244, G42-N244, I43-N244, P44-N244, G45-N244, H46-N244, P47-N244, G48-N244, H49-N244, N50-N244, G51-N244, A52-N244, P53-N244, G54-N244, R55-N244, D56-N244, G57-N244, R58-N244, D59-N244, G60-N244, T61 -N244, P62-N244, G63-N244, E64-N244, K65-N244, G66-N244, E67-N244, K68-N244, G69-N244, D70-N244, P71-N244, G72-N244, L73-N244, I74-N244, G75-N244, P76-N244, K77-N244, G78-N244, D79-N244, I80-N244, G81-N244, E82-N244, T83-N244, G84-N244, V85-N244, P86-N244, G87-N244, A88-N244, E89-N244, G90-N244, P91 -N244, R92-N244, G93-N244, F94-N244, P95-N244, G96-N244, I97-N244, Q98-N244, G99-N244, R100-N244, K101-N244, G102-N244, E103-N244, P104-N244, G105-N244, E106-N244, G107-N244, A108-N244, Y109-N244, V110-N244, and/or H111-N244 of SEQ ID NO:29. Polynucleotide sequences encoding these polypeptides are also provided. The present invention also encompasses the use of these N-terminal Adiponectin variable allele deletion polypeptides as immunogenic and/or antigenic epitopes as described elsewhere herein.

In preferred embodiments, the following C-terminal Adiponectin variable allele deletion polypeptides are encompassed by the present invention: M1-N244, M1-T243, M1-D242, M1-H241, M1-Y240, M1-L239, M1-L238, M1-F237, M1-G236, M1-T235, M1-F234, M1-T233, M1-S232, M1-D231, M1-N230, M1-D229, M1-N228, M1-D227, M1-A226, M1-Y225, M1-L224, M1-G223, M1-N222, M1-R221, M1-E220, M1-G219, M1-E218, M1-G217, M1-Y216, M1-V215, M1-Q214, M1-L213, M1-W212, M1-V211, M1-Q210, M1-D209, M1-G208, M1-V207, M1-E206, M1-L205, M1-H204, M1-L203, M1-L202, M1-V201, M1-S200, M1-G199, M1-S198, M1-A197, M1-Q196, M1-D195, M1-V194, M1-N193, M1-N192, M1-E191, M1-Q190, M1-Y189, M1-Q188, M1-D187, M1-Y186, M1-T185, M1-F184, M1-L183, M1-M182, M1-A181, M1-K180, M1-D179, M1-K178, M1-K177, M1-F176, M1-L175, M1-S174, M1-V173, M1-K172, M1-V171, M1-D170, M1-K169, M1-M168, M1-Y167, M1-V166, M1-T165, M1-I164, M1-H163, M1-Y162, M1-A161, M1-F160, M1-Y159, M1-Y158, M1-L157, M1-G156, M1-P155, M1-I154, M1-N153, M1-C152, M1-H151, M1-F150, M1-K149, M1-G148, M1-T147, M1-S146, M1-G145, M1-D144, M1-Y143, M1-H142, M1-N141, M1-Q140, M1-Q139, M1-N138, M1-Y137, M1-F136, M1-I135, M1-K134, M1-T133, M1-F132, M1-R131, M1-I130, M1-P129, M1-M128, M1-N127, M1-P126, M1-I125, M1-T124, M1-V123, M1-Y122, M1-T121, M1-E120, M1-L119, M1-G118, M1-V117, M1-S116, M1-F115, M1-A114, M1-S113, M1-R112, and/or M1-H111 of SEQ ID NO:29. Polynucleotide sequences encoding these polypeptides are also provided. The present invention also encompasses the use of these C-terminal Adiponectin variable allele deletion polypeptides as immunogenic and/or antigenic epitopes as described elsewhere herein.

Alternatively, preferred polypeptides of the present invention may comprise polypeptide sequences corresponding to, for example, internal regions of the Adiponectin polypeptide (e.g., any combination of both N- and C-terminal Adiponectin polypeptide deletions) of SEQ ID NO:29. For example, internal regions could be defined by the equation: amino acid NX to amino acid CX, wherein NX refers to any N-terminal deletion polypeptide amino acid of Adiponectin (SEQ ID NO:29), and where CX refers to any C-terminal deletion polypeptide amino acid of Adiponectin (SEQ ID NO:29). Polynucleotides encoding these polypeptides are also provided. The present invention also encompasses the use of these polypeptides as an immunogenic and/or antigenic epitope as described elsewhere herein and are useful for creating allele-specific antibodies to discriminate between the reference and variable allele in a given sample, among other uses described herein. In addition such fragments may also be useful in designing allele-specific hybridization or other means probes to identify the allele to discriminate between the reference and variable allele in a given sample, among other uses described herein.

TABLE I

| | | | | Nucleotide | | | Amino Acid | | |
|---|---|---|---|---|---|---|---|---|---|
| | | | Poly- | Position of | Nucleotide | | Position | Amino Acid | |
| Poly- | | | morphic | Poly- | at Poly- | SEQ | of Poly- | at Poly- | SEQ |
| nucleotide | CDNA | | Locus | morphic | morphic | ID | morphic | morphic | ID |
| No. | CloneID | Allele | Number | Locus | Locus | NO: | Locus | Locus | NO: |
| 1 | Human | Reference | 1 | 754 | A | 1 | 180 | I | 2 |
| | MLR Gene- | | | | | | | | |
| | SNP1 | | | | | | | | |
| 2 | Human | Variable | 1 | 754 | G | 3 | 180 | V | 4 |
| | MLR Gene- | | | | | | | | |
| | SNP1 | | | | | | | | |
| 3 | Human | Reference | 1 | 167 | C | 13 | 12 | P | 14 |
| | PPAR- | | | | | | | | |
| | gamma | | | | | | | | |
| | Gene-SNP1 | | | | | | | | |
| 4 | Human | Variable | 1 | 167 | G | 15 | 12 | A | 16 |
| | PPAR- | | | | | | | | |
| | gamma | | | | | | | | |
| | Gene-SNP1 | | | | | | | | |
| 5 | Human | Reference | 1 | 415 | T | 26 | 111 | T | 27 |
| | Adiponcetin | | | | | | | | |
| | Gene-SNP1 | | | | | | | | |
| 6 | Human | Variable | 1 | 415 | C | 28 | 111 | H | 29 |
| | Adiponectin | | | | | | | | |
| | Gene-SNP1 | | | | | | | | |

The polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The polypeptides may be in the form of the protein, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

The polypeptides of the present invention are preferably provided in an isolated form, and preferably are substantially purified. A recombinantly produced version of a polypeptide, can be substantially purified using techniques described herein or otherwise known in the art, such as, for example, by the one-step method described in Smith and Johnson, Gene 67:31-40 (1988). Polypeptides of the invention also can be purified from natural, synthetic or recombinant sources using protocols described herein or otherwise known in the art, such as, for example, antibodies of the invention raised against the full-length form of the protein.

The present invention provides a polynucleotide comprising, or alternatively consisting of, the sequence identified as SEQ ID NO:1, 3, 13, 15, 26, and/or 28; or a fragment containing the polymorphic allele, wherein said fragment comprises at least 10 contiguous nucleotides of SEQ ID NO:1, 3, 13, 15, 26, and/or 28.

Preferably, the present invention is directed to a polynucleotide comprising, or alternatively consisting of, the sequence identified as SEQ ID NO:1, 3, 13, 15, 26, and/or 28, that is less than, or equal to, a polynucleotide sequence that is 5 mega basepairs, 1 mega basepairs, 0.5 mega basepairs, 0.1 mega basepairs, 50,000 basepairs, 20,000 basepairs, or 10,000 basepairs in length.

The present invention encompasses polynucleotides with sequences complementary to those of the polynucleotides of the present invention disclosed herein. Such sequences may be complementary to the sequence disclosed as SEQ ID NO:1, 3, 13, 15, 26, and/or 28, and/or the nucleic acid sequence encoding the sequences disclosed as SEQ ID NO:2, 4, 14, 16, 27, and/or 29.

The invention encompasses the application of PCR methodology to the polynucleotide sequences of the present invention, and/or the cDNA encoding the polypeptides of the present invention. PCR techniques for the amplification of nucleic acids are described in U.S. Pat. No. 4,683,195 and Saiki et al., Science, 239:487-491 (1988). PCR, for example, may include the following steps, of denaturation of template nucleic acid (if double-stranded), annealing of primer to target, and polymerization. The nucleic acid probed or used as a template in the amplification reaction may be genomic DNA, cDNA, RNA, or a PNA. PCR may be used to amplify specific sequences from genomic DNA, specific RNA sequence, and/or cDNA transcribed from mRNA. References for the general use of PCR techniques, including specific method parameters, include Mullis et al., Cold Spring Harbor Symp. Quant. Biol., 51:263, (1987), Ehrlich (ed), PCR Technology, Stockton Press, NY, 1989; Ehrlich et al., Science, 252:1643-1650, (1991); and “PCR Protocols, A Guide to Methods and Applications”, Eds., Innis et al., Academic Press, New York, (1990).

### Polynucleotide Variants

The present invention also encompasses variants (e.g., allelic variants, orthologs, etc.) of the polynucleotide sequence disclosed herein in SEQ ID NO:1, 3, 13, 15, 26, and/or 28, and the complementary strand thereto.

The present invention also encompasses variants of the polypeptide sequence, and/or fragments therein, disclosed in SEQ ID NO:2, 4, 14, 16, 27, and/or 29, a polypeptide encoded by the polynucleotide sequence in SEQ ID NO:1, 3, 13, 15, 26, and/or 28.

“Variant” refers to a polynucleotide or polypeptide differing from the polynucleotide or polypeptide of the present invention, but retaining essential properties thereof. Generally, variants are overall closely similar, and, in many regions, identical to the polynucleotide or polypeptide of the present invention.

In another embodiment, the invention encompasses nucleic acid molecules which comprise, or alternatively, consist of a polynucleotide which hybridizes under stringent conditions, or alternatively, under lower stringency conditions, to a polynucleotide in (a), (b), (c), or (d), above. Polynucleotides which hybridize to the complement of these nucleic acid molecules under stringent hybridization conditions or alternatively, under lower stringency conditions, are also encompassed by the invention, as are polypeptides encoded by these polypeptides.

### Polynucleotide Fragments

The present invention is directed to polynucleotide fragments of the polynucleotides of the invention, and polynucleotide sequences that hybridize thereto.

In the present invention, a “polynucleotide fragment” refers to a short polynucleotide having a nucleic acid sequence which: is a portion of that shown in SEQ ID NO:1, 3, 13, 15, 26, and/or 28 or the complementary strand thereto, or is a portion of a polynucleotide sequence encoding the polypeptide of SEQ ID NO:2, 4, 14, 16, 27, and/or 29. The nucleotide fragments of the invention are preferably at least about 15 nt, and more preferably at least about 20 nt, still more preferably at least about 30 nt, and even more preferably, at least about 40 nt, at least about 50 nt, at least about 75 nt, or at least about 150 nt in length, and comprise at least one polymorphic locus. A fragment “at least 20 nt in length,” for example, is intended to include 20 or more contiguous bases from the cDNA sequence shown in SEQ ID NO: 1, 3, 13, 15, 26, and/or 28. In this context “about” includes the particularly recited value, a value larger or smaller by several (5, 4, 3, 2, or 1) nucleotides, at either terminus, or at both termini. These nucleotide fragments have uses that include, but are not limited to, as diagnostic probes and primers as discussed herein. Of course, larger fragments (e.g., 50, 150, 500, 600, 2000 nucleotides) are preferred.

Moreover, representative examples of polynucleotide fragments of the invention, include, for example, isolated fragments comprising, or alternatively consisting of, a sequence from about nucleotide number 1-50, 51-100, 101-150, 151 -200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001 to the end of SEQ ID NO:1, 3, 13, 15, 26, and/or 28, or the complementary strand thereto. In this context “about” includes the particularly recited ranges, and ranges larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein. Also encompassed by the present invention are polynucleotides which hybridize to these nucleic acid molecules under stringent hybridization conditions or lower stringency conditions, as are the polypeptides encoded by these polynucleotides.

In the present invention, a “polypeptide fragment” refers to an amino acid sequence which is a portion of that contained in SEQ ID NOs:2, 4, 14, 16, 27, and/or 29. Protein (polypeptide) fragments may be “free-standing,” or comprised within a larger polypeptide of which the fragment forms a part or region, most preferably as a single continuous region. Representative examples of polypeptide fragments of the invention, include, for example, fragments comprising, or alternatively consisting of, from about amino acid number 1-20, 21-40, 41-60, 61-80, 81-100, 102-120, 121-140, 141 -160, or 161 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges or values, and ranges or values larger or smaller by several (5, 4, 3, 2, or 1) amino acids, at either extreme or at both extremes. Polynucleotides encoding these polypeptides are also encompassed by the invention.

Preferred polypeptide fragments include the full-length protein. Further preferred polypeptide fragments include the full-length protein having a continuous series of deleted residues from the amino or the carboxy terminus, or both. For example, any number of amino acids, ranging from 1-60, can be deleted from the amino terminus of the full-length polypeptide. Similarly, any number of amino acids, ranging from 1-30, can be deleted from the carboxy terminus of the full-length protein. Furthermore, any combination of the above amino and carboxy terminus deletions are preferred. Similarly, polynucleotides encoding these polypeptide fragments are also preferred.

Also preferred are polypeptide and polynucleotide fragments characterized by structural or functional domains, such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of SEQ ID NOs:2, 4, 14, 16, 27, and/or 29 falling within conserved domains are specifically contemplated by the present invention. Moreover, polynucleotides encoding these domains are also contemplated.

Other preferred polypeptide fragments are biologically active fragments. Biologically active fragments are those exhibiting activity similar, but not necessarily identical, to an activity of the polypeptide of the present invention. The biological activity of the fragments may include an improved desired activity, or a decreased undesirable activity. Polynucleotides encoding these polypeptide fragments are also encompassed by the invention.

In a preferred embodiment, the functional activity displayed by a polypeptide encoded by a polynucleotide fragment of the invention may be one or more biological activities typically associated with the full-length polypeptide of the invention. Illustrative of these biological activities includes the fragments ability to bind to at least one of the same antibodies which bind to the full-length protein, the fragments ability to interact with at lease one of the same proteins which bind to the full-length, the fragments ability to elicit at least one of the same immune responses as the full-length protein (i.e., to cause the immune system to create antibodies specific to the same epitope, etc.), the fragments ability to bind to at least one of the same polynucleotides as the full-length protein, the fragments ability to bind to a receptor of the full-length protein, the fragments ability to bind to a ligand of the full-length protein, and the fragments ability to multimerize with the full-length protein. However, the skilled artisan would appreciate that some fragments may have biological activities which are desirable and directly inapposite to the biological activity of the full-length protein. The functional activity of polypeptides of the invention, including fragments, variants, derivatives, and analogs thereof can be determined by numerous methods available to the skilled artisan, some of which are described elsewhere herein.

The present invention encompasses polypeptides comprising, or alternatively consisting of, an epitope of the polypeptide having an amino acid sequence of SEQ ID NOs:2, 4, 14, 16, 27, and/or 29, or encoded by a polynucleotide that hybridizes to the complement of the sequence of SEQ ID NOs:1, 3, 13, 15, 26, and/or 28 under stringent hybridization conditions or lower stringency hybridization conditions as defined supra. The present invention further encompasses polynucleotide sequences encoding an epitope of a polypeptide sequence of the invention (such as, for example, the sequence disclosed in SEQ ID NO:1, 3, 13, 15, 26, and/or 28), polynucleotide sequences of the complementary strand of a polynucleotide sequence encoding an epitope of the invention, and polynucleotide sequences which hybridize to the complementary strand under stringent hybridization conditions or lower stringency hybridization conditions defined supra.

The term “epitopes,” as used herein, refers to portions of a polypeptide having antigenic or immunogenic activity in an animal, preferably a mammal, and most preferably in a human. In a preferred embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defmed as a portion of a protein to which an antibody can immunospecifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic.

Fragments which function as epitopes may be produced by any conventional means. (See, e.g., Houghten, Proc. Natl. Acad. Sci. USA 82:5131-5135 (1985), further described in U.S. Pat. No. 4,631,211).

In the present invention, antigenic epitopes preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length, or longer. Additional non-exclusive preferred antigenic epitopes include the antigenic epitopes disclosed herein, as well as portions thereof. Antigenic epitopes are useful, for example, to raise antibodies, including monoclonal antibodies, that specifically bind the epitope. Preferred antigenic epitopes include the antigenic epitopes disclosed herein, as well as any combination of two, three, four, five or more of these antigenic epitopes. Antigenic epitopes can be used as the target molecules in immunoassays. (See, for instance, Wilson et al., Cell 37:767-778 (1984); Sutcliffe et al., Science 219:660-666 (1983)).

Similarly, immunogenic epitopes can be used, for example, to induce antibodies according to methods well known in the art. (See, for instance, Sutcliffe et al., supra; Wilson et al., supra; Chow et al., Proc. Natl. Acad. Sci. USA 82:910-914; and Bittle et al., J. Gen. Virol. 66:2347-2354 (1985). Preferred immunogenic epitopes include the immunogenic epitopes disclosed herein, as well as any combination of two, three, four, five or more of these immunogenic epitopes. The polypeptides comprising one or more immunogenic epitopes may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Epitope-bearing polypeptides of the present invention may be used to induce antibodies according to methods well known in the art including, but not limited to, in vivo immunization, in vitro immunization, and phage display methods. See, e.g., Sutcliffe et al., supra; Wilson et al., supra, and Bittle et al., J. Gen. Virol., 66:2347-2354 (1985). If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μg of peptide or carrier protein and Freund's adjuvant or any other adjuvant known for stimulating an immune response. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and as discussed above, the polypeptides of the present invention comprising an immunogenic or antigenic epitope can be fused to other polypeptide sequences. For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni2+ nitriloacetic acid-agarose column and histidine-tagged proteins can be selectively eluted with imidazole-containing buffers.

### Antibodies

Further polypeptides of the invention relate to antibodies and T-cell antigen receptors (TCR) which immunospecifically bind a polypeptide, polypeptide fragment, or variant of SEQ ID NOs:2, 4, 14, 16, 27, and/or 29, and/or an epitope, of the present invention (as determined by immunoassays well known in the art for assaying specific antibody-antigen binding). Antibodies of the invention include, but are not limited to, polyclonal, monoclonal, monovalent, bispecific, heteroconjugate, multispecific, human, humanized or chimeric antibodies, single chain antibodies, Fab fragments, F (ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Moreover, the term “antibody” (Ab) or “monoclonal antibody” (Mab) is meant to include intact molecules, as well as, antibody fragments (such as, for example, Fab and F (ab′)2 fragments) which are capable of specifically binding to protein. Fab and F (ab′)2 fragments lack the Fc fragment of intact antibody, clear more rapidly from the circulation of the animal or plant, and may have less non-specific tissue binding than an intact antibody (Wahl et al., J. Nucl. Med.. 24:316-325 (1983)). Thus, these fragments are preferred, as well as the products of a FAB or other immunoglobulin expression library. Moreover, antibodies of the present invention include chimeric, single chain, and humanized antibodies.

Most preferably the antibodies are human antigen-binding antibody fragments of the present invention and include, but are not limited to, Fab, Fab′ and F (ab′)2, Fd, single-chain Fvs (scFv), single-chain antibodies, disulfide-linked Fvs (sdfv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, by size in contiguous amino acid residues, or listed in the Tables and Figures. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homologue of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologues of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinity to a polypeptide of the invention. Preferred binding affinities include those with a dissociation constant or Kd less than 5×10-2 M, 10-2 M, 5×10-3 M, 10-3 M, 5×10-4 M, 10-4 M, 5×10-5 M, 10-5 M, 5×10-6 M, 10-6M, 5×10-7 M, 107 M, 5×10-8 M, 10-8 M, 5×10-9 M, 10-9 M, 5×10-10 M, 10-10 M, 5×10-11 M, 10-11 M, 5×10-12 M, 10-12 M, 5×10-13 M, 10-13 M, 5×10-14 M, 10-14 M, 5×10-15 M, or 10-15 M.

The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85 %, at least 80%, at least 75%, at least 70%, at least 60%, or at least 50%.

Antibodies of the present invention may act as agonists or antagonists of the polypeptides of the present invention. Preferably the antibodies of the present invention are specific for a single nucleotide polymorphism of any one of the angiocongestive heart failure candidate gene polypeptides of the present invention. More preferred are antibodies that are capable of specifically distinguishing between the variant and reference forms of a polypeptide of the present invention. Such antibodies are primarily useful in a kit to identify variant or normal forms of a polypeptide, and hence determining whether a particular individual is at a higher or lower risk of being susceptible to congestive heart failure or a congestive heart failure-like disorder upon the administration of a PPAR-agonist.

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988) (incorporated by reference herein in its entirety).

As discussed in more detail below, the antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionucleotides, or toxins. See, e.g., PCT publications WO 92/08495; WO 91/14438; WO 89/12624; U.S. Pat. No. 5,314,995; and EP 396,387.

The antibodies of the invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art.

The antibodies of the present invention may comprise polyclonal antibodies. Methods of preparing polyclonal antibodies are known to the skilled artisan (Harlow, et al., Antibodies: A Laboratory Manual, (Cold spring Harbor Laboratory Press, 2^{nd} ed. (1988), which is hereby incorporated herein by reference in its entirety). For example, a polypeptide of the invention can be administered to various host animals including, but not limited to, rabbits, mice, rats, etc. to induce the production of sera containing polyclonal antibodies specific for the antigen. The administration of the polypeptides of the present invention may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art. For the purposes of the invention, “immunizing agent” may be defmed as a polypeptide of the invention, including fragments, variants, and/or derivatives thereof, in addition to fusions with heterologous polypeptides and other forms of the polypeptides described herein.

Typically, the immunizing agent and/or adjuvant will be injected in the mammal by multiple subcutaneous or intraperitoneal injections, though they may also be given intramuscularly, and/or through IV). The immunizing agent may include polypeptides of the present invention or a fusion protein or variants thereof. Depending upon the nature of the polypeptides (i.e., percent hydrophobicity, percent hydrophilicity, stability, net charge, isoelectric point etc.), it may be useful to conjugate the immunizing agent to a protein known to be immunogenic in the mammal being immunized. Such conjugation includes either chemical conjugation by derivitizing active chemical functional groups to both the polypeptide of the present invention and the immunogenic protein such that a covalent bond is formed, or through fusion-protein based methodology, or other methods known to the skilled artisan. Examples of such immunogenic proteins include, but are not limited to keyhole limpet hemocyanin, serum albumin, bovine thyroglobulin, and soybean trypsin inhibitor. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Additional examples of adjuvants which may be employed includes the MPL-TDM adjuvant (monophosphoryl lipid A, synthetic trehalose dicorynomycolate). The immunization protocol may be selected by one skilled in the art without undue experimentation.

The antibodies of the present invention may comprise monoclonal antibodies. Monoclonal antibodies may be prepared using hybridoma methods, such as those described by Kohler and Milstein, Nature, 256:495 (1975) and U.S. Pat. No. 4,376,110, by Harlow, et al., Antibodies: A Laboratory Manual, (Cold spring Harbor Laboratory Press, 2^{nd} ed. (1988), by Hammerling, et al., Monoclonal Antibodies and T-Cell Hybridomas (Elsevier, N.Y., (1981)), or other methods known to the artisan. Other examples of methods which may be employed for producing monoclonal antibodies includes, but are not limited to, the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mabs in vivo makes this the presently preferred method of production.

In a hybridoma method, a mouse, a humanized mouse, a mouse with a human immune system, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include polypeptides of the present invention or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986), pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. As inferred throughout the specification, human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the polypeptides of the present invention. Preferably, the binding specificity of monoclonal antibodies produced by the hybridoma cells is determined by immunoprecipitation or by an in vitro binding assay, such as radioimmunoassay (RIA) or enzyme-linked immunoadsorbant assay (ELISA). Such techniques are known in the art and within the skill of the artisan. The binding affinity of the monoclonal antibody can, for example, be determined by the Scatchard analysis of Munson and Pollart, Anal. Biochem., 107:220 (1980).

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-sepharose, hydroxyapatite chromatography, gel exclusion chromatography, gel electrophoresis, dialysis, or affinity chromatography.

The skilled artisan would acknowledge that a variety of methods exist in the art for the production of monoclonal antibodies and thus, the invention is not limited to their sole production in hydridomas. For example, the monoclonal antibodies may be made by recombinant DNA methods, such as those described in U.S. Pat. No. 4,816,567. In this context, the term “monoclonal antibody” refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to genes encoding the heavy and light chains of murine antibodies, or such chains from human, humanized, or other sources). The hydridoma cells of the invention serve as a preferred source of such DNA. Once isolated, the DNA may be placed into expression vectors, which are then transformed into host cells such as Simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al, supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art and are discussed in detail in the Examples herein. In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

Antibody fragments which recognize specific epitopes may be generated by known techniques. For example, Fab and F (ab′)2 fragments of the invention may be produced by proteolytic cleavage of immunoglobulin molecules, using enzymes such as papain (to produce Fab fragments) or pepsin (to produce F (ab′)2 fragments). F (ab′)2 fragments contain the variable region, the light chain constant region and the CH1 domain of the heavy chain.

For example, the antibodies of the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F (ab′)2 fragments can also be employed using methods known in the art such as those disclosed in PCT publication WO 92/22324; Mullinax et al., BioTechniques 12 (6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816397, which are incorporated herein by reference in their entirety. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28 (4/5):489-498 (1991); Studnicka et al., Protein Engineering 7 (6):805-814 (1994); Roguska. et al., PNAS 91:969-973 (1994)), and chain shuffling (U.S. Pat. No. 5,565,332). Generally, a humanized antibody has one or more amino acid residues introduced into it from a source that is non-human. These non-human amino acid residues are often referred to as “import” residues, which are typically taken from an “import” variable domain. Humanization can be essentially performed following the methods of Winter and co-workers (Jones et al., Nature, 321:522-525 (1986); Reichmann et al., Nature, 332:323-327 (1988); Verhoeyen et al., Science, 239:1534-1536 (1988), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possible some FR residues are substituted from analogous sites in rodent antibodies.

In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature 332:323-329 (1988) and Presta, Curr. Op. Struct. Biol., 2:593-596 (1992).

Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. The techniques of cole et al., and Boerder et al., are also available for the preparation of human monoclonal antibodies (cole et al., Monoclonal Antibodies and Cancer Therapy, Alan R. Riss, (1985); and Boerner et al., J. Immunol., 147 (1):86-95, (1991)).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.), Genpharm (San Jose, Calif.), and Medarex, Inc. (Princeton, N.J.) can be engaged to provide human antibodies directed against a selected antigen using technology similar to that described above.

Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and creation of an antibody repertoire. This approach is described, for example, in U.S. Pat. Nos. 5,545,807; 5,545,806; 5,569,825; 5,625,126; 5,633,425; 5,661,106, and in the following scientific publications: Marks et al., Biotechnol., 10:779-783 (1992); Lonberg et al., Nature 368:856-859 (1994); Fishwild et al., Nature Biotechnol., 14:845-51 (1996); Neuberger, Nature Biotechnol., 14:826 (1996); Lonberg and Huszer, Intern. Rev. Immunol., 13:65-93 (1995).

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, antibodies to the polypeptides of the invention can, in turn, be utilized to generate anti-idiotype antibodies that “mimic” polypeptides of the invention using techniques well known to those skilled in the art. (See, e.g., Greenspan & Bona, FASEB J. 7 (5):437-444; (1989) and Nissinoff, J. Immunol. 147 (8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

The antibodies of the present invention may be bispecific antibodies. Bispecific antibodies are monoclonal, preferably human or humanized, antibodies that have binding specificities for at least two different antigens. In the present invention, one of the binding specificities may be directed towards a polypeptide of the present invention, the other may be for any other antigen, and preferably for a cell-surface protein, receptor, receptor subunit, tissue-specific antigen, virally derived protein, virally encoded envelope protein, bacterially derived protein, or bacterial surface protein, etc.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transformed into a suitable host organism. For further details of generating bispecific antibodies see, for example Suresh et al., Meth. In Enzym., 121:210 (1986).

Heteroconjugate antibodies are also contemplated by the present invention. Heteroconjugate antibodies are composed of two covalently joined antibodies. Such antibodies have, for example, been proposed to target immune system cells to unwanted cells (U.S. Pat. No. 4,676,980), and for the treatment of HIV infection (WO 91/00360; WO 92/20373; and EP03089). It is contemplated that the antibodies may be prepared in vitro using known methods in synthetic protein chemistry, including those involving crosslinking agents. For example, immunotoxins may be constructed using a disulfide exchange reaction or by forming a thioester bond. Examples of suitable reagents for this purpose include iminothiolate and methyl-4-mercaptobutyrimidate and those disclosed, for example, in U.S. Pat. No. 4,676,980.

### Methods of Producing Antibodies

The antibodies of the invention can be produced by any method known in the art for the synthesis of antibodies, in particular, by chemical synthesis or preferably, by recombinant expression techniques.

Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt- cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, 1993, TIB TECH 11 (5):155-215); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalently and non-covalently conjugations) to a polypeptide (or portion thereof, preferably at least 10, 20, 30, 40, 50, 60, 70, 80, 90 or 100 amino acids of the polypeptide) of the present invention to generate fusion proteins. The fusion does not necessarily need to be direct, but may occur through linker sequences. The antibodies may be specific for antigens other than polypeptides (or portion thereof, preferably at least 10, 20, 30, 40, 50, 60, 70, 80, 90 or 100 amino acids of the polypeptide) of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in vitro immunoassays and purification methods using methods known in the art. See e.g., Harbor et al., supra, and PCT publication WO 93/21232; EP 439,095; Naramura et al., Immunol. Lett. 39:91-99 (1994); U.S. Pat. No. 5,474,981; Gillies et al., PNAS 89:1428-1432 (1992); Fell et al., J. Immunol. 146:2446-2452 (1991), which are incorporated by reference in their entireties.

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the constant region, hinge region, CH1 domain, CH2 domain, and CH3 domain or any combination of whole domains or portions thereof. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88:10535-10539 (1991); Zheng et al., J. Immunol. 154:5590-5600 (1995); and Vil et al., Proc. Natl. Acad. Sci. USA 89:11337-11341 (1992) (said references incorporated by reference in their entireties).

As discussed, supra, the polypeptides corresponding to a polypeptide, polypeptide fragment, or a variant of SEQ ID NOs:2, 4, 14, 16, 27, and/or 29 may be fused or conjugated to the above antibody portions to increase the in vivo half life of the polypeptides or for use in immunoassays using methods known in the art. Further, the polypeptides corresponding to SEQ ID NOs:2, 4, 14, 16, 27, and/or 29 may be fused or conjugated to the above antibody portions to facilitate purification. One reported example describes chimeric proteins consisting of the first two domains of the human CD4-polypeptide and various domains of the constant regions of the heavy or light chains of mammalian immunoglobulins. (EP 394,827; Traunecker et al., Nature 331:84-86 (1988). The polypeptides of the present invention fused or conjugated to an antibody having disulfide-linked dimeric structures (due to the IgG) may also be more efficient in binding and neutralizing other molecules, than the monomeric secreted protein or protein fragment alone. (Fountoulakis et al., J. Biochem. 270:3958-3964 (1995)). In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, Bennett et al., J. Molecular Recognition 8:52-58 (1995); Johanson et al., J. Biol. Chem. 270:9459-9471 (1995).

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

The present invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidinibiotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, 111In or 99Tc.

Further, an antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980, which is incorporated herein by reference in its entirety.

An antibody, with or without a therapeutic moiety conjugated to it, administered alone or in combination with cytotoxic factor(s) and/or cytokine(s) can be used as a therapeutic.

### Uses for Antibodies Directed Against Polypeptides of the Invention

The antibodies of the present invention have various utilities. For example, such antibodies may be used in diagnostic assays to detect the presence or quantification of a variant or reference form of a polypeptides of the invention in a sample. Such a diagnostic assay may be comprised of at least two steps. The first, subjecting a sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278 (2):123-131 (2000)), or a chromatography column, etc. And a second step involving the quantification of antibody bound to the substrate. Alternatively, the method may additionally involve a first step of attaching the antibody, either covalently, electrostatically, or reversibly, to a solid support, and a second step of subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); Dafvid et al., Biochem., 13:1014 (1974); Pain et al., J. Immunol. Metho., 40:219 (1981); and Nygren, J. Histochem. And Cytochem., 30:407 (1982).

### Assays for Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors. (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%- 20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I).diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 125I) in the presence of increasing amounts of an unlabeled second antibody.

### Therapeutic/Prophylactic Administration and Compositions

The invention provides methods of treatment, inhibition and prophylaxis by administration to a subject of an effective amount of a compound or pharmaceutical composition of the invention, including an antibody, antisense reagent, RNAi reagent, and/or a zinc-finger protein of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem.. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Diagnosis and Imaging with Antibodies

Labeled antibodies, and derivatives and analogs thereof, which specifically bind to a variant or reference allele of a polypeptide of interest can be used for diagnostic purposes to detect, diagnose, or monitor diseases, disorders, and/or conditions associated with the aberrant expression and/or activity of a polypeptide of the invention. The invention provides for the detection of aberrant expression of a polypeptide of interest, comprising: (a) assaying the expression of the polypeptide of interest in cells or body fluid of an individual using one or more antibodies specific to the polypeptide interest; and (b) comparing the level of gene expression with a standard gene expression level, whereby an increase or decrease in the assayed polypeptide gene expression level compared to the standard expression level is indicative of aberrant expression.

The invention provides a diagnostic assay for diagnosing a disorder, comprising: (a) assaying the expression of the polypeptide of interest in cells or body fluid of an individual using one or more antibodies specific to the polypeptide interest; and (b) comparing the level of gene expression with a standard gene expression level, whereby an increase or decrease in the assayed polypeptide gene expression level compared to the standard expression level is indicative of a particular disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121), carbon (14C), sulfur (35S), tritium (3H), indium (112In), and technetium (99Tc); luminescent labels, such as luminol; and fluorescent labels, such as fluorescein and rhodamine, and biotin.

One aspect of the invention is the detection and diagnosis of a disease or disorder associated with aberrant expression of a polypeptide of interest in an animal, preferably a mammal and most preferably a human. In one embodiment, diagnosis comprises: (a) administering (for example, parenterally, subcutaneously, or intraperitoneally) to a subject an effective amount of a labeled molecule which specifically binds to the polypeptide of interest; (b) waiting for a time interval following the administering for permitting the labeled molecule to preferentially concentrate at sites in the subject where the polypeptide is expressed (and for unbound labeled molecule to be cleared to background level); (c) determining background level; and (d) detecting the labeled molecule in the subject, such that detection of labeled molecule above the background level indicates that the subject has a particular disease or disorder associated with aberrant expression of the polypeptide of interest. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

### Kits

The invention further provides kits comprising at least one agent for identifying which alleleic form of the SNPs identified herein is present in a sample. For example, suitable kits can comprise at least one antibody specific for a particular protein or peptide encoded by one alleleic form of the gene, or allele-specific oligonucleotide as described herein. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least 1, 10, 100 or all of the polymorphisms shown in Table I. Optional additional components of the kit include, for example, reagents, buffers, restriction enzymes, reverse-transcriptase or polymerase, the substrate nucleoside triphosphates, means used to label (for example, an avidin-enzyme conjugate and enzyme substrate and chromogen if the label is biotin, fluophores, and others as described herein), and the appropriate buffers for reverse transcription, PCR, or hybridization reactions. Usually, the kit also contains instructions for carrying out the methods.

The present invention provides kits that can be used in the methods described herein. In one embodiment, a kit comprises a single primer or probe of the invention comprising at least one polymorphic locus, preferably a purified primer or probe, in one or more containers. Such a primer or probe may further comprise a detectable label such as a fluorescent compound, an enzymatic substrate, a radioactive compound, a luminescent compound, a fluorophore, and/or a fluorophore linked to a terminator contained therein. Such a kit may further comprise reagents required to enable adequate hybridization of said single primer or probe to a DNA test sample, such that under suitable conditions, the primer or probe is capable of binding to said DNA test sample and signaling whether the variant or reference allele at the polymorphic locus is present in said DNA test sample.

In one example, the kit comprises a method for detecting the presence of a polymorphic locus comprising one specific allele of at least one polynucleotide in a DNA test sample which serves as a template nucleic acid comprising: (a) forming an oligonucleotide bound to the polymorphic locus wherein the oligonucleotide comprises a fluorophore linked to a terminator contained therein; and (b) detecting fluorescence polarization of the fluorophore of the fluorescently-labeled oligonucleotide, wherein the oligonucleotide is formed from a primer bound to said DNA sample immediately 3′ to the polymorphic locus and a terminator covalently linked to a fluorophore, and wherein said terminator-linked fluorophore binds to the polymorphic locus and reacts with the primer to produce an extended primer which is said fluorescently labeled oligonucleotide, wherein an increase in fluorescence polarization indicates the presence of the specific allele at the polymorphic locus, thereby detecting the presence of the specific allele at the polymorphic locus by said increase in fluorescence polarization.

The kit of the present invention may comprise the following non-limiting examples of flurophores linked to a primer or probe of the present invention: 5-carboxyfluorescein (FAM-ddNTPs); 6-carboxy-X-rhodamine (ROX-ddNTPs); N,N,N′,N′-tetramethyl-6-carboxyrhodamine (TMR-ddNTPs); and BODIPY-Texas Red (BTR-ddNTPs).

In a specific embodiment, the kits of the present invention contain a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

The present invention provides kits that can be used in the above methods. In one embodiment, a kit comprises an antibody of the invention, preferably a purified antibody, in one or more containers. In a specific embodiment, the kits of the present invention contain a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another specific embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In a more specific embodiment the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or calorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface- bound recombinant antigens, and a reporter-labeled anti-human antibody for detecting surface-bound anti-antigen antibody.

### Vectors, Host Cells, and Protein Production

The present invention also relates to vectors containing the polynucleotide of the present invention, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PAO815 (all available from Invitrogen, Carlsbad, Calif.). Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

A polypeptide of this invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides of the present invention, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In one embodiment, the yeast Pichia pastoris is used to express the polypeptide of the present invention in a eukaryotic system. Pichia pastoris is a methylotrophic yeast which can metabolize methanol as its sole carbon source. A main step in the methanol metabolization pathway is the oxidation of methanol to formaldehyde using O2. This reaction is catalyzed by the enzyme alcohol oxidase. In order to metabolize methanol as its sole carbon source, Pichia pastoris must generate high levels of alcohol oxidase due, in part, to the relatively low affinity of alcohol oxidase for O2. Consequently, in a growth medium depending on methanol as a main carbon source, the promoter region of one of the two alcohol oxidase genes (AOX1) is highly active. In the presence of methanol, alcohol oxidase produced from the AOX1 gene comprises up to approximately 30% of the total soluble protein in Pichia pastoris. See, Ellis, S. B., et al., Mol. Cell. Biol. 5:1111-21 (1985); Koutz, P. J, et al., Yeast 5:167-77 (1989); Tschopp, J. F., et al., Nucl. Acids Res. 15:3859-76 (1987). Thus, a heterologous coding sequence, such as, for example, a polynucleotide of the present invention, under the transcriptional regulation of all or part of the AOX1 regulatory sequence is expressed at exceptionally high levels in Pichia yeast grown in the presence of methanol.

In one example, the plasmid vector pPIC9K is used to express DNA encoding a polypeptide of the invention, as set forth herein, in a Pichea yeast system essentially as described in “Pichia Protocols: Methods in Molecular Biology,” D. R. Higgins and J. Cregg, eds. The Humana Press, Totowa, N.J., 1998. This expression vector allows expression and secretion of a protein of the invention by virtue of the strong AOX1 promoter linked to the Pichia pastoris alkaline phosphatase (PHO) secretory signal peptide (i.e., leader) located upstream of a multiple cloning site.

Many other yeast vectors could be used in place of pPIC9K, such as, pYES2, pYD1 , pTEF1/Zeo, pYES2/GS, pPICZ, pGAPZ, pGAPZalpha, pPIC9, pPIC3.5, pHIL-D2, pHIL-S1, pPIC3.5K, and PA0815, as one skilled in the art would readily appreciate, as long as the proposed expression construct provides appropriately located signals for transcription, translation, secretion (if desired), and the like, including an in-frame AUG, as required.

In another embodiment, high-level expression of a heterologous coding sequence, such as, for example, a polynucleotide of the present invention, may be achieved by cloning the heterologous polynucleotide of the invention into an expression vector such as, for example, pGAPZ or pGAPZalpha, and growing the yeast culture in the absence of methanol.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., coding sequence), and/or to include genetic material (e.g., heterologous polynucleotide sequences) that is operably associated with the polynucleotides of the invention, and which activates, alters, and/or amplifies endogenous polynucleotides. For example, techniques known in the art may be used to operably associate heterologous control regions (e.g., promoter and/or enhancer) and endogenous polynucleotide sequences via homologous recombination, resulting in the formation of a new transcription unit (see, e.g., U.S. Pat. No. 5,641,670, issued Jun. 24, 1997; U.S. Pat. No. 5,733,761, issued Mar. 31, 1998; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

In addition, polypeptides of the invention can be chemically synthesized using techniques known in the art (e.g., see Creighton, 1983, Proteins: Structures and Molecular Principles, W.H. Freeman & Co., N.Y., and Hunkapiller et al., Nature, 310:105-111 (1984)). For example, a polypeptide corresponding to a fragment of a polypeptide sequence of the invention can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention encompasses polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptides may also be modified with a detectable label, such as an enzymatic, fluorescent, isotopic or affinity label to allow for detection and isolation of the protein, the addition of epitope tagged peptide fragments (e.g., FLAG, HA, GST, thioredoxin, maltose binding protein, etc.), attachment of affinity tags such as biotin and/or streptavidin, the covalent attachment of chemical moieties to the amino acid backbone, N- or C-terminal processing of the polypeptides ends (e.g., proteolytic processing), deletion of the N-terminal methionine residue, etc.

Also provided by the invention are chemically modified derivatives of the polypeptides of the invention which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The invention further encompasses chemical derivitization of the polypeptides of the present invention, preferably where the chemical is a hydrophilic polymer residue. Exemplary hydrophilic polymers, including derivatives, may be those that include polymers in which the repeating units contain one or more hydroxy groups (polyhydroxy polymers), including, for example, poly (vinyl alcohol); polymers in which the repeating units contain one or more amino groups (polyamine polymers), including, for example, peptides, polypeptides, proteins and lipoproteins, such as albumin and natural lipoproteins; polymers in which the repeating units contain one or more carboxy groups (polycarboxy polymers), including, for example, carboxymethylcellulose, alginic acid and salts thereof, such as sodium and calcium alginate, glycosaminoglycans and salts thereof, including salts of hyaluronic acid, phosphorylated and sulfonated derivatives of carbohydrates, genetic material, such as interleukin-2 and interferon, and phosphorothioate oligomers; and polymers in which the repeating units contain one or more saccharide moieties (polysaccharide polymers), including, for example, carbohydrates.

The molecular weight of the hydrophilic polymers may vary, and is generally about 50 to about 5,000,000, with polymers having a molecular weight of about 100 to about 50,000 being preferred. The polymers may be branched or unbranched. More preferred polymers have a molecular weight of about 150 to about 10,000, with molecular weights of 200 to about 8,000 being even more preferred.

For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

Additional preferred polymers which may be used to derivatize polypeptides of the invention, include, for example, poly (ethylene glycol) (PEG), poly (vinylpyrrolidine), polyoxomers, polysorbate and poly (vinyl alcohol), with PEG polymers being particularly preferred. Preferred among the PEG polymers are PEG polymers having a molecular weight of from about 100 to about 10,000. More preferably, the PEG polymers have a molecular weight of from about 200 to about 8,000, with PEG 2,000, PEG 5,000 and PEG 8,000, which have molecular weights of 2,000, 5,000 and 8,000, respectively, being even more preferred. Other suitable hydrophilic polymers, in addition to those exemplified above, will be readily apparent to one skilled in the art based on the present disclosure. Generally, the polymers used may include polymers that can be attached to the polypeptides of the invention via alkylation or acylation reactions.

The polyethylene glycol molecules (or other chemical moieties) should be attached to the protein with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminus) available for derivatization in a particular protein. Under the appropriate reaction conditions, substantially selective derivatization of the protein at the N-terminus with a carbonyl group containing polymer is achieved.

As with the various polymers exemplified above, it is contemplated that the polymeric residues may contain functional groups in addition, for example, to those typically involved in linking the polymeric residues to the polypeptides of the present invention. Such functionalities include, for example, carboxyl, amine, hydroxy and thiol groups. These functional groups on the polymeric residues can be further reacted, if desired, with materials that are generally reactive with such functional groups and which can assist in targeting specific tissues in the body including, for example, diseased tissue. Exemplary materials which can be reacted with the additional functional groups include, for example, proteins, including antibodies, carbohydrates, peptides, glycopeptides, glycolipids, lectins, and nucleosides.

In addition to residues of hydrophilic polymers, the chemical used to derivatize the polypeptides of the present invention can be a saccharide residue. Exemplary saccharides which can be derived include, for example, monosaccharides or sugar alcohols, such as erythrose, threose, ribose, arabinose, xylose, lyxose, fructose, sorbitol, mannitol and sedoheptulose, with preferred monosaccharides being fructose, mannose, xylose, arabinose, mannitol and sorbitol; and disaccharides, such as lactose, sucrose, maltose and cellobiose. Other saccharides include, for example, inositol and ganglioside head groups. Other suitable saccharides, in addition to those exemplified above, will be readily apparent to one skilled in the art based on the present disclosure. Generally, saccharides which may be used for derivitization include saccharides that can be attached to the polypeptides of the invention via alkylation or acylation reactions.

Moreover, the invention also encompasses derivitization of the polypeptides of the present invention, for example, with lipids (including cationic, anionic, polymerized, charged, synthetic, saturated, unsaturated, and any combination of the above, etc.). stabilizing agents.

The invention encompasses derivitization of the polypeptides of the present invention, for example, with compounds that may serve a stabilizing function (e.g., to increase the polypeptides half-life in solution, to make the polypeptides more water soluble, to increase the polypeptides hydrophilic or hydrophobic character, etc.). Polymers useful as stabilizing materials may be of natural, semi-synthetic (modified natural) or synthetic origin. Exemplary natural polymers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrin, dextrose, glucose, polyglucose, polydextrose, pustulan, chitin, agarose, keratin, chondroitin, dermatan, hyaluronic acid, alginic acid, xanthin gum, starch and various other natural homopolymer or heteropolymers, such as those containing one or more of the following aldoses, ketoses, acids or amines: erythose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof Accordingly, suitable polymers include, for example, proteins, such as albumin, polyalginates, and polylactide-coglycolide polymers. Exemplary semi-synthetic polymers include carboxymethylcellulose, hydroxymethylcellulose, hydroxypropylmethylcellulose, methylcellulose, and methoxycellulose. Exemplary synthetic polymers include polyphosphazenes, hydroxyapatites, fluoroapatite polymers, polyethylenes (such as, for example, polyethylene glycol (including for example, the class of compounds referred to as Pluronics.RTM., commercially available from BASF, Parsippany, N.J.), polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof. Methods for the preparation of derivatized polypeptides of the invention which employ polymers as stabilizing compounds will be readily apparent to one skilled in the art, in view of the present disclosure, when coupled with information known in the art, such as that described and referred to in Unger, U.S. Pat. No. 5,205,290, the disclosure of which is hereby incorporated by reference herein in its entirety.

Moreover, the invention encompasses additional modifications of the polypeptides of the present invention. Such additional modifications are known in the art, and are specifically provided, in addition to methods of derivitization, etc., in U.S. Pat. No. 6,028,066, which is hereby incorporated in its entirety herein.

The polypeptides of the invention may be in monomers or multimers (i.e., dimers, trimers, tetramers and higher multimers). Accordingly, the present invention relates to monomers and multimers of the polypeptides of the invention, their preparation, and compositions (preferably, Therapeutics) containing them. In specific embodiments, the polypeptides of the invention are monomers, dimers, trimers or tetramers. In additional embodiments, the multimers of the invention are at least dimers, at least trimers, or at least tetramers.

Multimers encompassed by the invention may be homomers or heteromers. As used herein, the term homomer, refers to a multimer containing only polypeptides corresponding to the amino acid sequence of SEQ ID NOs:2, 4, 14, 16, 27, and/or 29 (including fragments, variants, splice variants, and fusion proteins, corresponding to these polypeptides as described herein). These homomers may contain polypeptides having identical or different amino acid sequences. In a specific embodiment, a homomer of the invention is a multimer containing only polypeptides having an identical amino acid sequence. In another specific embodiment, a homomer of the invention is a multimer containing polypeptides having different amino acid sequences. In specific embodiments, the multimer of the invention is a homodimer (e.g., containing polypeptides having identical or different amino acid sequences) or a homotrimer (e.g., containing polypeptides having identical and/or different amino acid sequences). In additional embodiments, the homomeric multimer of the invention is at least a homodimer, at least a homotrimer, or at least a homotetramer.

As used herein, the term heteromer refers to a multimer containing one or more heterologous polypeptides (i.e., polypeptides of different proteins) in addition to the polypeptides of the invention. In a specific embodiment, the multimer of the invention is a heterodimer, a heterotrimer, or a heterotetramer. In additional embodiments, the heteromeric multimer of the invention is at least a heterodimer, at least a heterotrimer, or at least a heterotetramer.

Multimers of the invention may be the result of hydrophobic, hydrophilic, ionic and/or covalent associations and/or may be indirectly linked, by for example, liposome formation. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention are formed by covalent associations with and/or between the polypeptides of the invention. Such covalent associations may involve one or more amino acid residues contained in the polypeptide sequence (e.g., that recited in the Sequence Listing). In one instance, the covalent associations are cross-linking between cysteine residues located within the polypeptide sequences which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a fusion protein of the invention.

In one example, covalent associations are between the heterologous sequence contained in a fusion protein of the invention (see, e.g., U.S. Pat. No. 5,478,925). In a specific example, the covalent associations are between the heterologous sequence contained in an Fc fusion protein of the invention (as described herein). In another specific example, covalent associations of fusion proteins of the invention are between heterologous polypeptide sequence from another protein that is capable of forming covalently associated multimers, such as for example, osteoprotegerin (see, e.g., International Publication NO: WO 98/49305, the contents of which are herein incorporated by reference in its entirety). In another embodiment, two or more polypeptides of the invention are joined through peptide linkers. Examples include those peptide linkers described in U.S. Pat. No. 5,073,627 (hereby incorporated by reference). Proteins comprising multiple polypeptides of the invention separated by peptide linkers may be produced using conventional recombinant DNA technology.

Another method for preparing multimer polypeptides of the invention involves use of polypeptides of the invention fused to a leucine zipper or isoleucine zipper polypeptide sequence. Leucine zipper and isoleucine zipper domains are polypeptides that promote multimerization of the proteins in which they are found. Leucine zippers were originally identified in several DNA-binding proteins (Landschulz et al., Science 240:1759, (1988)), and have since been found in a variety of different proteins. Among the known leucine zippers are naturally occurring peptides and derivatives thereof that dimerize or trimerize. Examples of leucine zipper domains suitable for producing soluble multimeric proteins of the invention are those described in PCT application WO 94/10308, hereby incorporated by reference. Recombinant fusion proteins comprising a polypeptide of the invention fused to a polypeptide sequence that dimerizes or trimerizes in solution are expressed in suitable host cells, and the resulting soluble multimeric fusion protein is recovered from the culture supernatant using techniques known in the art.

Trimeric polypeptides of the invention may offer the advantage of enhanced biological activity. Preferred leucine zipper moieties and isoleucine moieties are those that preferentially form trimers. One example is a leucine zipper derived from lung surfactant protein D (SPD), as described in Hoppe et al. (FEBS Letters 344:191, (1994)) and in U.S. patent application Ser. No. 08/446,922, hereby incorporated by reference. Other peptides derived from naturally occurring trimeric proteins may be employed in preparing trimeric polypeptides of the invention.

In another example, proteins of the invention are associated by interactions between Flag® polypeptide sequence contained in fusion proteins of the invention containing Flag® polypeptide sequence. In a further embodiment, associations proteins of the invention are associated by interactions between heterologous polypeptide sequence contained in Flag® fusion proteins of the invention and anti-Flag® antibody.

The multimers of the invention may be generated using chemical techniques known in the art. For example, polypeptides desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers of the invention may be generated using techniques known in the art to form one or more inter-molecule cross-links between the cysteine residues located within the sequence of the polypeptides desired to be contained in the multimer (see, e.g., U.S. Pat. No. 5,478,925, which is herein incorporated by reference in its entirety). Further, polypeptides of the invention may be routinely modified by the addition of cysteine or biotin to the C terminus or N-terminus of the polypeptide and techniques known in the art may be applied to generate multimers containing one or more of these modified polypeptides (see, e.g., U.S. Pat. No. 5,478,925, which is herein incorporated by reference in its entirety). Additionally, techniques known in the art may be applied to generate liposomes containing the polypeptide components desired to be contained in the multimer of the invention (see, e.g., U.S. Pat. No. 5,478,925, which is herein incorporated by reference in its entirety).

Alternatively, multimers of the invention may be generated using genetic engineering techniques known in the art. In one embodiment, polypeptides contained in multimers of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer of the invention are generated by ligating a polynucleotide sequence encoding a polypeptide of the invention to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant polypeptides of the invention which contain a transmembrane domain (or hydrophobic or signal peptide) and which can be incorporated by membrane reconstitution techniques into liposomes (see, e.g., U.S. Pat. No. 5,478,925, which is herein incorporated by reference in its entirety).

In addition, the polynucleotide insert of the present invention could be operatively linked to “artificial” or chimeric promoters and transcription factors. Specifically, the artificial promoter could comprise, or alternatively consist, of any combination of cis-acting DNA sequence elements that are recognized by trans-acting transcription factors. Preferably, the cis acting DNA sequence elements and trans-acting transcription factors are operable in mammals. Further, the trans-acting transcription factors of such “artificial” promoters could also be “artificial” or chimeric in design themselves and could act as activators or repressors to said “artificial” promoter.

### Methods of Use of the Allelic Polynucleotides of the Present Invention

The determination of the polymorphic form(s) present in an individual at one or more polymorphic sites defined herein can be used in a number of methods.

In preferred embodiments, the polynucleotides and polypeptides of the present invention, including allelic and variant forms thereof, have uses which include, but are not limited to diagnosing individuals to identify whether a given individual has increased susceptibility or risk for developing dose-dependent congestive heart failure or congestive heart failure-like disorder using the genotype assays of the present invention. In addition, the polynucleotides and polypeptides of the present invention, including allelic and variant forms thereof, have uses which include, but are not limited to, diagnosing individuals to identify whether a given individual, upon administration of either the prescribed dose, or an increased dose of a PPAR-agonist, has increased susceptibility or risk for developing dose-dependent congestive heart failure or congestive heart failure-like disorder using the genotype assays of the present invention.

In preferred embodiments, the polynucleotides and polypeptides of the present invention, including allelic and variant forms thereof, have uses which include, but are not limited to diagnosing individuals to identify whether a given individual is at a higher risk of developing dose-dependent congestive heart failure. A representative administered dose of the PPAR-agonist Com. A may be 2.5 mg, 5 mg, 10 mg, 15 mg, or 20 mg. An acceptable higher or lower level of a pharmaceutically acceptable dose of a PPAR-agonist for a patient identified as being at low risk of developing dose-dependent congestive heart failure may be about 5%, 10%, 15%, 20%, 25%, 30%, 35%, 40%, 45%, 50%, 55%, 60%, 65%, 75%, 80%, 85%, 90%, or 95% higher or lower than the prescribed or typical dose, as may be the case.

In another preferred embodiment, the polynucleotides and polypeptides of the present invention, including allelic and variant forms thereof, have uses which include, but are not limited to diagnosing individuals to identify whether a given individual should be administered a correspondingly higher dose of a PPAR-agonist in order to ameliorate an individuals susceptibility or risk for developing dose-dependent congestive heart failure or congestive heart failure-like disorder using the genotype assays of the present invention.

In preferred embodiments, the polynucleotides and polypeptides of the present invention, including allelic and variant forms thereof, have uses which include, but are not limited to use in methods of screening to identify compounds, particularly PPAR-agonist compounds, that have a lower risk of inducing dose-dependent congestive heart failure or related disorder in a patient. Such identified compounds would be expected to retain all the benefits of a PPAR-agonist but would have diminished ability of inducing increased mineralocorticoid receptor, PPAR-gamma, or Adiponectin expression and/or function, or alternatively, capable of increasing mineralocorticoid receptor, PPAR-gamma, or Adiponectin expression and/or function to a lesser extent than a reference compound known to be capable of inducing dose-dependent congestive heart failure. Such compounds would be expected to be less likely to result in the development of dose-dependent congestive heart failure or related congestive heart failure-like disorder.

In another embodiment, the polynucleotides and polypeptides of the present invention, including allelic and variant forms thereof, either alone, or in combination with other polymorphic polynucleotides (haplotypes) are useful as genetic markers for predicting an individuals suspectability to develop dose-dependent congestive heart failure or congestive heart failure-like disorder, and particularly to predicting an individuals suspectability to develop dose-dependent congestive heart failure or congestive heart failure-like disorder upon the administration of either the prescribed dose, or an increased dose of a PPAR-agonist.

Additionally, the polynucleotides and polypeptides of the present invention, including allelic and/or variant forms thereof, are useful for creating additional antagonists directed against these polynucleotides and polypeptides, which include, but are not limited to the design of antisense RNA, ribozymes, PNAs, recombinant zinc finger proteins (Wolfe, S A., Ramm, E I., Pabo, C O, Structure, Fold, Des., 8 (7):739-50, (2000); Kang, J S., Kim, J S, J. Biol, Chem., 275 (12):8742-8, (2000); Wang, B S., Pabo, C O, Proc, Natl, Acad, Sci, U,S,A., 96 (17):9568-73, (1999); McColl, D J., Honchell, C D., Frankel, A D, Proc, Natl, Acad, Sci, U,S,A., 96 (17):9521-6, (1999); Segal, D J., Dreier, B., Beerli, R R., Barbas, CF-3rd, Proc, Natl, Acad, Sci, U,S,A., 96 (6):2758-63, (1999); Wolfe, S A., Greisman, H A., Ramm, E I., Pabo, C O, J. Mol, Biol., 285 (5):1917-34, (1999); Pomerantz, J L., Wolfe, S A., Pabo, C O, Biochemistry., 37 (4):965-70, (1998); Leon, O., Roth, M., Biol. Res. 33 (1):21-30 (2000); Berg, J M., Godwin, H A, Ann. Rev. Biophys. Biomol. Struct., 26:357-71 (1997)), in addition to other types of antagonists which are either described elsewhere herein, or known in the art.

The polynucleotides and polypeptides of the present invention, including allelic and/or variant forms thereof, are useful for identifying small molecule antagonists directed against the variant forms of these polynucleotides and polypeptides, preferably wherein such small molecules are useful as therapeutic and/or pharmaceutical compounds for the treatment, detection, prognosis, and/or prevention of the following, nonlimiting diseases and/or disorders, congestive heart failure, congestive heart failure, susceptibility to acquiring a congestive heart failure or a congestive heart failure-like disorder upon the administration of a pharmaceutically acceptable amount of a PPAR-agonist, adverse reactions associated with PPAR-agonist, disorders associated with aberrant mineralocorticoid receptor, PPAR-gamma, or Adiponectin expression, disorders associated with aberrant mineralocorticoid receptor, PPAR-gamma, or Adiponectin regulation, disorders associated with aberrant mineralocorticoid receptor, PPAR-gamma, or Adiponectin activity, disorders associated with aberrant regulation of mineralocorticoid receptor by aldosterone, disorders associated with aberrant angiotensin II peptide levels, disorders associated with aberrant adrenal and renal vascular responses to angiotensin II, disorders associated with refractory responses to angiotensin II antagonists, hypertension, high blood pressure, hypotension, low-mineralocorticoid receptor essential hypertension, high-mineralocorticoid receptor essential hypertension, atherosclerosis, congestive heart failure, pulmonary congestive heart failure, beta blocker induced congestive heart failure, and beta-1 blocker induced congestive heart failure.

Additional disorders which may be detected, diagnosed, identified, treated, prevented, and/or ameliorated by the SNPs and methods of the present invention include, the following, non-limiting diseases and disorders: diabetes, especially Type 2 diabetes, and related diseases such as insulin resistance, hyperglycemia, hyperinsulinemia, elevated blood levels of fatty acids or glycerol, hyperlipidemia, obesity, hypertriglyceridemia, inflammation, Syndrome X, diabetic complications, dysmetabolic syndrome, and related diseases.

The polynucleotides and polypeptides of the present invention, including allelic and/or variant forms thereof, are useful for the treatment of angiocongestive heart failure, hypertension, and congestive heart failure, in addition to other diseases and/or conditions referenced elsewhere herein, through the application of gene therapy based regimens.

Additional uses of the polynucleotides and polypeptides of the present invention are provided herein.

### Modified Polypeptides and Gene Sequences

The invention further provides variant forms of nucleic acids and corresponding proteins. The nucleic acids comprise one of the sequences described in Table I, in which the polymorphic position is occupied by one of the alternative bases for that position. Some nucleic acids encode full-length variant forms of proteins. Variant genes can be expressed in an expression vector in which a variant gene is operably linked to a native or other promoter. Usually, the promoter is a eukaryotic promoter for expression in a mammalian cell. The transcription regulation sequences typically include a heterologous promoter and optionally an enhancer which is recognized by the host. The selection of an appropriate promoter, for example trp, lac, phage promoters, glycolytic enzyme promoters and tRNA promoters, depends on the host selected. Commercially available expression vectors can be used. Vectors can include host-recognized replication systems, amplifiable genes, selectable markers, host sequences useful for insertion into the host genome, and the like.

The means of introducing the expression construct into a host cell varies depending upon the particular construction and the target host. Suitable means include fusion, conjugation, transfection, transduction, electroporation or injection, as described in Sambrook, supra. A wide variety of host cells can be employed for expression of the variant gene, both prokaryotic and eukaryotic. Suitable host cells include bacteria such as E. coli, yeast, filamentous fungi, insect cells, mammalian cells, typically immortalized, e.g. , mouse, CHO, human and monkey cell lines and derivatives thereof. Preferred host cells are able to process the variant gene product to produce an appropriate mature polypeptide. Processing includes glycosylation, ubiquitination, disulfide bond formation, general post-translational modification, and the like. As used herein, “gene product” includes mRNA, peptide and protein products.

The protein may be isolated by conventional means of protein biochemistry and purification to obtain a substantially pure product, i.e., 80, 95 or 99% free of cell component contaminants, as described in Jacoby, Methods in Enzymology Volume 104, Academic Press, New York (1984); Scopes, Protein Purification, Principles and Practice, 2nd Edition, Springer-Verlag, New York (1987); and Deutscher (ed), Guide to Protein Purification, Methods in Enzymology, Vol. 182 (1990). If the protein is secreted, it can be isolated from the supernatant in which the host cell is grown. If not secreted, the protein can be isolated from a lysate of the host cells.

### Haplotype Based Genetic Analysis

The invention further provides methods of applying the polynucleotides of the present invention to the elucidation of haplotypes. Such haplotypes may be associated with any one or more of the disease conditions referenced elsewhere herein. A “haplotype” is defmed as the pattern of a set of alleles of single nucleotide polymorphisms along a chromosome. For example, consider the case of three single nucleotide polymorphisms (SNP1, SNP2, and SNP3) in one chromosome region, of which SNP1 is an A/G polymorphism, SNP2 is a G/C polymorphism, and SNP3 is an A/C polymorphism. A and G are the alleles for the first, G and C for the second and A and C for the third SNP. Given two alleles for each SNP, there are three possible genotypes for individuals at each SNP. For example, for the first SNP, A/A, A/G and G/G are the possible genotypes for individuals. When an individual has a genotype for a SNP in which the alleles are not the same, for example A/G for the first SNP, then the individual is a heterozygote. When an individual has an A/G genotype at SNP1, G/C genotype at SNP2, and A/C genotype at SNP3, there are four possible combinations of haplotypes (A, B, C, and D) for this individual. The set of SNP genotypes of this individual alone would not provide sufficient information to resolve which combination of haplotypes this individual possesses. However, when this individual's parents' genotypes are available, haplotypes could then be assigned unambiguously. For example, if one parent had an A/A genotype at SNP1, a G/C genotype at SNP2, and an A/A genotype at SNP3, and the other parent had an A/G genotype at SNP1, C/C genotype at SNP2, and C/C genotype at SNP3, while the child was a heterozygote at all three SNPs, there is only one possible haplotype combination, assuming there was no crossing over in this region during meiosis.

When the genotype information of relatives is not available, haplotype assignment can be done using the long range-PCR method (Clark, A. G.. Mol Biol Evol 7 (2): 111-22 (1990); Clark, A. G., K. M. Weiss, et al. Am J Hum Genet 63 (2): 595-612 (1998); Fullerton, S. M., A. G. Clark, et al., Am J Hum. Genet 67 (4): 881-900 (2000); Templeton, A. R., A. G. Clark, et al., Am J Hum Genet 66 (1): 69-83 (2000)). When the genotyping result of the SNPs of interest are available from general population samples, the most likely haplotypes can also be assigned using statistical methods (Excoffier, L. and M. Slatkin. Mol Biol Evol 12 (5): 921-7 (1995); Fallin, D. and N. J. Schork, Am J Hum Genet 67 (4): 947-59 (2000); Long, J. C., R. C. Williams, et al., Am J Hum Genet 56 (3): 799-810 (1995)).

Once an individual's haplotype in a certain chromosome region (i.e., locus) has been determined, it can be used as a tool for genetic association studies using different methods, which include, for example, haplotype relative risk analysis (Knapp, M., S. A. Seuchter, et al., Am J Hum Genet 52 (6): 1085-93 (1993); Li, T., M. Arranz, et al., Schizophr Res 32 (2): 87-92 (1998); Matise, T. C., Genet Epidemiol 12 (6): 641-5 (1995); Ott, J., Genet Epidemiol 6 (1): 127-30 (1989); Terwilliger, J. D. and J. Ott, Hum Hered 42 (6): 337-46 (1992)). Haplotype based genetic analysis, using a combination of SNPs, provides increased detection sensitivity, and hence statistical significance, for genetic associations of diseases, as compared to analyses using individual SNPs as markers. Multiple SNPs present in a single gene or a continuous chromosomal region are useful for such haplotype-based analyses.

### Uses of the Polynucleotides

Each of the polynucleotides identified herein can be used in numerous ways as reagents. The following description should be considered exemplary and utilizes known techniques.

Increased or decreased expression of the gene in affected organisms as compared to unaffected organisms can be assessed using polynucleotides of the present invention. Any of these alterations, including altered expression, or the presence of at least one SNP of the present invention within the gene, can be used as a diagnostic or prognostic marker.

The invention provides a diagnostic method useful during diagnosis of a disorder, involving measuring the presence or expression level of polynucleotides of the present invention in cells or body fluid from an organism and comparing the measured gene expression level with a standard level of polynucleotide expression level, whereby an increase or decrease in the gene expression level compared to the standard is indicative of a disorder.

By “measuring the expression level of a polynucleotide of the present invention” is intended qualitatively or quantitatively measuring or estimating the level of the polypeptide of the present invention or the level of the mRNA encoding the polypeptide in a first biological sample either directly (e.g., by determining or estimating absolute protein level or mRNA level) or relatively (e.g., by comparing to the polypeptide level or mRNA level in a second biological sample). Preferably, the polypeptide level or mRNA level in the first biological sample is measured or estimated and compared to a standard polypeptide level or mRNA level, the standard being taken from a second biological sample obtained from an individual not having the disorder or being determined by averaging levels from a population of organisms not having a disorder. As will be appreciated in the art, once a standard polypeptide level or mRNA level is known, it can be used repeatedly as a standard for comparison.

By “biological sample” is intended any biological sample obtained from an organism, body fluids, cell line, tissue culture, or other source which contains the polypeptide of the present invention or mRNA. As indicated, biological samples include body fluids (such as the following non-limiting examples, sputum, amniotic fluid, urine, saliva, breast milk, secretions, interstitial fluid, blood, serum, spinal fluid, etc.) which contain the polypeptide of the present invention, and other tissue sources found to express the polypeptide of the present invention. Methods for obtaining tissue biopsies and body fluids from organisms are well known in the art. Where the biological sample is to include mRNA, a tissue biopsy is the preferred source.

The method(s) provided above may Preferably be applied in a diagnostic method and/or kits in which polynucleotides and/or polypeptides are attached to a solid support. In one exemplary method, the support may be a “gene chip” or a “biological chip” as described in U.S. Pat. Nos. 5,837,832, 5,874,219, and 5,856,174. Further, such a gene chip with polynucleotides of the present invention attached may be used to identify polymorphisms between the polynucleotide sequences, with polynucleotides isolated from a test subject. The knowledge of such polymorphisms (i.e. their location, as well as, their existence) would be beneficial in identifying disease loci for many disorders, including proliferative diseases and conditions. Such a method is described in U.S. Pat. Nos. 5,858,659 and 5,856,104. The US Patents referenced supra are hereby incorporated by reference in their entirety herein.

The present invention encompasses polynucleotides of the present invention that are chemically synthesized, or reproduced as peptide nucleic acids (PNA), or according to other methods known in the art. The use of PNAs would serve as the preferred form if the polynucleotides are incorporated onto a solid support, or gene chip. For the purposes of the present invention, a peptide nucleic acid (PNA) is a polyamide type of DNA analog and the monomeric units for adenine, guanine, thymine and cytosine are available commercially (Perceptive Biosystems). Certain components of DNA, such as phosphorus, phosphorus oxides, or deoxyribose derivatives, are not present in PNAs. As disclosed by P. E. Nielsen, M. Egholm, R. H. Berg and O. Buchardt, Science 254, 1497 (1991); and M. Egholm, O. Buchardt, L. Christensen, C. Behrens, S. M. Freier, D. A. Driver, R. H. Berg, S. K. Kim, B. Norden, and P. E. Nielsen, Nature 365, 666 (1993), PNAs bind specifically and tightly to complementary DNA strands and are not degraded by nucleases. In fact, PNA binds more strongly to DNA than DNA itself does. This is probably because there is no electrostatic repulsion between the two strands, and also the polyamide backbone is more flexible. Because of this, PNA/DNA duplexes bind under a wider range of stringency conditions than DNA/DNA duplexes, making it easier to perform multiplex hybridization. Smaller probes can be used than with DNA due to the stronger binding characteristics of PNA:DNA hybrids. In addition, it is more likely that single base mismatches can be determined with PNA/DNA hybridization because a single mismatch in a PNA/DNA 15-mer lowers the melting point (T.sub.m) by 8°-20° C., vs. 4°-16° C. for the DNA/DNA 15-mer duplex. Also, the absence of charge groups in PNA means that hybridization can be done at low ionic strengths and reduce possible interference by salt during the analysis.

Polynucleotides of the present invention are also useful in gene therapy. One goal of gene therapy is to insert a normal gene into an organism having a defective gene, in an effort to correct the genetic defect. The polynucleotides disclosed in the present invention offer a means of targeting such genetic defects in a highly accurate manner. Another goal is to insert a new gene that was not present in the host genome, thereby producing a new trait in the host cell. In one example, polynucleotide sequences of the present invention may be used to construct chimeric RNA/DNA oligonucleotides corresponding to said sequences, specifically designed to induce host cell mismatch repair mechanisms in an organism upon systemic injection, for example (Bartlett, R. J., et al., Nat. Biotech, 18:615-622 (2000), which is hereby incorporated by reference herein in its entirety). Such RNA/DNA oligonucleotides could be designed to correct genetic defects in certain host strains, and/or to introduce desired phenotypes in the host (e.g., introduction of a specific polymorphism within an endogenous gene corresponding to a polynucleotide of the present invention that may ameliorate and/or prevent a disease symptom and/or disorder, etc.). Alternatively, the polynucleotide sequence of the present invention may be used to construct duplex oligonucleotides corresponding to said sequence, specifically designed to correct genetic defects in certain host strains, and/or to introduce desired phenotypes into the host (e.g., introduction of a specific polymorphism within an endogenous gene corresponding to a polynucleotide of the present invention that may ameliorate and/or prevent a disease symptom and/or disorder, etc). Such methods of using duplex oligonucleotides are known in the art and are encompassed by the present invention (see EP1007712, which is hereby incorporated by reference herein in its entirety).

### REFERENCES

Arai K, Nakagomi Y, Iketani M, Shimura Y, Amemiya S, Ohyama K, Shibasaki T (2003) Functional polymorphisms in the mineralocorticoid receptor and amirolide-sensitive sodium channel genes in a patient with sporadic pseudohypoaldosteronism. Hum Genet 112: 91-97.

Berger J and Moller D E (2002) The mechanism of action of PPARs Annu Rev Med 53:409-435.

Berger J and Wagner J A (2002) Physiological and therapeutic roles of peroxisome proliferator-activated receptors Diabetes Technology & Therapeutics 4(2): 163-174.

Chakrabarti R, Vikramadithyan R K, Misra P, Jagadheshan H, Raichur S, Damarla R K, Gershome C, Suresh J, Rajagopalan R (2003) Ragaglitazar: a novel PPARa & PPARg agonist with potent lipid-lowering and insulin-sensitizing efficacy in animal models British J Pharmacol 140:527-537.

Deeb S S, Fajas L, Nemoto M, Pihlajamaki J, Mykkanen L, Kuusisto J, Laakso M, Fujimoto W, Auwerx J. (1998) A Pro12Ala substitution in PPARgamma2 associated with decreased receptor activity, lower body mass index and improved insulin sensitivity. Nat Genet. 20:284-287.

Fisher L D, van Belle G (1993) Biostatistics. John Wiley & Sons, New York, N.Y.

Giles T (2003) The patient with Diabetes mellitus and heart failure: at-risk issues Am J Med 115(8A):107-110.

Hollenberg N K (2003) Considerations for management of fluid dynamic issues associated with thiazolidinediones Am J Med 115(8A):111-115.

Inzucchi S E (2002) Oral antihyperglycemic therapy for type 2 Diabetes JAMA 287(3):360-372.

Laasko M (2004) Gene variants, insulin resistance, and dyslipidemia. Curr Opin Lipidology, 15:115-120.

Maeda N, Takahashi M, Funahashi T, Kihara S, Nishizawa H, Kishida K, Nagaretani H, Matsuda M, Komuro R, Ouchi N, Kuriyama H, Hotta K, Nakamura T, Shimomura I, Matsuzawa Y. (2001). PPARgamma ligands increase expression and plasma concentrations of adiponectin, an adipose-derived protein. Diabetes. 50:2094-9.

Scherer P E, Williams S, Fogliano M, Baldini G, Lodish H F (1995) A novel serum protein similar to Clq, produced exclusively in adipocytes. J Biol Chem. 270:26746-9.

Shibata R, Ouchi N, Ito M, Kihara S, Shiojima I, Pimentel D R, Kumada M, Sato K, Schiekofer S, Ohashi K, Funahashi T, Colucci W S, Walsh K. (2004). Adiponectin-mediated modulation of hypertrophic signals in the heart. Nat Med.10:1384-9.

Skrumsager B K, Nielsen K K, Muller M, Pabst G, Drake P G, Edsberg B (2003) Ragaglitazar: the pharmacokinetics, pharmacodynamics, and tolerability of a novel dual PPARa and y agonist in healthy subjects and patients with type 2 Diabetes. J Clin Pharmacol 43:1244-1256.

Vasseur F, Helbecque N, Dina C, Lobbens S, Delannoy V, Gaget S, Boutin P, Vaxillaire M, Lepretre F, Dupont S, Hara K, Clement K, Bihain B, Kadowaki T, Froguel P. (2002). Single-nucleotide polymorphism haplotypes in the both proximal promoter and exon 3 of the APM1 gene modulate adipocyte-secreted adiponectin hormone levels and contribute to the genetic risk for type 2 diabetes in French Caucasians. Hum Mol Genet. 11:2607-14.

Weber K T (2001). Aldosterone in congestive heart failure. N Engl J Med 345(23): 1689-1697.

Yamauchi T, Kamon J, Waki H, Terauchi Y, Kubota N, Hara K, Mori Y, Ide T, Murakami K, Tsuboyama-Kasaoka N, Ezaki O, Akanuma Y, Gavrilova O, Vinson C, Reitman M L, Kagechika H, Shudo K, Yoda M, Nakano Y, Tobe K, Nagai R, Kimura S, Tomita M, Froguel P, Kadowaki T. (2001). The fat-derived hormone adiponectin reverses insulin resistance associated with both lipoatrophy and obesity. Nat Med. 7:941-6.

### EXAMPLES

### Example 1

### Method of Discovering the Single Nucleotide Polymorphisms (SNPs) of the Present Invention

SNP discovery was based on comparative DNA sequencing of PCR products directed to the human mineralocorticoid receptor gene derived from genomic DNA from multiple individuals. All genomic DNA samples for SNP discovery were obtained from a panel of thirty-two human subjects obtained from the Coriell Institute (Camden, N.J.), panel # M44PDR. A total of 1 SNP within the human mineralocorticoid receptor gene was identified, and found to be associated with the incidence of dose-dependent congestive heart failure:

MLR-SNP 1: A/G at nucleotide 754 of SEQ ID NO:1 and 3

MLR SNP 1 was identified using the following protocol. Briefly, portions of the human mineralocorticoid receptor genomic sequence were PCR amplified using the standard Platinum Taq DNA protocol (Invitrogen, Product #10966-083):

The following sequencing primers (20 uM each) were used to confirm the presence of the above SNPs, as well as to sequence across the PCR amplicons:

| | | |
|---|---|---|
| SNP | Forward Primer | Reverse Primer |

| | | |
|---|---|---|
| MLR SNP1 | CCCCTGCCCTCTTCTTTCTAGT | CGTATTTTCTGAACACGCCTAAATAA |
| | (SEQ ID NO: 5) | (SEQ ID NO: 6) |

All the samples amplified from genomic DNA (50 ng) in reactions (50 ul) containing 50 mM Tris-Acetate pH 8.4, 75 mM KAcetate, 8 mM MgAcetate, 200 uM dNTPs, 0.2 uM of each PCR primer, and 2.5 U Platinum Taq DNA polymerase (Invitrogen).

PCR amplification was performed in Perkin Elmer 9700 machines under the following cycling conditions: 1.) 94 degrees Celsius for 2 minutes; 2.) 94 degrees Celsius for 30 seconds; 3.) 59 degrees Celsius for 1 minute; 4.) 72 degrees Celsius for 30 seconds; 5.) 72 degrees Celsius for 5 minutes; and 6.) 4 degrees Celsius on hold. Steps 2 to 4 were cycled 35 times.

PCR products were sequenced using ABI BigDye Terminator v3.1 Cycle Sequencing chemistry on the 3730-XL capillary sequencers using the same primers used for identifying the SNPs, as described above.

Sequence editing and contig assembly was performed using CONSED software (Genome Res. 1998 March; 8(3): 195-202). Chromatograms were visually inspected for each Coriell DNA and SNPs identified by comparing sequence traces to the reference mineralocorticoid receptor provided as SEQ ID NO:1 herein. In the instant study, the “V” allele at amino acid 180 of SEQ ID NO:2 was found to be the rare allele and thus has been termed the variable allele accordingly, with the “I” allele at amino acid 180 of SEQ ID NO:2 serving as the reference allele. The coding region of the variable mineralocorticoid receptor is provided as SEQ ID NO:3, encoding the reference mineralocorticoid receptor polypeptide sequence provided as SEQ ID NO:4.

The nucleotide sequence of the mineralocorticoid receptor gene containing the reference allele (“a”) for SNP1 at nucleotide 754 is provided in FIGS. 1A-F (SEQ ID NO:1); while the nucleotide sequence of the mineralocorticoid receptor gene containing the variable allele (“g”) for SNP 1 at nucleotide 754 is provided in FIGS. 2A-F (SEQ ID NO:3).

The polypeptide sequence of mineralocorticoid receptor containing the reference allele (“I”) for SNP1 at amino acid 180 is provided in FIGS. 1A-F (SEQ ID NO:2); while the polypeptide sequence of mineralocorticoid receptor containing the variable allele (“V”) for SNP1 at amino acid 180 is provided in FIGS. 2A-F (SEQ ID NO:4).

### Example 2

### Method of Genotyping Each SNP of the Present Invention

Genomic DNA samples from patients enrolled in two Bristol-Myers Squibb Company Phase II clinical trials CV168-006 and CV168-008 trials were genotyped for 1 SNP identified in the human mineralocorticoid receptor gene (see Example 1) and evaluated for association with congestive heart failure.

498 subjects enrolled in the CV168-006 trial and 232 subjects from the CV168-008 trial were analyzed in this study. All analyses were based on data collected up to 24 weeks, which was the duration of the short-term phase of the trials. DNA was extracted from frozen blood by a third-party (Genaissance Inc, North Carolina) using a salting-out method (Gentra Systems). All subjects gave written informed consent.

At the time of this analysis four individuals were diagnosed as having developed congestive heart failure. All four were in the CV168-006 trial; three were being treated with 10 mg of compound A and one with 20 mg of compound A. All SNPs that were analyzed above were also tested for association with CHF using contingency tables. Since CHF was observed only at high doses of compound A, this analysis was restricted to the 10 and 20 mg treatment arms of the CV168-006 trial. As shown below, polymorphisms in the mineralocorticoid receptor (MLR; P=0.03) gene showed evidence of association with CHF.

Genotyping was performed using the 5′ nuclease assay, essentially as described (Ranade K et al., Genome Research 11: 1262-1268 (2001); which is hereby incorporated by reference herein in its entirety), with the following modifications: six nanograms of genomic DNA were used in a 8 ul reaction. All PCR reactions were performed in an ABI 9700 machine and fluorescence was measured using an ABI 7900 machine.

Genotyping of the SNPs of the present invention was performed using sets of Taqman probes (100 uM each) and primers (100 uM each) specific to the SNP. Each probe/primer set was manually designed using ABI Primer Express software (Applied Biosystems). Genomic samples were prepared as described in Example 1. The following Taqman probes and primers were utilized:

| | Taqman Forward | Taqman Reverse | Reference Taqman | Variable Taqman |
|---|---|---|---|---|
| SNP | Primer | Primer | Probe | Probe |
| SNP1 | CCCCTGCCCTCTTC | CGTATTTTCTGAAC | TCTATCCTTTGGGTCC | TCTATCCTTTGGGCCC |
| | TTTCTAGT | ACGCCTAAATAA | TACGGCCGT | TACGGCC |
| | (SEQ ID NO: 5) | (SEQ ID NO: 6) | (SEQ ID NO: 7) | (SEQ ID NO: 8) |

** The allelic nucleotide in each probe sequence is shown in bold and underlined.

The genotype assay conditions are provided below.

| Components: | Final Concentration: |
|---|---|
| 2x PE Master Mix (#4318157) | 1X |
| 100 uM FAM labeled probe | 200 nmol |
| 100 uM VIC labeled probe | 200 nmol |
| Forward PCR primer | 600 nmol |
| Reverse PCR primer | 600 nmol |
| 6 ng template DNA | as required |
| ddH20 | volume to 8 ul |

Taqman thermo-cycling was performed on Perkin Elmer PE 9700 machines using the following cycling conditions below:
1) 50 C for 2 minutes
2) 95 C for 10 seconds*
3) 94 C for 15 seconds
4) 62 C for 1 minute
5) 4 C hold
   *Steps 2-4 were cycled 40 times

Analysis of genotypes was performed by using the Applied Biosystems ABI 7900 HT sequence detection system.

### Example 3

### Statistical Analysis of the Association Between Dose-dependent Peripheral Congestive Heart Failure and the SNPs of the Present Invention

The association between congestive heart failure and the single nucleotide polymorphisms of the present invention were investigated by applying statistical analysis to the results of the genotyping assays described elsewhere herein. The central hypothesis of this analysis is that a predisposition to develop dose-dependent congestive heart failure may be conferred by specific genomic factors. The analysis attempted to identify one or more of these factors in genomic DNA samples from index cases and matched control subjects who were exposed to Compound A in two Bristol-Myers Squibb (BMS) clinical studies (see Example 2).

SNPs of the present invention were examined for association with congestive heart failure using 3 (genotypes)×2 (congestive heart failure and no congestive heart failure) contingency tables. Analyses were performed using S-plus (version 6.0; Insightful Corp. Seattle, Wash.) or SPSS (version 12.0; SPSS Inc. Chicago, Ill.).

### Methods

Sample. Investigators in the BMS clinical trials diagnosed dose-dependent congestive heart failure in some subjects.

Measures. Single nucleotide polymorphisms (SNPs) in human mineralocorticoid receptor were genotyped on all subjects essentially as described in Example 2 herein. The SNPs that are genotyped likely represent a sample of the polymorphic variation in each gene and are not exhaustive with regard to coverage of the total genetic variation that may be present in each gene. Specifically, only the 1 SNP referenced herein was genotyped and statistically analyzed, as described. The SNP for which a statistical association to congestive heart failure susceptibility was confirmed are provided and refered to as SNP 1.

Statistical Analyses. Association between SNP1 with the incidence of congestive heart failure was performed using the Fisher test (Fisher L D, van Belle G (1993) Biostatistics. John Wiley & Sons, New York, N.Y.). Analysis was done by comparing the genotype frequencies of SNPs in patients who developed CHF to those who were not affected. Analysis was restricted to patients treated with 10 and 20 mg of compound A in CV168-006 and CV168-008 Phase II trials.

Since the SNP coverage within the human mineralocorticoid receptor gene was not exhaustive of the genetic variation that may be present and possibly related to event susceptibility in this gene, inferences about these SNP associations with dose-dependent congestive heart failure events for mineralocorticoid receptor are therefore related to the hypothesis that genetic variation in this gene may be involved in susceptibility to such events.

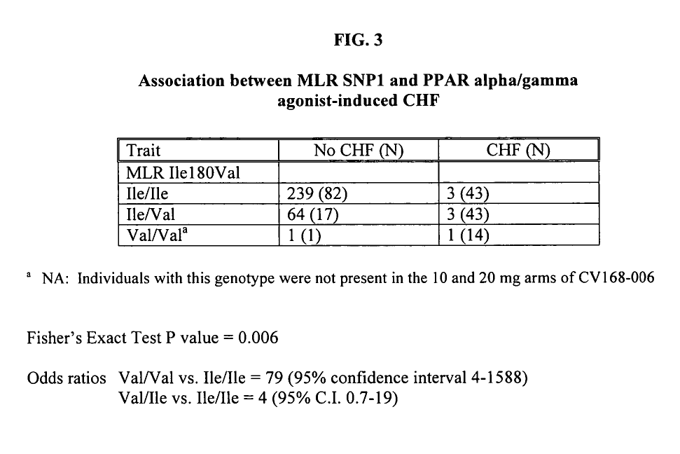

The association statistics for SNP1 are provided in FIG. 3.

### Genetic Associations with Congestive Heart Failure

All subjects for whom sufficient DNA was available were genotyped for 218 SNPs in 65 candidate genes with 1 of these SNPs being specific for the human mineralocorticoid receptor gene. Of these 218 SNPs, subjects enrolled in these trials were polymorphic for 153 SNPs (minor allele frequency >0.1%). In 3×2 contingency table analysis, SNPs in the mineralocorticoid receptor genes were significantly associated with congestive heart failure status (P<0.05).

The mineralocorticoid receptor SNP1 was determined to be in Hardy-Weinberg equilibrium. These results suggest that polymorphisms in the mineralocorticoid receptor gene contributes to differences in susceptibility to dose-dependent congestive heart failure independent of other significant predictors such as age, sex and body mass index.

SNPs in the mineralocorticoid receptor gene increased risk of CHF. The mineralocorticoid receptor binds to aldosterone and activates transcription of a variety of genes involved in sodium and water re-absorption by the kidney. Heart failure medicines like spironolactone and eplerenone target the mineralocorticoid receptor and prevent aldosterone from activating it. Furthermore, transgenic mice overexpressing MLR are susceptible to mild dilated cardiomyopathy. In vitro studies suggest that the high risk Valine180 allele increases the activity of the receptor in vitro, at physiological concentrations of aldosterone (Arie et al). Taken together, these observations suggest that increased activity of MLR contributes to susceptibility to CHF.

The utility, in general, of each of these significant SNP-dose-dependent congestive heart failure event associations is that they suggest (1) such SNPs may be causally involved, alone or in combination with other SNPs in the respective gene regions with susceptibility to dose-dependent congestive heart failure events resulting from exposure to a PPAR-agonist; (2) such SNPs, if not directly causally involved, are reflective of an association because of linkage disequilibrium with one or more other SNPs that may be causally involved, alone or in combination with other SNPs in the respective gene regions with susceptibility to dose-dependent congestive heart failure resulting from exposure to a PPAR-agonist; (3) such SNPs may be useful in establishing haplotypes that may be used to narrow the search for and identify polymorphisms or combinations of polymorphisms that may be causally, alone or in combination with other SNPs in the respective gene regions with susceptibility to dose-dependent congestive heart failure resulting from exposure to a PPAR-agonist; and (4) such SNPs, if used to establish haplotypes that are identified as causally involved in such event susceptibility, may be used to predict which subjects are most likely to experience such events when exposed to a dose-dependent congestive heart failure resulting from exposure to a PPAR-agonist. The term “respective gene regions” shall be construed to refer to those regions of each gene which have been used to identify the SNPs of the present invention.

### Example 4

### Method of Isolating the Native Forms of the Human MLR Gene

A number of methods have been described in the art that may be utilized in isolating the native forms of the human mineralocorticoid receptor gene. Specific methods are referenced below and are hereby incorporated by reference herein in their entireties. The artisan, skilled in the molecular biology arts, would be able to isolate the native form of human mineralocorticoid receptor based upon the methods and information contained, and/or referenced, therein.

Human Variable MLR (gi|NP_{—}000892; SEQ ID NO:3 and 4)

1) Walther, R. F et al., J. Biol. Chem. 280 (17), 17549-17561 (2005).
2) Yokota, K. et al, Endocr. Res. 30 (4), 611-616 (2004).
3) Sartorato, P. et al, Mol. Endocrinol. 18 (9), 2151-2165 (2004).
4) Tirard, M. et al, J. Mol. Endocrinol. 32 (3), 825-841 (2004).
5) Pascual-Le Tallec, L. et al, Eur. J. Endocrinol. 150 (4), 585-590 (2004).
5) Leo, J. C. et al, Endocrinology 145 (3), 1314-1321 (2004).
6) Schmider-Ross, A. Et al, Mol. Endocrinol. 23 (1), 113-119 (2004).
7) Tallec, L. P. et al, Mol. Endocrinol. 17 (12), 2529-2542 (2003).
8) Sartorato, P. et al, J. Clin. Endocrinol. Metab. 88 (6), 2508-2517 (2003).
9) Rafestin-Oblin, M. E. et al, Endocrinology 144 (2), 528-533 (2003).
10) Campion, J. et al, Cell Biochem. Funct. 20 (3), 237-245 (2002).
11) Stauffer, A. T. et al, J. Biol. Chem. 277 (29), 26286-26292 (2002).
12) Sheppard, K. E., Am. J. Physiol. Gastrointest. Liver Physiol. 282 (5), G742-G746 (2002).
13) Zennaro, M. C. et al, Mol. Endocrinol. 15 (9), 1586-1598 (2001).
14) Savory, J. G. et al, Mol. Cell. Biol. 21 (3), 781-793 (2001).
15) Watzka, M. et al, Neurosci. Lett. 290 (2), 121-124 (2000).
16) Freeman, B. C. et al, Genes Dev. 14 (4), 422-434 (2000).
17) Geller, D. S. et al, Nat. Genet. 19 (3), 279-281 (1998).
18) Thenot, S. et al, J. Biol. Chem. 272 (18), 12062-12068 (1997).
19) Zennaro, M. C. et al, J. Clin. Endocrinol. Metab. 82 (5), 1345-1352 (1997).
20) Bruner, K. L. et al, J. Clin. Endocrinol. Metab. 7 (2), 85-98 (1997).
21) Jalaguier, S. et al, FEBS Lett. 384 (2), 112-116 (1996).
22) Morrison, N. et al, Hum. Genet. 85 (1), 130-132 (1990).
23) Fan, Y. S. et al, Cytogenet. Cell Genet. 52 (1-2), 83-84 (1989).
24) Arriza, J. L. et al, Science 237 (4812), 268-275 (1987).

Methods of isolation for the human mineralocorticoid receptor gene of the present invention may also be found in reference to the references cited in the Genbank accession nos. for each gene provided herein which are hereby incorporated by reference herein.

### Example 5

### Method of Isolating the Polymorphic Forms of the Human MLR Gene of the Present Invention

Since the allelic genes of the present invention represent genes present within at least a subset of the human population, these genes may be isolated using the methods provided in Example 4 above. For example, the source DNA used to isolate the allelic gene may be obtained through a random sampling of the human population and repeated until the allelic form of the gene is obtained. Preferably, random samples of source DNA from the human population are screened using the SNPs and methods of the present invention to identify those sources that comprise the allelic form of the gene. Once identified, such a source may be used to isolate the allelic form of the gene(s). The invention encompasses the isolation of such allelic genes from both genomic and/or cDNA libraries created from such source(s).

In reference to the specific methods provided in Example 4 above, it is expected that isolating the polymorphic alleles of the human mineralocorticoid receptor gene would be within the skill of an artisan trained in the molecular biology arts. Nonetheless, a detailed exemplary method of isolating at least one of the mineralocorticoid receptor polymorphic alleles, in this case the variant form of SNP1 (“g” nucleotide at 754 of SEQ ID NO:3) is provided. Briefly,

First, the individuals with the a754g variation are identified by genotyping the genomic DNA samples using the method outlined in Example 2 herein. Other methods of genotyping may be employed, such as the FP-SBE method (Chen et al., Genome Res., 9(5):492-498 (1999)), or other methods described herein. DNA samples publicly available (e.g., from the Coriell Institute (Collingswood, N.J.) or from the Bristol-Myers Squibb clinical samples described herein may be used. Oligonucleotide primers that are used for this genotyping assay are provided in Example 2.

By analyzing genomic DNA samples, individuals with the a754g form of the SNP1 variant may be identified. Once identified, clones comprising the genomic sequence may be obtained using methods well known in the art (see Sambrook, J., E. F. Fritsch, and T. Maniatis, 1989, Molecular Cloning: A Laboratory Manual, Cold Spring Harbor Laboratory Press, Cold Spring Harbor, N.Y.; and Current Protocols in Molecular Biology, 1995, F. M., Ausubel et al., eds, John Wiley and Sons, Inc., which are hereby incorporated by reference herein.).

If cDNA clones of the coding sequence of this allele of the gene are of interest, such clones may be obtained in accordance with the following steps. Next, Lymphoblastoid cell lines from these individuals may be obtained from the Coriell Institute. These cells can be grown in RPMI-1640 medium with L-glutamine plus 10% FCS at 37 degrees. PolyA+ RNA are then isolated from these cells using Oligotex Direct Kit (Life Technologies).

First strand cDNA (complementary DNA) is produced using Superscript Preamplification System for First Strand cDNA Synthesis (Life Technologies, Cat No 18089-011) using these polyA+ RNA as templates, as specified in the users manual which is hereby incorporated herein by reference in its entirety. Specific cDNA encoding the human mineralocorticoid receptor protein is amplified by polymerase chain reaction (PCR) using a forward primer which hybridizes to the 5′-UTR region, a reverse primer which hybridizes to the 3′-UTR region, and these first strand cDNA as templates (Sambrook, Fritsch et al. 1989). Alternatively, these primers may be designed using Primer3 program (Rozen S 2000). Restriction enzyme sites (example: SalI for the forward primer, and NotI for reverse primer) are added to the 5′-end of these primer sequences to facilitate cloning into expression vectors after PCR amplification. PCR amplification may be performed essentially as described in the owner's manual of the Expand Long Template PCR System (Roche Molecular Biochemicals) following manufacturer's standard protocol, which is hereby incorporated herein by reference in its entirety.

PCR amplification products are digested with restriction enzymes (such as SalI and NotI, for example) and ligated with expression vector DNA cut with the same set of restriction enzymes. pSPORT (Invitrogen) is one example of such an expression vector. After ligated DNA is introduced into E. coli cells (Sambrook, Fritsch et al. 1989), plasmid DNA is isolated from these bacterial cells. This plasmid DNA is sequenced to confirm the presence an intact (full-length) coding region of the human mineralocorticoid receptor protein with the variation, if the variation results in changes in the encoded amino acid sequence, using methods well known in the art and described elsewhere herein.

The skilled artisan would appreciate that the above method may be applied to isolating the other novel human mineralocorticoid receptor genes of the present invention through the simple substitution of applicable PCR and sequencing primers. Such primers may be selected from any one of the applicable primers provided in herein, or may be designed using the Primer3 program (Rozen S 2000) as described. Such primers may preferably comprise at least a portion of any one of the polynucleotide sequences of the present invention.

### Example 6

### Method of Engineering the Allelic Forms of the Human MLR Gene of the Present Invention

Aside from isolating the allelic genes of the present invention from DNA samples obtained from the human population, Bristol-Myers Squibb Company clinical trials, and/or the Coriell Institute, as described in Example 5 above, the invention also encompasses methods of engineering the allelic genes of the present invention through the application of site-directed mutagenesis to the isolated native forms of the genes. Such methodology could be applied to synthesize allelic forms of the genes comprising at least one, or more, of the encoding SNPs of the present invention (e.g., silent, missense)—preferably at least 1, 2, 3, or 4 encoding SNPs for each gene.

In reference to the specific methods provided in Example 5 above, it is expected that isolating the novel polymorphic mineralocorticoid receptor genes of the present invention would be within the skill of an artisan trained in the molecular biology arts. Nonetheless, a detailed exemplary method of engineering at least one of the mineralocorticoid receptor polymorphic alleles to comprise the encoding and/or non-coding polymorphic nucleic acid sequence, in this case the variant form (a754g) of SNP1 (SEQ ID NO:3) is provided. Briefly,

cDNA clones encoding the human mineralocorticoid receptor protein may be identified by homology searches with the BLASTN program (Altschul SF 1990) against the Genbank non-redundant nucleotide sequence database using the published variable human mineralocorticoid receptor cDNA sequence (GenBank Accession No.: gi| NP_{—}000892). Alternatively, the genomic sequence of the human mineralocorticoid receptor gene may be obtained as described herein. After obtaining these clones, they are sequenced to confirm the validity of the DNA sequences.

However, in the case of the reference form (a754) of SNP1, genomic clones would need to be obtained and may be identified by homology searches with the BLASTN program (Altschul S F 1990) against the Genbank non-redundant nucleotide sequence database using the published human mineralocorticoid receptor cDNA sequence (GenBank Accession No.: gi| NP_{—}000892). Alternatively, the genomic sequence of the human reference mineralocorticoid receptor gene may be obtained as described herein. After obtaining these clones, they are sequenced to confirm the validity of the DNA sequences.

Once these clones are confirmed to contain the intact wild type cDNA or genomic sequence of the human mineralocorticoid receptor coding and/or non-coding region, the a754g polymorphism (mutation) may be introduced into the native sequence using PCR directed in vitro mutagenesis (Cormack, B., Directed Mutagenesis Using the Polymerase Chain Reaction. Current Protocols in Molecular Biology, John Wiley & Sons, Inc. Supplement 37: 8.5.1-8.5.10, (2000)). In this method, synthetic oligonucleotides are designed to incorporate a point mutation at one end of an amplified fragment. Following PCR, the amplified fragments are made blunt-ended by treatment with Klenow Fragment. These fragments are then ligated and subcloned into a vector to facilitate sequence analysis. This method consists of the following steps.
1. Subcloning of cDNA or genomic insert into a plasmid vector, or BAC sequence if the clone is a genomic sequence, containing multiple cloning sites and M13 flanking sequences, such as pUC19 (Sambrook, Fritsch et al. 1989), in the forward orientation. The skilled artisan would appreciate that other plasmids could be equally substituted, and may be desirable in certain circumstances.
2. Introduction of a mutation by PCR amplification of the cDNA region downstream of the mutation site using a primer including the mutation. (FIG. 8.5.2 in Cormack 2000)). In the case of introducing the reference a754g (i.e., I180V) sequence mutation into the human mineralocorticoid receptor protein, the following two primers may be used.

M13 Reverse Sequencing Primer:

| | |
|---|---|
| 5′-AGCGGATAACAATTTCACACAGGA-3′. | (SEQ ID NO: 9) |

Mutation Primer:

(SEQ ID NO: 11)

5′- GAATGGTGGCGTCATGCGCGCCATTGTTAAAAGCCCTATCATG -
3′

Mutation primer contains the mutation (a754g) at the center (in bold and underlined) and a portion of its flanking sequence. M13 reverse sequencing primer hybridizes to the pUC19 vector. Subcloned cDNA or genomic clone comprising the human mineralocorticoid receptor cDNA or genomic sequence is used as a template (described in Step 1). A 100 ul PCR reaction mixture is prepared using long of the template DNA, 200 uM 4dNTPs, 1 uM primers, 0.25U Taq DNA polymerase (PE), and standard Taq DNA polymerase buffer. Typical PCR cycling condition are as follows:

| | |
|---|---|
| 20-25 cycles: | 45 sec, 93 degrees |
| | 2 min, 50 degrees |
| | 2 min, 72 degrees |
| 1 cycle: | 10 min, 72 degrees |

After the final extension step of PCR, 5U Klenow Fragment is added and incubated for 15 min at 30 degrees. The PCR product is then digested with the restriction enzyme, EcoRI.
3. PCR amplification of the upstream region is then performed, using subcloned cDNA or genomic clone as a template (the product of Step 1). This PCR is done using the following two primers:

M13 Forward Sequencing Primer:

| | |
|---|---|
| 5′-CGCCAGGGTTTTCCCAGTCACGAC-3′. | (SEQ ID NO: 10) |

Flanking Primer:

(SEQ ID NO: 12)

5′- CATGATAGGGCTTTTAACAATGGCGCGCATGACGCCACCATTC -
3′.

Flanking primer is complimentary to the upstream flanking sequence and mutation locus of the a754g mutation (in bold and underlined). M13 forward sequencing primer hybridizes to the pUC19 vector. PCR conditions and Klenow treatments follow the same procedures as provided in Step 2, above. The PCR product is then digested with the restriction enzyme, HindIII.
4. Prepare the pUC19 vector for cloning the cDNA or genomic clone comprising the polymorphic locus. Digest pUC19 plasmid DNA with EcoRI and HindIII. The resulting digested vector fragment may then be purified using techniques well known in the art, such as gel purification, for example.
5. Combine the products from Step 2 (PCR product containing mutation), Step 3 (PCR product containing the upstream region), and Step 4 (digested vector), and ligate them together using standard blunt-end ligation conditions (Sambrook, Fritsch et al. 1989).
6. Transform the resulting recombinant plasmid from Step 5 into E.coli competent cells using methods known in the art, such as, for example, the transformation methods described in Sambrook, Fritsch et al. 1989.
7. Analyze the amplified fragment portion of the plasmid DNA by DNA sequencing to confirm the point mutation, and absence of any other mutations introduced during PCR. The method of sequencing the insert DNA, including the primers utilized, are described herein or are otherwise known in the art.

Moreover, the skilled artisan would appreciate that the above method may be applied to engineering more than one polymorphic nucleic acid sequence of the present invention into the novel mineralocorticoid receptor genes of the present invention. Such an engineered gene could be created through succesive rounds of site-directed mutagenesis, as described in Steps 1 thru 7 above, or consolidated into a single round of mutagenesis. For example, Step 2 above could be performed for each mutation, then the products of both mutation amplifications could be combined with the product of Step 3 and 4, and the procedure followed as described.

### Example 7

### Method of Discovering the Single Nucleotide Polymorphisms (SNPs) of the Present Invention

SNP discovery was based on comparative DNA sequencing of PCR products directed to the human PPAR-gamma gene derived from genomic DNA from multiple individuals. All genomic DNA samples for SNP discovery were obtained from a panel of thirty-two human subjects obtained from the Coriell Institute (Camden, N.J.), panel # M44PDR. A total of 1 SNP within the human PPAR-gamma gene was identified, and found to be associated with the incidence of dose-dependent congestive heart failure:

PPAR-gamma-SNP1: C/G at nucleotide 167 of SEQ ID NO:13 and 15

PPAR-gamma SNP1 was identified using the following protocol. Briefly, portions of the human PPAR-gamma genomic sequence (SEQ ID NO:13) were PCR amplified using the standard Platinum Taq DNA protocol (Invitrogen, Product #10966-083):

The following sequencing primers (20 uM each) were used to confirm the presence of the above SNP(s), as well as to sequence across the PCR amplicons:

| | | |
|---|---|---|
| SNP | Forward Primer | Reverse Primer |

| | | |
|---|---|---|
| PPAR- | AGACAAAATATCAGTGTGAATTACAG | GTGATATGTTTGCAGACAGTGTATCAG |
| gamma | CA | T |
| SNP1 | (SEQ ID NO: 17) | (SEQ ID NO: 18) |

All the samples amplified from genomic DNA (50 ng) in reactions (50 ul) containing 50 mM Tris-Acetate pH 8.4, 75 mM KAcetate, 8 mM MgAcetate, 200 uM dNTPs, 0.2 uM of each PCR primer, and 2.5 U Platinum Taq DNA polymerase (Invitrogen).

PCR amplification was performed in Perkin Elmer 9700 machines under the following cycling conditions: 1.) 94 degrees Celsius for 2 minutes; 2.) 94 degrees Celsius for 30 seconds; 3.) 59 degrees Celsius for 1 minute; 4.) 72 degrees Celsius for 30 seconds; 5.) 72 degrees Celsius for 5 minutes; and 6.) 4 degrees Celsius on hold. Steps 2 to 4 were cycled 35 times.

PCR products were sequenced using ABI BigDye Terminator v3.1 Cycle Sequencing chemistry on the 3730-XL capillary sequencers using the same primers used for identifying the SNPs, as described above.

Sequence editing and contig assembly was performed using CONSED software (Genome Res. 1998 March; 8(3):195-202). Chromatograms were visually inspected for each Coriell DNA and SNPs identified by comparing sequence traces to the reference PPAR-gamma provided as SEQ ID NO:13 and 14 herein.

In the instant study, the “Ala” allele at amino acid 12 of SEQ ID NO:14 was found to be the rare allele and thus has been termed the variable allele accordingly, with the “Pro” allele at amino acid 12 of SEQ ID NO:14 serving as the reference allele.

The nucleotide sequence of the PPAR-gamma gene containing the reference allele (“c”) for SNP1 at nucleotide 167 is provided in FIGS. 4A-B (SEQ ID NO:13); while the nucleotide sequence of the PPAR-gamma gene containing the variable allele (“g”) for SNP1 at nucleotide 167 is provided in FIGS. 5A-B (SEQ ID NO:15).

The polypeptide sequence of PPAR-gamma containing the reference allele (“Pro”) for SNP1 at amino acid 12 is provided in FIGS. 4A-B (SEQ ID NO:14); while the polypeptide sequence of PPAR-gamma containing the variable allele (“Ala”) for SNP 1 at amino acid 12 is provided in FIGS. 5A-B (SEQ ID NO:16).

### Example 8

### Method of Genotyping Each SNP of the Present Invention

Genomic DNA samples from patients enrolled in two Bristol-Myers Squibb Company Phase II clinical trials CV168-006 and CV168-008 trials were genotyped for 1 SNP identified in the human PPAR-gamma gene (see Example 7) and evaluated for association with congestive heart failure.

498 subjects enrolled in the CV168-006 trial and 232 subjects from the CV168-008 trial were analyzed in this study. All analyses were based on data collected up to 24 weeks, which was the duration of the short-term phase of the trials. DNA was extracted from frozen blood by a third-party (Genaissance Inc, North Carolina) using a salting-out method (Gentra Systems). All subjects gave written informed consent.

At the time of this analysis four individuals were diagnosed as having developed congestive heart failure. All four were in the CV 168-006 trial; three were being treated with 10 mg of compound A and one with 20 mg of compound A. All SNPs that were analyzed above were also tested for association with CHF using contingency tables. Since CHF was observed only at high doses of compound A, this analysis was restricted to the 10 and 20 mg treatment arms of the CV168-006 trial. As shown below, polymorphisms in the PPAR-gamma (PPAR-gamma; P=0.002) gene showed evidence of association with CHF.

Genotyping was performed using the 5′ nuclease assay, essentially as described (Ranade K et al., Genome Research 11: 1262-1268 (2001); which is hereby incorporated by reference herein in its entirety), with the following modifications: six nanograms of genomic DNA were used in a 8 ul reaction. All PCR reactions were performed in an ABI 9700 machine and fluorescence was measured using an ABI 7900 machine.

Genotyping of the SNPs of the present invention was performed using sets of Taqman probes (100 uM each) and primers (100 uM each) specific to the SNP. Each probe/primer set was manually designed using ABI Primer Express software (Applied Biosystems). Genomic samples were prepared as described in Example 7. The following Taqman probes and primers were utilized:

| | Taqman Forward | Taqman Reverse | Reference Taqman | Variable Taqman |
|---|---|---|---|---|
| SNP | Primer | Primer | Probe | Probe |
| SNP1 | AGACAAAATATCAGTG | GTGATATGTTTGCAGA | AGATTCTCCTATTGAC | ATTCTCCTATTGACGC |
| | TGAATTACAGCA | CAGTGTATCAGT | CCAGAAAGCGATTCC | AGAAAGCGATTCCT |
| | (SEQ ID NO: 17) | (SEQ ID NO: 18) | (SEQ ID NO: 19) | (SEQ ID No: 20) |

** The allelic nucleotide in each probe sequence is shown in bold and underlined.

The genotype assay conditions are provided below.

| Components: | Final Concentration: |
|---|---|
| 2x PE Master Mix (#4318157) | 1X |
| 100 uM FAM labeled probe | 200 nmol |
| 100 uM VIC labeled probe | 200 nmol |
| Forward PCR primer | 600 nmol |
| Reverse PCR primer | 600 nmol |
| 6 ng template DNA | as required |
| ddH20 | volume to 8 ul |

Taqman thermo-cycling was performed on Perkin Elmer PE 9700 machines using the following cycling conditions below:
1) 50 C for 2 minutes
2) 95 C for 10 seconds*
3) 94 C for 15 seconds
4) 62 C for 1 minute
5) 4 C hold
   *Steps 2-4 were cycled 40 times

Analysis of genotypes was performed by using the Applied Biosystems ABI 7900 HT sequence detection system.

### Example 9

### Statistical Analysis of the Association Between Dose-dependent Peripheral Congestive Heart Failure and the SNPs of the Present Invention

The association between congestive heart failure and the single nucleotide polymorphisms of the present invention were investigated by applying statistical analysis to the results of the genotyping assays described elsewhere herein. The central hypothesis of this analysis is that a predisposition to develop dose-dependent congestive heart failure may be conferred by specific genomic factors. The analysis attempted to identify one or more of these factors in genomic DNA samples from index cases and matched control subjects who were exposed to Compound A in two Bristol-Myers Squibb (BMS) clinical studies (see Example 8).

SNPs of the present invention were examined for association with congestive heart failure using 3 (genotypes)×2 (congestive heart failure and no congestive heart failure) contingency tables. Analyses were performed using S-plus (version 6.0; Insightful Corp. Seattle, Wash.) or SPSS (version 12.0; SPSS Inc. Chicago, Ill.).

### Methods

Sample. Investigators in the BMS clinical trials diagnosed dose-dependent congestive heart failure in some subjects.

Measures. Single nucleotide polymorphisms (SNPs) in human PPAR-gamma were genotyped on all subjects essentially as described in Example 8 herein. The SNPs that are genotyped likely represent a sample of the polymorphic variation in each gene and are not exhaustive with regard to coverage of the total genetic variation that may be present in each gene. Specifically, only the 1 SNP referenced herein was genotyped and statistically analyzed, as described. The SNP for which a statistical association to corigestive heart failure susceptibility was confirmed are provided and refered to as SNP1.

Statistical Analyses. Association between SNP1 with the incidence of congestive heart failure was performed using the Fisher test (Fisher L D, van Belle G (1993) Biostatistics. John Wiley & Sons, New York, N.Y.). Analysis was done by comparing the genotype frequencies of SNPs in patients who developed CHF to those who were not affected. Analysis was restricted to patients treated with 10 and 20 mg of compound A in CV168-006 and CV168-008 Phase II trials.

Since the SNP coverage within the human PPAR-gamma gene was not exhaustive of the genetic variation that may be present and possibly related to event susceptibility in this gene, inferences about these SNP associations with dose-dependent congestive heart failure events for PPAR-gamma are therefore related to the hypothesis that genetic variation in this gene may be involved in susceptibility to such events.

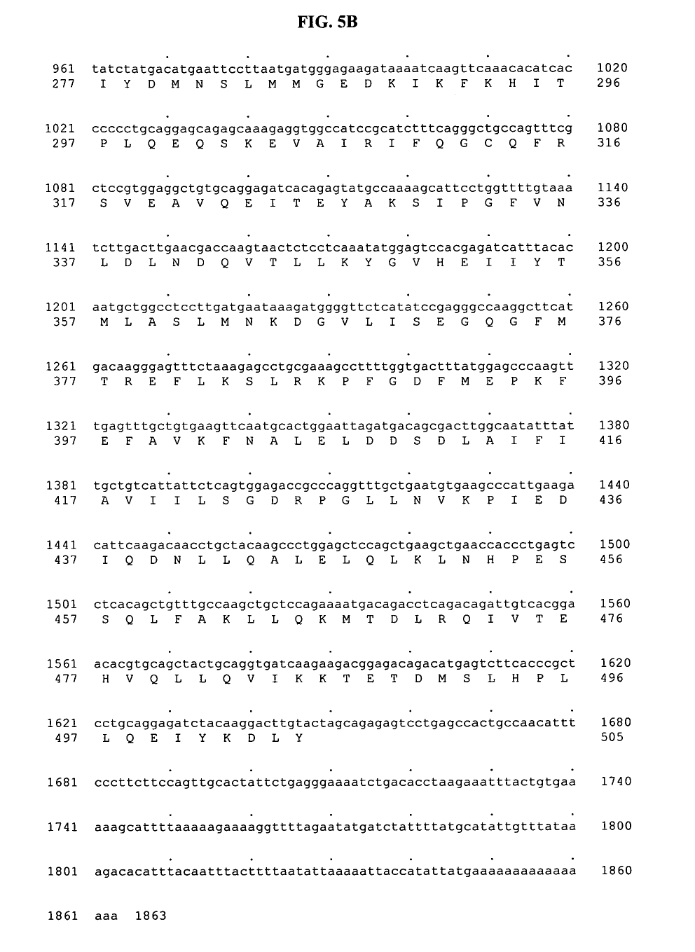

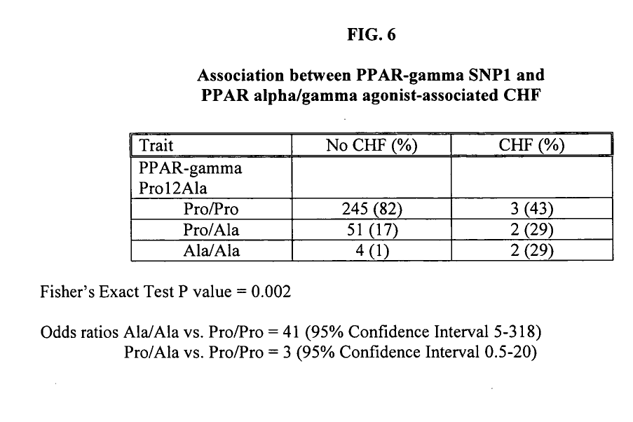

The association statistics for SNP1 are provided in FIG. 6.

### Genetic Associations with Congestive Heart Failure

All subjects for whom sufficient DNA was available were genotyped for 218 SNPs in 65 candidate genes with 1 of these SNPs being specific for the human PPAR-gamma gene. Of these 218 SNPs, subjects enrolled in these trials were polymorphic for 153 SNPs (minor allele frequency >0.1%). In 3×2 contingency table analysis, SNPs in the PPAR-gamma genes were significantly associated with congestive heart failure status (P<0.05).

The PPAR-gamma SNP1 was determined to be in Hardy-Weinberg equilibrium. These results suggest that polymorphisms in the PPAR-gamma gene contributes to differences in susceptibility to dose-dependent congestive heart failure independent of other significant predictors such as age, sex and body mass index.

SNPs in the PPAR-gamma gene increased risk of CHF. Recently, Deeb et al determined that the variable allele at amino acid 12 of SEQ ID NO:14 increases a patients risk of developing diabetes and that this SNP appears to affect the function of the protein. While the diabetes association is not causally related to the association of this SNP to the incidence of CHF, is does help illustrate the importance of identifying patients that harbor this SNP to identify those patients at risk of developing diabetes, on one hand, and identifying patients at risk of developing dose dependent CHF upon the administration of a PPAR-agonist.

The utility, in general, of each of these significant SNP-dose-dependent congestive heart failure event associations is that they suggest (1) such SNPs may be causally involved, alone or in combination with other SNPs in the respective gene regions with susceptibility to dose-dependent congestive heart failure events resulting from exposure to a PPAR-agonist; (2) such SNPs, if not directly causally involved, are reflective of an association because of linkage disequilibrium with one or more other SNPs that may be causally involved, alone or in combination with other SNPs in the respective gene regions with susceptibility to dose-dependent congestive heart failure resulting from exposure to a PPAR-agonist; (3) such SNPs may be useful in establishing haplotypes that may be used to narrow the search for and identify polymorphisms or combinations of polymorphisms that may be causally, alone or in combination with other SNPs in the respective gene regions with susceptibility to dose-dependent congestive heart failure resulting from exposure to a PPAR-agonist; and (4) such SNPs, if used to establish haplotypes that are identified as causally involved in such event susceptibility, may be used to predict which subjects are most likely to experience such events when exposed to a dose-dependent congestive heart failure resulting from exposure to a PPAR-agonist. The term “respective gene regions” shall be construed to refer to those regions of each gene which have been used to identify the SNPs of the present invention.

### Example 10

### Method of Isolating the Native Forms of the Human PPAR-gamma Gene

A number of methods have been described in the art that may be utilized in isolating the native forms of the human PPAR-gamma gene. Specific methods are referenced below and are hereby incorporated by reference herein in their entireties. The artisan, skilled in the molecular biology arts, would be able to isolate the native form of human PPAR-gamma based upon the methods and information contained, and/or referenced, therein.

Human Reference PPAR-gamma (gi|NM_{—}138712; SEQ ID NO:13 and 14)

1) Fajas, L. et al., J. Biol. Chem. 272(30), 18779-18789 (1997).
2) Beamer, B. A. et al., Biochem. Biophys. Res. Commun. 233(3), 756-759 (1997).
3) Mukherjee, R. et al., J. Biol. Chem. 272(12), 8071-8076 (1997).
4) Adams, M. et al., J. Biol. Chem. 272(8), 5128-5132 (1997).
5) Chu, R. et al., J. Biol. Chem. 271(44), 27670-27676 (1996).
6) Elbrecht, A. et al., Biochem. Biophys. Res. Commun. 224(2), 431-437 (1996).
7) Zhu, Y. et al., Gene Expr. 6(3), 185-195 (1996).
8) Greene, M. E. et al., Gene Expr. 4(4-5), 281-299 (1995).
9) Tontonoz, P. et al., Cell 79(7), 1147-1156 (1994).
10) Tontonoz, P. et al., Nucleic Acids Res. 22(25), 5628-5634 (1994).
11) Soares-da-Silva, P. and Fernandes, M. H., Br. J. Pharmacol. 105(4), 811-816 (1992).

Methods of isolation for the human PPAR-gamma gene of the present invention may also be found in reference to the references cited in the Genbank accession nos. for each gene provided herein which are hereby incorporated by reference herein.

### Example 11

### Method of Isolating the Polymorphic Forms of the Human PPAR-gamma Gene of the Present Invention

Since the allelic genes of the present invention represent genes present within at least a subset of the human population, these genes may be isolated using the methods provided in Example 10 above. For example, the source DNA used to isolate the allelic gene may be obtained through a random sampling of the human population and repeated until the allelic form of the gene is obtained. Preferably, random samples of source DNA from the human population are screened using the SNPs and methods of the present invention to identify those sources that comprise the allelic form of the gene. Once identified, such a source may be used to isolate the allelic form of the gene(s). The invention encompasses the isolation of such allelic genes from both genomic and/or cDNA libraries created from such source(s).

In reference to the specific methods provided in Example 10 above, it is expected that isolating the polymorphic alleles of the human PPAR-gamma gene would be within the skill of an artisan trained in the molecular biology arts. Nonetheless, a detailed exemplary method of isolating at least one of the PPAR-gamma polymorphic alleles, in this case the variant form of SNP1 (“g” nucleotide at 167 of SEQ ID NO:15) is provided. Briefly,

First, the individuals with the c167g variation are identified by genotyping the genomic DNA samples using the method outlined in Example 7 herein. Other methods of genotyping may be employed, such as the FP-SBE method (Chen et al., Genome Res., 9(5):492-498 (1999)), or other methods described herein. DNA samples publicly available (e.g., from the Coriell Institute (Collingswood, N.J.) or from the Bristol-Myers Squibb clinical samples described herein may be used. Oligonucleotide primers that are used for this genotyping assay are provided in Example 7.

By analyzing genomic DNA samples, individuals with the c167g form of the SNP1 variant may be identified. Once identified, clones comprising the genomic sequence may be obtained using methods well known in the art (see Sambrook, J., E. F. Fritsch, and T. Maniatis, 1989, Molecular Cloning: A Laboratory Manual, Cold Spring Harbor Laboratory Press, Cold Spring Harbor, N.Y.; and Current Protocols in Molecular Biology, 1995, F. M., Ausubel et al., eds., John Wiley and Sons, Inc., which are hereby incorporated by reference herein.).

If cDNA clones of the coding sequence of this allele of the gene are of interest, such clones may. be obtained in accordance with the following steps. Next, Lymphoblastoid cell lines from these individuals may be obtained from the Coriell Institute. These cells can be grown in RPMI-1640 medium with L-glutamine plus 10% FCS at 37degrees. PolyA+ RNA are then isolated from these cells using Oligotex Direct Kit (Life Technologies).

First strand cDNA (complementary DNA) is produced using Superscript Preamplification System for First Strand cDNA Synthesis (Life Technologies, Cat No 18089-011) using these polyA+ RNA as templates, as specified in the users manual which is hereby incorporated herein by reference in its entirety. Specific cDNA encoding the human PPAR-gamma protein is amplified by polymerase chain reaction (PCR) using a forward primer which hybridizes to the 5′-UTR region, a reverse primer which hybridizes to the 3′-UTR region, and these first strand cDNA as templates (Sambrook, Fritsch et al. 1989). Alternatively, these primers may be designed using Primer3 program (Rozen S 2000). Restriction enzyme sites (example: SalI for the forward primer, and NotI for reverse primer) are added to the 5′-end of these primer sequences to facilitate cloning into expression vectors after PCR amplification. PCR amplification may be performed essentially as described in the owner's manual of the Expand Long Template PCR System (Roche Molecular Biochemicals) following manufacturer's standard protocol, which is hereby incorporated herein by reference in its entirety.

PCR amplification products are digested with restriction enzymes (such as SalI and NotI, for example) and ligated with expression vector DNA cut with the same set of restriction enzymes. pSPORT (Invitrogen) is one example of such an expression vector. After ligated DNA is introduced into E. coli cells (Sambrook, Fritsch et al. 1989), plasmid DNA is isolated from these bacterial cells. This plasmid DNA is sequenced to confirm the presence an intact (full-length) coding region of the human PPAR-gamma protein with the variation, if the variation results in changes in the encoded amino acid sequence, using methods well known in the art and described elsewhere herein.

The skilled artisan would appreciate that the above method may be applied to isolating the other novel human PPAR-gamma genes of the present invention through the simple substitution of applicable PCR and sequencing primers. Such primers may be selected from any one of the applicable primers provided in herein, or may be designed using the Primer3 program (Rozen S 2000) as described. Such primers may preferably comprise at least a portion of any one of the polynucleotide sequences of the present invention.

### EXAMPLE 12

### Method of Engineering the Allelic Forms of the Human PPAR-gamma Gene of the Present Invention

Aside from isolating the allelic genes of the present invention from DNA samples obtained from the human population, Bristol-Myers Squibb Company clinical trials, and/or the Coriell Institute, as described in Example 11 above, the invention also encompasses methods of engineering the allelic genes of the present invention through the application of site-directed mutagenesis to the isolated native forms of the genes. Such methodology could be applied to synthesize allelic forms of the genes comprising at least one, or more, of the encoding SNPs of the present invention (e.g., silent, missense)—preferably at least 1, 2, 3, or 4 encoding SNPs for each gene.

In reference to the specific methods provided in Example 11 above, it is expected that isolating the novel polymorphic PPAR-gamma genes of the present invention would be within the skill of an artisan trained in the molecular biology arts. Nonetheless, a detailed exemplary method of engineering at least one of the PPAR-gamma polymorphic alleles to comprise the encoding and/or non-coding polymorphic nucleic acid sequence, in this case the variant form (c167g) of SNP1 (SEQ ID NO:15) is provided. Briefly,

cDNA clones encoding the human PPAR-gamma protein may be identified by homology searches with the BLASTN program (Altschul SF 1990) against the Genbank non-redundant nucleotide sequence database using the published reference human PPAR-gamma cDNA sequence (GenBank Accession No.: gi| NM_{—}138712). Alternatively, the genomic sequence of the human PPAR-gamma gene may be obtained as described herein. After obtaining these clones, they are sequenced to confirm the validity of the DNA sequences.

However, in the case of the reference form (c167) of SNP1, genomic clones would need to be obtained and may be identified by homology searches with the BLASTN program (Altschul SF 1990) against the Genbank non-redundant nucleotide sequence database using the published human PPAR-gamma cDNA sequence (GenBank Accession No.: gi| NM_{—}138712). Alternatively, the genomic sequence of the human reference PPAR-gamma gene may be obtained as described herein. After obtaining these clones, they are sequenced to confirm the validity of the DNA sequences.

Once these clones are confirmed to contain the intact wild type cDNA or genomic sequence of the human PPAR-gamma coding and/or non-coding region, the c167g polymorphism (mutation) may be introduced into the native sequence using PCR directed in vitro mutagenesis (Cormack, B., Directed Mutagenesis Using the Polymerase Chain Reaction. Current Protocols in Molecular Biology, John Wiley & Sons, Inc. Supplement 37: 8.5.1-8.5.10, (2000)). In this method, synthetic oligonucleotides are designed to incorporate a point mutation at one end of an amplified fragment. Following PCR, the amplified fragments are made blunt-ended by treatment with Klenow Fragment. These fragments are then ligated and subcloned into a vector to facilitate sequence analysis. This method consists of the following steps.
1. Subcloning of cDNA or genomic insert into a plasmid vector, or BAC sequence if the clone is a genomic sequence, containing multiple cloning sites and M13 flanking sequences, such as pUC19 (Sambrook, Fritsch et al. 1989), in the forward orientation. The skilled artisan would appreciate that other plasmids could be equally substituted, and may be desirable in certain circumstances.
2. Introduction of a mutation by PCR amplification of the cDNA region downstream of the mutation site using a primer including the mutation. (FIG. 8.5.2 in Cormack 2000)). In the case of introducing the reference c167g (i.e., P12A) sequence mutation into the human PPAR-gamma protein, the following two primers may be used.

M13 Reverse Sequencing Primer:

| | |
|---|---|
| 5′- CGCCAGGGTTTTCCCAGTCACGAC -3′. | (SEQ ID NO: 21) |

Mutation Primer:

(SEQ ID NO: 23)

5′- GGGAGATTCTCCTATTGACGCAGAAAGCGATTCCTTCAC -3′

Mutation primer contains the mutation (c167g) at the center (in bold and underlined) and a portion of its flanking sequence. M13 reverse sequencing primer hybridizes to the pUC19 vector. Subcloned cDNA or genomic clone comprising the human PPAR-gamma cDNA or genomic sequence is used as a template (described in Step 1). A 100 ul PCR reaction mixture is prepared using 10 ng of the template DNA, 200 uM 4dNTPs, 1 uM primers, 0.25U Taq DNA polymerase (PE), and standard Taq DNA polymerase buffer. Typical PCR cycling condition are as follows:

| | |
|---|---|
| 20-25 cycles: | 45 sec, 93 degrees |
| | 2 min, 50 degrees |
| | 2 min, 72 degrees |
| 1 cycle: | 10 min, 72 degrees |

After the final extension step of PCR, 5U Klenow Fragment is added and incubated for 15 min at 30 degrees. The PCR product is then digested with the restriction enzyme, EcoRI.
3. PCR amplification of the upstream region is then performed, using subcloned cDNA or genomic clone as a template (the product of Step 1). This PCR is done using the following two primers:

M13 Forward Sequencing Primer:

| | |
|---|---|
| 5′- CGCCAGGGTTTTCCCAGTCACGAC -3′. | (SEQ ID NO: 22) |

Flanking Primer:

(SEQ ID NO: 24)

5′- GTGAAGGAATCGCTTTCTGCGTCAATAGGAGAATCTCCC -3′.

Flanking primer is complimentary to the upstream flanking sequence and mutation locus of the c167g mutation (in bold and underlined). M13 forward sequencing primer hybridizes to the pUC19 vector. PCR conditions and Klenow treatments follow the same procedures as provided in Step 2, above. The PCR product is then digested with the restriction enzyme, HindIII.
4. Prepare the pUC19 vector for cloning the cDNA or genomic clone comprising the polymorphic locus. Digest pUC19 plasmid DNA with EcoRI and HindII. The resulting digested vector fragment may then be purified using techniques well known in the art, such as gel purification, for example.
5. Combine the products from Step 2 (PCR product containing mutation), Step 3 (PCR product containing the upstream region), and Step 4 (digested vector), and ligate them together using standard blunt-end ligation conditions (Sambrook, Fritsch et al. 1989).
6. Transform the resulting recombinant plasmid from Step 5 into E. coli competent cells using methods known in the art, such as, for example, the transformation methods described in Sambrook, Fritsch et al. 1989.
7. Analyze the amplified fragment portion of the plasmid DNA by DNA sequencing to confirm the point mutation, and absence of any other mutations introduced during PCR. The method of sequencing the insert DNA, including the primers utilized, are described herein or are otherwise known in the art.

Moreover, the skilled artisan would appreciate that the above method may be applied to engineering more than one polymorphic nucleic acid sequence of the present invention into the novel PPAR-gamma genes of the present invention. Such an engineered gene could be created through succesive rounds of site-directed mutagenesis, as described in Steps 1 thru 7 above, or consolidated into a single round of mutagenesis. For example, Step 2 above could be performed for each mutation, then the products of both mutation amplifications could be combined with the product of Step 3 and 4, and the procedure followed as described.

### EXAMPLE 13

### Method of Discovering the Single Nucleotide Polymorphisms (SNPs) of the Present Invention

SNP discovery was based on comparative DNA sequencing of PCR products directed to the human Adiponectin gene derived from genomic DNA from multiple individuals. All genomic DNA samples for SNP discovery were obtained from a panel of thirty-two human subjects obtained from the Coriell Institute (Camden, N.J.), panel # M44PDR. A total of 1 SNP within the human Adiponectin gene was identified, and found to be associated with the incidence of dose-dependent congestive heart failure:

Adiponectin-SNP1: T/C at nucleotide 415 of SEQ ID NO:26 and 27

Adiponectin SNP1 was identified using the following protocol. Briefly, portions of the human Adiponectin genomic sequence (SEQ ID NO:38) were PCR amplified using the standard Platinum Taq DNA protocol (Invitrogen, Product #10966-083):

The following sequencing primers (20 uM each) were used to confirm the presence of the above SNP(s), as well as to sequence across the PCR amplicons:

| | | |
|---|---|---|
| SNP | Forward Primer | Reverse Primer |

| | | |
|---|---|---|
| Adiponectin | AGGTCCCCGAGGCTTTCC | AAGCGAATGGGCATGTTGG |
| SNP1 | (SEQ ID NO: 30) | (SEQ ID NO: 31) |

All the samples amplified from genomic DNA (50 ng) in reactions (50 ul) containing 50 mM Tris-Acetate pH 8.4, 75 mM KAcetate, 8 mM MgAcetate, 200 uM dNTPs, 0.2 uM of each PCR primer, and 2.5 U Platinum Taq DNA polymerase (Invitrogen).

PCR amplification was performed in Perkin Elmer 9700 machines under the following cycling conditions: 1.) 94 degrees Celsius for 2 minutes; 2.) 94 degrees Celsius for 30 seconds; 3.) 59 degrees Celsius for 1 minute; 4.) 72 degrees Celsius for 30 seconds; 5.) 72 degrees Celsius for 5 minutes; and 6.) 4 degrees Celsius on hold. Steps 2 to 4 were cycled 35 times.

PCR products were sequenced using ABI BigDye Terminator v3.1 Cycle Sequencing chemistry on the 3730-XL capillary sequencers using the same primers used for identifying the SNPs, as described above.

Sequence editing and contig assembly was performed using CONSED software (Genome Res. 1998 March; 8(3):195-202). Chromatograms were visually inspected for each Coriell DNA and SNPs identified by comparing sequence traces to the reference Adiponectin provided as SEQ ID NO:26 and 27 herein.

In the instant study, the “His” allele at amino acid 111 of SEQ ID NO:27 was found to be the rare allele and thus has been termed the variable allele accordingly, with the “Tyr” allele at amino acid 111 of SEQ ID NO:27 serving as the reference allele.

The nucleotide sequence of the Adiponectin gene containing the reference allele (“t”) for SNP1 at nucleotide 415 is provided in FIGS. 7A-D (SEQ ID NO:26); while the nucleotide sequence of the Adiponectin gene containing the variable allele (“c”) for SNP1 at nucleotide 415 is provided in FIGS. 8A-D (SEQ ID NO:28).

The polypeptide sequence of Adiponectin containing the reference allele (“Tyr”) for SNP1 at amino acid 111 is provided in FIGS. 7A-D (SEQ ID NO:27); while the polypeptide sequence of Adiponectin containing the variable allele (“His”) for SNP1 at amino acid 111 is provided in FIGS. 8A-D (SEQ ID NO:29).

### Example 14

### Method of Genotyping Each SNP of the Present Invention

Genomic DNA samples from patients enrolled in two Bristol-Myers Squibb Company Phase II clinical trials CV168-006 and CV168-008 trials were genotyped for 1 SNP identified in the human Adiponectin gene (see Example 13) and evaluated for association with congestive heart failure.

498 subjects enrolled in the CV168-006 trial and 232 subjects from the CV168-008 trial were analyzed in this study. All analyses were based on data collected up to 24 weeks, which was the duration of the short-term phase of the trials. DNA was extracted from frozen blood by a third-party (Genaissance Inc, North Carolina) using a salting-out method (Gentra Systems). All subjects gave written informed consent.

At the time of this analysis four individuals were diagnosed as having developed congestive heart failure. All four were in the CV168-006 trial; three were being treated with 10 mg of compound A and one with 20 mg of compound A. All SNPs that were analyzed above were also tested for association with CHF using contingency tables. Since CHF was observed only at high doses of compound A, this analysis was restricted to the 10 and 20 mg treatment arms of the CV168-006 trial. As shown below, polymorphisms in the Adiponectin (APM-1; P=0.015) gene showed evidence of association with CHF.

Genotyping was performed using the 5′ nuclease assay, essentially as described (Ranade K et al., Genome Research 11: 1262-1268 (2001); which is hereby incorporated by reference herein in its entirety), with the following modifications: six nanograms of genomic DNA were used in a 8 ul reaction. All PCR reactions were performed in an ABI 9700 machine and fluorescence was measured using an ABI 7900 machine.

Genotyping of the SNPs of the present invention was performed using sets of Taqman probes (100 uM each) and primers (100 uM each) specific to the SNP. Each probe/primer set was manually designed using ABI Primer Express software (Applied Biosystems). Genomic samples were prepared as described in Example 13. The following Taqman probes and primers were utilized:

| | Taqman Forward | Taqman Reverse | Reference Taqman | Variable Taqman |
|---|---|---|---|---|
| SNP | Primer | Primer | Probe | Probe |
| SNP1 | AGGTCCCCGAGGCTTT | AAGCGAATGGGCATGT | AGGTGCCTATGTAtAC | AGGTGCCTATGTAcAC |
| | CC | TGG | CGCTCAGCATTCA | CGCTCAGCATT |
| | (SEQ ID NO: 30) | (SEQ ID NO: 31) | (SEQ ID NO: 32) | (SEQ ID NO: 33) |

** The allelic nucleotide in each probe sequence is shown in bold and underlined.

The genotype assay conditions are provided below.

| Components: | Final Concentration: |
|---|---|
| 2x PE Master Mix (#4318157) | 1X |
| 100 uM FAM labeled probe | 200 nmol |
| 100 uM VIC labeled probe | 200 nmol |
| Forward PCR primer | 600 nmol |
| Reverse PCR primer | 600 nmol |
| 6 ng template DNA | as required |
| ddH20 | volume to 8 ul |

Taqman thermo-cycling was performed on Perkin Elmer PE 9700 machines using the following cycling conditions below:
1) 50 C for 2 minutes
2) 95 C for 10 seconds*
3) 94 C for 15 seconds
4) 62 C for 1 minute
5) 4 C hold
   *Steps 2-4 were cycled 40 times

Analysis of genotypes was performed by using the Applied Biosystems ABI 7900 HT sequence detection system.

### EXAMPLE 15

### Statistical Analysis of the Association Between Dose-Dependent Peripheral Congestive Heart Failure and the SNPs of the Present Invention

The association between congestive heart failure and the single nucleotide polymorphisms of the present invention were investigated by applying statistical analysis to the results of the genotyping assays described elsewhere herein. The central hypothesis of this analysis is that a predisposition to develop dose-dependent congestive heart failure may be conferred by specific genomic factors. The analysis attempted to identify one or more of these factors in genomic DNA samples from index cases and matched control subjects who were exposed to Compound A in two Bristol-Myers Squibb (BMS) clinical studies (see Example 14).

SNPs of the present invention were examined for association with congestive heart failure using 3 (genotypes)×2 (congestive heart failure and no congestive heart failure) contingency tables. Analyses were performed using S-plus (version 6.0; Insightful Corp. Seattle, Wash.) or SPSS (version 12.0; SPSS Inc. Chicago, Ill.).

### Methods

Sample. Investigators in the BMS clinical trials diagnosed dose-dependent congestive heart failure in some subjects.

Measures. Single nucleotide polymorphisms (SNPs) in human Adiponectin were genotyped on all subjects essentially as described in Example 14 herein. The SNPs that are genotyped likely represent a sample of the polymorphic variation in each gene and are not exhaustive with regard to coverage of the total genetic variation that may be present in each gene. Specifically, only the 1 SNP referenced herein was genotyped and statistically analyzed, as described. The SNP for which a statistical association to congestive heart failure susceptibility was confirmed are provided and refered to as SNP 1.

Statistical Analyses. Association between SNP1 with the incidence of congestive heart failure was performed using the Fisher test (Fisher L D, van Belle G (1993) Biostatistics. John Wiley & Sons, New York, N.Y.). Analysis was done by comparing the genotype frequencies of SNPs in patients who developed CHF to those who were not affected. Analysis was restricted to patients treated with 10 and 20 mg of compound A in CV168-006 and CV168-008 Phase II trials.

Since the SNP coverage within the human Adiponectin gene was not exhaustive of the genetic variation that may be present and possibly related to event susceptibility in this gene, inferences about these SNP associations with dose-dependent congestive heart failure events for Adiponectin are therefore related to the hypothesis that genetic variation in this gene may be involved in susceptibility to such events.

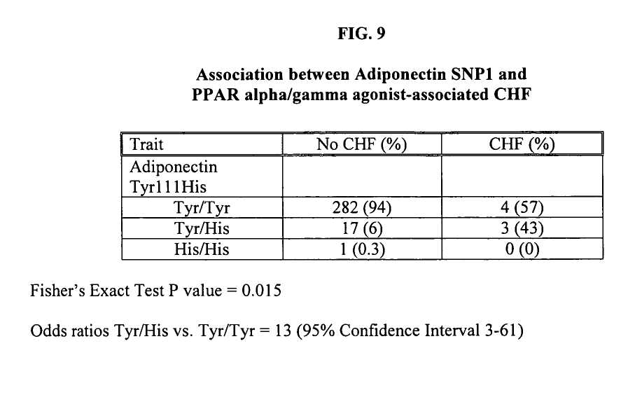

The association statistics for SNP1 are provided in FIG. 9.

### Genetic Associations with Congestive Heart Failure

All subjects for whom sufficient DNA was available were genotyped for 218 SNPs in 65 candidate genes with 1 of these SNPs being specific for the human Adiponectin gene. Of these 218 SNPs, subjects enrolled in these trials were polymorphic for 153 SNPs (minor allele frequency >0.1%). In 3×2 contingency table analysis, SNPs in the Adiponectin genes were significantly associated with congestive heart failure status (P<0.05).

The Adiponectin SNP1 was determined to be in Hardy-Weinberg equilibrium. These results suggest that polymorphisms in the Adiponectin gene contributes to differences in susceptibility to dose-dependent congestive heart failure independent of other significant predictors such as age, sex and body mass index.

The utility, in general, of each of these significant SNP-dose-dependent congestive heart failure event associations is that they suggest (1) such SNPs may be causally involved, alone or in combination with other SNPs in the respective gene regions with susceptibility to dose-dependent congestive heart failure events resulting from exposure to a PPAR-agonist; (2) such SNPs, if not directly causally involved, are reflective of an association because of linkage disequilibrium with one or more other SNPs that may be causally involved, alone or in combination with other SNPs in the respective gene regions with susceptibility to dose-dependent congestive heart failure resulting from exposure to a PPAR-agonist; (3) such SNPs may be useful in establishing haplotypes that may be used to narrow the search for and identify polymorphisms or combinations of polymorphisms that may be causally, alone or in combination with other SNPs in the respective gene regions with susceptibility to dose-dependent congestive heart failure resulting from exposure to a PPAR-agonist; and (4) such SNPs, if used to establish haplotypes that are identified as causally involved in such event susceptibility, may be used to predict which subjects are most likely to experience such events when exposed to a dose-dependent congestive heart failure resulting from exposure to a PPAR-agonist. The term “respective gene regions” shall be construed to refer to those regions of each gene which have been used to identify the SNPs of the present invention.

### Example 16

### Method of Isolating the Native Forms of the Human Adiponectin Gene

A number of methods have been described in the art that may be utilized in isolating the native forms of the human Adiponectin gene. Specific methods are referenced below and are hereby incorporated by reference herein in their entireties. The artisan, skilled in the molecular biology arts, would be able to isolate the native form of human Adiponectin based upon the methods and information contained, and/or referenced, therein.

Human Reference Adiponectin (gi| NM_{—}004797.2; SEQ ID NO:26 and 27)

1) Halleux, C. M. et al., Biochem. Biophys. Res. Commun. 288 (5), 1102-1107 (2001).
2) Das, K. et al., Biochem. Biophys. Res. Commun. 280 (4), 1120-1129 (2001).
3) Okamoto, Y. et al., Horm. Metab. Res. 32 (2), 47-50 (2000).
4) Schaffler, A. et al., Biochem. Biophys. Res. Commun. 260 (2), 416-425 (1999).
5) Saito, K. et al., Gene 229 (1-2), 67-73 (1999).
6) Schaffler, A. et al., Biochim. Biophys. Acta 1399 (2-3), 187-197 (1998).
7) Hu, E. et al., J. Biol. Chem. 271 (18), 10697-10703 (1996).
8) Maeda, K. et al., Biochem. Biophys. Res. Commun. 221 (2), 286-289 (1996).
9) Scherer, P. E. et al., J. Biol. Chem. 270 (45), 26746-26749 (1995).

Methods of isolation for the human Adiponectin gene of the present invention may also be found in reference to the references cited in the Genbank accession nos. for each gene provided herein which are hereby incorporated by reference herein.

### Example 17

### Method of Isolating the Polymorphic Forms of the Human Adiponectin Gene of the Present Invention

Since the allelic genes of the present invention represent genes present within at least a subset of the human population, these genes may be isolated using the methods provided in Example 16 above. For example, the source DNA used to isolate the allelic gene may be obtained through a random sampling of the human population and repeated until the allelic form of the gene is obtained. Preferably, random samples of source DNA from the human population are screened using the SNPs and methods of the present invention to identify those sources that comprise the allelic form of the gene. Once identified, such a source may be used to isolate the allelic form of the gene(s). The invention encompasses the isolation of such allelic genes from both genomic and/or cDNA libraries created from such source(s).

In reference to the specific methods provided in Example 16 above, it is expected that isolating the polymorphic alleles of the human Adiponectin gene would be within the skill of an artisan trained in the molecular biology arts. Nonetheless, a detailed exemplary method of isolating at least one of the Adiponectin polymorphic alleles, in this case the variant form of SNP1 (“c” nucleotide at 415 of SEQ ID NO:28) is provided. Briefly,

First, the individuals with the t415c variation are identified by genotyping the genomic DNA samples using the method outlined in Example 14 herein. Other methods of genotyping may be employed, such as the FP-SBE method (Chen et al., Genome Res., 9(5):492-498 (1999)), or other methods described herein. DNA samples publicly available (e.g., from the Coriell Institute (Collingswood, N.J.) or from the Bristol-Myers Squibb clinical samples described herein may be used. Oligonucleotide primers that are used for this genotyping assay are provided in Example 14.

By analyzing genomic DNA samples, individuals with the t415c form of the SNP1 variant may be identified. Once identified, clones comprising the genomic sequence may be obtained using methods well known in the art (see Sambrook, J., E. F. Fritsch, and T. Maniatis, 1989, Molecular Cloning: A Laboratory Manual, Cold Spring Harbor Laboratory Press, Cold Spring Harbor, N.Y.; and Current Protocols in Molecular Biology, 1995, F. M., Ausubel et al., eds, John Wiley and Sons, Inc., which are hereby incorporated by reference herein.).

If cDNA clones of the coding sequence of this allele of the gene are of interest, such clones may be obtained in accordance with the following steps. Next, Lymphoblastoid cell lines from these individuals may be obtained from the Coriell Institute. These cells can be grown in RPMI-1640 medium with L-glutamine plus 10% FCS at 37 degrees. PolyA+ RNA are then isolated from these cells using Oligotex Direct Kit (Life Technologies).

First strand cDNA (complementary DNA) is produced using Superscript Preamplification System for First Strand cDNA Synthesis (Life Technologies, Cat No 18089-011) using these polyA+ RNA as templates, as specified in the users manual which is hereby incorporated herein by reference in its entirety. Specific cDNA encoding the human Adiponectin protein is amplified by polymerase chain reaction (PCR) using a forward primer which hybridizes to the 5′-UTR region, a reverse primer which hybridizes to the 3′-UTR region, and these first strand cDNA as templates (Sambrook, Fritsch et al. 1989). Alternatively, these primers may be designed using Primer3 program (Rozen S 2000). Restriction enzyme sites (example: SalI for the forward primer, and NotI for reverse primer) are added to the 5′-end of these primer sequences to facilitate cloning into expression vectors after PCR amplification. PCR amplification may be performed essentially as described in the owner's manual of the Expand Long Template PCR System (Roche Molecular Biochemicals) following manufacturer's standard protocol, which is hereby incorporated herein by reference in its entirety.

PCR amplification products are digested with restriction enzymes (such as SalI and NotI, for example) and ligated with expression vector DNA cut with the same set of restriction enzymes. pSPORT (Invitrogen) is one example of such an expression vector. After ligated DNA is introduced into E. coli cells (Sambrook, Fritsch et al. 1989), plasmid DNA is isolated from these bacterial cells. This plasmid DNA is sequenced to confirm the presence an intact (full-length) coding region of the human Adiponectin protein with the variation, if the variation results in changes in the encoded amino acid sequence, using methods well known in the art and described elsewhere herein.

The skilled artisan would appreciate that the above method may be applied to isolating the other novel human Adiponectin genes of the present invention through the simple substitution of applicable PCR and sequencing primers. Such primers may be selected from any one of the applicable primers provided in herein, or may be designed using the Primer3 program (Rozen S 2000) as described. Such primers may preferably comprise at least a portion of any one of the polynucleotide sequences of the present invention.

### Example 18

### Method of Engineering the Allelic Forms of the Human Adiponectin Gene of the Present Invention

Aside from isolating the allelic genes of the present invention from DNA samples obtained from the human population, Bristol-Myers Squibb Company clinical trials, and/or the Coriell Institute, as described in Example 17 above, the invention also encompasses methods of engineering the allelic genes of the present invention through the application of site-directed mutagenesis to the isolated native forms of the genes. Such methodology could be applied to synthesize allelic forms of the genes comprising at least one, or more, of the encoding SNPs of the present invention (e.g., silent, missense)—preferably at least 1, 2, 3, or 4 encoding SNPs for each gene.

In reference to the specific methods provided in Example 17 above, it is expected that isolating the novel polymorphic Adiponectin genes of the present invention would be within the skill of an artisan trained in the molecular biology arts. Nonetheless, a detailed exemplary method of engineering at least one of the Adiponectin polymorphic alleles to comprise the encoding and/or non-coding polymorphic nucleic acid sequence, in this case the variant form (t415c) of SNP1 (SEQ ID NO:28) is provided. Briefly,

cDNA clones encoding the human Adiponectin protein may be identified by homology searches with the BLASTN program (Altschul S F 1990) against the Genbank non-redundant nucleotide sequence database using the published reference human Adiponectin cDNA sequence (GenBank Accession No.: gi| NM_{—}004797). Alternatively, the genomic sequence of the human Adiponectin gene may be obtained as described herein. After obtaining these clones, they are sequenced to confirm the validity of the DNA sequences.

However, in the case of the reference form (t415c) of SNP1, genomic clones would need to be obtained and may be identified by homology searches with the BLASTN program (Altschul S F 1990) against the Genbank non-redundant nucleotide sequence database using the published human Adiponectin cDNA sequence (GenBank Accession No.: gi| NM_{—}004797.2). Alternatively, the genomic sequence of the human reference Adiponectin gene may be obtained as described herein. After obtaining these clones, they are sequenced to confirm the validity of the DNA sequences.

Once these clones are confirmed to contain the intact wild type cDNA or genomic sequence of the human Adiponectin coding and/or non-coding region, the t415c polymorphism (mutation) may be introduced into the native sequence using PCR directed in vitro mutagenesis (Cormack, B., Directed Mutagenesis Using the Polymerase Chain Reaction. Current Protocols in Molecular Biology, John Wiley & Sons, Inc. Supplement 37: 8.5.1-8.5.10, (2000)). In this method, synthetic oligonucleotides are designed to incorporate a point mutation at one end of an amplified fragment. Following PCR, the amplified fragments are made blunt-ended by treatment with Klenow Fragment. These fragments are then ligated and subcloned into a vector to facilitate sequence analysis. This method consists of the following steps.
1. Subcloning of cDNA or genomic insert into a plasmid vector, or BAC sequence if the clone is a genomic sequence, containing multiple cloning sites and M13 flanking sequences, such as pUC19 (Sambrook, Fritsch et al. 1989), in the forward orientation. The skilled artisan would appreciate that other plasmids could be equally substituted, and may be desirable in certain circumstances.
2. Introduction of a mutation by PCR amplification of the cDNA region downstream of the mutation site using a primer including the mutation. (FIG. 8.5.2 in Cormack 2000)). In the case of introducing the reference t415c (i.e., T111H) sequence mutation into the human Adiponectin protein, the following two primers may be used.

M13 Reverse Sequencing Primer:

| | |
|---|---|
| 5′- CGCCAGGGTTTTCCCAGTCACGAC -3′. | (SEQ ID NO: 34) |

Mutation Primer:

(SEQ ID NO: 36)

5′- GGAGAAGGTGCCTATGTACACCGCTCAGCATTCAGTG -3′

Mutation primer contains the mutation (c167g) at the center (in bold and underlined) and a portion of its flanking sequence. M13 reverse sequencing primer hybridizes to the pUC19 vector. Subcloned cDNA or genomic clone comprising the human Adiponectin cDNA or genomic sequence is used as a template (described in Step 1). A 100 ul PCR reaction mixture is prepared using 10 ng of the template DNA, 200 uM 4dNTPs, 1 uM primers, 0.25U Taq DNA polymerase (PE), and standard Taq DNA polymerase buffer. Typical PCR cycling condition are as follows:

| | |
|---|---|
| 20-25 cycles: | 45 sec, 93 degrees |
| | 2 min, 50 degrees |
| | 2 min, 72 degrees |
| 1 cycle: | 10 min, 72 degrees |

After the final extension step of PCR, 5U Klenow Fragment is added and incubated for 15 min at 30 degrees. The PCR product is then digested with the restriction enzyme, EcoRI.
3. PCR amplification of the upstream region is then performed, using subcloned cDNA or genomic clone as a template (the product of Step 1). This PCR is done using the following two primers:

M13 Forward Sequencing Primer:

| | |
|---|---|
| 5′- CGCCAGGGTTTTCCCAGTCACGAC -3′. | (SEQ ID NO: 35) |

Flanking Primer:

(SEQ ID NO: 37)

5′- CACTGAATGCTGAGCGGTGTACATAGGCACCTTCTCC -3′.

Flanking primer is complimentary to the upstream flanking sequence and mutation locus of the c167g mutation (in bold and underlined). M13 forward sequencing primer hybridizes to the pUC19 vector. PCR conditions and Klenow treatments follow the same procedures as provided in Step 2, above. The PCR product is then digested with the restriction enzyme, HindIII.
4. Prepare the pUC19 vector for cloning the cDNA or genomic clone comprising the polymorphic locus. Digest pUC19 plasmid DNA with EcoRI and HindII. The resulting digested vector fragment may then be purified using techniques well known in the art, such as gel purification, for example.
5. Combine the products from Step 2 (PCR product containing mutation), Step 3 (PCR product containing the upstream region), and Step 4 (digested vector), and ligate them together using standard blunt-end ligation conditions (Sambrook, Fritsch et al. 1989).
6. Transform the resulting recombinant plasmid from Step 5 into E. coli competent cells using methods known in the art, such as, for example, the transformation methods described in Sambrook, Fritsch et al. 1989.
7. Analyze the amplified fragment portion of the plasmid DNA by DNA sequencing to confirm the point mutation, and absence of any other mutations introduced during PCR. The method of sequencing the insert DNA, including the primers utilized, are described herein or are otherwise known in the art.

Moreover, the skilled artisan would appreciate that the above method may be applied to engineering more than one polymorphic nucleic acid sequence of the present invention into the novel Adiponectin genes of the present invention. Such an engineered gene could be created through succesive rounds of site-directed mutagenesis, as described in Steps 1 thru 7 above, or consolidated into a single round of mutagenesis. For example, Step 2 above could be performed for each mutation, then the products of both mutation amplifications could be combined with the product of Step 3 and 4, and the procedure followed as described.

### Example 19

### Alternative Methods of Genotyping Polymorphisms Encompassed by the Present Invention

### Preparation of Samples

Polymorphisms are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed. For example, if the target nucleic acid is a cytochrome P450, the liver is a suitable source.

Many of the methods described below require amplification of DNA from target samples. This can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86, 1173 (1989), and self-sustained sequence replication (Guatelli et al., Proc. Nat. Acad. Sci. USA, 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

Additional methods of amplification are known in the art or are described elsewhere herein.

### Detection of Polymorphisms in Target DNA

There are two distinct types of analysis of target DNA for detecting polymorphisms. The first type of analysis, sometimes referred to as de novo characterization, is carried out to identify polymorphic sites not previously characterized (i.e., to identify new polymorphisms). This analysis compares target sequences in different individuals to identify points of variation, i.e., polymorphic sites. By analyzing groups of individuals representing the greatest ethnic diversity among humans and greatest breed and species variety in plants and animals, patterns characteristic of the most common alleles/haplotypes of the locus can be identified, and the frequencies of such alleles/haplotypes in the population can be determined. Additional allelic frequencies can be determined for subpopulations characterized by criteria such as geography, race, or gender. The de novo identification of polymorphisms of the invention is described in the Examples section.

The second type of analysis determines which form(s) of a characterized (known) polymorphism are present in individuals under test. Additional methods of analysis are known in the art or are described elsewhere herein.

### Allele-Specific Probes

The design and use of allele-specific probes for analyzing polymorphisms is described by e.g., Saiki et al., Nature 324, 163-166 (1986); Dattagupta, EP 235, 726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic locus aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

### Tiling Arrays

The polymorphisms can also be identified by hybridization to nucleic acid arrays, some examples of which are described in WO 95/11995. The same arrays or different arrays can be used for analysis of characterized polymorphisms. WO 95/11995 also describes sub arrays that are optimized for detection of a variant form of a precharacterized polymorphism. Such a sub array contains probes designed to be complementary to a second reference sequence, which is an allelic variant of the first reference sequence. The second group of probes is designed by the same principles as described, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to bases).

### Allele-Specific Primers

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic locus and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing elongation from the primer (see, e.g., WO 93/22456).

### Direct-Sequencing

The direct analysis of the sequence of polymorphisms of the present invention can be accomplished using either the dideoxy chain termination method or the Maxam—Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

### Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology. Principles and Applications for DNA Amplification, (W.H. Freeman and Co, New York, 1992), Chapter 7.

### Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

### Single Base Extension

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., (PNAS 94:10756-61 (1997), uses a locus-specific oligonucleotide primer labeled on the 5′ terminus with 5-carboxyfluorescein (F AM). This labeled primer is designed so that the 3′ end is immediately adjacent to the polymorphic locus of interest. The labeled primer is hybridized to the locus, and single base extension of the labeled primer is performed with fluorescently-labeled dideoxyribonucleotides (ddNTPs) in dye-terminator sequencing fashion. An increase in fluorescence of the added ddNTP in response to excitation at the wavelength of the labeled primer is used to infer the identity of the added nucleotide.

### Example 20

### Additional Methods of Genotyping the SNPs of the Present Invention

The skilled artisan would acknowledge that there are a number of methods that may be employed for genotyping a SNP of the present invention, aside from the preferred methods described herein. The present invention encompasses the following non-limiting types of genotype assays: PCR-free genotyping methods, Single-step homogeneous methods, Homogeneous detection with fluorescence polarization, Pyrosequencing, “Tag” based DNA chip system, Bead-based methods, fluorescent dye chemistry, Mass spectrometry based genotyping assays, TaqMan genotype assays, Invader genotype assays, and microfluidic genotype assays, among others.

Specifically encompassed by the present invention are the following, non-limiting genotyping methods: Landegren, U., Nilsson, M. & Kwok, P. Genome Res 8, 769-776 (1998); Kwok, P., Pharmacogenomics 1, 95-100 (2000); Gut, I., Hum Mutat 17, 475-492 (2001); Whitcombe, D., Newton, C. & Little, S., Curr Opin Biotechnol 9, 602-608 (1998); Tillib, S. & Mirzabekov, A., Curr Opin Biotechnol 12, 53-58 (2001); Winzeler, E. et al., Science 281, 1194-1197 (1998); Lyamichev, V. et al., Nat Biotechnol 17, 292-296 (1999); Hall, J. et al., Proc Natl Acad Sci U S A 97, 8272-8277 (2000); Mein, C. et al., Genome Res 10, 333-343 (2000); Ohnishi, Y. et al., J Hum Genet 46, 471-477 (2001); Nilsson, M. et al., Science 265, 2085-2088 (1994); Baner, J., Nilsson, M., Mendel-Hartvig, M. & Landegren, U., Nucleic Acids Res 26, 5073-5078 (1998); Baner, J. et al., Curr Opin Biotechnol 12, 11-15 (2001); Hatch, A., Sano, T., Misasi, J. & Smith, C., Genet Anal 15, 35-40 (1999); Lizardi, P. et al., Nat Genet 19, 225-232 (1998); Zhong, X., Lizardi, P., Huang, X., Bray-Ward, P. & Ward, D., Proc Natl Acad Sci USA 98, 3940-3945 (2001); Faruqi, F. et al. BMC Genomics 2, 4 (2001); Livak, K., Gnet Anal 14, 143-149 (1999); Marras, S., Kramer, F. & Tyagi, S., Genet Anal 14, 151-156 (1999); Ranade, K. et al., Genome Res 11, 1262-1268 (2001); Myakishev, M., Khripin, Y., Hu, S. & Hamer, D., Genome Re 11, 163-169 (2001); Beaudet, L., Bedard, J., Breton, B., Mercuri, R. & Budarf, M., Genome Res 11, 600-608 (2001); Chen, X., Levine, L. & PY, K., Genome Res 9, 492-498 (1999); Gibson, N. et al., Clin Chem 43, 1336-1341 (1997); Latif, S., Bauer-Sardina, I., Ranade, K., Livak, K. & P Y, K., Genome Res 11, 436-440 (2001); Hsu, T., Law, S., Duan, S., Neri, B. & Kwok, P., Clin Chem 47, 1373-1377 (2001); Alderborn, A., Kristofferson, A. & Hammerling, U., Genome Res 10, 1249-1258 (2000); Ronaghi, M., Uhlen, M. & Nyren, P., Science 281, 363, 365 (1998); Ronaghi, M., Genome Res 11, 3-11 (2001); Pease, A. et al., Proc Natl Acad Sci USA 91, 5022-5026 (1994); Southern, E., Maskos, U. & Elder, J., Genomics 13, 1008-1017 (1993); Wang, D. et al., Science 280, 1077-1082 (1998); Brown, P. & Botstein, D., Nat Genet 21, 33-37 (1999); Cargill, M. et al. Nat Genet 22, 231-238 (1999); Dong, S. et al., Genome Res 11, 1418-1424 (2001); Halushka, M. et al., Nat Genet 22, 239-247 (1999); Hacia, J., Nat Genet 21, 42-47 (1999); Lipshutz, R., Fodor, S., Gingeras, T. & Lockhart, D., Nat Genet 21, 20-24 (1999); Sapolsky, R. et al., Genet Anal 14, 187-192 (1999); Tsuchihashi, Z. & Brown, P., J Virol 68, 5863 (1994); Herschlag, D., J Biol Chem 270, 20871-20874 (1995); Head, S. et al., Nucleic Acids Res 25, 5065-5071 (1997); Nikiforov, T. et al., Nucleic Acids Res 22, 4167-4175 (1994); Syvanen, A. et al., Genomics 12, 590-595 (1992); Shumaker, J., Metspalu, A. & Caskey, C., Hum Mutat 7, 346-354 (1996); Lindroos, K., Liljedahl, U., Raitio, M. & Syvanen, A., Nucleic Acids Res 29, E69-9 (2001); Lindblad-Toh, K. et al., Nat Genet 24, 381-386 (2000); Pastinen, T. et al., Genome Res 10, 1031-1042 (2000); Fan, J. et al., Genome Res 10, 853-860 (2000); Hirschhorn, J. et al., Proc Natl Acad Sci USA 97, 12164-12169 (2000); Bouchie, A., Nat Biotechnol 19, 704 (2001); Hensel, M. et al., Science 269, 400-403 (1995); Shoemaker, D., Lashkari, D., Morris, D., Mittmann, M. & Davis, R. Nat Genet 14, 450-456 (1996); Gerry, N. et al., J Mol Biol 292, 251-262 (1999); Ladner, D. et al., Lab Invest 81, 1079-1086 (2001); Iannone, M. et al. ,Cytometry 39, 131-140 (2000); Fulton, R., McDade, R., Smith, P., Kienker, L. & Kettman, J. J., Clin Chem 43, 1749-1756 (1997); Armstrong, B., Stewart, M. & Mazumder, A., Cytometry 40, 102-108 (2000); Cai, H. et al., Genomics 69, 395 (2000); Chen, J. et al., Genome Res 10, 549-557 (2000); Ye, F. et al. Hum Mutat 17, 305-316 (2001); Michael, K., Taylor, L., Schultz, S. & Walt, D., Anal Chem 70, 1242-1248 (1998); Steemers, F., Ferguson, J. & Walt, D., Nat Biotechnol 18, 91-94 (2000); Chan, W. & Nie, S., Science 281, 2016-2018 (1998); Han, M., Gao, X., Su, J. & Nie, S., Nat Biotechnol 19, 631-635 (2001); Griffin, T. & Smith, L., Trends Biotechnol 18, 77-84 (2000); Jackson, P., Scholl, P. & Groopman, J., Mol Med Today 6, 271-276 (2000); Haff, L. & Smirnov, I., Genome Res 7, 378-388 (1997); Ross, P., Hall, L., Smirnov, I. & Haff, L., Nat Biotechnol 16, 1347-1351 (1998); Bray, M., Boerwinkle, E. & Doris, P. Hum Mutat 17, 296-304 (2001); Sauer, S. et al., Nucleic Acids Res 28, E13 (2000); Sauer, S. et al., Nucleic Acids Res 28, E100 (2000); Sun, X., Ding, H., Hung, K. & Guo, B., Nucleic Acids Res 28, E68 (2000); Tang, K. et al., Proc Natl Acad Sci USA 91, 10016-10020 (1999); Li, J. et al., Electrophoresis 20, 1258-1265 (1999); Little, D., Braun, A., O'Donnell, M. & Koster, H., Nat Med 3, 1413-1416 (1997); Little, D. et al. Anal Chem 69, 4540-4546 (1997); Griffin, T., Tang, W. & Smith, L., Nat Biotechnol 15, 1368-1372 (1997); Ross, P., Lee, K. & Belgrader, P., Anal Chem 69, 4197-4202 (1997); Jiang-Baucom, P., Girard, J., Butler, J. & Belgrader, P., Anal Chem 69, 4894-4898 (1997); Griffin, T., Hall, J., Prudent, J. & Smith, L., Proc Natl Acad Sci USA 96, 6301-6306 (1999); Kokoris, M. et al., Mol Diagn 5, 329-340 (2000); Jurinke, C., van den Boom, D., Cantor, C. & Koster, H. (2001); and/or Taranenko, N. et al., Genet Anal 13, 87-94 (1996).

The following additional genotyping methods are also encompassed by the present invention: the methods described and/or claimed in U.S. Pat. No. 6,458,540; and the methods described and/or claimed in U.S. Pat. No. 6,440,707.

### Example 21

### Bacterial Expression of a Polypeptide

A polynucleotide encoding a polypeptide of the present invention is amplified using PCR oligonucleotide primers corresponding to the 5′ and 3′ ends of the DNA sequence, as outlined in the Examples above or otherwise known in the art, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Ampr), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, that expresses the lacI repressor and also confers kanamycin resistance (Kanr). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.600) of between 0.4 and 0.6. IPTG (Isopropyl-B-D-thiogalacto pyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 mins at 6000×g). The cell pellet is solubilized in the chaotropic agent 6 Molar Guanidine HCl by stirring for 3-4 hours at 4 degree C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6×His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCI pH 7.4, containing protease inhibitors. The renaturation should be performed over a period of 1.5 hours or more. After renaturation the proteins are eluted by the addition of 250 mM imidazole. Imidazole is removed by a final dialyzing step against PBS or 50 mM sodium acetate pH 6 buffer plus 200 mM NaCl. The purified protein is stored at 4 degree C. or frozen at −80 degree C.

### Example 22

### Purification of a Polypeptide from an Inclusion Body

The following alternative method can be used to purify a polypeptide expressed in E coli when it is present in the form of inclusion bodies. Unless otherwise specified, all of the following steps are conducted at 4-10 degree C.

Upon completion of the production phase of the E. coli fermentation, the cell culture is cooled to 4-10 degree C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfluidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4. hours. After 7000×g centrifugation for 15 min., the pellet is discarded and the polypeptide containing supernatant is incubated at 4 degree C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 degree C. without mixing for 12 hours prior to further purification steps.

To clarify the refolded polypeptide solution, a previously prepared tangential filtration unit equipped with 0.16 um membrane filter with appropriate surface area (e.g., Filtron), equilibrated with 40 mM sodium acetate, pH 6.0 is employed. The filtered sample is loaded onto a cation exchange resin (e.g., Poros HS-50, Perceptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the polypeptide are then pooled and mixed with 4 volumes of water. The diluted sample is then loaded onto a previously prepared set of tandem columns of strong anion (Poros HQ-50, Perceptive Biosystems) and weak anion (Poros CM-20, Perceptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A280 monitoring of the effluent. Fractions containing the polypeptide (determined, for instance, by 16% SDS-PAGE) are then pooled.

The resultant polypeptide should exhibit greater than 95% purity after the above refolding and purification steps. No major contaminant bands should be observed from Coomassie blue stained 16% SDS-PAGE gel when 5 ug of purified protein is loaded. The purified protein can also be tested for endotoxin/LPS contamination, and typically the LPS content is less than 0.1 ng/ml according to LAL assays.

### Example 23

### Cloning and Expression of a Polypeptide in a Baculovirus Expression System

In this example, the plasmid shuttle vector pAc373 is used to insert a polynucleotide into a baculovirus to express a polypeptide. A typical baculovirus expression vector contains the strong polyhedrin promoter of the Autographa californica nuclear polyhedrosis virus (AcMNPV) followed by convenient restriction sites, which may include, for example BamHI, Xba I and Asp718. The polyadenylation site of the simian virus 40 (“SV40”) is often used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors can be used in place of the vector above, such as pVL941 and. pAcIM 1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

A polynucleotide encoding a polypeptide of the present invention is amplified using PCR oligonucleotide primers corresponding to the 5′ and 3′ ends of the DNA sequence, as outlined in the Examples above or otherwise known in the art, to synthesize insertion fragments. The primers used to amplify the cDNA insert should preferably contain restriction sites at the 5′ end of the primers in order to clone the amplified product into the expression vector. Specifically, the cDNA sequence contained in the deposited clone, including the AUG initiation codon and the naturally associated leader sequence identified elsewhere herein (if applicable), is amplified using the PCR protocol described herein. If the naturally occurring signal sequence is used to produce the protein, the vector used does not need a second signal peptide. Alternatively, the vector can be modified to include a baculovirus leader sequence, using the standard methods described in Summers et al., “A Manual of Methods for Baculovirus Vectors and Insect Cell Culture Procedures” Texas Agricultural Experimental Station Bulletin No. 1555 (1987).

The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five ug of a plasmid containing the polynucleotide is co-transformed with 1.0 ug of a commercially available linearized baculovirus DNA (“BaculoGoldtm baculovirus DNA”, Pharmingen, San Diego, Calif.), using the lipofection method described by Felgner et al., Proc. Natl. Acad. Sci. USA 84:7413-7417 (1987). One ug of BaculoGoldtm virus DNA and 5 ug of the plasmid are mixed in a sterile well of a microtiter plate containing 50 ul of serum-free Grace's medium (Life Technologies Inc., Gaithersburg, Md.). Afterwards, 10 ul Lipofectin plus 90 ul Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27 degrees C. The transfection solution is then removed from the plate and 1 ml of Grace's insect medium supplemented with 10% fetal calf serum is added. Cultivation is then continued at 27 degrees C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 ul of Grace's medium and the suspension containing the recombinant baculovirus is used to infect Sf9 cells seeded in 35 mm dishes. Four days later the supernatants of these culture dishes are harvested and then they are stored at 4 degree C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 uCi of 35S-methionine and 5 uCi 35S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced protein.

### Example 24

### Expression of a Polypeptide in Mammalian Cells

The polypeptide of the present invention can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transformation with a selectable marker such as dhfr, gpt, neomycin, hygromycin allows the identification and isolation of the transformed cells.

The transformed gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. . . . 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

A polynucleotide of the present invention is amplified according to the protocol outlined in herein. If the naturally occurring signal sequence is used to produce the protein, the vector does not need a second signal peptide. Alternatively, if the naturally occurring signal sequence is not used, the vector can be modified to include a heterologous signal sequence. (See, e.g., WO 96/34891.) The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transformation. Five μg of an expression plasmid is cotransformed with 0.5 ug of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of methotrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 uM, 2 uM, 5 uM, 10 mM, 20 mM). The same procedure is repeated until clones are obtained which grow at a concentration of 100-200 uM. Expression of the desired gene product is analyzed, for instance, by SDS-PAGE and Western blot or by reversed phase HPLC analysis.

### Example 25

### Production of an Antibody from a Polypeptide

The antibodies of the present invention can be prepared by a variety of methods. (See, Current Protocols, Chapter 2.) As one example of such methods, cells expressing a polypeptide of the present invention are administered to an animal to induce the production of sera containing polyclonal antibodies. In a preferred method, a preparation of the protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with polypeptide or, more preferably, with a polypeptide-expressing cell. Such cells may be cultured in any suitable tissue culture medium; however, it is preferable to culture cells in Earle's modified Eagle's medium supplemented with 10% fetal bovine serum (inactivated at about 56 degrees C.), and supplemented with about 10 g/l of nonessential amino acids, about 1,000 U/ml of penicillin, and about 100 ug/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated that Fab and F (ab′)2 and other fragments of the antibodies of the present invention may be used according to the methods disclosed herein. Such fragments are typically produced by proteolytic cleavage, using enzymes such as papain (to produce Fab fragments) or pepsin (to produce F (ab′)2 fragments). Alternatively, protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

Moreover, in another preferred method, the antibodies directed against the polypeptides of the present invention may be produced in plants. Specific methods are disclosed in U.S. Pat. Nos. 5,959,177, and 6,080,560, which are hereby incorporated in their entirety herein. The methods not only describe methods of expressing antibodies, but also the means of assembling foreign multimeric proteins in plants (i.e., antibodies, etc,), and the subsequent secretion of such antibodies from the plant.

### Example 26

### Method of Creating N- and C-Terminal Deletion Mutants Corresponding to the Reference and Variable Polypeptide Alleles of the Present Invention

As described elsewhere herein, the present invention encompasses the creation of N- and C-terminal deletion mutants, in addition to any combination of N- and C-terminal deletions thereof, corresponding to the mineralocorticoid receptor, PPAR-gamma, or Adiponectin polypeptide of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutant of the present invention, exemplary methods are described below.

Briefly, using the isolated cDNA clone encoding the full-length mineralocorticoid receptor, PPAR-gamma, or Adiponectin polypeptide sequence (as described elsewhere herein), appropriate primers of about 15-25 nucleotides derived from the desired 5′ and 3′ positions of SEQ ID NO:1, 3, 13, 15, 26, or 28 may be designed to PCR amplify, and subsequently clone, the intended N- and/or C-terminal deletion mutant. Such primers could comprise, for example, an initiation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, kozac sequences, or other sequences discussed and/or referenced herein.

Representative PCR amplification conditions are provided below, although the skilled artisan would appreciate that other conditions may be required for efficient amplification. A 100 ul PCR reaction mixture may be prepared using 10 ng of the template DNA (cDNA clone of mineralocorticoid receptor, PPAR-gamma, or Adiponectin), 200 uM 4dNTPs, 1 uM primers, 0.25U Taq DNA polymerase (PE), and standard Taq DNA polymerase buffer. Typical PCR cycling condition are as follows:

| | |
|---|---|
| 20-25 cycles: | 45 sec, 93 degrees |
| | 2 min, 50 degrees |
| | 2 min, 72 degrees |
| 1 cycle: | 10 min, 72 degrees |

After the final extension step of PCR, 5U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). The skilled artisan would appreciate that other plasmids could be equally substituted, and may be desirable in certain circumstances. The digested fragment and vector are then ligated using a DNA ligase, and then used to transform competent E.coli cells using methods provided herein and/or otherwise known in the art.

The 5′ primer sequence for amplifying any additional N-terminal deletion mutants may be determined by reference to the following formula: (S+(X*3)) to ((S+(X*3))+25), wherein ‘S’ is equal to the nucleotide position of the initiating start codon of the mineralocorticoid receptor, PPAR-gamma, or Adiponectin gene (SEQ ID NO:1, 3, 13, 15, 26, and/or 28), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer corresponding to sense strand of SEQ ID NO:1, 3, 13, 15, 26, and/or 28. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozac sequences, etc.).

The 3′ primer sequence for amplifying any additional N-terminal deletion mutants may be determined by reference to the following formula: (S+(X*3)) to ((S+(X*3))−25), wherein ‘S’ is equal to the nucleotide position of the initiating start codon of the mineralocorticoid receptor, PPAR-gamma, or Adiponectin gene (SEQ ID NO:1, 3, 13, 15, 26, and/or 28), and ‘X’ is equal to the most C-terminal amino acid of the intended N-terminal deletion mutant. The first term will provide the start 5′ nucleotide position of the 3′ primer, while the second term will provide the end 3′ nucleotide position of the 3′ primer corresponding to the anti-sense strand of SEQ ID NO:1, 3, 13, 15, 26, and/or 28. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the Sequence Listing submitted herewith and the corresponding computer readable form are both incorporated herein by reference in their entireties.

While this invention has been particularly shown and described with references to preferred embodiments thereof, it will be understood by those skilled in the art that various changes in form and details may be made therein without departing from the scope of the invention encompassed by the appended claims.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the Sequence Listing submitted herewith and the corresponding computer readable form are both incorporated herein by reference in their entireties.

While this invention has been particularly shown and described with references to preferred embodiments thereof, it will be understood by those skilled in the art that various changes in form and details may be made therein without departing from the scope of the invention encompassed by the appended claims.

What is claimed is:

1. An isolated nucleic acid molecule comprising a polynuclelotide sequence selected from the group consisting of the polynucleotide sequence provided as SEQ ID NO:1, 3, 13, 15, 26, and 28.

2. The isolated nucleic acid molecule according to claim 1, wherein said nucleic acid comprises at least one polymorphic locus selected from the group consisting of:
(a) nucleotide position 754 of SEQ ID NO:1;
(b) nucleotide position 754 of SEQ ID NO:3;
(c) nucleotide position 167 of SEQ ID NO:13;
(d) nucleotide position 167 of SEQ ID NO:15;
(e) nucleotide position 415 of SEQ ID NO:26; and
(f) nucleotide position 415 of SEQ ID NO:28.

3. A method of identifying a patient who may be at risk of developing dose-dependent congestive heart failure upon administration of a PPAR-agonist comprising the step of determining whether said patient has a reference or variable allele at one or more polymorphic loci of the human mineralocorticoid gene.

4. The method according to claim 3, wherein the presence of a variable allele at the polymorphic locus at nucleotide position 754 of SEQ ID NO:1 or 3 is indicative of an increased risk of developing dose-dependent congestive heart failure in a patient receiving PPAR-agonist therapy, whereas the presence of the reference allele at said polymorphic position is indicative of a decreased risk of developing dose-dependent congestive heart failure in a patient receiving PPAR-agonist therapy.

5. A method of identifying a patient who may be at risk of developing dose-dependent congestive heart failure upon administration of a PPAR-agonist comprising the step of determining whether said patient has a reference or variable allele at one or more polymorphic loci of the human PPAR-gamma gene.

6. The method according to claim 5, wherein the presence of a variable allele at the polymorphic locus at nucleotide position 167 of SEQ ID NO:13 or 15 is indicative of an increased risk of developing dose-dependent congestive heart failure in a patient receiving PPAR-agonist therapy, whereas the presence of the reference allele at said polymorphic position is indicative of a decreased risk of developing dose-dependent congestive heart failure in a patient receiving PPAR-agonist therapy.

7. A method of identifying a patient who may be at risk of developing dose-dependent congestive heart failure upon administration of a PPAR-agonist comprising the step of determining whether said patient has a reference or variable allele at one or more polymorphic loci of the human adiponectin gene.

8. The method according to claim 7, wherein the presence of a variable allele at the polymorphic locus at nucleotide position 415 of SEQ ID NO:26 or 28 is indicative of an increased risk of developing dose-dependent congestive heart failure in a patient receiving PPAR-agonist therapy, whereas the presence of the reference allele at said polymorphic position is indicative of a decreased risk of developing dose-dependent congestive heart failure in a patient receiving PPAR-agonist therapy.

9. An isolated polypeptide comprising a sequence selected from the group consisting of the polypeptide sequence provided as SEQ ID NO:2, and SEQ ID NO:4, wherein said polypeptide comprises at least one polymorphic locus, wherein said polymorphic locus is located at amino acid position 180.

10. A method of identifying a patient who may be at risk of developing dose-dependent congestive heart failure upon administration of a PPAR-agonist comprising the step of determining whether said patient has a reference or variable allele at one or more polymorphic loci of the human mineralocorticoid receptor polypeptide.

11. The method according to claim 10, wherein the presence of a variable allele at the polymorphic locus at amino acid position 180 of SEQ ID NO:2 or 4, is indicative of an increased risk of developing dose-dependent congestive heart failure in a patient receiving PPAR-agonist therapy, whereas the presence of the reference allele at said polypeptide polymorphic position is indicative of a decreased risk of developing dose-dependent congestive heart failure in a patient receiving PPAR-agonist therapy.

12. An isolated polypeptide comprising a sequence selected from the group consisting of the polypeptide sequence provided as SEQ ID NO:14, and SEQ ID NO:16, wherein said polypeptide comprises at least one polymorphic locus, wherein said polymorphic locus is located at amino acid position 180.

13. A method of identifying a patient who may be at risk of developing dose-dependent congestive heart failure upon administration of a PPAR-agonist comprising the step of determining whether said patient has a reference or variable allele at one or more polymorphic loci of the human PPAR-gamma polypeptide.

14. The method according to claim 13, wherein the presence of a variable allele at the polymorphic locus at amino acid position 12 of SEQ ID NO:14 or 16, is indicative of an increased risk of developing dose-dependent congestive heart failure in a patient receiving PPAR-agonist therapy, whereas the presence of the reference allele at said polypeptide polymorphic position is indicative of a decreased risk of developing dose-dependent congestive heart failure in a patient receiving PPAR-agonist therapy.

15. An isolated polypeptide comprising a sequence selected from the group consisting of the polypeptide sequence provided as SEQ ID NO:27, and SEQ ID NO:29, wherein said polypeptide comprises at least one polymorphic locus, wherein said polymorphic locus is located at amino acid position 111.

16. A method of identifying a patient who may be at risk of developing dose-dependent congestive heart failure upon administration of a PPAR-agonist comprising the step of determining whether said patient has a reference or variable allele at one or more polymorphic loci of the human adiponectin polypeptide.

17. The method according to claim 16, wherein the presence of a variable allele at the polymorphic locus at amino acid position 111 of SEQ ID NO:27 or 29, is indicative of an increased risk of developing dose-dependent congestive heart failure in a patient receiving PPAR-agonist therapy, whereas the presence of the reference allele at said polypeptide polymorphic position is indicative of a decreased risk of developing dose-dependent congestive heart failure in a patient receiving PPAR-agonist therapy.

18. A kit comprising the method of claim 3, 4, 5, 6, 7, 8, 10, 11, 13, 14, 16, or 17.
